SEQUENCE LISTING (71 sequences)

SEQ ID NO: 1 - protein, 10 aa, Artificial Sequence
  Synthetic Sequence
  VARIANT (3)..(3): X is N or W
GFXIKDTYIH

SEQ ID NO: 2 - protein, 17 aa, Artificial Sequence
  Synthetic Sequence
  Variant (1)..(1): X is R or K
  Variant (3)..(3): X is Y or K or D
  Variant (6)..(6): X is N or A
  Variant (7)..(7): X is G or K
  Variant (9)..(9): X is T or D
  Variant (10)..(10): X is R or E
XIXPTXXYXXYADSVKG

SEQ ID NO: 3 - protein, 11 aa, Artificial Sequence
  Synthetic Sequence
  Variant (8)..(8): X is A or E
WGGDGFYXMDY

SEQ ID NO: 4 - protein, 11 aa, Artificial Sequence
  Synthetic Sequence
  Variant (9)..(9): X is A or D
RASQDVNTXVA

SEQ ID NO: 5 - protein, 7 aa, Artificial Sequence
  Synthetic Sequence
SASFLYS

SEQ ID NO: 6 - protein, 9 aa, Artificial Sequence
  Synthetic Sequence
  Variant (3)..(3): X is H or D or E
QQXYTTPPT

SEQ ID NO: 7 - protein, 10 aa, Artificial Sequence
  Synthetic Sequence
GFWIKDTYIH

SEQ ID NO: 8 - protein, 17 aa, Artificial Sequence
  Synthetic Sequence
KIYPTNGYTRYADSVKG

SEQ ID NO: 9 - protein, 17 aa, Artificial Sequence
  Synthetic Sequence
RIKPTNGYTRYADSVKG

SEQ ID NO: 10 - protein, 17 aa, Artificial Sequence
  Synthetic Sequence
RIDPTNGYTRYADSVKG

SEQ ID NO: 11 - protein, 17 aa, Artificial Sequence
  Synthetic Sequence
RIYPTAGYTRYADSVKG

SEQ ID NO: 12 - protein, 17 aa, Artificial Sequence
  Synthetic Sequence
RIYPTNKYTRYADSVKG

SEQ ID NO: 13 - protein, 17 aa, Artificial Sequence
  Synthetic Sequence
RIYPTNGYDRYADSVKG

SEQ ID NO: 14 - protein, 17 aa, Artificial Sequence
  Synthetic Sequence
RIYPTNGYTEYADSVKG

SEQ ID NO: 15 - protein, 11 aa, Artificial Sequence
  Synthetic Sequence
WGGDGFYEMDY

SEQ ID NO: 16 - protein, 11 aa, Artificial Sequence
  Synthetic Sequence
RASQDVNTDVA

SEQ ID NO: 17 - protein, 9 aa, Artificial Sequence
  Synthetic Sequence
QQDYTTPPT

SEQ ID NO: 18 - protein, 9 aa, Artificial Sequence
  Synthetic Sequence
QQEYTTPPT

SEQ ID NO: 19 - protein, 120 aa, Artificial Sequence
  Synthetic Sequence
EVQLVESGGGLVQPGGSLRLSCAASGFNIKDTYIHWVRQAPGKGLEWVAKIYPTNGYTRYADSVKGRFTISADTSKNTAYLQMNSLRAEDTAVYYCSRWGGDGFYAMDYWGQGTLVTVSS

SEQ ID NO: 20 - protein, 120 aa, Artificial Sequence
  Synthetic Sequence
EVQLVESGGGLVQPGGSLRLSCAASGFNIKDTYIHWVRQAPGKGLEWVARIYPTNGYTEYADSVKGRFTISADTSKNTAYLQMNSLRAEDTAVYYCSRWGGDGFYAMDYWGQGTLVTVSS

SEQ ID NO: 21 - protein, 120 aa, Artificial Sequence
  Synthetic Sequence
EVQLVESGGGLVQPGGSLRLSCAASGFNIKDTYIHWVRQAPGKGLEWVARIKPTNGYTRYADSVKGRFTISADTSKNTAYLQMNSLRAEDTAVYYCSRWGGDGFYAMDYWGQGTLVTVSS

SEQ ID NO: 22 - protein, 120 aa, Artificial Sequence
  Synthetic Sequence
EVQLVESGGGLVQPGGSLRLSCAASGFNIKDTYIHWVRQAPGKGLEWVARIDPTNGYTRYADSVKGRFTISADTSKNTAYLQMNSLRAEDTAVYYCSRWGGDGFYAMDYWGQGTLVTVSS

SEQ ID NO: 23 - protein, 120 aa, Artificial Sequence
  Synthetic Sequence
EVQLVESGGGLVQPGGSLRLSCAASGFNIKDTYIHWVRQAPGKGLEWVARIYPTAGYTRYADSVKGRFTISADTSKNTAYLQMNSLRAEDTAVYYCSRWGGDGFYAMDYWGQGTLVTVSS

SEQ ID NO: 24 - protein, 120 aa, Artificial Sequence
  Synthetic Sequence
EVQLVESGGGLVQPGGSLRLSCAASGFNIKDTYIHWVRQAPGKGLEWVARIYPTNKYTRYADSVKGRFTISADTSKNTAYLQMNSLRAEDTAVYYCSRWGGDGFYAMDYWGQGTLVTVSS

SEQ ID NO: 25 - protein, 120 aa, Artificial Sequence
  Synthetic Sequence
EVQLVESGGGLVQPGGSLRLSCAASGFNIKDTYIHWVRQAPGKGLEWVARIYPTNGYDRYADSVKGRFTISADTSKNTAYLQMNSLRAEDTAVYYCSRWGGDGFYAMDYWGQGTLVTVSS

SEQ ID NO: 26 - protein, 120 aa, Artificial Sequence
  Synthetic Sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYEMDYWGQGTLVTVSS

SEQ ID NO: 27 - protein, 120 aa, Artificial Sequence
  Synthetic Sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVES

SEQ ID NO: 28 - protein, 120 aa, Artificial Sequence
  Synthetic Sequence
EVQLVESGGGLVQPGGSLRLSCAASGFWIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 29 - protein, 107 aa, Artificial Sequence [sequence omitted]

SEQ ID NO: 30 - protein, 107 aa, Artificial Sequence
  Synthetic Sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIK

SEQ ID NO: 31 - protein, 107 aa, Artificial Sequence
  Synthetic Sequence
DIQMTQSPSSLSASVGDRVTITCRASQDVNTAVAWYQQKPGKAPKLLIYSASFLYSGVPSRFSGSRSGTDFTLTISSLQPEDFATYYCQQDYTTPPTFGQGTKVEIK

SEQ ID NO: 32 - protein, 107 aa, Artificial Sequence
  Synthetic Sequence
DIQMTQSPSSLSASVGDRVTITCRASQDVNTAVAWYQQKPGKAPKLLIYSASFLYSGVPSRFSGSRSGTDFTLTISSLQPEDFATYYCQQEYTTPPTFGQGTKVEIK

SEQ ID NO: 33 - protein, 120 aa, Artificial Sequence [sequence omitted]

SEQ ID NO: 34 - protein, 450 aa, Artificial Sequence
  Synthetic sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 450 aa, Artificial Sequence
  Synthetic sequence
EVQLVESGGGLVQPGGSLRLSCAASGFNIKDTYIHWVRQAPGKGLEWVARIKPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 450 aa, Artificial Sequence
  Synthetic sequence
EVQLVESGGGLVQPGGSLRLSCAASGFNIKDTYIHWVRQAPGKGLEWVARIYPTNGKTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 450 aa, Artificial Sequence
  Synthetic sequence
EVQLVESGGGLVQPGGSLRLSCAASGFNIKDTYIHWVRQAPGKGLEWVARIYPTNGYDRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 450 aa, Artificial Sequence
  Synthetic sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYEMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 450 aa, Artificial Sequence
  Synthetic sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 471 aa, Artificial Sequence
  Synthetic sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGSGGSGGSGGSGGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLSR

SEQ ID NO: 41 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFGNSKVSWFAYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 42 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSKVSWFQYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 43 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFPNSKVSWFQYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 44 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHANFGNSKVSWFAYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 45 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFPNSKVSWFAYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 46 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHTNFGNSKVSWFAYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 47 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFGNSKVSWFAYWGQGTLVTVSSGGSGGSGGSGGSGGQAVVTQEPSLTVSPGGTVTLTCRSSAGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 48 - protein, 471 aa, Artificial Sequence
  Synthetic sequence
DIQMTQSPSSLSASVGDRVTITCRASQDVNTDVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHTNFGNSKVSWFAYWGQGTLVTVSSGGSGGSGGSGGSGGQAVVTQEPSLTVSPGGTVTLTCRSSAGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 49 - protein, 17 aa, Artificial Sequence
  Synthetic Sequence
RIYPTNGYTRYADSVKG

SEQ ID NO: 50 - protein, 10 aa, Artificial Sequence
  Synthetic Sequence
GFQIKDTYIH

SEQ ID NO: 51 - protein, 11 aa, Artificial Sequence
  Synthetic Sequence
WGGDGFYAMDY

SEQ ID NO: 52 - protein, 11 aa, Artificial Sequence
  Synthetic Sequence
RASQDVNTAVA

SEQ ID NO: 53 - protein, 9 aa, Artificial Sequence
  Synthetic Sequence
QQHYTTPPT

SEQ ID NO: 54 - protein, 10 aa, Artificial Sequence
  Synthetic Sequence
GFTFNTYAMN

SEQ ID NO: 55 - protein, 19 aa, Artificial Sequence
  Synthetic sequence
RIRSKYNNYATYYADSVKD

SEQ ID NO: 56 - protein, 14 aa, Artificial Sequence
  Synthetic sequence
HGNFGNSYVSWFAY

SEQ ID NO: 57 - protein, 14 aa, Artificial Sequence
  Synthetic sequence
RSSTGAVTTSNYAN

SEQ ID NO: 58 - protein, 7 aa, Artificial Sequence
  Synthetic sequence
GTNKRAP

SEQ ID NO: 59 - protein, 9 aa, Artificial Sequence
  Synthetic sequence
ALWYSNLWV

SEQ ID NO: 60 - protein, 14 aa, Artificial Sequence
  Synthetic sequence
HSNFGNSKVSWFAY

SEQ ID NO: 61 - protein, 14 aa, Artificial Sequence
  Synthetic sequence
HGNFGNSKVSWFQY

SEQ ID NO: 62 - protein, 14 aa, Artificial Sequence
  Synthetic sequence
HGNFPNSKVSWFQY

SEQ ID NO: 63 - protein, 14 aa, Artificial Sequence
  Synthetic sequence
HANFGNSKVSWFAY

SEQ ID NO: 64 - protein, 14 aa, Artificial Sequence
  Synthetic sequence
HGNFPNSKVSWFAY

SEQ ID NO: 65 - protein, 14 aa, Artificial Sequence
  Synthetic sequence
HTNFGNSKVSWFAY

SEQ ID NO: 66 - protein, 14 aa, Artificial Sequence
  Synthetic sequence
HSNFGNSKVSWFAY

SEQ ID NO: 67 - protein, 14 aa, Artificial Sequence
  Synthetic sequence
HTNFGNSKVSWFAY

SEQ ID NO: 68 - protein, 14 aa, Artificial Sequence
  Synthetic sequence
RSSTGAVTTSNYDN

SEQ ID NO: 69 - protein, 14 aa, Artificial Sequence
  Synthetic sequence
RSSAGAVTTSNYDN

SEQ ID NO: 70 - protein, 14 aa, Artificial Sequence
  Synthetic sequence
  Variant (2)..(2): X is G, S, A or T
  Variant (5)..(5): X is G or P
  Variant (13)..(13): X is A or Q
HXNFXNSKVSWFXY

SEQ ID NO: 71 - protein, 14 aa, Artificial Sequence
  Synthetic sequence
  Variant (4)..(4): X is A or T
RSSXGAVTTSNYDN

(19) United States
(12) Patent Application Publication
(10) Pub. No.: US 2023/0109218 A1
(43) Pub. Date: Apr. 6, 2023

(54) Conditionally Active Anti-Her2 Antibodies, Antibody Fragments Their Immunoconjugates And Uses Thereof

(71) Applicant: BioAtla, Inc., San Diego, CA (US)

(72) Inventors: Jay M. SHORT, Jackson, WY (US); Gerhard FREY, San Diego, CA (US); Hwai Wen Chang, San Marcos, CA (US)

(73) Assignee: BioAtla, Inc., San Diego, CA (US)

(21) Appl. No.: 17/759,195

(22) PCT Filed: Jan. 22, 2021
(86) PCT No.: PCT/US2021/014790

§ 371 Date: Jul. 21, 2022

Related U.S. Application Data
(60) Provisional application No. 62/964,747, filed on Jan. 23, 2020.

(51) Int. Cl. C07K 16/32; C07K 16/28; A61K 47/68; A61K 45/06; A61K 39/395; A61P 35/00

(52) U.S. Cl. CPC C07K 16/32; C07K 16/2809; A61K 47/6851; A61K 47/6849; A61K 45/06; A61K 39/39558; A61K 39/3955; A61P 35/00; A61K 2039/505

(57) ABSTRACT

A polypeptide having a heavy chain variable region and/or light chain variable region that specifically binds to HER2 protein as well as antibodies and antibody fragments containing the heavy chain variable region and/or the light chain variable region that bind to HER2 protein and multi-specific antibodies that bind to HER2 protein and CD3. Pharmaceutical compositions and kits comprising the polypeptides, antibodies and antibody fragments and multi-specific antibodies containing the polypeptides are also provided

### FIELD OF THE DISCLOSURE

This disclosure relates anti-HER2 antibodies, anti-HER2 antibody fragments, anti-HER2 multi-specific antibodies and immunoconjugates of such antibodies and antibody fragments and uses of the antibodies, antibody fragments, multi-specific antibodies and immunoconjugates in diagnostic and therapeutic methods.

### BACKGROUND OF THE DISCLOSURE

Human epidermal growth factor receptor 2 (HER2) is a member of the epidermal growth factor receptor family having tyrosine kinase activity. Dimerization of the receptor results in the autophosphorylation of tyrosine residues within the cytoplasmic domain of the receptors and initiates a variety of signaling pathways leading to cell proliferation and tumorigenesis. Details about the role of HER2 in cancers can be found in many articles such as “Human Epidermal Growth Factor Receptor 2 (HER2) in Cancers: Overexpression and Therapeutic Implications,” Iqbal, Nida and Iqbal, Naveed, Molecular Biology International, Volumn 2014, Article ID 852748.

Over-expression of the ERB-B2 gene (also called “the HER2 gene”), occurs in a significant proportion of breast cancers. Overexpression of HER2 protein is strongly associated with increased disease recurrence and a poor prognosis. Drug agents targeting HER2 protein in breast cancer have had a significant, positive effect in treatment of HER2-positive breast cancer. Over-expression of HER2 protein also occurs in ovarian cancer, stomach cancer, lung adenocarcincoma, aggressive forms of uterine cancer, gastric cancer and salivary duct carcinomas.

HER2 protein is the target of the Herceptin, the monoclonal antibody trastuzumab, and has been shown to be effective in cancers where HER2 protein is over-expressed. Trastuzumab binding to HER2 protein has been shown to increase p27, a protein that halts cell proliferation. Another monoclonal antibody, Pertuzumab, has been approved by the FDA for use in combination with trastuzumab. Pertuzumab inhibits dimerisation of HER2 and HERS receptors. Other therapies targeted to HER2 protein are available or in development.

There are at least four tests for HER2 overexpression. The ImmunoHistoChemistry test which determines if there is too much HER2 protein in the cancer cells, the Fluorescence In Situ Hybridization test which determines if there are too many copies of the HER2 gene in the cancer cells, the Subtraction Probe Technology Chromogenic In Situ Hybridization test which determines if there are too many copies of the HER2 gene in the cancer cells, and the Inform Dual In Situ Hybridization test which also determines if there are too many copies of the HER2 gene in the cancer cells.

The present invention aims at providing anti-HER2 antibodies or antibody fragments with reduced or minimal side effects suitable for therapeutic and diagnostic use, especially for diagnosis and treatment of cancers. Some of these anti-HER2 antibodies or antibody fragments may have a higher binding affinity to HER2 protein in a tumor microenvironment in comparison with HER2 protein present in normal tissues. These anti-HER2 antibodies or antibody fragments typically have at least comparable efficacy to known anti-HER2 antibodies. In addition, the present anti-HER2 antibodies or antibody fragments may exhibit reduced side effects in comparison with monoclonal anti-HER2 antibodies including those that may be known in the art by having a relatively low binding affinity to HER2 protein in normal tissues. These advantages may provide a more selective targeting of the HER2 protein and may permit use of higher dosages of these anti-HER2 antibodies or antibody fragments because of the selectivity of the antibodies for HER2 protein present in a tumor microenvironment, whereby more effective therapeutic treatments may be realized without a corresponding increase in undesirable side effects.

### SUMMARY OF THE DISCLOSURE

In one aspect, the present invention provides an isolated polypeptide that specifically binds to HER2 protein, said polypeptide comprising a heavy chain variable region including three anti-HER2 complementarity determining regions, said regions having sequences H1, H2, and H3, wherein:

the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;

wherein X₁ is N or W, X₂ is R or K, X₃ is Y or K or D, X₄ is N or A, X₅ is G or K, X₆ is T or D, X₇ is R or E and X₈ is A or E; and

a light chain variable region including three anti-HER2 complementarity determining regions having sequences L1, L2, and L3, wherein:

the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,

wherein X₉ is A or D and X₁₀ is H or D or E; and
provided that when X₁-X₈ are N, R, Y, N, G, T, R and A, respectively, X₉ is not A and X₁₀ is not H.

The isolated polypeptide of the present invention, wherein the H1 sequence is GFWIKDTYIH (SEQ ID NO: 7) or GFNIKDTYIH (SEQ ID NO: 50); the H2 sequence is any one of KIYPTNGYTRYADSVKG (SEQ ID NO: 8), RIKPTNGYTRYADSVKG (SEQ ID NO: 9), RIDPTNGYTRYADSVKG (SEQ ID NO: 10), RIYPTAGYTRYADSVKG (SEQ ID NO: 11), RIYPTNKYTRYADSVKG (SEQ ID NO: 12), RIYPTNGYDRYADSVKG (SEQ ID NO: 13), RIYPTNGYTEYADSVKG (SEQ ID NO: 14), and RIYPTNGYTRYADSVKG (SEQ ID NO: 49); the H3 sequence is WGGDGFYEMDY (SEQ ID NO: 15) or WGGDGFYAMDY (SEQ ID NO: 51); the L1 sequence is RASQDVNTDVA (SEQ ID NO: 16) or RASQDVNTAVA (SEQ ID NO: 52); the L2 sequence is SASFLYS (SEQ ID NO: 5); and the L3 sequence is QQDYTTPPT (SEQ ID NO: 17), QQEYTTPPT (SEQ ID NO: 18), or QQHYTTPPT (SEQ ID NO: 53).

In another aspect, the present invention provides an isolated polypeptide that specifically binds to HER2 protein, said polypeptide comprising a heavy chain variable region and a light chain variable region, the heavy chain variable region having an amino acid sequence selected from SEQ ID NOS: 19-28; and a light chain variable region having an amino acid sequence selected from SEQ ID NOS: 29-32.

In still another aspect, the present invention provides an isolated polypeptide that specifically binds to HER2 protein, said polypeptide comprising a heavy chain variable region and a light chain variable region, the heavy chain variable region having an amino acid sequence selected from SEQ ID NOS: 33 and 19-28; and a light chain variable region having an amino acid sequence selected from SEQ ID NOS: 30-32.

In still another aspect, the present invention provides an isolated polypeptide that specifically binds to HER2 protein, said polypeptide comprising a heavy chain variable region and a light chain variable region, the heavy chain variable region having an amino acid sequence selected from SEQ ID NOS: 35-39; and a light chain variable region having an amino acid sequence selected from SEQ ID NOS: 41-48.

In a certain aspect, present invention provides an isolated polypeptide that specifically binds to HER2 protein, said polypeptide comprising a heavy chain variable region having an amino acid sequence of SEQ ID NO: 35; and a light chain variable region having an amino acid sequence selected from any one of SEQ ID NOS: 41-48.

In another certain aspect, present invention provides an isolated polypeptide that specifically binds to HER2 protein, said polypeptide comprising a heavy chain variable region having an amino acid sequence of SEQ ID NO: 36; and a light chain variable region having an amino acid sequence selected from any one of SEQ ID NOS: 41-48.

In another aspect, present invention provides an isolated polypeptide that specifically binds to HER2 protein, said polypeptide comprising a heavy chain variable region having an amino acid sequence of SEQ ID NO: 37; and a light chain variable region having an amino acid sequence selected from any one of SEQ ID NOS: 41-48.

In still another aspect, present invention provides an isolated polypeptide that specifically binds to HER2 protein, said polypeptide comprising a heavy chain variable region having an amino acid sequence of SEQ ID NO: 38; and a light chain variable region having an amino acid sequence selected from any one of SEQ ID NOS: 41-48.

In still another aspect, present invention provides an isolated polypeptide that specifically binds to HER2 protein, said polypeptide comprising a heavy chain variable region having an amino acid sequence of SEQ ID NO: 39; and a light chain variable region having an amino acid sequence selected from any one of SEQ ID NOS: 41-48.

In a yet another aspect the isolated polypeptide comprises a heavy chain variable region and a light chain variable region, said heavy chain variable region including three anti-HER2 complementarity determining regions, H1, H2, and H3, wherein:

the H1 sequence is SEQ ID NO: 50,
the H2 sequence is SEQ ID NO: 49, SEQ ID NO: 9, SEQ ID NO: 12, or SEQ ID NO: 13, and
the H3 sequence is SEQ ID NO: 51; and
said light chain variable region including three anti-HER2 complementarity determining regions, L1, L2, and L3, and six anti-CD3 complementarity determining regions L4, L5, L6, L7, L8, and L9 wherein:

the L4 sequence is
(SEQ ID NO: 54)
GFTFNTYAMN,
the L5 sequence is
(SEQ ID NO: 55)
RIRSKYNNYATYYADSVKD,
the L6 sequence is
(SEQ ID NO: 70)
HX₁₁NFX₁₂NSKVSWFX₁₃Y,
the L7 sequence is
(SEQ ID NO: 71)
RSSX₁₄GAVTTSNYDN,
the L8 sequence is
(SEQ ID NO: 58)
GTNKRAP,
and
the L9 sequence is
(SEQ ID NO: 59)
ALWYSNLWV,

wherein X₁₁ is G, S, A or T, X₁₂ is G or P, X₁₃ is A or Q, and X₁₄ is T or A.

In another aspect of the isolated polypeptide with nine CDRs, the L6 sequence is any one of SEQ ID NOs: 56 and 60-67, and the L7 sequence is SEQ ID NO: 57, 68 or 69.

In another certain aspect of the isolated polypeptide, the L4 sequence is selected from SEQ ID NO: 57, 68 and 69.

In another aspect, the present invention relates to an anti-HER2 antibody or antibody fragment comprising the isolated polypeptide of each of the foregoing embodiments.

In the foregoing embodiments, the antibody or antibody fragment may have a higher binding affinity to HER2 protein at a pH in a tumor microenvironment in comparison with a pH that occurs in a non-tumor microenvironment. The pH in the tumor microenvironment may range of from 5.0 to 7.0 and the pH in the non-tumor microenvironment may range from 7.2 to 7.8.

In another aspect, the invention relates to an antibody or antibody fragment comprising a heavy chain variable region and a light chain variable region, wherein the heavy chain variable region includes three anti-HER2 complementarity determining regions, said regions having sequences H1, H2, and H3, wherein:

the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;

wherein X₁ is N or W, X₂ is R or K, X₃ is Y or K or D, X₄ is N or A, X₅ is G or K, X₆ is T or D, X₇ is R or E and X₈ is A or E; and

a light chain variable region including three anti-HER2 complementarity determining regions having sequences L1, L2, and L3, wherein:

the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,

wherein X₉ is A or D and X₁₀ is H or D or E; and
provided that when X₁-X₈ are N, R, Y, N, G, T, R and A, respectively, X₉ is not A and X₁₀ is not H.

In certain aspects of the antibody or antibody fragment, the H1 sequence may be GFWIKDTYIH (SEQ ID NO: 7) or GFNIKDTYIH (SEQ ID NO: 50); the H2 sequence may be KIYPTNGYTRYADSVKG (SEQ ID NO: 8), RIKPTNGYTRYADSVKG (SEQ ID NO: 9), RIDPTNGYTRYADSVKG (SEQ ID NO: 10), RIYPTAGYTRYADSVKG (SEQ ID NO: 11), RIYPTNKYTRYADSVKG (SEQ ID NO: 12), RIYPTNGYDRYADSVKG (SEQ ID NO: 13), RIYPTNGYTEYADSVKG (SEQ ID NO: 14) or RIYPTNGYTRYADSVKG (SEQ ID NO: 49); and the H3 sequence may be WGGDGFYEMDY (SEQ ID NO: 15) or WGGDGFYAMDY (SEQ ID NO: 51).

In each of the foregoing embodiments of this aspect of the antibody or antibody fragment, the L1 sequence may be RASQDVNTDVA (SEQ ID NO: 16) or RASQDVNTAVA (SEQ ID NO: 52), the L2 sequence is SASFLYS (SEQ ID NO: 5); and the L3 sequence may be QQDYTTPPT (SEQ ID NO: 17), QQEYTTPPT (SEQ ID NO: 18), or QQHYTTPPT (SEQ ID NO: 53).

In certain of the foregoing embodiments of this aspect of the antibody or antibody fragment, the H1 sequence is SEQ ID NO: 50; the H2 sequence is SEQ ID NO: 49, and the H3 sequence is SEQ ID NO: 51.

In certain of the foregoing embodiments of this aspect of the antibody or antibody fragment, the L1 sequence is RASQDVNTAVA (SEQ ID NO: 52), and the L3 sequence is QQHYTTPPT (SEQ ID NO: 53).

In certain aspects of the antibody or antibody fragment, the H1 sequence is SEQ ID NO: 50; the H2 sequence is RIKPTNGYTRYADSVKG (SEQ ID NO: 9), RIYPTNKYTRYADSVKG (SEQ ID NO: 12), RIYPTNGYDRYADSVKG (SEQ ID NO: 13) or RIYPTNGYTRYADSVKG (SEQ ID NO: 49), and the H3 sequence is WGGDGFYEMDY (SEQ ID NO: 15) or WGGDGFYAMDY (SEQ ID NO: 51).

In each of the foregoing embodiments of this aspect of the antibody or antibody fragment, the heavy chain variable region may be any one of SEQ ID NOS: 19-28 and the light chain variable region may be any one of SEQ ID NOS: 29-32.

In each of the foregoing embodiments of this aspect of the antibody or antibody fragment, the heavy chain variable region may be any one of SEQ ID NOS: 33 and 19-28 and the light chain variable region may be any one of SEQ ID NOS: 30-32.

In each of the foregoing embodiments of this aspect of the antibody or antibody fragment, the heavy chain variable region may be any one of SEQ ID NOS: 35-39 and the light chain variable region may be any one of SEQ ID NOS: 41-48.

In certain embodiments, the antibody or antibody fragment has a heavy chain variable region of SEQ ID NO: 19 or 20 and a light chain variable region of SEQ ID NO: 29.

In other embodiments, the antibody or antibody fragment has a heavy chain variable is SEQ ID NO: 33 and the light chain variable regions is one of SEQ ID NOS: 30-32.

In other embodiments, the antibody or antibody fragment has a light chain variable region of SEQ ID NO: 30 and a heavy chain variable region of any one of SEQ ID NOS: 33 and 19-28.

In certain embodiments, the antibody or antibody fragment has a heavy chain variable region of SEQ ID NO: 35 and a light chain variable region selected from any one of SEQ ID NOs: 41-48.

In certain embodiments, the antibody or antibody fragment has a heavy chain variable region of SEQ ID NO: 36 and a light chain variable region selected from any one of SEQ ID NOs: 41-48.

In other embodiments, the antibody or antibody fragment has a heavy chain variable region of SEQ ID NO: 37 and a light chain variable region selected from any one of SEQ ID NOs: 41-48.

In other embodiments, the antibody or antibody fragment has a heavy chain variable region of SEQ ID NO: 38 and a light chain variable region selected from any one of SEQ ID NOs: 41-48.

In still other embodiments, the antibody or antibody fragment has a heavy chain variable region of SEQ ID NO: 39 and a light chain variable region selected from any one of SEQ ID NOs: 41-48.

In some embodiments, the antibody or antibody fragment is a multi-specific antibody or antibody fragment which comprises a heavy chain variable region including three anti-HER2 complementarity determining regions, H1, H2, and H3, wherein:

the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;

wherein X₁ is N or W, X₂ is R or K, X₃ is Y or K or D, X₄ is N or A, X₅ is G or K, X₆ is T or D, X₇ is R or E and X₈ is A or E; and

a light chain variable region including three anti-HER2 complementarity determining regions having sequences L1, L2, and L3, and six anti-CD3 complementarity determining regions, L4, L5, L6, L7, L8, and L9 wherein:

the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,

wherein X₉ is A or D and X₁₀ is H or D or E; and
provided that when X₁-X₈ are N, R, Y, N, G, T, R and A, respectively, X₉ is not A and X₁₀ is not H; and

the L4 sequence is
(SEQ ID NO: 54)
GFTFNTYAMN,
the L5 sequence is
(SEQ ID NO: 55)
RIRSKYNNYATYYADSVKD,
the L6 sequence is
(SEQ ID NO: 70)
HX₁₁NFX₁₂NSKVSWFX₁₃Y,
the L7 sequence is
(SEQ ID NO: 71)
RSSX₁₄GAVTTSNYDN,
the L8 sequence is
(SEQ ID NO: 58)
GTNKRAP,
and
the L9 sequence is
(SEQ ID NO: 59)
ALWYSNLWV,

wherein X₁₁ is G, S, A or T, X₁₂ is G or P, X₁₃ is A or Q, and X₁₄ is T or A.

In another aspect of the antibody or antibody fragment, said antibody or antibody fragment is multi-specific and comprises a heavy chain variable region including three anti-HER2 complementarity determining regions, H1, H2, and H3, wherein:

the H1 sequence is SEQ ID NO: 50,
the H2 sequence is selected from SEQ ID NO: 49, SEQ ID NO: 9, SEQ ID NO: 12, or SEQ ID NO: 13, and
the H3 sequence is SEQ ID NO: 51 and a light chain variable region including three anti-HER2 complementarity determining regions L1, L2, and L3, and six anti-CD3 complementarity determining regions L4, L5, L6, L7, L8, and L9 wherein:

the L1 sequence is
SEQ ID NO: 52 or SEQ ID NO: 16,
the L2 sequence is
SEQ ID NO: 5,
the L3 sequence is
SEQ ID NO: 53,
the L4 sequence is
(SEQ ID NO: 54)
GFTFNTYAMN,
the L5 sequence is
(SEQ ID NO: 55)
RIRSKYNNYATYYADSVKD,
the L6 sequence is
(SEQ ID NO: 70)
HX₁₁NFX₁₂NSKVSWFX₁₃Y,
the L7 sequence is
(SEQ ID NO: 71)
RSSX₁₄GAVTTSNYDN,
the L8 sequence is
(SEQ ID NO: 58)
GTNKRAP,
and
the L9 sequence is
(SEQ ID NO: 59)
ALWYSNLWV,

wherein X₁₁ is G, S, A or T, X₁₂ is G or P, X₁₃ is A or Q, and X₁₄ is T or A.

In another aspect of the multi-specific antibody or antibody fragment, the L6 sequence is any one of SEQ ID NOs: 56 and 60-67, and the L7 sequence is SEQ ID NO: 57, 68 or 69.

Each of the foregoing embodiments of the antibody or antibody fragment of this aspect may have a higher binding affinity to HER2 protein at a pH in a tumor microenvironment in comparison with a different pH that occurs in a non-tumor microenvironment. The pH in the tumor microenvironment may be in a range of from 5.0 to 7.0 and the pH in the non-tumor microenvironment may be in a range of from 7.2 to 7.8.

Each of the foregoing embodiments of the antibody or antibody fragment of this aspect may have a ratio of binding affinity to the HER2 protein at a pH in a tumor microenvironment to a binding affinity to the HER2 protein at a different pH in a non-tumor microenvironment of at least about 1.5:1, at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 50:1, at least about 70:1, or at least about 100:1.

In a further aspect, the present invention relates to an immunoconjugate comprising any of the foregoing embodiments of the antibody or antibody fragment. This immunoconjugate may include at least one agent selected from a chemotherapeutic agent, a radioactive atom, a cytostatic agent and a cytotoxic agent, or at least two said agents.

In each of the foregoing embodiments of the immunoconjugate, the at least one agent may be a radioactive agent and the radioactive agent may be selected from an alpha emitter, a beta emitter and a gamma emitter.

In each of the foregoing embodiments of the immunoconjugate, the at least one agent may be covalently bonded to a linker molecule. In each of the foregoing embodiments of the immunoconjugate, the at least one agent may be selected from maytansinoids, auristatins, dolastatins, calicheamicin, pyrrolobenzodiazepines, and anthracyclines.

In yet another aspect, the present invention relates to a pharmaceutical composition including the polypeptide of each of the foregoing embodiments, the antibody or antibody fragment of each of the foregoing embodiments, or the immunoconjugate of each of the foregoing embodiments; and a pharmaceutically acceptable carrier.

The foregoing embodiment of the pharmaceutical may include a tonicity agent.

Each of the foregoing embodiments of the pharmaceutical composition may further include an immune checkpoint inhibitor molecule. The immune checkpoint inhibitor molecule may be an antibody or antibody fragment against an immune checkpoint. The immune checkpoint may be selected from CTLA4, LAG3, TIM3, TIGIT, VISTA, BTLA, OX40, CD40, 4-1BB, PD-1, PD-L1, GITR, B7-H3, B7-H4, KIR, A2aR, CD27, CD70, DR3, and ICOS. Alternatively, the immune checkpoint may be one of CTLA4, PD-1 or PD-L1.

Each of the foregoing embodiments of the pharmaceutical composition may further include an antibody or antibody fragment against an antigen selected from CTLA4, PD1, PD-L1, AXL, ROR2, CD3, EpCAM, B7-H3, ROR1, SFRP4 and a WNT protein.

In another aspect, the present invention relates to a method of treating cancer comprising a step of administering the polypeptide of each of the foregoing embodiments, the antibody or antibody fragment of each of the foregoing embodiments, the immunoconjugate of each of the foregoing embodiments or the pharmaceutical composition of each of the foregoing embodiments to a patient with cancer.

In yet another aspect, the present invention provides a kit for diagnosis or treatment including any of the polypeptides, the antibody or antibody fragments, or the immunoconjugates of the present invention described above.

### BRIEF DESCRIPTION OF THE DRAWINGS

FIG. 1 shows a sequence alignment of exemplary light chain variable regions of anti-HER2 antibodies of the present invention.

FIG. 2 shows a sequence alignment of exemplary heavy chain variable regions of anti-HER2 antibodies of the present invention.

FIGS. 3A-3E show the binding activity of HER2 Benchmark antibody compared to exemplary conditionally active anti-HER2 antibodies of the present invention to human HER2 protein at pH 6.0 and pH 7.4, as measured by enzyme linked immunosorbent assay (ELISA). The Benchmark antibody is indicated by BM. For each of the conditionally active antibodies, one of the heavy chain (HC) and the light chain (LC) is specified in each figure. The unspecified heavy or light chain is the heavy or light chain of the Benchmark antibody. The Y-axis is the optical density (OD) at 450 nm. The X-axis shows the antibody concentration (log ng/mL) with a starting concentration of 300 ng/mL.

FIG. 4 shows the binding activity of HER2 Benchmark antibody (BM) compared to exemplary conditionally active anti-HER2 antibodies of the present invention to human HER2 protein over a range of pH values, as measured by enzyme linked immunosorbent assay (ELISA). For each of the conditionally active antibodies, the heavy chain (HC) and the light chain (LC) are specified in the figure. The Y-axis is the optical density (OD) at 450 nm. The X-axis shows the pH of the incubation and wash buffers (pH 5.0, 5.5, 6.0, 6.5, 7.0 and 7.4).

FIG. 5 shows binding activities of conditionally active anti-HER2 antibodies of the present invention to human cancer cell line (SKBR3) expressing human HER2 protein on cell surface in pH 6.0 (blue) or pH 7.4 (orange) for four different concentrations of the antibodies, as measured by fluorescence activated cell sorting (FACS).

FIGS. 6A-6B show binding activities of the HER2 Benchmark antibody (BM) and conditionally active anti-HER2 of the present invention to human HER2 protein at pH 6.0 (FIG. 6A) and pH 7.4 (FIG. 6B), determined by pH affinity ELISA assay. The numbering of the substitutions referenced in this Figure is based on the BAP-130 benchmark antibody of FIG. 2.

FIGS. 7A-7B show the binding activities of the HER2 Benchmark antibody (BM) and conditionally active anti-HER2 antibodies of the present invention to cynoHER2 protein at pH 6.0 (FIG. 7A) and pH 7.4 (FIG. 7B), determined by pH affinity ELISA assay. The numbering of the substitutions referenced in this Figure is based on the BAP-130 benchmark antibody of FIG. 2.

FIG. 8 shows the binding activity of the HER2 Benchmark antibody (BM) and conditionally active antibodies to human HER2 protein at various pH values, determined by pH range ELISA assay. The numbering of the substitutions referenced in this Figure is based on the BAP-130 benchmark antibody of FIG. 2.

FIG. 9A shows the mean body weights in grams of different treatment groups of the mice of Example 7. Data is presented as mean±SEM.

FIG. 9B shows relative body weight changes in percent of different treatment groups of the mice of Example 7. The percent changes were calculated based on the animal weight on the first day of dosing. Data is presented as mean±SEM.

FIG. 9C shows tumor growth curves of different treatment groups of the mice of Example 7. Data is presented as mean±SEM.

FIG. 10A shows a sequence alignment of exemplary heavy chain variable regions of anti-HER2 antibodies of the present invention. Exemplary heavy chain variable regions BAP150.24-WT-HC (SEQ ID NO: 34), BAP150.24-02-HC (SEQ ID NO: 35), BAP150.24-05-HC (SEQ ID NO: 36), BAP150.24-06-HC (SEQ ID NO: 37), BAP150.24-07-HC (SEQ ID NO: 38), an BAP150.24-08-HC (SEQ ID NO: 39) are shown. The H1, H2, and H3 CDR's, respectively, are underlined.

FIG. 11A shows a sequence alignment of exemplary light chain variable regions of anti-HER2 antibodies of the present invention. Exemplary light chain variable regions BAP150.24-WT-LC (SEQ ID NO: 40), BAP150.24-BF11-LC (SEQ ID NO: 41), BAP150.24-BF15-LC (SEQ ID NO: 42), BAP150.24-BF19-LC (SEQ ID NO: 43), BAP150.24-BF39-LC (SEQ ID NO: 44), BAP150.24-BF40-LC (SEQ ID NO: 45), BAP150.24-BF42-LC (SEQ ID NO: 46), BAP150.24-BF45-LC (SEQ ID NO: 47), and BAP150.24-BF46-LC (SEQ ID NO: 48) are shown. The L1, L2, L3, L4, L5, L6, L7, L8 and L9 CDR's, respectively, are underlined.

FIG. 12 shows that the bi-specific antibody may be a tetravalent homodimer “butterfly” including a CAB CD3 and that such an antibody can be detected by binding to CD3 on a plate.

FIGS. 13A-13D show the binding activities of WT HER2×WT CD3, WT HER2×CAB CD3-BF45 and CAB HER2-24-06×CAB CD3-BF19 bispecific antibodies compared to istotype×WT CD3 at pH 6.0 (FIGS. 13A and 13C) and pH 7.4 (FIGS. 13B and 13D), determined by pH sandwich ELISA assay.

FIG. 14 shows the binding activities of WT HER2×WT CD3, WT HER2×CAB CD3-BF45 and CAB HER2-24-06×CAB CD3-BF19 bispecific antibodies at various pH values, determined by a pH range ELISA assay.

FIGS. 15A-15I show surface plasmon resonance (SPR) binding analyses for WT HER2×WT CD3 with ligands huHER2-His, cyno-HER2-His, and huCD3-His, respectively, at pH 6.0 (FIGS. 15A-15C), pH 6.5 (FIGS. 15D-15F), and pH 7.4 (FIGS. 15G-15I).

FIGS. 16A-16I show surface plasmon resonance (SPR) binding analyses for WT HER2×CAB CD3-BF-45 with ligands huHER2-His, cyno-HER2-His, and huCD3-His, respectively, at pH 6.0 (FIGS. 16A-16C), pH 6.5 (FIGS. 16D-16F), and pH 7.4 (FIGS. 16G-16I).

FIGS. 17A-17I show surface plasmon resonance (SPR) binding analyses for CAB HER2-24-06×CAB CD3-BF-19 with ligands huHER2-His, cyno-HER2-His, and huCD3-His, respectively, at pH 6.0 (FIGS. 17A-17C), pH 6.5 (FIGS. 17D-17F), and pH 7.4 (FIGS. 17G-17I).

FIG. 18 is a schematic structure of a tetra-valent multi-specific antibody that is a homo-dimer with each arm having a binding site to an antigen (Ag) and a binding site to CD3.

### DEFINITIONS

To facilitate understanding of the examples provided herein, certain frequently occurring terms are defined herein.

In connection with a measured quantity, the term “about” as used herein refers to the normal variation in that measured quantity that would be expected by a skilled person making the measurement and exercising a level of care commensurate with the objective of the measurement and the precision of the measuring equipment used. Unless otherwise indicated, “about” refers to a variation of +/−10% of the value provided.

The term “affinity” as used herein refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

The term “affinity matured” antibody as used herein refers to an antibody with one or more alterations in one or more heavy chain or light chain variable regions, compared to a parent antibody which does not possess such alterations, such alterations resulting in an improvement in the affinity of the antibody for antigen.

The term “amino acid” as used herein refers to any organic compound that contains an amino group (—NH2) and a carboxyl group (—COOH); preferably either as free groups or alternatively after condensation as part of peptide bonds. The “twenty naturally encoded polypeptide-forming alpha-amino acids” are understood in the art and refer to: alanine (ala or A), arginine (arg or R), asparagine (asn or N), aspartic acid (asp or D), cysteine (cys or C), gluatamic acid (glu or E), glutamine (gin or Q), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (tip or W), tyrosine (tyr or Y), and valine (val or V).

The term “antibody” as used herein refers to intact immunoglobulin molecules, as well as fragments of immunoglobulin molecules, such as Fab, Fab′, (Fab′)2, Fv, and SCA fragments, that are capable of binding to an epitope of an antigen. These antibody fragments, which retain some ability to selectively bind to an antigen (e.g., a polypeptide antigen) of the antibody from which they are derived, can be made using well known methods in the art (see, e.g., “Antibodies: A Laboratory Manual, Second Edition,” Greenfield, Edward A., Ed., ISBN 978-1-936113-81-1 (2014)), and are described further, as follows. Antibodies can be used to isolate preparative quantities of the antigen by immunoaffinity chromatography. Various other uses of such antibodies are to diagnose and/or stage disease (e.g., neoplasia) and for therapeutic application to treat disease, such as for example: neoplasia, autoimmune disease, AIDS, cardiovascular disease, infections, and the like. Chimeric, human-like, humanized or fully human antibodies are particularly useful for administration to human patients.

An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner

An (Fab′)2 fragment of an antibody can be obtained by treating a whole antibody molecule with the enzyme pepsin, without subsequent reduction. A (Fab′)2 fragment is a dimer of two Fab′ fragments, held together by two disulfide bonds.

An Fv fragment is defined as a genetically engineered fragment containing the variable region of a light chain and the variable region of a heavy chain expressed as two chains.

The term “antibody fragment” as used herein refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “anti-HER2 antibody,” “HER2 antibody” and “an antibody that binds to HER2” as used herein refer to an antibody that is capable of binding HER2 protein with sufficient affinity such that the antibody is useful as a diagnostic and/or therapeutic agent in targeting HER2 protein. In one embodiment, the extent of binding of an anti-HER2 antibody to an unrelated, non-HER2 protein is less than about 10% of the binding of the antibody to HER2 protein as measured, e.g., by a radioimmunoassay (RIA). In certain embodiments, an antibody that binds to HER2 protein has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³M). In certain embodiments, an anti-HER2 antibody binds to an epitope of HER2 protein that is conserved among HER2 protein from different species, for example, the extracellular domain of HER2 protein.

The term “binding” as used herein refers to interaction of the variable region or an Fv of an antibody with an antigen with the interaction depending upon the presence of a particular structure (e.g., an antigenic determinant or epitope) on the antigen. For example, an antibody variable region or Fv recognizes and binds to a specific protein structure rather than to proteins generally. As used herein, the term “specifically binding” or “binding specifically” means that an antibody variable region or Fv binds to or associates with more frequently, more rapidly, with greater duration and/or with greater affinity with a particular antigen than with other proteins. For example, an antibody variable region or Fv specifically binds to its antigen with greater affinity, avidity, more readily, and/or with greater duration than it binds to other antigens. For another example, an antibody variable region or Fv binds to a cell surface protein (antigen) with materially greater affinity than it does to related proteins or other cell surface proteins or to antigens commonly recognized by polyreactive natural antibodies (i.e., by naturally occurring antibodies known to bind a variety of antigens naturally found in humans). However, “specifically binding” does not necessarily require exclusive binding or non-detectable binding of another antigen, this is meant by the term “selective binding”. In one example, “specific binding” of an antibody variable region or Fv (or other binding region) binds to an antigen, means that the an antibody variable region or Fv binds to the antigen with an equilibrium constant (KD) of 100 nM or less, such as 50 nM or less, for example 20 nM or less, such as, 15 nM or less, or 10 nM or less, or 5 nM or less, 2 nM or less, or 1 nM or less.

The terms “cancer” and “cancerous” as used herein refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The terms “cell proliferative disorder” and “proliferative disorder” as used herein refer to disorders that are associated with some degree of abnormal cell proliferation. In one embodiment, the cell proliferative disorder is cancer.

The term “chemotherapeutic agent” as used herein refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMF®); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics,” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “chimeric” antibody as used herein refers to an antibody in which a portion of the heavy and/or light chain is derived from a particular source or species, while the remainder of the heavy and/or light chain is derived from a different source or species.

The term “conditionally active antibody” as used herein refers to an anti-HER2 antibody which is more active under a condition in the tumor microenvironment compared to under a condition in the non-tumor microenvironment. The conditions in the tumor microenvironment include lower pH, higher concentrations of lactate and pyruvate, hypoxia, lower concentration of glucose, and slightly higher temperature in comparison with non-tumor microenvironment. For example, a conditionally active antibody is virtually inactive at normal body temperature but is active at a higher temperature in a tumor microenvironment. In yet another aspect, the conditionally active antibody is less active in normal oxygenated blood, but more active under a less oxygenated environment exists in tumor. In yet another aspect, the conditionally active antibody is less active in normal physiological pH 7.2-7.8, but more active under an acidic pH 5.0-7.0 that exists in a tumor microenvironment. There are other conditions in the tumor microenvironment know to a person skilled in the field may also be used as the condition in the present invention under which the anti-HER2 antibodies to have different binding affinity to HER2 protein.

The term “cytostatic agent” as used herein refers to a compound or composition which arrests growth of a cell either in vitro or in vivo. Thus, a cytostatic agent may be one which significantly reduces the percentage of cells in S phase. Further examples of cytostatic agents include agents that block cell cycle progression by inducing G0/G1 arrest or M-phase arrest. The humanized anti-HER2 antibody trastuzumab (HERCEPTIN®) is an example of a cytostatic agent that induces G0/G1 arrest. Classical M-phase blockers include the vincas (vincristine and vinblastine), taxanes, and topoisomerase II inhibitors such as doxorubicin, epirubicin, daunorubicin, etoposide, and bleomycin. Certain agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “cytotoxic agent” as used herein refers to a substance that inhibits or prevents a cellular function and/or causes cell death or destruction. Cytotoxic agents include, but are not limited to radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “diabodies” as used herein refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites.

The term “detectably label” as used herein refers to any substance whose detection or measurement, either directly or indirectly, by physical or chemical means, is indicative of the presence of an antigen in a sample. Representative examples of useful detectable labels include, but are not limited to, the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

The term “diagnostics” as used herein refers to determination of a subject's susceptibility to a disease or disorder, determination as to whether a subject is presently affected by a disease or disorder, prognosis of a subject affected by a disease or disorder (e. g., identification of pre-metastatic or metastatic cancerous states, stages of cancer, or responsiveness of cancer to therapy), and therametrics (e. g., monitoring a subject's condition to provide information as to the effect or efficacy of therapy). In some embodiments, the diagnostic method of this invention is particularly useful in detecting early stage cancers.

The term “diagnostic agent” as used herein refers to a molecule which can be directly or indirectly detected and is used for diagnostic purposes. The diagnostic agent may be administered to a subject or a sample. The diagnostic agent can be provided per se or may be conjugated to a vehicle such as a conditionally active antibody.

The term “effector functions” as used herein refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “effective amount” of an agent as used herein, e.g., a pharmaceutical formulation, refers to an amount effective, at dosages and for periods of time necessary, to achieve the desired therapeutic or prophylactic result.

The term “Fc region” as used herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “framework” or “FR” as used herein refers to variable domain residues other than complementarity determining regions (CDRs or H1-3 in the heavy chain and L1-3 in the light chain) residues. The FR of a variable domain generally consists of four FR domains: FR1, FR2, FR3, and FR4. Accordingly, the CDR and FR sequences generally appear in the following sequence in V_{H} (or V_{L}): FR1-H1(L1)-FR2-H2(L2)-1-R3-H3(L3)-FR4.

The term “full length antibody,” “intact antibody,” or “whole antibody” refers to an antibody which comprises an antigen-binding variable region (V_{H} or V_{L}) as well as a light chain constant domain (CL) and heavy chain constant domains, CH1, CH2 and CH3. The constant domains may be native sequence constant domains (e.g. human native sequence constant domains) or amino acid sequence variants thereof. Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “function-conservative variants” as used herein refers a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like) Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The terms “host cell,” “host cell line,” and “host cell culture” as used herein are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The term “human antibody” as used herein is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized” antibody as used herein refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “immunoconjugate” as used herein is an antibody conjugated to one or more heterologous molecule(s), including but not limited to a cytotoxic agent.

The term “individual” or “subject” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human

The term “inhibiting cell growth or proliferation” as used herein means decreasing a cell's growth or proliferation by at least 10%, 20%, 30%, 40%, 50%, 60%, 70%, 80%, 90%, 95%, or 100%, and includes inducing cell death.

The term “isolated” antibody as used herein is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase High Performance Liquid Chromatography (HPLC)). For review of methods for assessment of antibody purity, see, e.g., Flatman et al., J. Chromatogr. B, vol. 848, pp. 79-87, 2007.

The term “isolated nucleic acid encoding an anti-HER2 antibody” as used herein refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “metastasis” as used herein refers to all HER2-involving processes that support cancer cells to disperse from a primary tumor, penetrate into lymphatic and/or blood vessels, circulate through the bloodstream, and grow in a distant focus (metastasis) in normal tissues elsewhere in the body. In particular, it refers to cellular events of tumor cells such as proliferation, migration, anchorage independence, evasion of apoptosis, or secretion of angiogenic factors, that underlie metastasis and are stimulated or mediated by HER2 protein.

The term “microenvironment” as used herein means any portion or region of a tissue or body that has constant or temporal, physical or chemical differences from other regions of the tissue or regions of the body. For tumors, the term “tumor microenvironment” as used herein refers to the environment in which a tumor exists, which is the non-cellular area within the tumor and the area directly outside the tumorous tissue but does not pertain to the intracellular compartment of the cancer cell itself. The tumor and the tumor microenvironment are closely related and interact constantly. A tumor can change its microenvironment, and the microenvironment can affect how a tumor grows and spreads. Typically, the tumor microenvironment has a low pH in the range of 5.0 to 7.0, or in the range of 5.0 to 6.8, or in the range of 5.8 to 6.8, or in the range of 6.2-6.8. On the other hand, a normal physiological pH is in the range of 7.2-7.8 for most tissues. The tumor microenvironment is also known to have lower concentration of glucose and other nutrients, but higher concentration of lactic acid, in comparison with blood plasma. Furthermore, the tumor microenvironment can have a temperature that is 0.3 to 1° C. higher than the normal physiological temperature. The tumor microenvironment has been discussed in Gillies et al., “MRI of the Tumor Microenvironment,” Journal of Magnetic Resonance Imaging, vol. 16, pp. 430-450, 2002, hereby incorporated by reference herein its entirety. The term “non-tumor microenvironment” refers to a microenvironment at a site other than a tumor.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “naked antibody” as used herein refers to an antibody that is not conjugated to a heterologous moiety (e.g., a cytotoxic moiety) or radiolabel. The naked antibody may be present in a pharmaceutical formulation.

The term “package insert” as used herein is used to refer to instructions customarily included in commercial packages of therapeutic products, that contain information about the indications, usage, dosage, administration, combination therapy, contraindications and/or warnings concerning the use of such therapeutic products.

The term “percent (%) amino acid sequence identity” with respect to a reference polypeptide sequence as used herein is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The term “pharmaceutically acceptable carrier” as used herein refers to an ingredient in a pharmaceutical formulation, other than an active ingredient, which is nontoxic to a subject., A pharmaceutically acceptable carrier includes, but is not limited to, a buffer, excipient, stabilizer, or preservative.

The terms “purified” and “isolated” used herein refer to an antibody according to the invention or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

The term “recombinant antibody” as used herein refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia. coli cells or Bacillus subtilis cells, etc.

The term “single chain Fv” (“scFv”) as used herein is a covalently linked V_{H}::V_{L} heterodimer which is usually expressed from a gene fusion including V_{H} and V_{L} encoding genes linked by a peptide-encoding linker. “dsFv” is a V_{H}::V_{L} heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “therapeutically effective amount” of the antibody of the invention is meant a sufficient amount of the antibody to treat said cancer, at a reasonable benefit/risk ratio applicable to any medical treatment. It will be understood, however, that the total daily usage of the antibodies and compositions of the present invention will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

The term “treatment,” “treat,” or “treating” as used herein refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “tumor” as used herein refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The term “variable region” or “variable domain” as used herein refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three complementarity determining regions (CDRs). (See, e.g., Kindt et al. Kuby Immunology, 6th ed., W.H. Freeman and Co., page 91 (2007).) A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol., vol. 150, pp. 880-887, 1993; Clarkson et al., Nature, vol. 352, pp. 624-628, 1991.

The term “vector” as used herein refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### DETAILED DESCRIPTION

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in, other systems and methods. Before explaining the disclosed embodiments of the present invention in detail, it is to be understood that the invention is not limited in its application to the details of any particular embodiment shown. Additionally, the terminology used herein is for the purpose of description and not for limitation. Furthermore, although certain methods are described with reference to steps that are presented herein in a certain order, in many instances, these steps can be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more”, and “at least one” can be used interchangeably herein. The terms “comprising”, “including”, “having” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It is to be understood that each component, compound, substituent, or parameter disclosed herein is to be interpreted as being disclosed for use alone or in combination with one or more of each and every other component, compound, substituent, or parameter disclosed herein.

It is also to be understood that each amount/value or range of amounts/values for each component, compound, substituent, or parameter disclosed herein is to be interpreted as also being disclosed in combination with each amount/value or range of amounts/values disclosed for any other component(s), compounds(s), substituent(s), or parameter(s) disclosed herein and that any combination of amounts/values or ranges of amounts/values for two or more component(s), compounds(s), substituent(s), or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, substituent, or parameter disclosed in the description or an example is to be interpreted as a disclosure of either a lower or an upper limit of a range and thus can be combined with any other lower or upper limit of a range or specific amount/value for the same component, compound, substituent, or parameter disclosed elsewhere in the application to form a range for that component, compound, substituent, or parameter.

### A. Isolated Polypeptides

In one aspect, the present invention provides an isolated polypeptide that specifically binds to HER2 protein, said polypeptide comprising a heavy chain variable region including three complementarity determining regions, said regions having sequences H1, H2, and H3, wherein:

the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;

wherein X₁ is N or W, X₂ is R or K, X₃ is Y or K or D, X₄ is N or A, X₅ is G or K, X₆ is T or D, X₇ is R or E and X₈ is A or E; and:

a light chain variable region including three complementarity determining regions having sequences L1, L2, and L3, wherein:

the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQXYTTPPT,

wherein X₉ is A or D and X₁₀ is H or D or E; and
provided that when X₁-X₈ are N, R, Y, N, G, T, R and A, respectively, X₉ is not A and X₁₀ is not H.

In certain aspects of this embodiment, the H1 sequence may be GFWIKDTYIH (SEQ ID NO: 7) or GFNIKDTYIH (SEQ ID NO: 50); the H2 sequence may be any one of KIYPTNGYTRYADSVKG (SEQ ID NO: 8), RIKPTNGYTRYADSVKG (SEQ ID NO: 9), RIDPTNGYTRYADSVKG (SEQ ID NO: 10), RIYPTAGYTRYADSVKG (SEQ ID NO: 11), RIYPTNKYTRYADSVKG (SEQ ID NO: 12), RIYPTNGYDRYADSVKG (SEQ ID NO: 13), RIYPTNGYTEYADSVKG (SEQ ID NO: 14) and RIYPTNGYTRYADSVKG (SEQ ID NO: 49); the H3 sequence may be WGGDGFYEMDY (SEQ ID NO: 15) or WGGDGFYAMDY (SEQ ID NO: 51). The L1 sequence may be RASQDVNTDVA (SEQ ID NO: 16) or RASQDVNTAVA (SEQ ID NO: 52). The L2 sequence is SASFLYS (SEQ ID NO: 5). The L3 sequence may be sequence QQDYTTPPT (SEQ ID NO: 17), QQEYTTPPT (SEQ ID NO: 18), or QQHYTTPPT (SEQ ID NO: 53).

In specific embodiments of the invention, the anti-HER2 isolated polypeptide may be selected from any of the following anti-HER2 isolated polypeptides which comprise each specific combination of six CDRs H1, H2, H3, L1, L2 and L3 set forth below.

Exemplary Anti-HER2 Isolated Polypeptides

| H1, H2, H3 CDRs | L1, L2, L3 CDRs |
|---|---|
| SEQ ID NOs: 50 + 8 + 51 | SEQ ID NOs: 52 + 5 + 53 |
| SEQ ID NOs: 50 + 9 + 51 | SEQ ID NOs: 52 + 5 + 53 |
| SEQ ID NOs: 50 + 10 + 51 | SEQ ID NOs: 52 + 5 + 53 |
| SEQ ID NOs: 50 + 11 + 51 | SEQ ID NOs: 52 + 5 + 53 |
| SEQ ID NOs: 50 + 12 + 51 | SEQ ID NOs: 52 + 5 + 53 |
| SEQ ID NOs: 50 + 13 + 51 | SEQ ID NOs: 52 + 5 + 53 |
| SEQ ID NOs: 50 + 14 + 51 | SEQ ID NOs: 52 + 5 + 53 |
| SEQ ID NOs: 50 + 49 + 51 | SEQ ID NOs: 52 + 5 + 53 |
| SEQ ID NOs: 50 + 8 + 15 | SEQ ID NOs: 52 + 5 + 53 |
| SEQ ID NOs: 50 + 9 + 15 | SEQ ID NOs: 52 + 5 + 53 |
| SEQ ID NOs: 50 + 10 + 15 | SEQ ID NOs: 52 + 5 + 53 |
| SEQ ID NOs: 50 + 11 + 15 | SEQ ID NOs: 52 + 5 + 53 |
| SEQ ID NOs: 50 + 12 + 15 | SEQ ID NOs: 52 + 5 + 53 |
| SEQ ID NOs: 50 + 13 + 15 | SEQ ID NOs: 52 + 5 + 53 |
| SEQ ID NOs: 50 + 14 + 15 | SEQ ID NOs: 52 + 5 + 53 |
| SEQ ID NOs: 50 + 49 + 15 | SEQ ID NOs: 52 + 5 + 53 |
| SEQ ID NOs: 7 + 8 + 51 | SEQ ID NOs: 52 + 5 + 53 |
| SEQ ID NOs: 7 + 9 + 51 | SEQ ID NOs: 52 + 5 + 53 |
| SEQ ID NOs: 7 + 10 + 51 | SEQ ID NOs: 52 + 5 + 53 |
| SEQ ID NOs: 7 + 11 + 51 | SEQ ID NOs: 52 + 5 + 53 |
| SEQ ID NOs: 7 + 12 + 51 | SEQ ID NOs: 52 + 5 + 53 |
| SEQ ID NOs: 7 + 13 + 51 | SEQ ID NOs: 52 + 5 + 53 |
| SEQ ID NOs: 7 + 14 + 51 | SEQ ID NOs: 52 + 5 + 53 |
| SEQ ID NOs: 7 + 49 + 51 | SEQ ID NOs: 52 + 5 + 53 |
| SEQ ID NOs: 7 + 8 + 15 | SEQ ID NOs: 52 + 5 + 53 |
| SEQ ID NOs: 7 + 9 + 15 | SEQ ID NOs: 52 + 5 + 53 |
| SEQ ID NOs: 7 + 10 + 15 | SEQ ID NOs: 52 + 5 + 53 |
| SEQ ID NOs: 7 + 11 + 15 | SEQ ID NOs: 52 + 5 + 53 |
| SEQ ID NOs: 7 + 12 + 15 | SEQ ID NOs: 52 + 5 + 53 |
| SEQ ID NOs: 7 + 13 + 15 | SEQ ID NOs: 52 + 5 + 53 |
| SEQ ID NOs: 7 + 14 + 15 | SEQ ID NOs: 52 + 5 + 53 |
| SEQ ID NOs: 7 + 49 + 15 | SEQ ID NOs: 52 + 5 + 53 |
| SEQ ID NOs: 50 + 8 + 51 | SEQ ID NOs: 52 + 5 + 18 |
| SEQ ID NOs: 50 + 9 + 51 | SEQ ID NOs: 52 + 5 + 18 |
| SEQ ID NOs: 50 + 10 + 51 | SEQ ID NOs: 52 + 5 + 18 |
| SEQ ID NOs: 50 + 11 + 51 | SEQ ID NOs: 52 + 5 + 18 |
| SEQ ID NOs: 50 + 12 + 51 | SEQ ID NOs: 52 + 5 + 18 |
| SEQ ID NOs: 50 + 13 + 51 | SEQ ID NOs: 52 + 5 + 18 |
| SEQ ID NOs: 50 + 14 + 51 | SEQ ID NOs: 52 + 5 + 18 |
| SEQ ID NOs: 50 + 49 + 51 | SEQ ID NOs: 52 + 5 + 18 |
| SEQ ID NOs: 50 + 8 + 15 | SEQ ID NOs: 52 + 5 + 18 |
| SEQ ID NOs: 50 + 9 + 15 | SEQ ID NOs: 52 + 5 + 18 |
| SEQ ID NOs: 50 + 10 + 15 | SEQ ID NOs: 52 + 5 + 18 |
| SEQ ID NOs: 50 + 11 + 15 | SEQ ID NOs: 52 + 5 + 18 |
| SEQ ID NOs: 50 + 12 + 15 | SEQ ID NOs: 52 + 5 + 18 |
| SEQ ID NOs: 50 + 13 + 15 | SEQ ID NOs: 52 + 5 + 18 |
| SEQ ID NOs: 50 + 14 + 15 | SEQ ID NOs: 52 + 5 + 18 |
| SEQ ID NOs: 50 + 49 + 15 | SEQ ID NOs: 52 + 5 + 18 |
| SEQ ID NOs: 7 + 8 + 51 | SEQ ID NOs: 52 + 5 + 18 |
| SEQ ID NOs: 7 + 9 + 51 | SEQ ID NOs: 52 + 5 + 18 |
| SEQ ID NOs: 7 + 10 + 51 | SEQ ID NOs: 52 + 5 + 18 |
| SEQ ID NOs: 7 + 11 + 51 | SEQ ID NOs: 52 + 5 + 18 |
| SEQ ID NOs: 7 + 12 + 51 | SEQ ID NOs: 52 + 5 + 18 |
| SEQ ID NOs: 7 + 13 + 51 | SEQ ID NOs: 52 + 5 + 18 |
| SEQ ID NOs: 7 + 14 + 51 | SEQ ID NOs: 52 + 5 + 18 |
| SEQ ID NOs: 7 + 49 + 51 | SEQ ID NOs: 52 + 5 + 18 |
| SEQ ID NOs: 7 + 8 + 15 | SEQ ID NOs: 52 + 5 + 18 |
| SEQ ID NOs: 7 + 9 + 15 | SEQ ID NOs: 52 + 5 + 18 |
| SEQ ID NOs: 7 + 10 + 15 | SEQ ID NOs: 52 + 5 + 18 |
| SEQ ID NOs: 7 + 11 + 15 | SEQ ID NOs: 52 + 5 + 18 |
| SEQ ID NOs: 7 + 12 + 15 | SEQ ID NOs: 52 + 5 + 18 |
| SEQ ID NOs: 7 + 13 + 15 | SEQ ID NOs: 52 + 5 + 18 |
| SEQ ID NOs: 7 + 14 + 15 | SEQ ID NOs: 52 + 5 + 18 |
| SEQ ID NOs: 7 + 49 + 15 | SEQ ID NOs: 52 + 5 + 18 |
| SEQ ID NOs: 50 + 8 + 51 | SEQ ID NOs: 52 + 5 + 17 |
| SEQ ID NOs: 50 + 9 + 51 | SEQ ID NOs: 52 + 5 + 17 |
| SEQ ID NOs: 50 + 10 + 51 | SEQ ID NOs: 52 + 5 + 17 |
| SEQ ID NOs: 50 + 11 + 51 | SEQ ID NOs: 52 + 5 + 17 |
| SEQ ID NOs: 50 + 12 + 51 | SEQ ID NOs: 52 + 5 + 17 |
| SEQ ID NOs: 50 + 13 + 51 | SEQ ID NOs: 52 + 5 + 17 |
| SEQ ID NOs: 50 + 14 + 51 | SEQ ID NOs: 52 + 5 + 17 |
| SEQ ID NOs: 50 + 49 + 51 | SEQ ID NOs: 52 + 5 + 17 |
| SEQ ID NOs: 50 + 8 + 15 | SEQ ID NOs: 52 + 5 + 17 |
| SEQ ID NOs: 50 + 9 + 15 | SEQ ID NOs: 52 + 5 + 17 |
| SEQ ID NOs: 50 + 10 + 15 | SEQ ID NOs: 52 + 5 + 17 |
| SEQ ID NOs: 50 + 11 + 15 | SEQ ID NOs: 52 + 5 + 17 |
| SEQ ID NOs: 50 + 12 + 15 | SEQ ID NOs: 52 + 5 + 17 |
| SEQ ID NOs: 50 + 13 + 15 | SEQ ID NOs: 52 + 5 + 17 |
| SEQ ID NOs: 50 + 14 + 15 | SEQ ID NOs: 52 + 5 + 17 |
| SEQ ID NOs: 50 + 49 + 15 | SEQ ID NOs: 52 + 5 + 17 |
| SEQ ID NOs: 7 + 8 + 51 | SEQ ID NOs: 52 + 5 + 17 |
| SEQ ID NOs: 7 + 9 + 51 | SEQ ID NOs: 52 + 5 + 17 |
| SEQ ID NOs: 7 + 10 + 51 | SEQ ID NOs: 52 + 5 + 17 |
| SEQ ID NOs: 7 + 11 + 51 | SEQ ID NOs: 52 + 5 + 17 |
| SEQ ID NOs: 7 + 12 + 51 | SEQ ID NOs: 52 + 5 + 17 |
| SEQ ID NOs: 7 + 13 + 51 | SEQ ID NOs: 52 + 5 + 17 |
| SEQ ID NOs: 7 + 14 + 51 | SEQ ID NOs: 52 + 5 + 17 |
| SEQ ID NOs: 7 + 49 + 51 | SEQ ID NOs: 52 + 5 + 17 |
| SEQ ID NOs: 7 + 8 + 15 | SEQ ID NOs: 52 + 5 + 17 |
| SEQ ID NOs: 7 + 9 + 15 | SEQ ID NOs: 52 + 5 + 17 |
| SEQ ID NOs: 7 + 10 + 15 | SEQ ID NOs: 52 + 5 + 17 |
| SEQ ID NOs: 7 + 11 + 15 | SEQ ID NOs: 52 + 5 + 17 |
| SEQ ID NOs: 7 + 12 + 15 | SEQ ID NOs: 52 + 5 + 17 |
| SEQ ID NOs: 7 + 13 + 15 | SEQ ID NOs: 52 + 5 + 17 |
| SEQ ID NOs: 7 + 14 + 15 | SEQ ID NOs: 52 + 5 + 17 |
| SEQ ID NOs: 7 + 49 + 15 | SEQ ID NOs: 52 + 5 + 17 |
| SEQ ID NOs: 50 + 8 + 51 | SEQ ID NOs: 16 + 5 + 53 |
| SEQ ID NOs: 50 + 9 + 51 | SEQ ID NOs: 16 + 5 + 53 |
| SEQ ID NOs: 50 + 10 + 51 | SEQ ID NOs: 16 + 5 + 53 |
| SEQ ID NOs: 50 + 11 + 51 | SEQ ID NOs: 16 + 5 + 53 |
| SEQ ID NOs: 50 + 12 + 51 | SEQ ID NOs: 16 + 5 + 53 |
| SEQ ID NOs: 50 + 13 + 51 | SEQ ID NOs: 16 + 5 + 53 |
| SEQ ID NOs: 50 + 14 + 51 | SEQ ID NOs: 16 + 5 + 53 |
| SEQ ID NOs: 50 + 49 + 51 | SEQ ID NOs: 16 + 5 + 53 |
| SEQ ID NOs: 50 + 8 + 15 | SEQ ID NOs: 16 + 5 + 53 |
| SEQ ID NOs: 50 + 9 + 15 | SEQ ID NOs: 16 + 5 + 53 |
| SEQ ID NOs: 50 + 10 + 15 | SEQ ID NOs: 16 + 5 + 53 |
| SEQ ID NOs: 50 + 11 + 15 | SEQ ID NOs: 16 + 5 + 53 |
| SEQ ID NOs: 50 + 12 + 15 | SEQ ID NOs: 16 + 5 + 53 |
| SEQ ID NOs: 50 + 13 + 15 | SEQ ID NOs: 16 + 5 + 53 |
| SEQ ID NOs: 50 + 14 + 15 | SEQ ID NOs: 16 + 5 + 53 |
| SEQ ID NOs: 50 + 49 + 15 | SEQ ID NOs: 16 + 5 + 53 |
| SEQ ID NOs: 7 + 8 + 51 | SEQ ID NOs: 16 + 5 + 53 |
| SEQ ID NOs: 7 + 9 + 51 | SEQ ID NOs: 16 + 5 + 53 |
| SEQ ID NOs: 7 + 10 + 51 | SEQ ID NOs: 16 + 5 + 53 |
| SEQ ID NOs: 7 + 11 + 51 | SEQ ID NOs: 16 + 5 + 53 |
| SEQ ID NOs: 7 + 12 + 51 | SEQ ID NOs: 16 + 5 + 53 |
| SEQ ID NOs: 7 + 13 + 51 | SEQ ID NOs: 16 + 5 + 53 |
| SEQ ID NOs: 7 + 14 + 51 | SEQ ID NOs: 16 + 5 + 53 |
| SEQ ID NOs: 7 + 49 + 51 | SEQ ID NOs: 16 + 5 + 53 |
| SEQ ID NOs: 7 + 8 + 15 | SEQ ID NOs: 16 + 5 + 53 |
| SEQ ID NOs: 7 + 9 + 15 | SEQ ID NOs: 16 + 5 + 53 |
| SEQ ID NOs: 7 + 10 + 15 | SEQ ID NOs: 16 + 5 + 53 |
| SEQ ID NOs: 7 + 11 + 15 | SEQ ID NOs: 16 + 5 + 53 |
| SEQ ID NOs: 7 + 12 + 15 | SEQ ID NOs: 16 + 5 + 53 |
| SEQ ID NOs: 7 + 13 + 15 | SEQ ID NOs: 16 + 5 + 53 |
| SEQ ID NOs: 7 + 14 + 15 | SEQ ID NOs: 16 + 5 + 53 |
| SEQ ID NOs: 7 + 49 + 15 | SEQ ID NOs: 16 + 5 + 53 |
| SEQ ID NOs: 50 + 8 + 51 | SEQ ID NOs: 16 + 5 + 18 |
| SEQ ID NOs: 50 + 9 + 51 | SEQ ID NOs: 16 + 5 + 18 |
| SEQ ID NOs: 50 + 10 + 51 | SEQ ID NOs: 16 + 5 + 18 |
| SEQ ID NOs: 50 + 11 + 51 | SEQ ID NOs: 16 + 5 + 18 |
| SEQ ID NOs: 50 + 12 + 51 | SEQ ID NOs: 16 + 5 + 18 |
| SEQ ID NOs: 50 + 13 + 51 | SEQ ID NOs: 16 + 5 + 18 |
| SEQ ID NOs: 50 + 14 + 51 | SEQ ID NOs: 16 + 5 + 18 |
| SEQ ID NOs: 50 + 49 + 51 | SEQ ID NOs: 16 + 5 + 18 |
| SEQ ID NOs: 50 + 8 + 15 | SEQ ID NOs: 16 + 5 + 18 |
| SEQ ID NOs: 50 + 9 + 15 | SEQ ID NOs: 16 + 5 + 18 |
| SEQ ID NOs: 50 + 10 + 15 | SEQ ID NOs: 16 + 5 + 18 |
| SEQ ID NOs: 50 + 11 + 15 | SEQ ID NOs: 16 + 5 + 18 |
| SEQ ID NOs: 50 + 12 + 15 | SEQ ID NOs: 16 + 5 + 18 |
| SEQ ID NOs: 50 + 13 + 15 | SEQ ID NOs: 16 + 5 + 18 |
| SEQ ID NOs: 50 + 14 + 15 | SEQ ID NOs: 16 + 5 + 18 |
| SEQ ID NOs: 50 + 49 + 15 | SEQ ID NOs: 16 + 5 + 18 |
| SEQ ID NOs: 7 + 8 + 51 | SEQ ID NOs: 16 + 5 + 18 |
| SEQ ID NOs: 7 + 9 + 51 | SEQ ID NOs: 16 + 5 + 18 |
| SEQ ID NOs: 7 + 10 + 51 | SEQ ID NOs: 16 + 5 + 18 |
| SEQ ID NOs: 7 + 11 + 51 | SEQ ID NOs: 16 + 5 + 18 |
| SEQ ID NOs: 7 + 12 + 51 | SEQ ID NOs: 16 + 5 + 18 |
| SEQ ID NOs: 7 + 13 + 51 | SEQ ID NOs: 16 + 5 + 18 |
| SEQ ID NOs: 7 + 14 + 51 | SEQ ID NOs: 16 + 5 + 18 |
| SEQ ID NOs: 7 + 49 + 51 | SEQ ID NOs: 16 + 5 + 18 |
| SEQ ID NOs: 7 + 8 + 15 | SEQ ID NOs: 16 + 5 + 18 |
| SEQ ID NOs: 7 + 9 + 15 | SEQ ID NOs: 16 + 5 + 18 |
| SEQ ID NOs: 7 + 10 + 15 | SEQ ID NOs: 16 + 5 + 18 |
| SEQ ID NOs: 7 + 11 + 15 | SEQ ID NOs: 16 + 5 + 18 |
| SEQ ID NOs: 7 + 12 + 15 | SEQ ID NOs: 16 + 5 + 18 |
| SEQ ID NOs: 7 + 13 + 15 | SEQ ID NOs: 16 + 5 + 18 |
| SEQ ID NOs: 7 + 14 + 15 | SEQ ID NOs: 16 + 5 + 18 |
| SEQ ID NOs: 7 + 49 + 15 | SEQ ID NOs: 16 + 5 + 18 |
| SEQ ID NOs: 50 + 8 + 51 | SEQ ID NOs: 16 + 5 + 17 |
| SEQ ID NOs: 50 + 9 + 51 | SEQ ID NOs: 16 + 5 + 17 |
| SEQ ID NOs: 50 + 10 + 51 | SEQ ID NOs: 16 + 5 + 17 |
| SEQ ID NOs: 50 + 11 + 51 | SEQ ID NOs: 16 + 5 + 17 |
| SEQ ID NOs: 50 + 12 + 51 | SEQ ID NOs: 16 + 5 + 17 |
| SEQ ID NOs: 50 + 13 + 51 | SEQ ID NOs: 16 + 5 + 17 |
| SEQ ID NOs: 50 + 14 + 51 | SEQ ID NOs: 16 + 5 + 17 |
| SEQ ID NOs: 50 + 49 + 51 | SEQ ID NOs: 16 + 5 + 17 |
| SEQ ID NOs: 50 + 8 + 15 | SEQ ID NOs: 16 + 5 + 17 |
| SEQ ID NOs: 50 + 9 + 15 | SEQ ID NOs: 16 + 5 + 17 |
| SEQ ID NOs: 50 + 10 + 15 | SEQ ID NOs: 16 + 5 + 17 |
| SEQ ID NOs: 50 + 11 + 15 | SEQ ID NOs: 16 + 5 + 17 |
| SEQ ID NOs: 50 + 12 + 15 | SEQ ID NOs: 16 + 5 + 17 |
| SEQ ID NOs: 50 + 13 + 15 | SEQ ID NOs: 16 + 5 + 17 |
| SEQ ID NOs: 50 + 14 + 15 | SEQ ID NOs: 16 + 5 + 17 |
| SEQ ID NOs: 50 + 49 + 15 | SEQ ID NOs: 16 + 5 + 17 |
| SEQ ID NOs: 7 + 8 + 51 | SEQ ID NOs: 16 + 5 + 17 |
| SEQ ID NOs: 7 + 9 + 51 | SEQ ID NOs: 16 + 5 + 17 |
| SEQ ID NOs: 7 + 10 + 51 | SEQ ID NOs: 16 + 5 + 17 |
| SEQ ID NOs: 7 + 11 + 51 | SEQ ID NOs: 16 + 5 + 17 |
| SEQ ID NOs: 7 + 12 + 51 | SEQ ID NOs: 16 + 5 + 17 |
| SEQ ID NOs: 7 + 13 + 51 | SEQ ID NOs: 16 + 5 + 17 |
| SEQ ID NOs: 7 + 14 + 51 | SEQ ID NOs: 16 + 5 + 17 |
| SEQ ID NOs: 7 + 49 + 51 | SEQ ID NOs: 16 + 5 + 17 |
| SEQ ID NOs: 7 + 8 + 15 | SEQ ID NOs: 16 + 5 + 17 |
| SEQ ID NOs: 7 + 9 + 15 | SEQ ID NOs: 16 + 5 + 17 |
| SEQ ID NOs: 7 + 10 + 15 | SEQ ID NOs: 16 + 5 + 17 |
| SEQ ID NOs: 7 + 11 + 15 | SEQ ID NOs: 16 + 5 + 17 |
| SEQ ID NOs: 7 + 12 + 15 | SEQ ID NOs: 16 + 5 + 17 |
| SEQ ID NOs: 7 + 13 + 15 | SEQ ID NOs: 16 + 5 + 17 |
| SEQ ID NOs: 7 + 14 + 15 | SEQ ID NOs: 16 + 5 + 17 |
| SEQ ID NOs: 7 + 49 + 15 | SEQ ID NOs: 16 + 5 + 17 |

Preferred isolated polypeptides may be selected from isolated polypeptides which comprise each specific combination of six CDRs set forth below.

Preferred Anti-HER2 Isolated Polypeptides

| H1, H2, H3 CDRs | L1, L2, L3 CDRs |
|---|---|
| SEQ ID NOs: 50 + 9 + 51 | SEQ ID NOs: 16 + 5 + 53 |
| SEQ ID NOs: 50 + 12 + 51 | SEQ ID NOs: 16 + 5 + 53 |
| SEQ ID NOs: 50 + 13 + 51 | SEQ ID NOs: 16 + 5 + 53 |
| SEQ ID NOs: 50 + 49 + 51 | SEQ ID NOs: 16 + 5 + 53 |
| SEQ ID NOs: 50 + 49 + 15 | SEQ ID NOs: 16 + 5 + 53 |

In another aspect, the current disclosure provides an isolated polypeptide that specifically binds to HER2 protein, said polypeptide comprising a heavy chain variable region and a light chain variable region the heavy chain variable region having an amino acid sequence selected from SEQ ID NOS: 19-28; and a light chain variable region having an amino acid sequence selected from SEQ ID NOS: 29-32.

Still another aspect provides an isolated polypeptide that specifically binds to HER2 protein, said polypeptide comprising a heavy chain variable region and a light chain variable region the heavy chain variable region having an amino acid sequence selected from SEQ ID NOS: 33 and 19-28; and a light chain variable region having an amino acid sequence selected from SEQ ID NOS: 30-32.

In still another aspect, the current disclosure provides an isolated polypeptide that specifically binds to HER2 protein, said polypeptide comprising a heavy chain variable region and a light chain variable region, the heavy chain variable region having an amino acid sequence selected from SEQ ID NOS: 34-39; and a light chain variable region having an amino acid sequence selected from SEQ ID NOS: 40-48.

In a certain aspect, the current disclosure provides an isolated polypeptide that specifically binds to HER2 protein, said polypeptide comprising a heavy chain variable region and a light chain variable region, the heavy chain variable region having an amino acid sequence of SEQ ID NO: 35; and a light chain variable region having an amino acid sequence selected from SEQ ID NOS: 41-48.

In one aspect, the current disclosure provides an isolated polypeptide that specifically binds to HER2 protein, said polypeptide comprising a heavy chain variable region and a light chain variable region, the heavy chain variable region having an amino acid sequence of SEQ ID NO: 36; and a light chain variable region having an amino acid sequence selected from SEQ ID NOS: 41-48.

In a aspect, the current disclosure provides an isolated polypeptide that specifically binds to HER2 protein, said polypeptide comprising a heavy chain variable region and a light chain variable region, the heavy chain variable region having an amino acid sequence of SEQ ID NO: 37; and a light chain variable region having an amino acid sequence selected from SEQ ID NOS: 41-48.

In another aspect, the current disclosure provides an isolated polypeptide that specifically binds to HER2 protein, said polypeptide comprising a heavy chain variable region and a light chain variable region, the heavy chain variable region having an amino acid sequence of SEQ ID NO: 38; and a light chain variable region having an amino acid sequence selected from SEQ ID NOS: 41-48.

In another aspect, the current disclosure provides an isolated polypeptide that specifically binds to HER2 protein, said polypeptide comprising a heavy chain variable region and a light chain variable region, the heavy chain variable region having an amino acid sequence of SEQ ID NO: 39; and a light chain variable region having an amino acid sequence selected from SEQ ID NOS: 41-48.

In specific embodiments of the invention, the anti-HER2 isolated polypeptide may be selected from any of the following anti-HER2 isolated polypeptides which comprise each heavy chain variable region and a light chain variable region combination as set forth below.

Exemplary anti-HER2 Isolated Polvpeptides

| Heavy chain region | + | Light Chain region |
|---|---|---|
| SEQ ID NO: 34 | | SEQ ID NO: 29 |
| SEQ ID NO: 34 | | SEQ ID NO: 30 |
| SEQ ID NO: 34 | | SEQ ID NO: 31 |
| SEQ ID NO: 34 | | SEQ ID NO: 32 |
| SEQ ID NO: 34 | | SEQ ID NO: 40 |
| SEQ ID NO: 34 | | SEQ ID NO: 41 |
| SEQ ID NO: 34 | | SEQ ID NO: 42 |
| SEQ ID NO: 34 | | SEQ ID NO: 43 |
| SEQ ID NO: 34 | | SEQ ID NO: 44 |
| SEQ ID NO: 34 | | SEQ ID NO: 45 |
| SEQ ID NO: 34 | | SEQ ID NO: 46 |
| SEQ ID NO: 34 | | SEQ ID NO: 47 |
| SEQ ID NO: 34 | | SEQ ID NO: 48 |
| SEQ ID NO: 35 | | SEQ ID NO: 29 |
| SEQ ID NO: 35 | | SEQ ID NO: 30 |
| SEQ ID NO: 35 | | SEQ ID NO: 31 |
| SEQ ID NO: 35 | | SEQ ID NO: 32 |
| SEQ ID NO: 35 | | SEQ ID NO: 40 |
| SEQ ID NO: 35 | | SEQ ID NO: 41 |
| SEQ ID NO: 35 | | SEQ ID NO: 42 |
| SEQ ID NO: 35 | | SEQ ID NO: 43 |
| SEQ ID NO: 35 | | SEQ ID NO: 44 |
| SEQ ID NO: 35 | | SEQ ID NO: 45 |
| SEQ ID NO: 35 | | SEQ ID NO: 46 |
| SEQ ID NO: 35 | | SEQ ID NO: 47 |
| SEQ ID NO: 35 | | SEQ ID NO: 48 |
| SEQ ID NO: 36 | | SEQ ID NO: 29 |
| SEQ ID NO: 36 | | SEQ ID NO: 30 |
| SEQ ID NO: 36 | | SEQ ID NO: 31 |
| SEQ ID NO: 36 | | SEQ ID NO: 32 |
| SEQ ID NO: 36 | | SEQ ID NO: 40 |
| SEQ ID NO: 36 | | SEQ ID NO: 41 |
| SEQ ID NO: 36 | | SEQ ID NO: 42 |
| SEQ ID NO: 36 | | SEQ ID NO: 43 |
| SEQ ID NO: 36 | | SEQ ID NO: 44 |
| SEQ ID NO: 36 | | SEQ ID NO: 45 |
| SEQ ID NO: 36 | | SEQ ID NO: 46 |
| SEQ ID NO: 36 | | SEQ ID NO: 47 |
| SEQ ID NO: 36 | | SEQ ID NO: 48 |
| SEQ ID NO: 37 | | SEQ ID NO: 29 |
| SEQ ID NO: 37 | | SEQ ID NO: 30 |
| SEQ ID NO: 37 | | SEQ ID NO: 31 |
| SEQ ID NO: 37 | | SEQ ID NO: 32 |
| SEQ ID NO: 37 | | SEQ ID NO: 40 |
| SEQ ID NO: 37 | | SEQ ID NO: 41 |
| SEQ ID NO: 37 | | SEQ ID NO: 42 |
| SEQ ID NO: 37 | | SEQ ID NO: 43 |
| SEQ ID NO: 37 | | SEQ ID NO: 44 |
| SEQ ID NO: 37 | | SEQ ID NO: 45 |
| SEQ ID NO: 37 | | SEQ ID NO: 46 |
| SEQ ID NO: 37 | | SEQ ID NO: 47 |
| SEQ ID NO: 37 | | SEQ ID NO: 48 |
| SEQ ID NO: 38 | | SEQ ID NO: 29 |
| SEQ ID NO: 38 | | SEQ ID NO: 30 |
| SEQ ID NO: 38 | | SEQ ID NO: 31 |
| SEQ ID NO: 38 | | SEQ ID NO: 32 |
| SEQ ID NO: 38 | | SEQ ID NO: 40 |
| SEQ ID NO: 38 | | SEQ ID NO: 41 |
| SEQ ID NO: 38 | | SEQ ID NO: 42 |
| SEQ ID NO: 38 | | SEQ ID NO: 43 |
| SEQ ID NO: 38 | | SEQ ID NO: 44 |
| SEQ ID NO: 38 | | SEQ ID NO: 45 |
| SEQ ID NO: 38 | | SEQ ID NO: 46 |
| SEQ ID NO: 38 | | SEQ ID NO: 47 |
| SEQ ID NO: 38 | | SEQ ID NO: 48 |
| SEQ ID NO: 39 | | SEQ ID NO: 29 |
| SEQ ID NO: 39 | | SEQ ID NO: 30 |
| SEQ ID NO: 39 | | SEQ ID NO: 31 |
| SEQ ID NO: 39 | | SEQ ID NO: 32 |
| SEQ ID NO: 39 | | SEQ ID NO: 40 |
| SEQ ID NO: 39 | | SEQ ID NO: 41 |
| SEQ ID NO: 39 | | SEQ ID NO: 42 |
| SEQ ID NO: 39 | | SEQ ID NO: 43 |
| SEQ ID NO: 39 | | SEQ ID NO: 44 |
| SEQ ID NO: 39 | | SEQ ID NO: 45 |
| SEQ ID NO: 39 | | SEQ ID NO: 46 |
| SEQ ID NO: 39 | | SEQ ID NO: 47 |
| SEQ ID NO: 39 | | SEQ ID NO: 48 |
| SEQ ID NO: 19 | | SEQ ID NO: 29 |
| SEQ ID NO: 19 | | SEQ ID NO: 30 |
| SEQ ID NO: 19 | | SEQ ID NO: 31 |
| SEQ ID NO: 19 | | SEQ ID NO: 32 |
| SEQ ID NO: 19 | | SEQ ID NO: 40 |
| SEQ ID NO: 19 | | SEQ ID NO: 41 |
| SEQ ID NO: 19 | | SEQ ID NO: 42 |
| SEQ ID NO: 19 | | SEQ ID NO: 43 |
| SEQ ID NO: 19 | | SEQ ID NO: 44 |
| SEQ ID NO: 19 | | SEQ ID NO: 45 |
| SEQ ID NO: 19 | | SEQ ID NO: 46 |
| SEQ ID NO: 19 | | SEQ ID NO: 47 |
| SEQ ID NO: 19 | | SEQ ID NO: 48 |
| SEQ ID NO: 20 | | SEQ ID NO: 29 |
| SEQ ID NO: 20 | | SEQ ID NO: 30 |
| SEQ ID NO: 20 | | SEQ ID NO: 31 |
| SEQ ID NO: 20 | | SEQ ID NO: 32 |
| SEQ ID NO: 20 | | SEQ ID NO: 40 |
| SEQ ID NO: 20 | | SEQ ID NO: 41 |
| SEQ ID NO: 20 | | SEQ ID NO: 42 |
| SEQ ID NO: 20 | | SEQ ID NO: 43 |
| SEQ ID NO: 20 | | SEQ ID NO: 44 |
| SEQ ID NO: 20 | | SEQ ID NO: 45 |
| SEQ ID NO: 20 | | SEQ ID NO: 46 |
| SEQ ID NO: 20 | | SEQ ID NO: 47 |
| SEQ ID NO: 20 | | SEQ ID NO: 48 |
| SEQ ID NO: 21 | | SEQ ID NO: 29 |
| SEQ ID NO: 21 | | SEQ ID NO: 30 |
| SEQ ID NO: 21 | | SEQ ID NO: 31 |
| SEQ ID NO: 21 | | SEQ ID NO: 32 |
| SEQ ID NO: 21 | | SEQ ID NO: 40 |
| SEQ ID NO: 21 | | SEQ ID NO: 41 |
| SEQ ID NO: 21 | | SEQ ID NO: 42 |
| SEQ ID NO: 21 | | SEQ ID NO: 43 |
| SEQ ID NO: 21 | | SEQ ID NO: 44 |
| SEQ ID NO: 21 | | SEQ ID NO: 45 |
| SEQ ID NO: 21 | | SEQ ID NO: 46 |
| SEQ ID NO: 21 | | SEQ ID NO: 47 |
| SEQ ID NO: 21 | | SEQ ID NO: 48 |
| SEQ ID NO: 22 | | SEQ ID NO: 29 |
| SEQ ID NO: 22 | | SEQ ID NO: 30 |
| SEQ ID NO: 22 | | SEQ ID NO: 31 |
| SEQ ID NO: 22 | | SEQ ID NO: 32 |
| SEQ ID NO: 22 | | SEQ ID NO: 40 |
| SEQ ID NO: 22 | | SEQ ID NO: 41 |
| SEQ ID NO: 22 | | SEQ ID NO: 42 |
| SEQ ID NO: 22 | | SEQ ID NO: 43 |
| SEQ ID NO: 22 | | SEQ ID NO: 44 |
| SEQ ID NO: 22 | | SEQ ID NO: 45 |
| SEQ ID NO: 22 | | SEQ ID NO: 46 |
| SEQ ID NO: 22 | | SEQ ID NO: 47 |
| SEQ ID NO: 22 | | SEQ ID NO: 48 |
| SEQ ID NO: 23 | | SEQ ID NO: 29 |
| SEQ ID NO: 23 | | SEQ ID NO: 30 |
| SEQ ID NO: 23 | | SEQ ID NO: 31 |
| SEQ ID NO: 23 | | SEQ ID NO: 32 |
| SEQ ID NO: 23 | | SEQ ID NO: 40 |
| SEQ ID NO: 23 | | SEQ ID NO: 41 |
| SEQ ID NO: 23 | | SEQ ID NO: 42 |
| SEQ ID NO: 23 | | SEQ ID NO: 43 |
| SEQ ID NO: 23 | | SEQ ID NO: 44 |
| SEQ ID NO: 23 | | SEQ ID NO: 45 |
| SEQ ID NO: 23 | | SEQ ID NO: 46 |
| SEQ ID NO: 23 | | SEQ ID NO: 47 |
| SEQ ID NO: 23 | | SEQ ID NO: 48 |
| SEQ ID NO: 24 | | SEQ ID NO: 29 |
| SEQ ID NO: 24 | | SEQ ID NO: 30 |
| SEQ ID NO: 24 | | SEQ ID NO: 31 |
| SEQ ID NO: 24 | | SEQ ID NO: 32 |
| SEQ ID NO: 24 | | SEQ ID NO: 40 |
| SEQ ID NO: 24 | | SEQ ID NO: 41 |
| SEQ ID NO: 24 | | SEQ ID NO: 42 |
| SEQ ID NO: 24 | | SEQ ID NO: 43 |
| SEQ ID NO: 24 | | SEQ ID NO: 44 |
| SEQ ID NO: 24 | | SEQ ID NO: 45 |
| SEQ ID NO: 24 | | SEQ ID NO: 46 |
| SEQ ID NO: 24 | | SEQ ID NO: 47 |
| SEQ ID NO: 24 | | SEQ ID NO: 48 |
| SEQ ID NO: 25 | | SEQ ID NO: 29 |
| SEQ ID NO: 25 | | SEQ ID NO: 30 |
| SEQ ID NO: 25 | | SEQ ID NO: 31 |
| SEQ ID NO: 25 | | SEQ ID NO: 32 |
| SEQ ID NO: 25 | | SEQ ID NO: 40 |
| SEQ ID NO: 25 | | SEQ ID NO: 41 |
| SEQ ID NO: 25 | | SEQ ID NO: 42 |
| SEQ ID NO: 25 | | SEQ ID NO: 43 |
| SEQ ID NO: 25 | | SEQ ID NO: 44 |
| SEQ ID NO: 25 | | SEQ ID NO: 45 |
| SEQ ID NO: 25 | | SEQ ID NO: 46 |
| SEQ ID NO: 25 | | SEQ ID NO: 47 |
| SEQ ID NO: 25 | | SEQ ID NO: 48 |
| SEQ ID NO: 26 | | SEQ ID NO: 29 |
| SEQ ID NO: 26 | | SEQ ID NO: 30 |
| SEQ ID NO: 26 | | SEQ ID NO: 31 |
| SEQ ID NO: 26 | | SEQ ID NO: 32 |
| SEQ ID NO: 26 | | SEQ ID NO: 40 |
| SEQ ID NO: 26 | | SEQ ID NO: 41 |
| SEQ ID NO: 26 | | SEQ ID NO: 42 |
| SEQ ID NO: 26 | | SEQ ID NO: 43 |
| SEQ ID NO: 26 | | SEQ ID NO: 44 |
| SEQ ID NO: 26 | | SEQ ID NO: 45 |
| SEQ ID NO: 26 | | SEQ ID NO: 46 |
| SEQ ID NO: 26 | | SEQ ID NO: 47 |
| SEQ ID NO: 26 | | SEQ ID NO: 48 |
| SEQ ID NO: 27 | | SEQ ID NO: 29 |
| SEQ ID NO: 27 | | SEQ ID NO: 30 |
| SEQ ID NO: 27 | | SEQ ID NO: 31 |
| SEQ ID NO: 27 | | SEQ ID NO: 32 |
| SEQ ID NO: 27 | | SEQ ID NO: 40 |
| SEQ ID NO: 27 | | SEQ ID NO: 41 |
| SEQ ID NO: 27 | | SEQ ID NO: 42 |
| SEQ ID NO: 27 | | SEQ ID NO: 43 |
| SEQ ID NO: 27 | | SEQ ID NO: 44 |
| SEQ ID NO: 27 | | SEQ ID NO: 45 |
| SEQ ID NO: 27 | | SEQ ID NO: 46 |
| SEQ ID NO: 27 | | SEQ ID NO: 47 |
| SEQ ID NO: 27 | | SEQ ID NO: 48 |
| SEQ ID NO: 28 | | SEQ ID NO: 29 |
| SEQ ID NO: 28 | | SEQ ID NO: 30 |
| SEQ ID NO: 28 | | SEQ ID NO: 31 |
| SEQ ID NO: 28 | | SEQ ID NO: 32 |
| SEQ ID NO: 28 | | SEQ ID NO: 40 |
| SEQ ID NO: 28 | | SEQ ID NO: 41 |
| SEQ ID NO: 28 | | SEQ ID NO: 42 |
| SEQ ID NO: 28 | | SEQ ID NO: 43 |
| SEQ ID NO: 28 | | SEQ ID NO: 44 |
| SEQ ID NO: 28 | | SEQ ID NO: 45 |
| SEQ ID NO: 28 | | SEQ ID NO: 46 |
| SEQ ID NO: 28 | | SEQ ID NO: 47 |
| SEQ ID NO: 28 | | SEQ ID NO: 48 |
| SEQ ID NO: 33 | | SEQ ID NO: 29 |
| SEQ ID NO: 33 | | SEQ ID NO: 30 |
| SEQ ID NO: 33 | | SEQ ID NO: 31 |
| SEQ ID NO: 33 | | SEQ ID NO: 32 |
| SEQ ID NO: 33 | | SEQ ID NO: 40 |
| SEQ ID NO: 33 | | SEQ ID NO: 41 |
| SEQ ID NO: 33 | | SEQ ID NO: 42 |
| SEQ ID NO: 33 | | SEQ ID NO: 43 |
| SEQ ID NO: 33 | | SEQ ID NO: 44 |
| SEQ ID NO: 33 | | SEQ ID NO: 45 |
| SEQ ID NO: 33 | | SEQ ID NO: 46 |
| SEQ ID NO: 33 | | SEQ ID NO: 47 |
| SEQ ID NO: 33 | | SEQ ID NO: 48 |

Preferred Anti-HER2 Isolated Polypeptides

| Heavy chain region | + | Light Chain region |
|---|---|---|
| SEQ ID NO: 19 | | SEQ ID NO: 29 |
| SEQ ID NO: 19 | | SEQ ID NO: 30 |
| SEQ ID NO: 20 | | SEQ ID NO: 29 |
| SEQ ID NO: 20 | | SEQ ID NO: 30 |
| SEQ ID NO: 21 | | SEQ ID NO: 30 |
| SEQ ID NO: 22 | | SEQ ID NO: 30 |
| SEQ ID NO: 23 | | SEQ ID NO: 30 |
| SEQ ID NO: 24 | | SEQ ID NO: 30 |
| SEQ ID NO: 25 | | SEQ ID NO: 30 |
| SEQ ID NO: 26 | | SEQ ID NO: 30 |
| SEQ ID NO: 27 | | SEQ ID NO: 30 |
| SEQ ID NO: 28 | | SEQ ID NO: 30 |
| SEQ ID NO: 33 | | SEQ ID NO: 30 |
| SEQ ID NO: 33 | | SEQ ID NO: 31 |
| SEQ ID NO: 33 | | SEQ ID NO: 32 |

### B. Anti-HER2 Antibodies

In another aspect, the present invention relates to an anti-HER2 antibody or antibody fragment comprising the isolated polypeptides described above.

The antibody or antibody fragment may have a higher binding affinity to HER2 protein at a pH in a tumor microenvironment in comparison with a pH that occurs in a non-tumor microenvironment. The pH in the tumor microenvironment may range of from 5.0 to 7.0 and the pH in the non-tumor microenvironment may range from 7.2 to 7.8.

In another aspect, the invention relates to an antibody or antibody fragment comprising a heavy chain variable region and a light chain variable region, wherein the heavy chain variable region includes three complementarity determining regions, said regions having sequences H1, H2, and H3, wherein:

the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;

wherein X₁ is N or W, X₂ is R or K, X₃ is Y or K or D, X₄ is N or A, X₅ is G or K, X₆ is T or D, X₇ is R or E and X₈ is A or E; and

a light chain variable region including three complementarity determining regions having sequences L1, L2, and L3, wherein:

the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,

wherein X₉ is A or D and X₁₀ is H or D or E; and
provided that when X₁-X₈ are N, R, Y, N, G, T, R and A, respectively, X₉ is not A and X₁₀ is not H.

In certain aspects of this embodiment, the H1 sequence may be GFWIKDTYIH (SEQ ID NO: 7) or GFNIKDTYIH (SEQ ID NO: 50); the H2 sequence may be KIYPTNGYTRYADSVKG (SEQ ID NO: 8), RIKPTNGYTRYADSVKG (SEQ ID NO: 9), RIDPTNGYTRYADSVKG (SEQ ID NO: 10), RIYPTAGYTRYADSVKG (SEQ ID NO: 11), RIYPTNKYTRYADSVKG (SEQ ID NO: 12), RIYPTNGYDRYADSVKG (SEQ ID NO: 13), RIYPTNGYTEYADSVKG (SEQ ID NO: 14) or RIYPTNGYTRYADSVKG (SEQ ID NO: 49); and the H3 sequence may be WGGDGFYEMDY (SEQ ID NO: 15) or WGGDGFYAMDY (SEQ ID NO: 51).

Also in certain aspects of this embodiment, the L1 sequence is RASQDVNTDVA (SEQ ID NO: 16) or RASQDVNTAVA (SEQ ID NO: 52); the L2 sequence is SASFLYS (SEQ ID NO: 5); and the L3 sequence may be QQDYTTPPT (SEQ ID NO: 17), QQEYTTPPT (SEQ ID NO: 18) or QQHYTTPPT (SEQ ID NO: 53).

In certain embodiments the anti-HER2 antibodies and antibody fragments of the present invention include the combinations of six CDR's set forth in the list above for the isolated polypeptides. Preferred anti-HER2 antibodies and antibody fragments of the present invention are those that include the preferred combinations of six CDR's set forth in the list above for the isolated polypeptides.

In certain embodiments the disclosure provides an antibody or antibody fragment that specifically binds to HER2 protein, said antibody or antibody fragment comprising a heavy chain variable region may be any one of SEQ ID NOS: 19-28 and 33 and the light chain variable region may be any one of SEQ ID NOS: 29-32.

In certain embodiments of the anti-HER2 antibody or antibody fragment, the heavy chain variable region may be any one of SEQ ID NOS: 33 and 19-28 and the light chain variable region may be any one of SEQ ID NOS: 30-32.

In each of the foregoing embodiments of this aspect of the antibody or antibody fragment, the heavy chain variable region may be any one of SEQ ID NOS: 35-39 and the light chain variable region may be any one of SEQ ID NOS: 41-48.

In one aspect, the disclosure provides an antibody or antibody fragment that specifically binds to HER2 protein, said antibody or antibody fragment comprising a heavy chain variable region and a light chain variable region the heavy chain variable region having an amino acid sequence selected from SEQ ID NOS: 35-39; and a light chain variable region having an amino acid sequence selected from SEQ ID NOS: 41-48.

In a certain aspect, the current disclosure provides an antibody or antibody fragment that specifically binds to HER2 protein, said antibody or antibody fragment comprising a heavy chain variable region and a light chain variable region, the heavy chain variable region having an amino acid sequence of SEQ ID NO: 35; and a light chain variable region having an amino acid sequence selected from SEQ ID NOS: 41-48.

In a certain aspect, the current disclosure provides an antibody or antibody fragment that specifically binds to HER2 protein, said antibody or antibody fragment comprising a heavy chain variable region and a light chain variable region, the heavy chain variable region having an amino acid sequence of SEQ ID NO: 36; and a light chain variable region having an amino acid sequence selected from SEQ ID NOS: 41-48.

In a certain aspect, the current disclosure provides an antibody or antibody fragment that specifically binds to HER2 protein, said antibody or antibody fragment comprising a heavy chain variable region and a light chain variable region, the heavy chain variable region having an amino acid sequence of SEQ ID NO: 37; and a light chain variable region having an amino acid sequence selected from SEQ ID NOS: 41-48.

In another certain aspect, the current disclosure provides an antibody or antibody fragment that specifically binds to HER2 protein, said antibody or antibody fragment comprising a heavy chain variable region and a light chain variable region, the heavy chain variable region having an amino acid sequence of SEQ ID NO: 38; and a light chain variable region having an amino acid sequence selected from SEQ ID NOS: 41-48.

In another certain aspect, the current disclosure provides an antibody or antibody fragment that specifically binds to HER2 protein, said antibody or antibody fragment comprising a heavy chain variable region and a light chain variable region, the heavy chain variable region having an amino acid sequence of SEQ ID NO: 39; and a light chain variable region having an amino acid sequence selected from SEQ ID NOS: 41-48.

In certain embodiments the anti-HER2 antibodies and antibody fragments of the present invention include the combinations of heavy and light chain variable regions set forth in the list above for the isolated polypeptides. Preferred anti-HER2 antibodies and antibody fragments of the present invention are those that include the preferred combinations of heavy and light chain variable regions set forth in the list above for the isolated polypeptides.

The antibody or antibody fragment of this aspect may also have a higher binding affinity to HER2 protein at a pH in a tumor microenvironment in comparison with a different pH that occurs in a non-tumor microenvironment. The pH in the tumor microenvironment may be in a range of from 5.0 to 7.0 and the pH in the non-tumor microenvironment may be in a range of from 7.2 to 7.8.

The antibody or antibody fragment of this aspect may have a ratio of binding affinity to the HER2 protein at a pH in a tumor microenvironment to a binding affinity to the HER2 protein at a different pH in a non-tumor microenvironment of at least about 1.5:1, at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 50:1, at least about 70:1, or at least about 100:1.

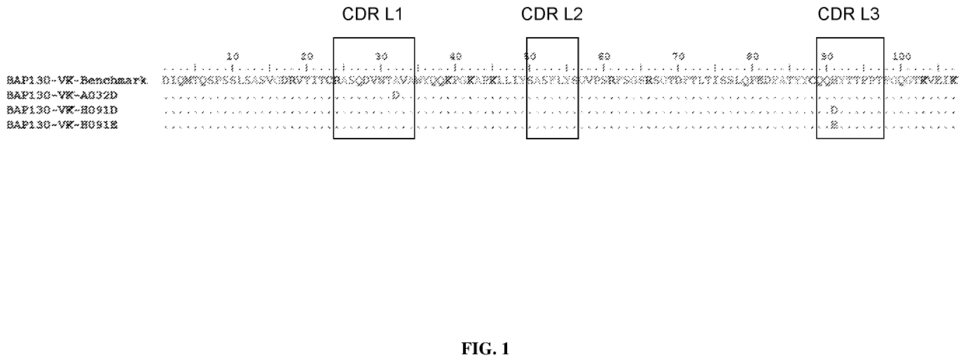

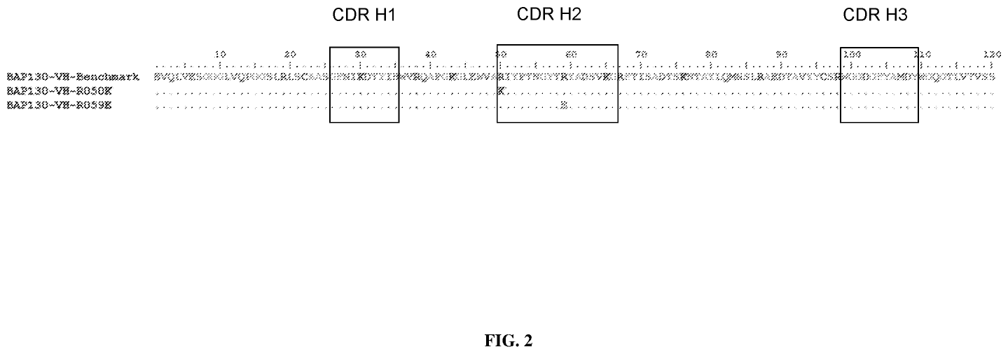

The alignments of exemplary light chain variable regions of the present invention are shown in FIG. 1, where the complementarity determining regions L1, L2, and L3 are enclosed in boxes. The alignments of exemplary heavy chain variable regions of the present invention are shown in FIG. 2, where the complementarity determining regions H1, H2, H3 are enclosed in boxes.

The heavy chain variable regions and the light chain variable regions of the present invention were each obtained from a parent antibody using a method disclosed in U.S. Pat. No. 8,709,755. This method of generating the heavy chain variable regions and the light chain variable regions, as well as the method of generating antibodies and antibody fragments disclosed in U.S. Pat. No. 8,709,755, are hereby incorporated by reference herein.

The amino acid sequences of the light chain variable regions of FIG. 1 are set forth in SEQ ID NOS: 29-32. The amino acid sequences of the heavy chain variable regions of FIG. 2 are set forth in SEQ ID NOS: 19, 20 and 33.

In one embodiment, the antibody or antibody fragment comprises a light chain variable region and a heavy chain variable region having any one pair of sequences selected from: SEQ ID NOS: 30 and 33, SEQ ID NOS: 31 and 33, SEQ ID NOS: 32 and 33, SEQ ID NOS: 29 and 19, SEQ ID NOS: 29 and 20, SEQ ID NOS: 30 and 21, SEQ ID NOS: 30 and 22, SEQ ID NOS: 30 and 23, SEQ ID NOS: 30 and 24, SEQ ID NOS: 30 and 25, SEQ ID NOS: 30 and 26, SEQ ID NOS: 30 and 27, and SEQ ID NOS: 30 and 28.

Antibodies and antibody fragments including these heavy chain variable regions and light chain variable regions can specifically bind to HER2 protein, especially human HER2 protein. Antibodies or antibody fragments comprising a combination of one of these heavy chain variable regions and one of these light chain variable regions have been found to have higher binding affinity to HER2 protein at a pH in the tumor microenvironment (e.g. pH 5.0-7.0) than at a pH in a non-tumor microenvironment (e.g. pH 7.2-7.8). As a result, the anti-HER2 antibodies or antibody fragments have a higher binding affinity to HER2 protein in a tumor microenvironment in comparison with their binding affinity to HER2 protein in a typical normal tissue microenvironment.

Anti-HER2 antibodies or antibody fragments of the present invention are thus expected to exhibit reduced side-effects, relative to non-conditionally active anti-HER2 antibodies, due to their reduced binding affinity to HER2 protein in the normal tissue microenvironment. Anti-HER2 antibodies or antibody fragments of the present invention are also expected to have a comparable or greater efficacy than monoclonal anti-HER2 antibodies known in the art. Several examples of anti-HER2 antibodies that exhibited essentially no side effects and comparable or greater efficacy than an isotype control antibody are demonstrated in Example 7 below in in vivo testing in a BALB/c mouse model. This combination of features permits use of a higher dosage of these anti-HER2 antibodies or antibody fragments due to the reduced side effects, which may provide a more effective therapy option.

In addition to the polypeptides and the antibodies or antibody fragments having the heavy chain variable regions and light chain variable regions described, the present invention also includes variants of these polypeptides, antibodies and antibody fragments, that can specifically bind to HER2 protein, especially human HER2 protein. In some embodiments, these variants have different H1, H2, H3, L1, L2 or L3 sequences. In other embodiments, the portion of the amino acid sequence of the heavy and light chain variable regions outside of the complementarity determining regions may be mutated in accordance with the principles of substitution, insertion and deletion, as discussed in this application to provide these variants. In still further embodiments, the constant regions may be modified to provide these variants. In still further embodiments, two or all three of these regions may be modified to provide these variants.

In deriving these variants, one is guided by the process as described herein. The variants of the heavy chain and light chain variable regions may be prepared by introducing appropriate modifications into the nucleotide sequence encoding the heavy and light chain variable regions, or by peptide synthesis. Such modifications include, for example, deletions from, and/or insertions into and/or substitutions of residues within the amino acid sequences of the heavy and light chain variable regions. Any combination of deletion, insertion, and substitution can be made to arrive at the antibodies or antibody fragments of the present invention, provided that they possess the desired characteristics, e.g., antigen-binding to human HER2 protein and conditional activity based on a variation in pH from a tumor microenvironment to a normal tissue environment.

### C. Substitution, Insertion, and Deletion Variants

In certain embodiments, antibody or antibody fragment variants having one or more amino acid substitutions are provided. Sites of interest for substitutional mutagenesis include the CDRs and framework regions (FRs). Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody or antibody fragment of interest and the products screened for a desired activity, e.g., retained/improved antigen binding, conditional activity and/or decreased immunogenicity.

TABLE 1
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more complementarity determining region residues of a parent antibody (e.g. a humanized or human antibody). Generally, the resulting variant(s) selected will have modifications (e.g., improvements) in certain biological properties (e.g., increased affinity, improved conditional activity or selectivity, reduced immunogenicity) relative to the parent antibody and/or will have substantially retained certain biological properties of the parent antibody. An exemplary substitutional variant is an affinity matured antibody, which may be generated, e.g., using phage display-based affinity maturation techniques such as those described herein.

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol., vol. 207, pp. 179-196, 2008), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology, vol. 178, pp. 1-37, 2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized CDR residues involved in antigen binding may be specifically identified, e.g., using alanine scanning mutagenesis or modeling. CDR-H3 and CDR-L3 are often targeted.

In certain embodiments, substitutions, insertions, or deletions may occur within one or more CDRs so long as such alterations do not substantially reduce the ability of the antibody or antibody fragment to bind to the HER2 antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant V_{H} and V_{L} sequences provided above, each CDR either is unaltered, or contains no more than one, two or three amino acid substitutions.

A useful method for identification of residues or regions of an antibody that may be targeted for mutagenesis is “alanine scanning mutagenesis” as described by Cunningham and Wells, Science, vol. 244, pp. 1081-1085, 1989. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or antibody fragment with antigen is affected. Further substitutions may be introduced at the amino acid locations demonstrating functional sensitivity to the initial substitutions. Alternatively, or additionally, a crystal structure of an antigen-antibody complex to identify contact points between the antibody or antibody fragment and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody include the fusion to the N- or C-terminus of the antibody to an enzyme (e.g. for ADEPT) or a polypeptide which increases the serum half-life of the antibody.

Amino acid sequence modification(s) of the antibodies described herein are contemplated. For example, it may be desirable to improve the binding affinity and/or other biological properties of the antibody. It is known that when a humanized antibody is produced by simply grafting only CDRs in V_{H} and V_{L} of an antibody derived from a non-human animal in FRs of the V_{H} and V_{L} of a human antibody, the antigen binding activity is reduced in comparison with that of the original antibody derived from a non-human animal. It is considered that several amino acid residues of the V_{H} and V_{L} of the non-human antibody, not only in CDRs but also in FRs, are directly or indirectly associated with the antigen binding activity. Hence, substitution of these amino acid residues with different amino acid residues derived from FRs of the V_{H} and V_{L} of the human antibody would reduce of the binding activity. In order to resolve the problem, in antibodies grafted with human CDR, attempts have to be made to identify, among amino acid sequences of the FR of the V_{H} and V_{L} of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present invention, and in the DNA sequences encoding them, and a functional molecule that encodes an antibody with desirable characteristics may still be obtained.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The present invention also encompasses function-conservative variants of the antibodies and antibody fragments of the present invention.

Two amino acid sequences are “substantially homologous” or “substantially similar” when greater than 80%, or greater than 85%, or preferably greater than 90%, or more preferably greater than 95%, or greater than 98% of the amino acids are identical. In some embodiments at least 90% or greater than 95% of the amino acids similar (functionally identical) over the whole length of the sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without expecting an appreciable loss of activity (see e.g. Table 1 above). Since the interactive capacity and nature of a protein define the protein's biological functional activity, certain amino acid substitutions can be made in a protein sequence, and, of course, in its DNA encoding sequence, while nevertheless obtaining a protein with similar properties. It is thus contemplated that various changes may be made in the sequences of the antibodies or antibody fragments of the invention, or corresponding DNA sequences which encode said antibodies or antibody fragments, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined above, amino acid substitutions may be based on the relative similarity of the amino acid side-chain substituents, for example, their hydrophobicity, hydrophilicity, charge, size, and the like. Exemplary replacements which take various of the foregoing characteristics into consideration are well known to those of skill in the art and include replacements using the following pairs: arginine and lysine; glutamate and aspartate; serine and threonine; glutamine and asparagine; and valine, leucine and isoleucine.

### D. Glycosylation Variants

In certain embodiments, the anti-HER2 antibodies or antibody fragments provided herein are altered to increase or decrease the extent to which the antibodies or antibody fragments are glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH, vol. 15, pp. 26-32, 1997. The oligosaccharide may include various carbohydrates, e g, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol., vol. 336, pp. 1239-1249, 2004; Yamane-Ohnuki et al. Biotech. Bioeng., vol. 87, pp. 614-622, 2004. Examples of cell lines capable of producing defucosylated antibodies include Lec13 CHO cells deficient in protein fucosylation (Ripka et al. Arch. Biochem. Biophys., vol. 249, pp. 533-545, 1986; US Pat Appl No US 2003/0157108 A; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng., vol. 87, pp. 614-622, 2004; Kanda, Y. et al., Biotechnol. Bioeng., vol. 94, pp. 680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### E. Fc Region Variants

In certain embodiments, one or more amino acid modifications may be introduced into the Fc region of the anti-HER2 antibodies or antibody fragments provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol., vol. 9, pp. 457-492, 1991. Non-limiting examples of in vitro assays to assess ADCC activity of a molecule of interest are described in U.S. Pat. No. 5,500,362 (see also, e.g. Hellstrom et al. Proc. Nat'l Acad. Sci. USA, vol. 83, pp. 7059-7063, 1986) and Hellstrom, I et al., Proc. Nat'l Acad. Sci. USA, vol. 82, pp. 1499-1502, 1985; U.S. Pat. No. 5,821,337 (see also Bruggemann et al., J. Exp. Med., vol. 166, pp. 1351-1361, 1987). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA, vol. 95, pp. 652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods, vol. 202, pp. 163-171, 1996; Cragg, M. S. et al., Blood, vol. 101, pp. 1045-1052, 2003; and Cragg, M. S, and M. J. Glennie, Blood, vol. 103, pp. 2738-2743, 2004). FcRn binding and in vivo clearance/half life determinations can also be performed using methods known in the art (see, e.g., Petkova, S. B. et al., Int'l. Immunol., vol. 18, pp. 1759-1769, 2006).

The variants of the antibodies or antibody fragments with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem., vol. 9, pp. 6591-6604, 2001).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol., vol. 164, pp. 4178-4184, 2000.

Antibodies with increased half lives and improved binding to the neonatal Fc receptor (FcRn), which is responsible for the transfer of maternal IgGs to the fetus (Guyer et al., J. Immunol., vol. 117, pp. 587-593, 1976 and Kim et al., J. Immunol., vol. 24, p. 249, 1994), are described in US2005/0014934. Those antibodies comprise an Fc region with one or more substitutions therein which improve binding of the Fc region to FcRn. Such Fc variants include/e those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature, vol. 322, pp. 738-740, 1988; U.S. Pat. Nos. 5,648,260; 5,624,821; and WO 94/29351 concerning other examples of Fc region variants.

### F. Cysteine Engineered Antibody Variants

In certain embodiments, it may be desirable to create cysteine engineered antibodies, e.g., “thioMAbs,” in which one or more residues of the anti-HER2 antibodies or antibody fragments are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### G. Antibody Derivatives

In certain embodiments, the anti-HER2 antibodies or antibody fragments provided herein may be further modified to contain additional nonproteinaceous moieties that are known in the art and readily available. The moieties suitable for derivatization of the antibody or antibody fragment include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody or antibody fragment may vary, and if more than one polymer is attached, they can be the same or different molecules. In general, the number and/or type of polymers used for derivatization can be determined based on considerations including, but not limited to, the particular properties or functions of the antibody or antibody fragment to be improved, whether the derivative will be used in a therapy under defined conditions, etc.

In another embodiment, conjugates of the antibodies or antibody fragments and nonproteinaceous moiety that may be selectively heated by exposure to radiation are provided. In one embodiment, the nonproteinaceous moiety is a carbon nanotube (Kam et al., Proc. Natl. Acad. Sci. USA, vol. 102, pp. 11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

The anti-HER2 antibodies or antibody fragments of the invention, or their variants, have a higher binding affinity to HER2 protein under a condition in a tumor microenvironment than under a condition in a non-tumor microenvironment. The condition in tumor microenvironment and the condition in the non-tumor microenvironment are both pH. The anti-HER2 antibodies or antibody fragments of the invention thus can selectively bind to HER2 protein at a pH about 5.0-7.0 or 5.0-6.8 but will have a lower binding affinity to HER2 protein at a pH about 7.2-7.8 encountered in a normal, non-tumor microenvironment. As shown Examples 3 and 6, the anti-HER2 antibodies or antibody fragments have higher binding affinity to HER2 protein at pH 6.0 than at pH 7.4.

In certain embodiments, the anti-HER2 antibodies or antibody fragments of the present invention have a dissociation constant (Kd) with HER2 protein under a condition in tumor microenvironment of about ≤1 μM, ≤100 nM, ≤10 nM, ≤1 nM, ≤0.1 nM, ≤0.01 nM, or ≤0.001 nM (e.g. 10⁻⁸M or less, or from 10⁻⁸M to 10⁻¹³M, or from 10⁻⁹M to 10⁻¹³M). In one embodiment, the ratio of the Kd of the antibody or antibody fragment with HER2 protein at the condition in tumor microenvironment to the Kd at the same condition in non-tumor microenvironment is at least about 1.5:1, at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 50:1, at least about 70:1, or at least about 100:1.

In one embodiment, Kd is measured by a radiolabeled antigen binding assay (RIA) performed with the Fab version of an antibody of interest and its antigen using the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CMS chips at about 10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

The anti-HER2 antibodies of the invention may be a chimeric, humanized or human antibody. In one embodiment, an anti-HER2 antibody fragment is employed, e.g., a Fv, Fab, Fab′, Fab′-SH, scFv, a diabody, a triabody, a tetrabody or an F(ab′)₂ fragment and multispecific antibodies formed from antibody fragments. In another embodiment, the antibody is a full-length antibody, e.g., an intact IgG antibody or other antibody class or isotype as defined herein. For a review of certain antibody fragments, see Hudson et al. Nat. Med., vol. 9, pp. 129-134, 2003. For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

The diabodies of the invention may be bivalent or bispecific. See, for example, EP 404,097; WO 1993/01161; Hudson et al., Nat. Med. 9:129-134 (2003); and Hollinger et al., Proc. Natl. Acad. Sci. USA, vol. 90, pp. 6444-6448, 1993 for examples of diabodies. Examples of triabodies and tetrabodies are also described in Hudson et al., Nat. Med., vol. 9, pp. 129-134, 2003.

In some embodiments, the invention comprises single-domain antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

In some embodiments, the anti-HER2 antibodies of the invention may be chimeric antibodies. Certain chimeric antibodies are described, e.g., in U.S. Pat. No. 4,816,567; and Morrison et al., Proc. Natl. Acad. Sci. USA, vol. 81, pp. 6851-6855, 1984). In one example, the chimeric antibody comprises a non-human variable region (e.g., a variable region derived from a mouse, rat, hamster, rabbit, or non-human primate, such as a monkey) and a human constant region. In a further example, the chimeric antibody is a “class switched” antibody in which the class or subclass of the antibody has been changed relative to the class or subclass of the parent antibody. Chimeric antibodies include antigen-binding fragments thereof.

In certain embodiments, the chimeric antibody of the invention is a humanized antibody. Typically, such a non-human antibody is humanized to reduce immunogenicity to humans, while retaining the specificity and affinity of the parental non-human antibody. Generally, a humanized antibody comprises one or more variable domains in which CDRs (or portions thereof) are derived from a non-human antibody, and FRs (or portions thereof) are derived from human antibody sequences. A humanized antibody may optionally also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci., vol. 13, pp. 1619-1633, 2008, and are further described, e.g., in Riechmann et al., Nature, vol. 332, pp. 323-329, 1988; Queen et al., Proc. Nat'l Acad. Sci. USA, vol. 86, pp. 10029-10033, 1989; U.S. Pat. Nos. 5,821,337, 7,527,791, 6,982,321, and 7,087,409; Kashmiri et al., Methods, vol. 36, pp. 25-34, 2005 (describing SDR (a-CDR) grafting); Padlan, Mol. Immunol., vol. 28, pp. 489-498, 1991 (describing “resurfacing”); Dall'Acqua et al., Methods, vol. 36, pp. 43-60, 2005 (describing “FR shuffling”); and Osbourn et al., Methods, vol. 36, pp. 61-68, 2005 and Klimka et al., Br. J. Cancer, vol. 83, pp. 252-260, 2000 (describing the “guided selection” approach to FR shuffling).

Human framework regions that may be used for humanization include but are not limited to: framework regions selected using the “best-fit” method (see, e.g., Sims et al. J. Immunol., vol. 151, p. 2296, 1993); framework regions derived from the consensus sequence of human antibodies of a particular subgroup of light or heavy chain variable regions (see, e.g., Carter et al. Proc. Natl. Acad. Sci. USA, vol. 89, p. 4285, 1992; and Presta et al. J. Immunol., vol. 151, p. 2623, 1993); human mature (somatically mutated) framework regions or human germline framework regions (see, e.g., Almagro and Fransson, Front. Biosci., vol. 13, pp. 1619-1633, 2008); and framework regions derived from screening FR libraries (see, e.g., Baca et al., J. Biol. Chem., vol. 272, pp. 10678-10684, 1997 and Rosok et al., J. Biol. Chem., vol. 271, pp. 22611-22618, 1996).

### Multi-Specific Antibodies

The disclosure provides a multi-specific antibody comprising at least one binding site for a cell antigen; and at least one binding site for a tumor-reactive lymphocyte antigen. The multi-specific antibody binds to at least one of the cell antigen and tumor-reactive lymphocyte antigen with a greater activity, affinity and/or avidity at a first physiological condition than at a second physiological condition.

In some embodiments, the first physiological condition is an aberrant condition and the second physiological condition is a normal physiological condition. For example, the aberrant condition may be a condition in a tumor microenvironment. The multi-specific antibody of the present invention may be referred to as a conditionally active multi-specific antibody.

In some embodiments, the conditionally active multi-specific antibody is virtually inactive at a normal physiological condition but is active at an aberrant condition, optionally having a level of activity that is higher than the activity of the conditionally active multi-specific antibody at a normal physiological condition or the activity at a normal physiological condition of the parent antibody from which it is derived. In another embodiment, the conditionally active multi-specific antibody is virtually inactive at a pH of 7.2-7.8, but is active at a lower pH of 5.0-7.0. In some cases, the conditionally active multi-specific antibody is reversibly or irreversibly inactivated at the normal physiological condition. In another example, the conditionally active multi-specific antibody may be more or less active in highly oxygenated blood, such as, for example, after passage through the lung or in the lower pH environments found in the tumor microenvironment. The conditionally active multi-specific antibody may be used as a drug, therapeutic agent or diagnostic agent.

Without wishing to be limited by theory, the multi-specific antibody of the present invention binds to both the target cell and tumor-reactive lymphocyte to thereby bring the target cell in close proximity to the tumor-reactive lymphocyte. This is believed to facilitate an attack by the tumor-reactive lymphocyte on the target cell to thereby inhibit, damage or destroy the target cell. A therapeutic effect of inhibition or removing tumor cells may be achieved by using the multi-specific antibody of the present invention to bring the reactive lymphocyte to tumor cells for inhibition, destruction and removal of the tumor cells from the subject.

The first and second physiological conditions are different numerical values of the same condition which may be selected from temperature, pH, osmotic pressure, osmolality, oxidative stress, oxygen concentration and electrolyte concentration. For example, the first physiological condition may be an acidic pH in a tumor microenvironment in the range of from 5.2 to 7.0 or from 5.8 to 7.0 or from 6.0 to 6.8. The second physiological condition may be a normal physiological pH in the blood of the subject in the range of from 7.2 to 7.8 or from 7.2 to 7.6.

In some embodiments, the first physiological condition is a lower oxygen concentration in a tumor microenvironment and the second physiological condition is a normal physiological oxygen concentration in the blood of the subject. In some embodiments, the conditionally active multi-specific antibody is virtually inactive at a normal physiological condition but is active at an aberrant condition, optionally having a level of activity that is higher than the activity of the conditionally active multi-specific antibody at a normal physiological condition or the activity at a normal physiological condition of the parent antibody from which it is derived. In another embodiment, the conditionally active multi-specific antibody is virtually inactive at a pH of 7.2-7.8, but is active at a lower pH of 5.0-7.0. In some cases, the conditionally active multi-specific antibody is reversibly or irreversibly inactivated at the normal physiological condition. In another example, the conditionally active multi-specific antibody may be more or less active in highly oxygenated blood, such as, for example, after passage through the lung or in the lower pH environments found in the tumor microenvironment. The conditionally active multi-specific antibody may be used as a drug, therapeutic agent or diagnostic agent.

In some embodiments, the binding of the multi-specific antibody to the cell antigen and/or tumor-reactive lymphocyte antigen is reversible. Meaning that the multi-specific antibody may bind to the cell antigen and/or tumor-reactive lymphocyte antigen, followed by separation of the two. The separated multi-specific antibody is capable of binding to the cell antigen and/or tumor-reactive lymphocyte antigen again.

In some embodiments, the cell antigen may be a cell surface antigen or an interior antigen of the cell. The cell may be targeted by the tumor-reactive lymphocyte for inhibition, damage, destruction or killing. The cell may be referred to as a target cell. Thus, the cell may be targeted in a treatment with the multi-specific antibody of the present invention. Specifically, for treatment of some diseases or conditions, cells may be targeted for removal.

In some embodiments, the cell antigen is an antigen preferentially associated with the target cell but less prevalent with other cell types. In this manner, the multi-specific antibody of the present invention can preferentially interact with the target cell. The target cell may be cancer cell. Example of a cancer cell specific antigens include CD3 and HER2.

In one embodiment, the targeted cancer cell is a breast cancer cell in which case the breast cancer cell specific antigen may be HER2 (Human Epidermal growth factor Receptor 2).

The multi-specific antibody binds to at least one cell specific antigen and the reactive lymphocyte antigen, with an increased affinity at the first physiological condition in comparison with the affinity at the second physiological condition. In some embodiments, the multi-specific antibody binds the at least one of the cell specific antigen and the reactive lymphocyte antigen with an increased affinity at the first physiological condition in comparison with the affinity at the second physiological condition. For example, the multi-specific antibody may bind the cell specific antigen with an increased binding affinity at the first physiological condition in comparison with the binding affinity at the second physiological condition, while still binding to the reactive lymphocyte antigen with a non-conditional activity. In another example, the multi-specific antibody binds to the reactive lymphocyte antigen with an increased binding affinity at the first physiological condition in comparison with the binding affinity at the second physiological condition, while still binding to the cell specific antigen with a non-conditional activity. In some embodiments, the multi-specific antibody binds both the cell specific antigen and the reactive lymphocyte antigen with a higher avidity at the first physiological condition in comparison with the avidity at the second physiological condition.

The structure/format of the multi-specific antibody may be any one of the structures/formats described in Brinkmann and Kontermann, “The making of bispecific antibodies,” MABs, vol. 9, pp. 182-212, 2017, or as described in Orcutt et al., Protein Engineering, Design & Selection, 23(4): 221-228 (2010). Specifically, FIG. 2 of Brinkmann and Kontermann describes 19 different structures/formats for bispecific antibodies. These structures/formats include: (1) bispecific antibody conjugates; (2) hybrid bispecific IgG2; (3) “variable domain only” bispecific antibody molecules; (4) CH1/CL fusion proteins; (5) Fab fusion proteins; (6) non-immunoglobulin fusion proteins; (7) Fc-modified IgGs; (8) appended and Fc-modified IgGs; (9) modified Fc and CH3 fusion proteins; (10) appended IgGs-HC fusions; (11) appended IgGs-LC fusions; (12) appended IgGs-HC&LC fusions; (13) Fc fusions; (14) CH3 fusions; (15) IgE/IgM CH2 fusions; (16) F(ab′)₂ fusion; (17) CH1/CL fusion proteins; (18) modified IgGs; and (19) non-immunoglobulin fusions. Similarly, Orcutt describes bispecific antibody (bsAb) format in which a disulfide-stabilized scFv is fused to the C-terminus of the light chain of an IgG to create an IgG-scFv bifunctional antibody. The structure of the heavy chain, light chain, and fully assembled bsAB with N- and C-termini indicated, is shown in FIG. 1 of Orcutt.

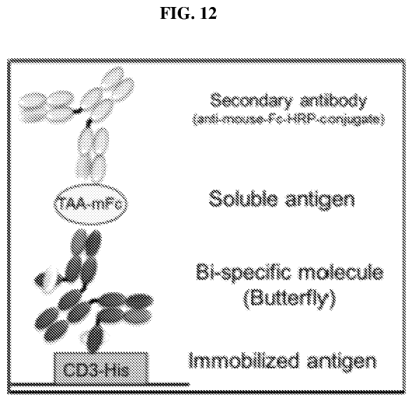

In particular embodiments, the multi-specific antibody may be a bi-valent scFv-Fc hetero-dimer or a tetra-valent homodimer “butterfly” as shown in FIG. 12. In these two structures, the reactive lymphocyte antigens are not limited to CD3, which is only depicted as a representative of a tumor-reactive lymphocyte antigen. The multi-specific antibody of FIG. 12 has a first binding site to a cell antigen (Ag), which is linked to a first heavy chain constant region (e.g., IgG) and a second binding site to a reactive lymphocyte antigen (e.g., CD3), which is linked to a second heavy chain constant region (e.g., IgG). The two heavy chains are engineered such that they can only form hetero dimers, for example, by using the knob-in-hole technique. The first and second binding sites are scFv antibodies binding to the cell antigen and reactive lymphocyte antigen, respectively. Either one or both of the first and second binding sites have a conditionally active binding activity to the respective antigen.

The multi-specific antibody of FIG. 12 may have a full-length IgG antibody binding to the cell specific antigen (Ag) and a scFv antibody binding to a reactive lymphocyte antigen (e.g., CD3). The scFv antibody is linked to the C terminus of the light chain of the IgG antibody via a linker. The linker may be a short Alanine linker (Ala)ₙ, a Serine linker (Ser)ₙ, a hydrophilic linker or a glycine-serine-rich linker. The heavy chain of the IgG antibody pairs with the light chain of the IgG antibody that has been linked to the scFv antibody, thus forming half of the homo-dimer. This multi-specific antibody has a “butterfly” configuration.

In some embodiments, the multi-specific antibody comprises an IgG antibody or fragment thereof that binds to a tumor-reactive lymphocyte antigen and a single chain antibody that binds to a tumor cell antigen, also forming a “butterfly” configuration as shown in FIG. 12. The single chain antibody may be an scFv antibody. The scFv antibody may be attached to a C terminus of the IgG antibody via a linker as described herein.

The binding sites of the multi-specific antibody of the invention each comprise a light chain variable region and a heavy chain variable region. The light chain variable region and the heavy chain variable region may be a single chain antibody format or may be a two-chain format as formed by pairing of a light chain and heavy chain. In a binding site that has a conditional activity, one of the light and heavy chain variable regions is conditionally active or both may be conditionally active.

In some embodiments, the anti-HER2 antibodies of the invention are multispecific, e.g. bispecific antibodies. Multispecific antibodies are monoclonal antibodies that have binding specificities for at least two different sites. In certain embodiments, one of the binding specificities is for HER2 protein and the other is for another antigen. In certain embodiments, bispecific antibodies may bind to two different epitopes of HER2 protein. Bispecific antibodies may also be used to localize cytotoxic agents to cells which express HER2 protein. Bispecific antibodies can be prepared as full length antibodies or antibody fragments.

In some embodiments, the multi-specific antibody or antibody fragmente comprises a heavy chain variable region including three anti-HER2 complementarity determining regions, H1, H2, and H3, wherein:

the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;

wherein X₁ is N or W, X₂ is R or K, X₃ is Y or K or D, X₄ is N or A, X₅ is G or K, X₆ is T or D, X₇ is R or E and X₈ is A or E; and

a light chain variable region including three anti-HER2 complementarity determining regions having sequences L1, L2, and L3, and six anti-CD3 complementarity determining regions, L4, L5, L6, L7, L8, and L9 wherein:

the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,

wherein X₉ is A or D and X₁₀ is H or D or E; and
provided that when X₁-X₈ are N, R, Y, N, G, T, R and A, respectively, X₉ is not A and X₁₀ is not H; and

the L4 sequence is
GFTFNTYAMN,
(SEQ ID NO: 54)
the L5 sequence is
(SEQ ID NO: 55)
RIRSKYNNYATYYADSVKD,
the L6 sequence is
(SEQ ID NO: 70)
HX₁₁NFX₁₂NSKVSWFX₁₃Y,
the L7 sequence is
(SEQ ID NO: 71)
RSSXGAVTTSNYDN,
the L8 sequence is
(SEQ ID NO: 58)
GTNKRAP,
and
the L9 sequence is
(SEQ ID NO: 59)
ALWYSNLWV,

wherein X₁₁ is G, S, A or T, X₁₂ is G or P, X₁₃ is A or Q, and X₁₄ is T or A.

In certain embodiments the multi-specific antibody or antibody fragment comprises a heavy chain variable region including three anti-HER2 complementarity determining regions, H1, H2, and H3, wherein:

the H1 sequence is SEQ ID NO: 50,
the H2 sequence is SEQ ID NO: 49, SEQ ID NO: 9, SEQ ID NO: 12, or SEQ ID NO: 13, and
the H3 sequence is SEQ ID NO: 51, and
a light chain variable region including three anti-HER2 complementarity determining regions L1, L2, and L3 and six anti-CD3 complementarity determining regions, L4, L5, L6, L7, L8, and L9 wherein:

the L4 sequence is
(SEQ ID NO: 54)
GFTFNTYAMN,
the L5 sequence is
(SEQ ID NO: 55)
RIRSKYNNYATYYADSVKD,
the L6 sequence is
(SEQ ID NO: 70)
HX₁₁NFX₁₂NSKVSWFX₁₃Y,
the L7 sequence is
(SEQ ID NO: 71)
RSSXGAVTTSNYDN,
the L8 sequence is
(SEQ ID NO: 58)
GTNKRAP,
and
the L9 sequence is
(SEQ ID NO: 59)
ALWYSNLWV,

wherein X₁₁ is G, S, A or T, X₁₂ is G or P, X₁₃ is A or Q, and X₁₄ is T or A.

In another aspect of the multi-specific antibody or antibody fragment, the L6 sequence is any one of SEQ ID NOs: 56 and 60-67, and the L7 sequence is SEQ ID NO: 57, 68 or 69.

In specific embodiments of the invention, the multi-specific antibodies or antibody fragments are bi-specific antibodies that bind to HER2 and CD3. Such multi-specific antibodies or antibody fragments may be selected from each of the following combinations of a heavy chain variable region and a light chain variable region as set forth below.

### Exemplary Bi-Specific Antibodies

All combinations of heavy and light chain variable regions set forth in Table 10 below as well as:

| Heavy chain region | + | Light Chain region |
|---|---|---|
| SEQ ID NO: 34 | | SEQ ID NO: 40 |
| SEQ ID NO: 34 | | SEQ ID NO: 41 |
| SEQ ID NO: 34 | | SEQ ID NO: 42 |
| SEQ ID NO: 34 | | SEQ ID NO: 43 |
| SEQ ID NO: 34 | | SEQ ID NO: 44 |
| SEQ ID NO: 34 | | SEQ ID NO: 45 |
| SEQ ID NO: 34 | | SEQ ID NO: 46 |
| SEQ ID NO: 34 | | SEQ ID NO: 47 |
| SEQ ID NO: 34 | | SEQ ID NO: 48 |
| SEQ ID NO: 35 | | SEQ ID NO: 40 |
| SEQ ID NO: 36 | | SEQ ID NO: 40 |
| SEQ ID NO: 37 | | SEQ ID NO: 40 |
| SEQ ID NO: 38 | | SEQ ID NO: 40 |
| SEQ ID NO: 39 | | SEQ ID NO: 40 |
| SEQ ID NO: 19 | | SEQ ID NO: 40 |
| SEQ ID NO: 19 | | SEQ ID NO: 41 |
| SEQ ID NO: 19 | | SEQ ID NO: 42 |
| SEQ ID NO: 19 | | SEQ ID NO: 43 |
| SEQ ID NO: 19 | | SEQ ID NO: 44 |
| SEQ ID NO: 19 | | SEQ ID NO: 45 |
| SEQ ID NO: 19 | | SEQ ID NO: 46 |
| SEQ ID NO: 19 | | SEQ ID NO: 47 |
| SEQ ID NO: 19 | | SEQ ID NO: 48 |
| SEQ ID NO: 20 | | SEQ ID NO: 40 |
| SEQ ID NO: 20 | | SEQ ID NO: 41 |
| SEQ ID NO: 20 | | SEQ ID NO: 42 |
| SEQ ID NO: 20 | | SEQ ID NO: 43 |
| SEQ ID NO: 20 | | SEQ ID NO: 44 |
| SEQ ID NO: 20 | | SEQ ID NO: 45 |
| SEQ ID NO: 20 | | SEQ ID NO: 46 |
| SEQ ID NO: 20 | | SEQ ID NO: 47 |
| SEQ ID NO: 20 | | SEQ ID NO: 48 |
| SEQ ID NO: 21 | | SEQ ID NO: 40 |
| SEQ ID NO: 21 | | SEQ ID NO: 41 |
| SEQ ID NO: 21 | | SEQ ID NO: 42 |
| SEQ ID NO: 21 | | SEQ ID NO: 43 |
| SEQ ID NO: 21 | | SEQ ID NO: 44 |
| SEQ ID NO: 21 | | SEQ ID NO: 45 |
| SEQ ID NO: 21 | | SEQ ID NO: 46 |
| SEQ ID NO: 21 | | SEQ ID NO: 47 |
| SEQ ID NO: 21 | | SEQ ID NO: 48 |
| SEQ ID NO: 22 | | SEQ ID NO: 40 |
| SEQ ID NO: 22 | | SEQ ID NO: 41 |
| SEQ ID NO: 22 | | SEQ ID NO: 42 |
| SEQ ID NO: 22 | | SEQ ID NO: 43 |
| SEQ ID NO: 22 | | SEQ ID NO: 44 |
| SEQ ID NO: 22 | | SEQ ID NO: 45 |
| SEQ ID NO: 22 | | SEQ ID NO: 46 |
| SEQ ID NO: 22 | | SEQ ID NO: 47 |
| SEQ ID NO: 22 | | SEQ ID NO: 48 |
| SEQ ID NO: 23 | | SEQ ID NO: 40 |
| SEQ ID NO: 23 | | SEQ ID NO: 41 |
| SEQ ID NO: 23 | | SEQ ID NO: 42 |
| SEQ ID NO: 23 | | SEQ ID NO: 43 |
| SEQ ID NO: 23 | | SEQ ID NO: 44 |
| SEQ ID NO: 23 | | SEQ ID NO: 45 |
| SEQ ID NO: 23 | | SEQ ID NO: 46 |
| SEQ ID NO: 23 | | SEQ ID NO: 47 |
| SEQ ID NO: 23 | | SEQ ID NO: 48 |
| SEQ ID NO: 24 | | SEQ ID NO: 40 |
| SEQ ID NO: 24 | | SEQ ID NO: 41 |
| SEQ ID NO: 24 | | SEQ ID NO: 42 |
| SEQ ID NO: 24 | | SEQ ID NO: 43 |
| SEQ ID NO: 24 | | SEQ ID NO: 44 |
| SEQ ID NO: 24 | | SEQ ID NO: 45 |
| SEQ ID NO: 24 | | SEQ ID NO: 46 |
| SEQ ID NO: 24 | | SEQ ID NO: 47 |
| SEQ ID NO: 24 | | SEQ ID NO: 48 |
| SEQ ID NO: 25 | | SEQ ID NO: 40 |
| SEQ ID NO: 25 | | SEQ ID NO: 41 |
| SEQ ID NO: 25 | | SEQ ID NO: 42 |
| SEQ ID NO: 25 | | SEQ ID NO: 43 |
| SEQ ID NO: 25 | | SEQ ID NO: 44 |
| SEQ ID NO: 25 | | SEQ ID NO: 45 |
| SEQ ID NO: 25 | | SEQ ID NO: 46 |
| SEQ ID NO: 25 | | SEQ ID NO: 47 |
| SEQ ID NO: 25 | | SEQ ID NO: 48 |
| SEQ ID NO: 26 | | SEQ ID NO: 40 |
| SEQ ID NO: 26 | | SEQ ID NO: 41 |
| SEQ ID NO: 26 | | SEQ ID NO: 42 |
| SEQ ID NO: 26 | | SEQ ID NO: 43 |
| SEQ ID NO: 26 | | SEQ ID NO: 44 |
| SEQ ID NO: 26 | | SEQ ID NO: 45 |
| SEQ ID NO: 26 | | SEQ ID NO: 46 |
| SEQ ID NO: 26 | | SEQ ID NO: 47 |
| SEQ ID NO: 26 | | SEQ ID NO: 48 |
| SEQ ID NO: 27 | | SEQ ID NO: 40 |
| SEQ ID NO: 27 | | SEQ ID NO: 41 |
| SEQ ID NO: 27 | | SEQ ID NO: 42 |
| SEQ ID NO: 27 | | SEQ ID NO: 43 |
| SEQ ID NO: 27 | | SEQ ID NO: 44 |
| SEQ ID NO: 27 | | SEQ ID NO: 45 |
| SEQ ID NO: 27 | | SEQ ID NO: 46 |
| SEQ ID NO: 27 | | SEQ ID NO: 47 |
| SEQ ID NO: 27 | | SEQ ID NO: 48 |
| SEQ ID NO: 28 | | SEQ ID NO: 40 |
| SEQ ID NO: 28 | | SEQ ID NO: 41 |
| SEQ ID NO: 28 | | SEQ ID NO: 42 |
| SEQ ID NO: 28 | | SEQ ID NO: 43 |
| SEQ ID NO: 28 | | SEQ ID NO: 44 |
| SEQ ID NO: 28 | | SEQ ID NO: 45 |
| SEQ ID NO: 28 | | SEQ ID NO: 46 |
| SEQ ID NO: 28 | | SEQ ID NO: 47 |
| SEQ ID NO: 28 | | SEQ ID NO: 48 |
| SEQ ID NO: 33 | | SEQ ID NO: 40 |
| SEQ ID NO: 33 | | SEQ ID NO: 41 |
| SEQ ID NO: 33 | | SEQ ID NO: 42 |
| SEQ ID NO: 33 | | SEQ ID NO: 43 |
| SEQ ID NO: 33 | | SEQ ID NO: 44 |
| SEQ ID NO: 33 | | SEQ ID NO: 45 |
| SEQ ID NO: 33 | | SEQ ID NO: 46 |
| SEQ ID NO: 33 | | SEQ ID NO: 47 |
| SEQ ID NO: 33 | | SEQ ID NO: 48 |

The preferred bi-specific antibodies are those having the combinations of heavy and light chain variable regions set forth in Table 10 below.

In another specific embodiment of the invention, the multi-specific antibodies are bi-specific antibodies that bind to Her2 and CD3, comprising a heavy chain variable region and a light chain variable region. The heavy chain variable region includes H1, H2, H3 sequences, in which each may be selected from any of the following combinations as set forth below. The light chain variable region includes L1, L2, L3, L4, L5, L6, L7, L8 and L9 sequences, in which each may be selected from any of the following combination as set forth below.

In specific embodiments of the invention, the multi-specific antibodies are bi-specific antibodies that may be selected from any of the following antibodies which comprise each specific combination of twelve CDRs H1, H2, H3, L1, L2, L3, L4, L5, L6, L7, L8, and L9 set forth below.

Exemplary Bi-Specific Antibodies

| H1, H2, H3 CDRs | L1, L2, L3, L4, L5, L6, L7, L8, L9 CDRs |
|---|---|
| All possible 50 + (8-14 or 49) + | All possible (52 or 16) + 5 + 53 + 54 + 55 + |
| 51 or 15 | (56 or 60-67) + (57, 68 or 69) + 58 + 59 |
| SEQ ID NOs: 50 + 8 + 51 | SEQ ID NOs: 52 + 5 + 53 + 54 + 55 + 56 + 57 + 58 + 59 |
| SEQ ID NOs: 50 + 9 + 51 | SEQ ID NOs: 52 + 5 + 53 + 54 + 55 + 56 + 57 + 58 + 59 |
| SEQ ID NOs: 50 + 10 + 51 | SEQ ID NOs: 52 + 5 + 53 + 54 + 55 + 56 + 57 + 58 + 59 |
| SEQ ID NOs: 50 + 11 + 51 | SEQ ID NOs: 52 + 5 + 53 + 54 + 55 + 56 + 57 + 58 + 59 |
| SEQ ID NOs: 50 + 12 + 51 | SEQ ID NOs: 52 + 5 + 53 + 54 + 55 + 56 + 57 + 58 + 59 |
| SEQ ID NOs: 50 + 13 + 51 | SEQ ID NOs: 52 + 5 + 53 + 54 + 55 + 56 + 57 + 58 + 59 |
| SEQ ID NOs: 50 + 14 + 51 | SEQ ID NOs: 52 + 5 + 53 + 54 + 55 + 56 + 57 + 58 + 59 |
| SEQ ID NOs: 50 + 49 + 51 | SEQ ID NOs: 52 + 5 + 53 + 54 + 55 + 56 + 57 + 58 + 59 |
| SEQ ID NOs: 50 + 8 + 51 | SEQ ID NOs: 52 + 5 + 53 + 54 + 55 + 56 + 68 + 58 + 59 |
| SEQ ID NOs: 50 + 9 + 51 | SEQ ID NOs: 52 + 5 + 53 + 54 + 55 + 56 + 68 + 58 + 59 |
| SEQ ID NOs: 50 + 10 + 51 | SEQ ID NOs: 52 + 5 + 53 + 54 + 55 + 56 + 68 + 58 + 59 |
| SEQ ID NOs: 50 + 11 + 51 | SEQ ID NOs: 52 + 5 + 53 + 54 + 55 + 56 + 68 + 58 + 59 |
| SEQ ID NOs: 50 + 12 + 51 | SEQ ID NOs: 52 + 5 + 53 + 54 + 55 + 56 + 68 + 58 + 59 |
| SEQ ID NOs: 50 + 13 + 51 | SEQ ID NOs: 52 + 5 + 53 + 54 + 55 + 56 + 68 + 58 + 59 |
| SEQ ID NOs: 50 + 14 + 51 | SEQ ID NOs: 52 + 5 + 53 + 54 + 55 + 56 + 68 + 58 + 59 |
| SEQ ID NOs: 50 + 49 + 51 | SEQ ID NOs: 52 + 5 + 53 + 54 + 55 + 56 + 68 + 58 + 59 |
| SEQ ID NOs: 50 + 8 + 51 | SEQ ID NOs: 52 + 5 + 53 + 54 + 55 + 56 + 69 + 58 + 59 |
| SEQ ID NOs: 50 + 9 + 51 | SEQ ID NOs: 52 + 5 + 53 + 54 + 55 + 56 + 69 + 58 + 59 |
| SEQ ID NOs: 50 + 10 + 51 | SEQ ID NOs: 52 + 5 + 53 + 54 + 55 + 56 + 69 + 58 + 59 |
| SEQ ID NOs: 50 + 11 + 51 | SEQ ID NOs: 52 + 5 + 53 + 54 + 55 + 56 + 69 + 58 + 59 |
| SEQ ID NOs: 50 + 12 + 51 | SEQ ID NOs: 52 + 5 + 53 + 54 + 55 + 56 + 69 + 58 + 59 |
| SEQ ID NOs: 50 + 13 + 51 | SEQ ID NOs: 52 + 5 + 53 + 54 + 55 + 56 + 69 + 58 + 59 |
| SEQ ID NOs: 50 + 14 + 51 | SEQ ID NOs: 52 + 5 + 53 + 54 + 55 + 56 + 69 + 58 + 59 |
| SEQ ID NOs: 50 + 49 + 51 | SEQ ID NOs: 52 + 5 + 53 + 54 + 55 + 56 + 69 + 58 + 59 |
| SEQ ID NOs: 50 + 8 + 51 | SEQ ID NOs: 52 + 5 + 53 + 54 + 55 + 60 + 57 + 58 + 59 |
| SEQ ID NOs: 50 + 9 + 51 | SEQ ID NOs: 52 + 5 + 53 + 54 + 55 + 60 + 57 + 58 + 59 |
| SEQ ID NOs: 50 + 10 + 51 | SEQ ID NOs: 52 + 5 + 53 + 54 + 55 + 60 + 57 + 58 + 59 |
| SEQ ID NOs: 50 + 11 + 51 | SEQ ID NOs: 52 + 5 + 53 + 54 + 55 + 60 + 57 + 58 + 59 |
| SEQ ID NOs: 50 + 12 + 51 | SEQ ID NOs: 52 + 5 + 53 + 54 + 55 + 60 + 57 + 58 + 59 |
| SEQ ID NOs: 50 + 13 + 51 | SEQ ID NOs: 52 + 5 + 53 + 54 + 55 + 60 + 57 + 58 + 59 |
| SEQ ID NOs: 50 + 14 + 51 | SEQ ID NOs: 52 + 5 + 53 + 54 + 55 + 60 + 57 + 58 + 59 |
| SEQ ID NOs: 50 + 49 + 51 | SEQ ID NOs: 52 + 5 + 53 + 54 + 55 + 60 + 57 + 58 + 59 |
| SEQ ID NOs: 50 + 8 + 51 | SEQ ID NOs: 52 + 5 + 53 + 54 + 55 + 60 + 68 + 58 + 59 |
| SEQ ID NOs: 50 + 9 + 51 | SEQ ID NOs: 52 + 5 + 53 + 54 + 55 + 60 + 68 + 58 + 59 |
| SEQ ID NOs: 50 + 10 + 51 | SEQ ID NOs: 52 + 5 + 53 + 54 + 55 + 60 + 68 + 58 + 59 |
| SEQ ID NOs: 50 + 11 + 51 | SEQ ID NOs: 52 + 5 + 53 + 54 + 55 + 60 + 68 + 58 + 59 |
| SEQ ID NOs: 50 + 12 + 51 | SEQ ID NOs: 52 + 5 + 53 + 54 + 55 + 60 + 68 + 58 + 59 |
| SEQ ID NOs: 50 + 13 + 51 | SEQ ID NOs: 52 + 5 + 53 + 54 + 55 + 60 + 68 + 58 + 59 |
| SEQ ID NOs: 50 + 14 + 51 | SEQ ID NOs: 52 + 5 + 53 + 54 + 55 + 60 + 68 + 58 + 59 |
| SEQ ID NOs: 50 + 49 + 51 | SEQ ID NOs: 52 + 5 + 53 + 54 + 55 + 60 + 68 + 58 + 59 |
| SEQ ID NOs: 50 + 8 + 51 | SEQ ID NOs: 52 + 5 + 53 + 54 + 55 + 60 + 69 + 58 + 59 |
| SEQ ID NOs: 50 + 9 + 51 | SEQ ID NOs: 52 + 5 + 53 + 54 + 55 + 60 + 69 + 58 + 59 |
| SEQ ID NOs: 50 + 10 + 51 | SEQ ID NOs: 52 + 5 + 53 + 54 + 55 + 60 + 69 + 58 + 59 |
| SEQ ID NOs: 50 + 11 + 51 | SEQ ID NOs: 52 + 5 + 53 + 54 + 55 + 60 + 69 + 58 + 59 |
| SEQ ID NOs: 50 + 12 + 51 | SEQ ID NOs: 52 + 5 + 53 + 54 + 55 + 60 + 69 + 58 + 59 |
| SEQ ID NOs: 50 + 13 + 51 | SEQ ID NOs: 52 + 5 + 53 + 54 + 55 + 60 + 69 + 58 + 59 |
| SEQ ID NOs: 50 + 14 + 51 | SEQ ID NOs: 52 + 5 + 53 + 54 + 55 + 60 + 69 + 58 + 59 |
| SEQ ID NOs: 50 + 49 + 51 | SEQ ID NOs: 52 + 5 + 53 + 54 + 55 + 60 + 69 + 58 + 59 |
| SEQ ID NOs: 50 + 8 + 51 | SEQ ID NOs: 52 + 5 + 53 + 54 + 55 + 61 + 57 + 58 + 59 |
| SEQ ID NOs: 50 + 9 + 51 | SEQ ID NOs: 52 + 5 + 53 + 54 + 55 + 61 + 57 + 58 + 59 |
| SEQ ID NOs: 50 + 10 + 51 | SEQ ID NOs: 52 + 5 + 53 + 54 + 55 + 61 + 57 + 58 + 59 |
| SEQ ID NOs: 50 + 11 + 51 | SEQ ID NOs: 52 + 5 + 53 + 54 + 55 + 61 + 57 + 58 + 59 |
| SEQ ID NOs: 50 + 12 + 51 | SEQ ID NOs: 52 + 5 + 53 + 54 + 55 + 61 + 57 + 58 + 59 |
| SEQ ID NOs: 50 + 13 + 51 | SEQ ID NOs: 52 + 5 + 53 + 54 + 55 + 61 + 57 + 58 + 59 |
| SEQ ID NOs: 50 + 14 + 51 | SEQ ID NOs: 52 + 5 + 53 + 54 + 55 + 61 + 57 + 58 + 59 |
| SEQ ID NOs: 50 + 49 + 51 | SEQ ID NOs: 52 + 5 + 53 + 54 + 55 + 61 + 57 + 58 + 59 |
| SEQ ID NOs: 50 + 8 + 51 | SEQ ID NOs: 52 + 5 + 53 + 54 + 55 + 61 + 68 + 58 + 59 |
| SEQ ID NOs: 50 + 9 + 51 | SEQ ID NOs: 52 + 5 + 53 + 54 + 55 + 61 + 68 + 58 + 59 |
| SEQ ID NOs: 50 + 10 + 51 | SEQ ID NOs: 52 + 5 + 53 + 54 + 55 + 61 + 68 + 58 + 59 |
| SEQ ID NOs: 50 + 11 + 51 | SEQ ID NOs: 52 + 5 + 53 + 54 + 55 + 61 + 68 + 58 + 59 |
| SEQ ID NOs: 50 + 12 + 51 | SEQ ID NOs: 52 + 5 + 53 + 54 + 55 + 61 + 68 + 58 + 59 |
| SEQ ID NOs: 50 + 13 + 51 | SEQ ID NOs: 52 + 5 + 53 + 54 + 55 + 61 + 68 + 58 + 59 |
| SEQ ID NOs: 50 + 14 + 51 | SEQ ID NOs: 52 + 5 + 53 + 54 + 55 + 61 + 68 + 58 + 59 |
| SEQ ID NOs: 50 + 49 + 51 | SEQ ID NOs: 52 + 5 + 53 + 54 + 55 + 61 + 68 + 58 + 59 |
| SEQ ID NOs: 50 + 8 + 51 | SEQ ID NOs: 52 + 5 + 53 + 54 + 55 + 61 + 69 + 58 + 59 |
| SEQ ID NOs: 50 + 9 + 51 | SEQ ID NOs: 52 + 5 + 53 + 54 + 55 + 61 + 69 + 58 + 59 |
| SEQ ID NOs: 50 + 10 + 51 | SEQ ID NOs: 52 + 5 + 53 + 54 + 55 + 61 + 69 + 58 + 59 |
| SEQ ID NOs: 50 + 11 + 51 | SEQ ID NOs: 52 + 5 + 53 + 54 + 55 + 61 + 69 + 58 + 59 |
| SEQ ID NOs: 50 + 12 + 51 | SEQ ID NOs: 52 + 5 + 53 + 54 + 55 + 61 + 69 + 58 + 59 |
| SEQ ID NOs: 50 + 13 + 51 | SEQ ID NOs: 52 + 5 + 53 + 54 + 55 + 61 + 69 + 58 + 59 |
| SEQ ID NOs: 50 + 14 + 51 | SEQ ID NOs: 52 + 5 + 53 + 54 + 55 + 61 + 69 + 58 + 59 |
| SEQ ID NOs: 50 + 49 + 51 | SEQ ID NOs: 52 + 5 + 53 + 54 + 55 + 61 + 69 + 58 + 59 |
| SEQ ID NOs: 50 + 8 + 51 | SEQ ID NOs: 52 + 5 + 53 + 54 + 55 + 62 + 57 + 58 + 59 |
| SEQ ID NOs: 50 + 9 + 51 | SEQ ID NOs: 52 + 5 + 53 + 54 + 55 + 62 + 57 + 58 + 59 |
| SEQ ID NOs: 50 + 10 + 51 | SEQ ID NOs: 52 + 5 + 53 + 54 + 55 + 62 + 57 + 58 + 59 |
| SEQ ID NOs: 50 + 11 + 51 | SEQ ID NOs: 52 + 5 + 53 + 54 + 55 + 62 + 57 + 58 + 59 |
| SEQ ID NOs: 50 + 12 + 51 | SEQ ID NOs: 52 + 5 + 53 + 54 + 55 + 62 + 57 + 58 + 59 |
| SEQ ID NOs: 50 + 13 + 51 | SEQ ID NOs: 52 + 5 + 53 + 54 + 55 + 62 + 57 + 58 + 59 |
| SEQ ID NOs: 50 + 14 + 51 | SEQ ID NOs: 52 + 5 + 53 + 54 + 55 + 62 + 57 + 58 + 59 |
| SEQ ID NOs: 50 + 49 + 51 | SEQ ID NOs: 52 + 5 + 53 + 54 + 55 + 62 + 57 + 58 + 59 |
| SEQ ID NOs: 50 + 8 + 51 | SEQ ID NOs: 52 + 5 + 53 + 54 + 55 + 62 + 68 + 58 + 59 |
| SEQ ID NOs: 50 + 9 + 51 | SEQ ID NOs: 52 + 5 + 53 + 54 + 55 + 62 + 68 + 58 + 59 |
| SEQ ID NOs: 50 + 10 + 51 | SEQ ID NOs: 52 + 5 + 53 + 54 + 55 + 62 + 68 + 58 + 59 |
| SEQ ID NOs: 50 + 11 + 51 | SEQ ID NOs: 52 + 5 + 53 + 54 + 55 + 62 + 68 + 58 + 59 |
| SEQ ID NOs: 50 + 12 + 51 | SEQ ID NOs: 52 + 5 + 53 + 54 + 55 + 62 + 68 + 58 + 59 |
| SEQ ID NOs: 50 + 13 + 51 | SEQ ID NOs: 52 + 5 + 53 + 54 + 55 + 62 + 68 + 58 + 59 |
| SEQ ID NOs: 50 + 14 + 51 | SEQ ID NOs: 52 + 5 + 53 + 54 + 55 + 62 + 68 + 58 + 59 |
| SEQ ID NOs: 50 + 49 + 51 | SEQ ID NOs: 52 + 5 + 53 + 54 + 55 + 62 + 68 + 58 + 59 |
| SEQ ID NOs: 50 + 8 + 51 | SEQ ID NOs: 52 + 5 + 53 + 54 + 55 + 62 + 69 + 58 + 59 |
| SEQ ID NOs: 50 + 9 + 51 | SEQ ID NOs: 52 + 5 + 53 + 54 + 55 + 62 + 69 + 58 + 59 |
| SEQ ID NOs: 50 + 10 + 51 | SEQ ID NOs: 52 + 5 + 53 + 54 + 55 + 62 + 69 + 58 + 59 |
| SEQ ID NOs: 50 + 11 + 51 | SEQ ID NOs: 52 + 5 + 53 + 54 + 55 + 62 + 69 + 58 + 59 |
| SEQ ID NOs: 50 + 12 + 51 | SEQ ID NOs: 52 + 5 + 53 + 54 + 55 + 62 + 69 + 58 + 59 |
| SEQ ID NOs: 50 + 13 + 51 | SEQ ID NOs: 52 + 5 + 53 + 54 + 55 + 62 + 69 + 58 + 59 |
| SEQ ID NOs: 50 + 14 + 51 | SEQ ID NOs: 52 + 5 + 53 + 54 + 55 + 62 + 69 + 58 + 59 |
| SEQ ID NOs: 50 + 49 + 51 | SEQ ID NOs: 52 + 5 + 53 + 54 + 55 + 62 + 69 + 58 + 59 |
| SEQ ID NOs: 50 + 8 + 51 | SEQ ID NOs: 52 + 5 + 53 + 54 + 55 + 63 + 57 + 58 + 59 |
| SEQ ID NOs: 50 + 9 + 51 | SEQ ID NOs: 52 + 5 + 53 + 54 + 55 + 63 + 57 + 58 + 59 |
| SEQ ID NOs: 50 + 10 + 51 | SEQ ID NOs: 52 + 5 + 53 + 54 + 55 + 63 + 57 + 58 + 59 |
| SEQ ID NOs: 50 + 11 + 51 | SEQ ID NOs: 52 + 5 + 53 + 54 + 55 + 63 + 57 + 58 + 59 |
| SEQ ID NOs: 50 + 12 + 51 | SEQ ID NOs: 52 + 5 + 53 + 54 + 55 + 63 + 57 + 58 + 59 |
| SEQ ID NOs: 50 + 13 + 51 | SEQ ID NOs: 52 + 5 + 53 + 54 + 55 + 63 + 57 + 58 + 59 |
| SEQ ID NOs: 50 + 14 + 51 | SEQ ID NOs: 52 + 5 + 53 + 54 + 55 + 63 + 57 + 58 + 59 |
| SEQ ID NOs: 50 + 49 + 51 | SEQ ID NOs: 52 + 5 + 53 + 54 + 55 + 63 + 57 + 58 + 59 |
| SEQ ID NOs: 50 + 8 + 51 | SEQ ID NOs: 52 + 5 + 53 + 54 + 55 + 63 + 68 + 58 + 59 |
| SEQ ID NOs: 50 + 9 + 51 | SEQ ID NOs: 52 + 5 + 53 + 54 + 55 + 63 + 68 + 58 + 59 |
| SEQ ID NOs: 50 + 10 + 51 | SEQ ID NOs: 52 + 5 + 53 + 54 + 55 + 63 + 68 + 58 + 59 |
| SEQ ID NOs: 50 + 11 + 51 | SEQ ID NOs: 52 + 5 + 53 + 54 + 55 + 63 + 68 + 58 + 59 |
| SEQ ID NOs: 50 + 12 + 51 | SEQ ID NOs: 52 + 5 + 53 + 54 + 55 + 63 + 68 + 58 + 59 |
| SEQ ID NOs: 50 + 13 + 51 | SEQ ID NOs: 52 + 5 + 53 + 54 + 55 + 63 + 68 + 58 + 59 |
| SEQ ID NOs: 50 + 14 + 51 | SEQ ID NOs: 52 + 5 + 53 + 54 + 55 + 63 + 68 + 58 + 59 |
| SEQ ID NOs: 50 + 49 + 51 | SEQ ID NOs: 52 + 5 + 53 + 54 + 55 + 63 + 68 + 58 + 59 |
| SEQ ID NOs: 50 + 8 + 51 | SEQ ID NOs: 52 + 5 + 53 + 54 + 55 + 63 + 69 + 58 + 59 |
| SEQ ID NOs: 50 + 9 + 51 | SEQ ID NOs: 52 + 5 + 53 + 54 + 55 + 63 + 69 + 58 + 59 |
| SEQ ID NOs: 50 + 10 + 51 | SEQ ID NOs: 52 + 5 + 53 + 54 + 55 + 63 + 69 + 58 + 59 |
| SEQ ID NOs: 50 + 11 + 51 | SEQ ID NOs: 52 + 5 + 53 + 54 + 55 + 63 + 69 + 58 + 59 |
| SEQ ID NOs: 50 + 12 + 51 | SEQ ID NOs: 52 + 5 + 53 + 54 + 55 + 63 + 69 + 58 + 59 |
| SEQ ID NOs: 50 + 13 + 51 | SEQ ID NOs: 52 + 5 + 53 + 54 + 55 + 63 + 69 + 58 + 59 |
| SEQ ID NOs: 50 + 14 + 51 | SEQ ID NOs: 52 + 5 + 53 + 54 + 55 + 63 + 69 + 58 + 59 |
| SEQ ID NOs: 50 + 49 + 51 | SEQ ID NOs: 52 + 5 + 53 + 54 + 55 + 63 + 69 + 58 + 59 |
| SEQ ID NOs: 50 + 8 + 51 | SEQ ID NOs: 52 + 5 + 53 + 54 + 55 + 64 + 57 + 58 + 59 |
| SEQ ID NOs: 50 + 9 + 51 | SEQ ID NOs: 52 + 5 + 53 + 54 + 55 + 64 + 57 + 58 + 59 |
| SEQ ID NOs: 50 + 10 + 51 | SEQ ID NOs: 52 + 5 + 53 + 54 + 55 + 64 + 57 + 58 + 59 |
| SEQ ID NOs: 50 + 11 + 51 | SEQ ID NOs: 52 + 5 + 53 + 54 + 55 + 64 + 57 + 58 + 59 |
| SEQ ID NOs: 50 + 12 + 51 | SEQ ID NOs: 52 + 5 + 53 + 54 + 55 + 64 + 57 + 58 + 59 |
| SEQ ID NOs: 50 + 13 + 51 | SEQ ID NOs: 52 + 5 + 53 + 54 + 55 + 64 + 57 + 58 + 59 |
| SEQ ID NOs: 50 + 14 + 51 | SEQ ID NOs: 52 + 5 + 53 + 54 + 55 + 64 + 57 + 58 + 59 |
| SEQ ID NOs: 50 + 49 + 51 | SEQ ID NOs: 52 + 5 + 53 + 54 + 55 + 64 + 57 + 58 + 59 |
| SEQ ID NOs: 50 + 8 + 51 | SEQ ID NOs: 52 + 5 + 53 + 54 + 55 + 64 + 68 + 58 + 59 |
| SEQ ID NOs: 50 + 9 + 51 | SEQ ID NOs: 52 + 5 + 53 + 54 + 55 + 64 + 68 + 58 + 59 |
| SEQ ID NOs: 50 + 10 + 51 | SEQ ID NOs: 52 + 5 + 53 + 54 + 55 + 64 + 68 + 58 + 59 |
| SEQ ID NOs: 50 + 11 + 51 | SEQ ID NOs: 52 + 5 + 53 + 54 + 55 + 64 + 68 + 58 + 59 |
| SEQ ID NOs: 50 + 12 + 51 | SEQ ID NOs: 52 + 5 + 53 + 54 + 55 + 64 + 68 + 58 + 59 |
| SEQ ID NOs: 50 + 13 + 51 | SEQ ID NOs: 52 + 5 + 53 + 54 + 55 + 64 + 68 + 58 + 59 |
| SEQ ID NOs: 50 + 14 + 51 | SEQ ID NOs: 52 + 5 + 53 + 54 + 55 + 64 + 68 + 58 + 59 |
| SEQ ID NOs: 50 + 49 + 51 | SEQ ID NOs: 52 + 5 + 53 + 54 + 55 + 64 + 68 + 58 + 59 |
| SEQ ID NOs: 50 + 8 + 51 | SEQ ID NOs: 52 + 5 + 53 + 54 + 55 + 64 + 69 + 58 + 59 |
| SEQ ID NOs: 50 + 9 + 51 | SEQ ID NOs: 52 + 5 + 53 + 54 + 55 + 64 + 69 + 58 + 59 |
| SEQ ID NOs: 50 + 10 + 51 | SEQ ID NOs: 52 + 5 + 53 + 54 + 55 + 64 + 69 + 58 + 59 |
| SEQ ID NOs: 50 + 11 + 51 | SEQ ID NOs: 52 + 5 + 53 + 54 + 55 + 64 + 69 + 58 + 59 |
| SEQ ID NOs: 50 + 12 + 51 | SEQ ID NOs: 52 + 5 + 53 + 54 + 55 + 64 + 69 + 58 + 59 |
| SEQ ID NOs: 50 + 13 + 51 | SEQ ID NOs: 52 + 5 + 53 + 54 + 55 + 64 + 69 + 58 + 59 |
| SEQ ID NOs: 50 + 14 + 51 | SEQ ID NOs: 52 + 5 + 53 + 54 + 55 + 64 + 69 + 58 + 59 |
| SEQ ID NOs: 50 + 49 + 51 | SEQ ID NOs: 52 + 5 + 53 + 54 + 55 + 64 + 69 + 58 + 59 |
| SEQ ID NOs: 50 + 8 + 51 | SEQ ID NOs: 52 + 5 + 53 + 54 + 55 + 65 + 57 + 58 + 59 |
| SEQ ID NOs: 50 + 9 + 51 | SEQ ID NOs: 52 + 5 + 53 + 54 + 55 + 65 + 57 + 58 + 59 |
| SEQ ID NOs: 50 + 10 + 51 | SEQ ID NOs: 52 + 5 + 53 + 54 + 55 + 65 + 57 + 58 + 59 |
| SEQ ID NOs: 50 + 11 + 51 | SEQ ID NOs: 52 + 5 + 53 + 54 + 55 + 65 + 57 + 58 + 59 |
| SEQ ID NOs: 50 + 12 + 51 | SEQ ID NOs: 52 + 5 + 53 + 54 + 55 + 65 + 57 + 58 + 59 |
| SEQ ID NOs: 50 + 13 + 51 | SEQ ID NOs: 52 + 5 + 53 + 54 + 55 + 65 + 57 + 58 + 59 |
| SEQ ID NOs: 50 + 14 + 51 | SEQ ID NOs: 52 + 5 + 53 + 54 + 55 + 65 + 57 + 58 + 59 |
| SEQ ID NOs: 50 + 49 + 51 | SEQ ID NOs: 52 + 5 + 53 + 54 + 55 + 65 + 57 + 58 + 59 |
| SEQ ID NOs: 50 + 8 + 51 | SEQ ID NOs: 52 + 5 + 53 + 54 + 55 + 65 + 68 + 58 + 59 |
| SEQ ID NOs: 50 + 9 + 51 | SEQ ID NOs: 52 + 5 + 53 + 54 + 55 + 65 + 68 + 58 + 59 |
| SEQ ID NOs: 50 + 10 + 51 | SEQ ID NOs: 52 + 5 + 53 + 54 + 55 + 65 + 68 + 58 + 59 |
| SEQ ID NOs: 50 + 11 + 51 | SEQ ID NOs: 52 + 5 + 53 + 54 + 55 + 65 + 68 + 58 + 59 |
| SEQ ID NOs: 50 + 12 + 51 | SEQ ID NOs: 52 + 5 + 53 + 54 + 55 + 65 + 68 + 58 + 59 |
| SEQ ID NOs: 50 + 13 + 51 | SEQ ID NOs: 52 + 5 + 53 + 54 + 55 + 65 + 68 + 58 + 59 |
| SEQ ID NOs: 50 + 14 + 51 | SEQ ID NOs: 52 + 5 + 53 + 54 + 55 + 65 + 68 + 58 + 59 |
| SEQ ID NOs: 50 + 49 + 51 | SEQ ID NOs: 52 + 5 + 53 + 54 + 55 + 65 + 68 + 58 + 59 |
| SEQ ID NOs: 50 + 8 + 51 | SEQ ID NOs: 52 + 5 + 53 + 54 + 55 + 65 + 69 + 58 + 59 |
| SEQ ID NOs: 50 + 9 + 51 | SEQ ID NOs: 52 + 5 + 53 + 54 + 55 + 65 + 69 + 58 + 59 |
| SEQ ID NOs: 50 + 10 + 51 | SEQ ID NOs: 52 + 5 + 53 + 54 + 55 + 65 + 69 + 58 + 59 |
| SEQ ID NOs: 50 + 11 + 51 | SEQ ID NOs: 52 + 5 + 53 + 54 + 55 + 65 + 69 + 58 + 59 |
| SEQ ID NOs: 50 + 12 + 51 | SEQ ID NOs: 52 + 5 + 53 + 54 + 55 + 65 + 69 + 58 + 59 |
| SEQ ID NOs: 50 + 13 + 51 | SEQ ID NOs: 52 + 5 + 53 + 54 + 55 + 65 + 69 + 58 + 59 |
| SEQ ID NOs: 50 + 14 + 51 | SEQ ID NOs: 52 + 5 + 53 + 54 + 55 + 65 + 69 + 58 + 59 |
| SEQ ID NOs: 50 + 49 + 51 | SEQ ID NOs: 52 + 5 + 53 + 54 + 55 + 65 + 69 + 58 + 59 |
| SEQ ID NOs: 50 + 8 + 51 | SEQ ID NOs: 52 + 5 + 53 + 54 + 55 + 66 + 57 + 58 + 59 |
| SEQ ID NOs: 50 + 9 + 51 | SEQ ID NOs: 52 + 5 + 53 + 54 + 55 + 66 + 57 + 58 + 59 |
| SEQ ID NOs: 50 + 10 + 51 | SEQ ID NOs: 52 + 5 + 53 + 54 + 55 + 66 + 57 + 58 + 59 |
| SEQ ID NOs: 50 + 11 + 51 | SEQ ID NOs: 52 + 5 + 53 + 54 + 55 + 66 + 57 + 58 + 59 |
| SEQ ID NOs: 50 + 12 + 51 | SEQ ID NOs: 52 + 5 + 53 + 54 + 55 + 66 + 57 + 58 + 59 |
| SEQ ID NOs: 50 + 13 + 51 | SEQ ID NOs: 52 + 5 + 53 + 54 + 55 + 66 + 57 + 58 + 59 |
| SEQ ID NOs: 50 + 14 + 51 | SEQ ID NOs: 52 + 5 + 53 + 54 + 55 + 66 + 57 + 58 + 59 |
| SEQ ID NOs: 50 + 49 + 51 | SEQ ID NOs: 52 + 5 + 53 + 54 + 55 + 66 + 57 + 58 + 59 |
| SEQ ID NOs: 50 + 8 + 51 | SEQ ID NOs: 52 + 5 + 53 + 54 + 55 + 66 + 68 + 58 + 59 |
| SEQ ID NOs: 50 + 9 + 51 | SEQ ID NOs: 52 + 5 + 53 + 54 + 55 + 66 + 68 + 58 + 59 |
| SEQ ID NOs: 50 + 10 + 51 | SEQ ID NOs: 52 + 5 + 53 + 54 + 55 + 66 + 68 + 58 + 59 |
| SEQ ID NOs: 50 + 11 + 51 | SEQ ID NOs: 52 + 5 + 53 + 54 + 55 + 66 + 68 + 58 + 59 |
| SEQ ID NOs: 50 + 12 + 51 | SEQ ID NOs: 52 + 5 + 53 + 54 + 55 + 66 + 68 + 58 + 59 |
| SEQ ID NOs: 50 + 13 + 51 | SEQ ID NOs: 52 + 5 + 53 + 54 + 55 + 66 + 68 + 58 + 59 |
| SEQ ID NOs: 50 + 14 + 51 | SEQ ID NOs: 52 + 5 + 53 + 54 + 55 + 66 + 68 + 58 + 59 |
| SEQ ID NOs: 50 + 49 + 51 | SEQ ID NOs: 52 + 5 + 53 + 54 + 55 + 66 + 68 + 58 + 59 |
| SEQ ID NOs: 50 + 8 + 51 | SEQ ID NOs: 52 + 5 + 53 + 54 + 55 + 66 + 69 + 58 + 59 |
| SEQ ID NOs: 50 + 9 + 51 | SEQ ID NOs: 52 + 5 + 53 + 54 + 55 + 66 + 69 + 58 + 59 |
| SEQ ID NOs: 50 + 10 + 51 | SEQ ID NOs: 52 + 5 + 53 + 54 + 55 + 66 + 69 + 58 + 59 |
| SEQ ID NOs: 50 + 11 + 51 | SEQ ID NOs: 52 + 5 + 53 + 54 + 55 + 66 + 69 + 58 + 59 |
| SEQ ID NOs: 50 + 12 + 51 | SEQ ID NOs: 52 + 5 + 53 + 54 + 55 + 66 + 69 + 58 + 59 |
| SEQ ID NOs: 50 + 13 + 51 | SEQ ID NOs: 52 + 5 + 53 + 54 + 55 + 66 + 69 + 58 + 59 |
| SEQ ID NOs: 50 + 14 + 51 | SEQ ID NOs: 52 + 5 + 53 + 54 + 55 + 66 + 69 + 58 + 59 |
| SEQ ID NOs: 50 + 49 + 51 | SEQ ID NOs: 52 + 5 + 53 + 54 + 55 + 66 + 69 + 58 + 59 |
| SEQ ID NOs: 50 + 8 + 51 | SEQ ID NOs: 52 + 5 + 53 + 54 + 55 + 67 + 57 + 58 + 59 |
| SEQ ID NOs: 50 + 9 + 51 | SEQ ID NOs: 52 + 5 + 53 + 54 + 55 + 67 + 57 + 58 + 59 |
| SEQ ID NOs: 50 + 10 + 51 | SEQ ID NOs: 52 + 5 + 53 + 54 + 55 + 67 + 57 + 58 + 59 |
| SEQ ID NOs: 50 + 11 + 51 | SEQ ID NOs: 52 + 5 + 53 + 54 + 55 + 67 + 57 + 58 + 59 |
| SEQ ID NOs: 50 + 12 + 51 | SEQ ID NOs: 52 + 5 + 53 + 54 + 55 + 67 + 57 + 58 + 59 |
| SEQ ID NOs: 50 + 13 + 51 | SEQ ID NOs: 52 + 5 + 53 + 54 + 55 + 67 + 57 + 58 + 59 |
| SEQ ID NOs: 50 + 14 + 51 | SEQ ID NOs: 52 + 5 + 53 + 54 + 55 + 67 + 57 + 58 + 59 |
| SEQ ID NOs: 50 + 49 + 51 | SEQ ID NOs: 52 + 5 + 53 + 54 + 55 + 67 + 57 + 58 + 59 |
| SEQ ID NOs: 50 + 8 + 51 | SEQ ID NOs: 52 + 5 + 53 + 54 + 55 + 67 + 68 + 58 + 59 |
| SEQ ID NOs: 50 + 9 + 51 | SEQ ID NOs: 52 + 5 + 53 + 54 + 55 + 67 + 68 + 58 + 59 |
| SEQ ID NOs: 50 + 10 + 51 | SEQ ID NOs: 52 + 5 + 53 + 54 + 55 + 67 + 68 + 58 + 59 |
| SEQ ID NOs: 50 + 11 + 51 | SEQ ID NOs: 52 + 5 + 53 + 54 + 55 + 67 + 68 + 58 + 59 |
| SEQ ID NOs: 50 + 12 + 51 | SEQ ID NOs: 52 + 5 + 53 + 54 + 55 + 67 + 68 + 58 + 59 |
| SEQ ID NOs: 50 + 13 + 51 | SEQ ID NOs: 52 + 5 + 53 + 54 + 55 + 67 + 68 + 58 + 59 |
| SEQ ID NOs: 50 + 14 + 51 | SEQ ID NOs: 52 + 5 + 53 + 54 + 55 + 67 + 68 + 58 + 59 |
| SEQ ID NOs: 50 + 49 + 51 | SEQ ID NOs: 52 + 5 + 53 + 54 + 55 + 67 + 68 + 58 + 59 |
| SEQ ID NOs: 50 + 8 + 51 | SEQ ID NOs: 52 + 5 + 53 + 54 + 55 + 67 + 69 + 58 + 59 |
| SEQ ID NOs: 50 + 9 + 51 | SEQ ID NOs: 52 + 5 + 53 + 54 + 55 + 67 + 69 + 58 + 59 |
| SEQ ID NOs: 50 + 10 + 51 | SEQ ID NOs: 52 + 5 + 53 + 54 + 55 + 67 + 69 + 58 + 59 |
| SEQ ID NOs: 50 + 11 + 51 | SEQ ID NOs: 52 + 5 + 53 + 54 + 55 + 67 + 69 + 58 + 59 |
| SEQ ID NOs: 50 + 12 + 51 | SEQ ID NOs: 52 + 5 + 53 + 54 + 55 + 67 + 69 + 58 + 59 |
| SEQ ID NOs: 50 + 13 + 51 | SEQ ID NOs: 52 + 5 + 53 + 54 + 55 + 67 + 69 + 58 + 59 |
| SEQ ID NOs: 50 + 14 + 51 | SEQ ID NOs: 52 + 5 + 53 + 54 + 55 + 67 + 69 + 58 + 59 |
| SEQ ID NOs: 50 + 49 + 51 | SEQ ID NOs: 52 + 5 + 53 + 54 + 55 + 67 + 69 + 58 + 59 |
| SEQ ID NOs: 50 + 8 + 15 | SEQ ID NOs: 52 + 5 + 53 + 54 + 55 + 56 + 57 + 58 + 59 |
| SEQ ID NOs: 50 + 9 + 15 | SEQ ID NOs: 52 + 5 + 53 + 54 + 55 + 56 + 57 + 58 + 59 |
| SEQ ID NOs: 50 + 10 + 15 | SEQ ID NOs: 52 + 5 + 53 + 54 + 55 + 56 + 57 + 58 + 59 |
| SEQ ID NOs: 50 + 11 + 15 | SEQ ID NOs: 52 + 5 + 53 + 54 + 55 + 56 + 57 + 58 + 59 |
| SEQ ID NOs: 50 + 12 + 15 | SEQ ID NOs: 52 + 5 + 53 + 54 + 55 + 56 + 57 + 58 + 59 |
| SEQ ID NOs: 50 + 13 + 15 | SEQ ID NOs: 52 + 5 + 53 + 54 + 55 + 56 + 57 + 58 + 59 |
| SEQ ID NOs: 50 + 14 + 15 | SEQ ID NOs: 52 + 5 + 53 + 54 + 55 + 56 + 57 + 58 + 59 |
| SEQ ID NOs: 50 + 49 + 15 | SEQ ID NOs: 52 + 5 + 53 + 54 + 55 + 56 + 57 + 58 + 59 |
| SEQ ID NOs: 50 + 8 + 15 | SEQ ID NOs: 52 + 5 + 53 + 54 + 55 + 56 + 68 + 58 + 59 |
| SEQ ID NOs: 50 + 9 + 15 | SEQ ID NOs: 52 + 5 + 53 + 54 + 55 + 56 + 68 + 58 + 59 |
| SEQ ID NOs: 50 + 10 + 15 | SEQ ID NOs: 52 + 5 + 53 + 54 + 55 + 56 + 68 + 58 + 59 |
| SEQ ID NOs: 50 + 11 + 15 | SEQ ID NOs: 52 + 5 + 53 + 54 + 55 + 56 + 68 + 58 + 59 |
| SEQ ID NOs: 50 + 12 + 15 | SEQ ID NOs: 52 + 5 + 53 + 54 + 55 + 56 + 68 + 58 + 59 |
| SEQ ID NOs: 50 + 13 + 15 | SEQ ID NOs: 52 + 5 + 53 + 54 + 55 + 56 + 68 + 58 + 59 |
| SEQ ID NOs: 50 + 14 + 15 | SEQ ID NOs: 52 + 5 + 53 + 54 + 55 + 56 + 68 + 58 + 59 |
| SEQ ID NOs: 50 + 49 + 15 | SEQ ID NOs: 52 + 5 + 53 + 54 + 55 + 56 + 68 + 58 + 59 |
| SEQ ID NOs: 50 + 8 + 15 | SEQ ID NOs: 52 + 5 + 53 + 54 + 55 + 56 + 69 + 58 + 59 |
| SEQ ID NOs: 50 + 9 + 15 | SEQ ID NOs: 52 + 5 + 53 + 54 + 55 + 56 + 69 + 58 + 59 |
| SEQ ID NOs: 50 + 10 + 15 | SEQ ID NOs: 52 + 5 + 53 + 54 + 55 + 56 + 69 + 58 + 59 |
| SEQ ID NOs: 50 + 11 + 15 | SEQ ID NOs: 52 + 5 + 53 + 54 + 55 + 56 + 69 + 58 + 59 |
| SEQ ID NOs: 50 + 12 + 15 | SEQ ID NOs: 52 + 5 + 53 + 54 + 55 + 56 + 69 + 58 + 59 |
| SEQ ID NOs: 50 + 13 + 15 | SEQ ID NOs: 52 + 5 + 53 + 54 + 55 + 56 + 69 + 58 + 59 |
| SEQ ID NOs: 50 + 14 + 15 | SEQ ID NOs: 52 + 5 + 53 + 54 + 55 + 56 + 68 + 58 + 59 |
| SEQ ID NOs: 50 + 49 + 15 | SEQ ID NOs: 52 + 5 + 53 + 54 + 55 + 56 + 69 + 58 + 59 |
| SEQ ID NOs: 50 + 8 + 15 | SEQ ID NOs: 52 + 5 + 53 + 54 + 55 + 60 + 57 + 58 + 59 |
| SEQ ID NOs: 50 + 9 + 15 | SEQ ID NOs: 52 + 5 + 53 + 54 + 55 + 60 + 57 + 58 + 59 |
| SEQ ID NOs: 50 + 10 + 15 | SEQ ID NOs: 52 + 5 + 53 + 54 + 55 + 60 + 57 + 58 + 59 |
| SEQ ID NOs: 50 + 11 + 15 | SEQ ID NOs: 52 + 5 + 53 + 54 + 55 + 60 + 57 + 58 + 59 |
| SEQ ID NOs: 50 + 12 + 15 | SEQ ID NOs: 52 + 5 + 53 + 54 + 55 + 60 + 57 + 58 + 59 |
| SEQ ID NOs: 50 + 13 + 15 | SEQ ID NOs: 52 + 5 + 53 + 54 + 55 + 60 + 57 + 58 + 59 |
| SEQ ID NOs: 50 + 14 + 15 | SEQ ID NOs: 52 + 5 + 53 + 54 + 55 + 60 + 57 + 58 + 59 |
| SEQ ID NOs: 50 + 49 + 15 | SEQ ID NOs: 52 + 5 + 53 + 54 + 55 + 60 + 57 + 58 + 59 |
| SEQ ID NOs: 50 + 8 + 15 | SEQ ID NOs: 52 + 5 + 53 + 54 + 55 + 60 + 68 + 58 + 59 |
| SEQ ID NOs: 50 + 9 + 15 | SEQ ID NOs: 52 + 5 + 53 + 54 + 55 + 60 + 68 + 58 + 59 |
| SEQ ID NOs: 50 + 10 + 15 | SEQ ID NOs: 52 + 5 + 53 + 54 + 55 + 60 + 68 + 58 + 59 |
| SEQ ID NOs: 50 + 11 + 15 | SEQ ID NOs: 52 + 5 + 53 + 54 + 55 + 60 + 68 + 58 + 59 |
| SEQ ID NOs: 50 + 12 + 15 | SEQ ID NOs: 52 + 5 + 53 + 54 + 55 + 60 + 68 + 58 + 59 |
| SEQ ID NOs: 50 + 13 + 15 | SEQ ID NOs: 52 + 5 + 53 + 54 + 55 + 60 + 68 + 58 + 59 |
| SEQ ID NOs: 50 + 14 + 15 | SEQ ID NOs: 52 + 5 + 53 + 54 + 55 + 60 + 68 + 58 + 59 |
| SEQ ID NOs: 50 + 49 + 15 | SEQ ID NOs: 52 + 5 + 53 + 54 + 55 + 60 + 68 + 58 + 59 |
| SEQ ID NOs: 50 + 8 + 15 | SEQ ID NOs: 52 + 5 + 53 + 54 + 55 + 60 + 69 + 58 + 59 |
| SEQ ID NOs: 50 + 9 + 15 | SEQ ID NOs: 52 + 5 + 53 + 54 + 55 + 60 + 69 + 58 + 59 |
| SEQ ID NOs: 50 + 10 + 15 | SEQ ID NOs: 52 + 5 + 53 + 54 + 55 + 60 + 69 + 58 + 59 |
| SEQ ID NOs: 50 + 11 + 15 | SEQ ID NOs: 52 + 5 + 53 + 54 + 55 + 60 + 69 + 58 + 59 |
| SEQ ID NOs: 50 + 12 + 15 | SEQ ID NOs: 52 + 5 + 53 + 54 + 55 + 60 + 69 + 58 + 59 |
| SEQ ID NOs: 50 + 13 + 15 | SEQ ID NOs: 52 + 5 + 53 + 54 + 55 + 60 + 69 + 58 + 59 |
| SEQ ID NOs: 50 + 14 + 15 | SEQ ID NOs: 52 + 5 + 53 + 54 + 55 + 60 + 68 + 58 + 59 |
| SEQ ID NOs: 50 + 49 + 15 | SEQ ID NOs: 52 + 5 + 53 + 54 + 55 + 60 + 69 + 58 + 59 |
| SEQ ID NOs: 50 + 8 + 15 | SEQ ID NOs: 52 + 5 + 53 + 54 + 55 + 60 + 57 + 58 + 59 |
| SEQ ID NOs: 50 + 9 + 15 | SEQ ID NOs: 52 + 5 + 53 + 54 + 55 + 60 + 57 + 58 + 59 |
| SEQ ID NOs: 50 + 10 + 15 | SEQ ID NOs: 52 + 5 + 53 + 54 + 55 + 60 + 57 + 58 + 59 |
| SEQ ID NOs: 50 + 11 + 15 | SEQ ID NOs: 52 + 5 + 53 + 54 + 55 + 60 + 57 + 58 + 59 |
| SEQ ID NOs: 50 + 12 + 15 | SEQ ID NOs: 52 + 5 + 53 + 54 + 55 + 60 + 57 + 58 + 59 |
| SEQ ID NOs: 50 + 13 + 15 | SEQ ID NOs: 52 + 5 + 53 + 54 + 55 + 60 + 57 + 58 + 59 |
| SEQ ID NOs: 50 + 14 + 15 | SEQ ID NOs: 52 + 5 + 53 + 54 + 55 + 60 + 57 + 58 + 59 |
| SEQ ID NOs: 50 + 49 + 15 | SEQ ID NOs: 52 + 5 + 53 + 54 + 55 + 60 + 57 + 58 + 59 |
| SEQ ID NOs: 50 + 8 + 15 | SEQ ID NOs: 52 + 5 + 53 + 54 + 55 + 61 + 68 + 58 + 59 |
| SEQ ID NOs: 50 + 9 + 15 | SEQ ID NOs: 52 + 5 + 53 + 54 + 55 + 61 + 68 + 58 + 59 |
| SEQ ID NOs: 50 + 10 + 15 | SEQ ID NOs: 52 + 5 + 53 + 54 + 55 + 61 + 68 + 58 + 59 |
| SEQ ID NOs: 50 + 11 + 15 | SEQ ID NOs: 52 + 5 + 53 + 54 + 55 + 61 + 68 + 58 + 59 |
| SEQ ID NOs: 50 + 12 + 15 | SEQ ID NOs: 52 + 5 + 53 + 54 + 55 + 61 + 68 + 58 + 59 |
| SEQ ID NOs: 50 + 13 + 15 | SEQ ID NOs: 52 + 5 + 53 + 54 + 55 + 61 + 68 + 58 + 59 |
| SEQ ID NOs: 50 + 14 + 15 | SEQ ID NOs: 52 + 5 + 53 + 54 + 55 + 61 + 68 + 58 + 59 |
| SEQ ID NOs: 50 + 49 + 15 | SEQ ID NOs: 52 + 5 + 53 + 54 + 55 + 61 + 68 + 58 + 59 |
| SEQ ID NOs: 50 + 8 + 15 | SEQ ID NOs: 52 + 5 + 53 + 54 + 55 + 61 + 69 + 58 + 59 |
| SEQ ID NOs: 50 + 9 + 15 | SEQ ID NOs: 52 + 5 + 53 + 54 + 55 + 61 + 69 + 58 + 59 |
| SEQ ID NOs: 50 + 10 + 15 | SEQ ID NOs: 52 + 5 + 53 + 54 + 55 + 61 + 69 + 58 + 59 |
| SEQ ID NOs: 50 + 11 + 15 | SEQ ID NOs: 52 + 5 + 53 + 54 + 55 + 61 + 69 + 58 + 59 |
| SEQ ID NOs: 50 + 12 + 15 | SEQ ID NOs: 52 + 5 + 53 + 54 + 55 + 61 + 69 + 58 + 59 |
| SEQ ID NOs: 50 + 13 + 15 | SEQ ID NOs: 52 + 5 + 53 + 54 + 55 + 61 + 69 + 58 + 59 |
| SEQ ID NOs: 50 + 14 + 15 | SEQ ID NOs: 52 + 5 + 53 + 54 + 55 + 61 + 68 + 58 + 59 |
| SEQ ID NOs: 50 + 49 + 15 | SEQ ID NOs: 52 + 5 + 53 + 54 + 55 + 61 + 69 + 58 + 59 |
| SEQ ID NOs: 50 + 8 + 15 | SEQ ID NOs: 52 + 5 + 53 + 54 + 55 + 62 + 57 + 58 + 59 |
| SEQ ID NOs: 50 + 9 + 15 | SEQ ID NOs: 52 + 5 + 53 + 54 + 55 + 62 + 57 + 58 + 59 |
| SEQ ID NOs: 50 + 10 + 15 | SEQ ID NOs: 52 + 5 + 53 + 54 + 55 + 62 + 57 + 58 + 59 |
| SEQ ID NOs: 50 + 11 + 15 | SEQ ID NOs: 52 + 5 + 53 + 54 + 55 + 62 + 57 + 58 + 59 |
| SEQ ID NOs: 50 + 12 + 15 | SEQ ID NOs: 52 + 5 + 53 + 54 + 55 + 62 + 57 + 58 + 59 |
| SEQ ID NOs: 50 + 13 + 15 | SEQ ID NOs: 52 + 5 + 53 + 54 + 55 + 62 + 57 + 58 + 59 |
| SEQ ID NOs: 50 + 14 + 15 | SEQ ID NOs: 52 + 5 + 53 + 54 + 55 + 62 + 57 + 58 + 59 |
| SEQ ID NOs: 50 + 49 + 15 | SEQ ID NOs: 52 + 5 + 53 + 54 + 55 + 62 + 57 + 58 + 59 |
| SEQ ID NOs: 50 + 8 + 15 | SEQ ID NOs: 52 + 5 + 53 + 54 + 55 + 62 + 68 + 58 + 59 |
| SEQ ID NOs: 50 + 9 + 15 | SEQ ID NOs: 52 + 5 + 53 + 54 + 55 + 62 + 68 + 58 + 59 |
| SEQ ID NOs: 50 + 10 + 15 | SEQ ID NOs: 52 + 5 + 53 + 54 + 55 + 62 + 68 + 58 + 59 |
| SEQ ID NOs: 50 + 11 + 15 | SEQ ID NOs: 52 + 5 + 53 + 54 + 55 + 62 + 68 + 58 + 59 |
| SEQ ID NOs: 50 + 12 + 15 | SEQ ID NOs: 52 + 5 + 53 + 54 + 55 + 62 + 68 + 58 + 59 |
| SEQ ID NOs: 50 + 13 + 15 | SEQ ID NOs: 52 + 5 + 53 + 54 + 55 + 62 + 68 + 58 + 59 |
| SEQ ID NOs: 50 + 14 + 15 | SEQ ID NOs: 52 + 5 + 53 + 54 + 55 + 62 + 68 + 58 + 59 |
| SEQ ID NOs: 50 + 49 + 15 | SEQ ID NOs: 52 + 5 + 53 + 54 + 55 + 62 + 68 + 58 + 59 |
| SEQ ID NOs: 50 + 8 + 15 | SEQ ID NOs: 52 + 5 + 53 + 54 + 55 + 62 + 69 + 58 + 59 |
| SEQ ID NOs: 50 + 9 + 15 | SEQ ID NOs: 52 + 5 + 53 + 54 + 55 + 62 + 69 + 58 + 59 |
| SEQ ID NOs: 50 + 10 + 15 | SEQ ID NOs: 52 + 5 + 53 + 54 + 55 + 62 + 69 + 58 + 59 |
| SEQ ID NOs: 50 + 11 + 15 | SEQ ID NOs: 52 + 5 + 53 + 54 + 55 + 62 + 69 + 58 + 59 |
| SEQ ID NOs: 50 + 12 + 15 | SEQ ID NOs: 52 + 5 + 53 + 54 + 55 + 62 + 69 + 58 + 59 |
| SEQ ID NOs: 50 + 13 + 15 | SEQ ID NOs: 52 + 5 + 53 + 54 + 55 + 62 + 69 + 58 + 59 |
| SEQ ID NOs: 50 + 14 + 15 | SEQ ID NOs: 52 + 5 + 53 + 54 + 55 + 62 + 68 + 58 + 59 |
| SEQ ID NOs: 50 + 49 + 15 | SEQ ID NOs: 52 + 5 + 53 + 54 + 55 + 62 + 69 + 58 + 59 |
| SEQ ID NOs: 50 + 8 + 15 | SEQ ID NOs: 52 + 5 + 53 + 54 + 55 + 63 + 57 + 58 + 59 |
| SEQ ID NOs: 50 + 9 + 15 | SEQ ID NOs: 52 + 5 + 53 + 54 + 55 + 63 + 57 + 58 + 59 |
| SEQ ID NOs: 50 + 10 + 15 | SEQ ID NOs: 52 + 5 + 53 + 54 + 55 + 63 + 57 + 58 + 59 |
| SEQ ID NOs: 50 + 11 + 15 | SEQ ID NOs: 52 + 5 + 53 + 54 + 55 + 63 + 57 + 58 + 59 |
| SEQ ID NOs: 50 + 12 + 15 | SEQ ID NOs: 52 + 5 + 53 + 54 + 55 + 63 + 57 + 58 + 59 |
| SEQ ID NOs: 50 + 13 + 15 | SEQ ID NOs: 52 + 5 + 53 + 54 + 55 + 63 + 57 + 58 + 59 |
| SEQ ID NOs: 50 + 14 + 15 | SEQ ID NOs: 52 + 5 + 53 + 54 + 55 + 63 + 57 + 58 + 59 |
| SEQ ID NOs: 50 + 49 + 15 | SEQ ID NOs: 52 + 5 + 53 + 54 + 55 + 63 + 57 + 58 + 59 |
| SEQ ID NOs: 50 + 8 + 15 | SEQ ID NOs: 52 + 5 + 53 + 54 + 55 + 63 + 68 + 58 + 59 |
| SEQ ID NOs: 50 + 9 + 15 | SEQ ID NOs: 52 + 5 + 53 + 54 + 55 + 63 + 68 + 58 + 59 |
| SEQ ID NOs: 50 + 10 + 15 | SEQ ID NOs: 52 + 5 + 53 + 54 + 55 + 63 + 68 + 58 + 59 |
| SEQ ID NOs: 50 + 11 + 15 | SEQ ID NOs: 52 + 5 + 53 + 54 + 55 + 63 + 68 + 58 + 59 |
| SEQ ID NOs: 50 + 12 + 15 | SEQ ID NOs: 52 + 5 + 53 + 54 + 55 + 63 + 68 + 58 + 59 |
| SEQ ID NOs: 50 + 13 + 15 | SEQ ID NOs: 52 + 5 + 53 + 54 + 55 + 63 + 68 + 58 + 59 |
| SEQ ID NOs: 50 + 14 + 15 | SEQ ID NOs: 52 + 5 + 53 + 54 + 55 + 63 + 68 + 58 + 59 |
| SEQ ID NOs: 50 + 49 + 15 | SEQ ID NOs: 52 + 5 + 53 + 54 + 55 + 63 + 68 + 58 + 59 |
| SEQ ID NOs: 50 + 8 + 15 | SEQ ID NOs: 52 + 5 + 53 + 54 + 55 + 63 + 69 + 58 + 59 |
| SEQ ID NOs: 50 + 9 + 15 | SEQ ID NOs: 52 + 5 + 53 + 54 + 55 + 63 + 69 + 58 + 59 |
| SEQ ID NOs: 50 + 10 + 15 | SEQ ID NOs: 52 + 5 + 53 + 54 + 55 + 63 + 69 + 58 + 59 |
| SEQ ID NOs: 50 + 11 + 15 | SEQ ID NOs: 52 + 5 + 53 + 54 + 55 + 63 + 69 + 58 + 59 |
| SEQ ID NOs: 50 + 12 + 15 | SEQ ID NOs: 52 + 5 + 53 + 54 + 55 + 63 + 69 + 58 + 59 |
| SEQ ID NOs: 50 + 13 + 15 | SEQ ID NOs: 52 + 5 + 53 + 54 + 55 + 63 + 69 + 58 + 59 |
| SEQ ID NOs: 50 + 14 + 15 | SEQ ID NOs: 52 + 5 + 53 + 54 + 55 + 63 + 68 + 58 + 59 |
| SEQ ID NOs: 50 + 49 + 15 | SEQ ID NOs: 52 + 5 + 53 + 54 + 55 + 63 + 69 + 58 + 59 |
| SEQ ID NOs: 50 + 8 + 15 | SEQ ID NOs: 52 + 5 + 53 + 54 + 55 + 64 + 57 + 58 + 59 |
| SEQ ID NOs: 50 + 9 + 15 | SEQ ID NOs: 52 + 5 + 53 + 54 + 55 + 64 + 57 + 58 + 59 |
| SEQ ID NOs: 50 + 10 + 15 | SEQ ID NOs: 52 + 5 + 53 + 54 + 55 + 64 + 57 + 58 + 59 |
| SEQ ID NOs: 50 + 11 + 15 | SEQ ID NOs: 52 + 5 + 53 + 54 + 55 + 64 + 57 + 58 + 59 |
| SEQ ID NOs: 50 + 12 + 15 | SEQ ID NOs: 52 + 5 + 53 + 54 + 55 + 64 + 57 + 58 + 59 |
| SEQ ID NOs: 50 + 13 + 15 | SEQ ID NOs: 52 + 5 + 53 + 54 + 55 + 64 + 57 + 58 + 59 |
| SEQ ID NOs: 50 + 14 + 15 | SEQ ID NOs: 52 + 5 + 53 + 54 + 55 + 64 + 57 + 58 + 59 |
| SEQ ID NOs: 50 + 49 + 15 | SEQ ID NOs: 52 + 5 + 53 + 54 + 55 + 64 + 57 + 58 + 59 |
| SEQ ID NOs: 50 + 8 + 15 | SEQ ID NOs: 52 + 5 + 53 + 54 + 55 + 64 + 68 + 58 + 59 |
| SEQ ID NOs: 50 + 9 + 15 | SEQ ID NOs: 52 + 5 + 53 + 54 + 55 + 64 + 68 + 58 + 59 |
| SEQ ID NOs: 50 + 10 + 15 | SEQ ID NOs: 52 + 5 + 53 + 54 + 55 + 64 + 68 + 58 + 59 |
| SEQ ID NOs: 50 + 11 + 15 | SEQ ID NOs: 52 + 5 + 53 + 54 + 55 + 64 + 68 + 58 + 59 |
| SEQ ID NOs: 50 + 12 + 15 | SEQ ID NOs: 52 + 5 + 53 + 54 + 55 + 64 + 68 + 58 + 59 |
| SEQ ID NOs: 50 + 13 + 15 | SEQ ID NOs: 52 + 5 + 53 + 54 + 55 + 64 + 68 + 58 + 59 |
| SEQ ID NOs: 50 + 14 + 15 | SEQ ID NOs: 52 + 5 + 53 + 54 + 55 + 64 + 68 + 58 + 59 |
| SEQ ID NOs: 50 + 49 + 15 | SEQ ID NOs: 52 + 5 + 53 + 54 + 55 + 64 + 68 + 58 + 59 |
| SEQ ID NOs: 50 + 8 + 15 | SEQ ID NOs: 52 + 5 + 53 + 54 + 55 + 64 + 69 + 58 + 59 |
| SEQ ID NOs: 50 + 9 + 15 | SEQ ID NOs: 52 + 5 + 53 + 54 + 55 + 64 + 69 + 58 + 59 |
| SEQ ID NOs: 50 + 10 + 15 | SEQ ID NOs: 52 + 5 + 53 + 54 + 55 + 64 + 69 + 58 + 59 |
| SEQ ID NOs: 50 + 11 + 15 | SEQ ID NOs: 52 + 5 + 53 + 54 + 55 + 64 + 69 + 58 + 59 |
| SEQ ID NOs: 50 + 12 + 15 | SEQ ID NOs: 52 + 5 + 53 + 54 + 55 + 64 + 69 + 58 + 59 |
| SEQ ID NOs: 50 + 13 + 15 | SEQ ID NOs: 52 + 5 + 53 + 54 + 55 + 64 + 69 + 58 + 59 |
| SEQ ID NOs: 50 + 14 + 15 | SEQ ID NOs: 52 + 5 + 53 + 54 + 55 + 64 + 68 + 58 + 59 |
| SEQ ID NOs: 50 + 49 + 15 | SEQ ID NOs: 52 + 5 + 53 + 54 + 55 + 64 + 69 + 58 + 59 |
| SEQ ID NOs: 50 + 8 + 15 | SEQ ID NOs: 52 + 5 + 53 + 54 + 55 + 65 + 57 + 58 + 59 |
| SEQ ID NOs: 50 + 9 + 15 | SEQ ID NOs: 52 + 5 + 53 + 54 + 55 + 65 + 57 + 58 + 59 |
| SEQ ID NOs: 50 + 10 + 15 | SEQ ID NOs: 52 + 5 + 53 + 54 + 55 + 65 + 57 + 58 + 59 |
| SEQ ID NOs: 50 + 11 + 15 | SEQ ID NOs: 52 + 5 + 53 + 54 + 55 + 65 + 57 + 58 + 59 |
| SEQ ID NOs: 50 + 12 + 15 | SEQ ID NOs: 52 + 5 + 53 + 54 + 55 + 65 + 57 + 58 + 59 |
| SEQ ID NOs: 50 + 13 + 15 | SEQ ID NOs: 52 + 5 + 53 + 54 + 55 + 65 + 57 + 58 + 59 |
| SEQ ID NOs: 50 + 14 + 15 | SEQ ID NOs: 52 + 5 + 53 + 54 + 55 + 65 + 57 + 58 + 59 |
| SEQ ID NOs: 50 + 49 + 15 | SEQ ID NOs: 52 + 5 + 53 + 54 + 55 + 65 + 57 + 58 + 59 |
| SEQ ID NOs: 50 + 8 + 15 | SEQ ID NOs: 52 + 5 + 53 + 54 + 55 + 65 + 68 + 58 + 59 |
| SEQ ID NOs: 50 + 9 + 15 | SEQ ID NOs: 52 + 5 + 53 + 54 + 55 + 65 + 68 + 58 + 59 |
| SEQ ID NOs: 50 + 10 + 15 | SEQ ID NOs: 52 + 5 + 53 + 54 + 55 + 65 + 68 + 58 + 59 |
| SEQ ID NOs: 50 + 11 + 15 | SEQ ID NOs: 52 + 5 + 53 + 54 + 55 + 65 + 68 + 58 + 59 |
| SEQ ID NOs: 50 + 12 + 15 | SEQ ID NOs: 52 + 5 + 53 + 54 + 55 + 65 + 68 + 58 + 59 |
| SEQ ID NOs: 50 + 13 + 15 | SEQ ID NOs: 52 + 5 + 53 + 54 + 55 + 65 + 68 + 58 + 59 |
| SEQ ID NOs: 50 + 14 + 15 | SEQ ID NOs: 52 + 5 + 53 + 54 + 55 + 65 + 68 + 58 + 59 |
| SEQ ID NOs: 50 + 49 + 15 | SEQ ID NOs: 52 + 5 + 53 + 54 + 55 + 65 + 68 + 58 + 59 |
| SEQ ID NOs: 50 + 8 + 15 | SEQ ID NOs: 52 + 5 + 53 + 54 + 55 + 65 + 69 + 58 + 59 |
| SEQ ID NOs: 50 + 9 + 15 | SEQ ID NOs: 52 + 5 + 53 + 54 + 55 + 65 + 69 + 58 + 59 |
| SEQ ID NOs: 50 + 10 + 15 | SEQ ID NOs: 52 + 5 + 53 + 54 + 55 + 65 + 69 + 58 + 59 |
| SEQ ID NOs: 50 + 11 + 15 | SEQ ID NOs: 52 + 5 + 53 + 54 + 55 + 65 + 69 + 58 + 59 |
| SEQ ID NOs: 50 + 12 + 15 | SEQ ID NOs: 52 + 5 + 53 + 54 + 55 + 65 + 69 + 58 + 59 |
| SEQ ID NOs: 50 + 13 + 15 | SEQ ID NOs: 52 + 5 + 53 + 54 + 55 + 65 + 69 + 58 + 59 |
| SEQ ID NOs: 50 + 14 + 15 | SEQ ID NOs: 52 + 5 + 53 + 54 + 55 + 65 + 68 + 58 + 59 |
| SEQ ID NOs: 50 + 49 + 15 | SEQ ID NOs: 52 + 5 + 53 + 54 + 55 + 65 + 69 + 58 + 59 |
| SEQ ID NOs: 50 + 8 + 15 | SEQ ID NOs: 52 + 5 + 53 + 54 + 55 + 66 + 57 + 58 + 59 |
| SEQ ID NOs: 50 + 9 + 15 | SEQ ID NOs: 52 + 5 + 53 + 54 + 55 + 66 + 57 + 58 + 59 |
| SEQ ID NOs: 50 + 10 + 15 | SEQ ID NOs: 52 + 5 + 53 + 54 + 55 + 66 + 57 + 58 + 59 |
| SEQ ID NOs: 50 + 11 + 15 | SEQ ID NOs: 52 + 5 + 53 + 54 + 55 + 66 + 57 + 58 + 59 |
| SEQ ID NOs: 50 + 12 + 15 | SEQ ID NOs: 52 + 5 + 53 + 54 + 55 + 66 + 57 + 58 + 59 |
| SEQ ID NOs: 50 + 13 + 15 | SEQ ID NOs: 52 + 5 + 53 + 54 + 55 + 66 + 57 + 58 + 59 |
| SEQ ID NOs: 50 + 14 + 15 | SEQ ID NOs: 52 + 5 + 53 + 54 + 55 + 66 + 57 + 58 + 59 |
| SEQ ID NOs: 50 + 49 + 15 | SEQ ID NOs: 52 + 5 + 53 + 54 + 55 + 66 + 57 + 58 + 59 |
| SEQ ID NOs: 50 + 8 + 15 | SEQ ID NOs: 52 + 5 + 53 + 54 + 55 + 66 + 68 + 58 + 59 |
| SEQ ID NOs: 50 + 9 + 15 | SEQ ID NOs: 52 + 5 + 53 + 54 + 55 + 66 + 68 + 58 + 59 |
| SEQ ID NOs: 50 + 10 + 15 | SEQ ID NOs: 52 + 5 + 53 + 54 + 55 + 66 + 68 + 58 + 59 |
| SEQ ID NOs: 50 + 11 + 15 | SEQ ID NOs: 52 + 5 + 53 + 54 + 55 + 66 + 68 + 58 + 59 |
| SEQ ID NOs: 50 + 12 + 15 | SEQ ID NOs: 52 + 5 + 53 + 54 + 55 + 66 + 68 + 58 + 59 |
| SEQ ID NOs: 50 + 13 + 15 | SEQ ID NOs: 52 + 5 + 53 + 54 + 55 + 66 + 68 + 58 + 59 |
| SEQ ID NOs: 50 + 14 + 15 | SEQ ID NOs: 52 + 5 + 53 + 54 + 55 + 66 + 68 + 58 + 59 |
| SEQ ID NOs: 50 + 49 + 15 | SEQ ID NOs: 52 + 5 + 53 + 54 + 55 + 66 + 68 + 58 + 59 |
| SEQ ID NOs: 50 + 8 + 15 | SEQ ID NOs: 52 + 5 + 53 + 54 + 55 + 66 + 69 + 58 + 59 |
| SEQ ID NOs: 50 + 9 + 15 | SEQ ID NOs: 52 + 5 + 53 + 54 + 55 + 66 + 69 + 58 + 59 |
| SEQ ID NOs: 50 + 10 + 15 | SEQ ID NOs: 52 + 5 + 53 + 54 + 55 + 66 + 69 + 58 + 59 |
| SEQ ID NOs: 50 + 11 + 15 | SEQ ID NOs: 52 + 5 + 53 + 54 + 55 + 66 + 69 + 58 + 59 |
| SEQ ID NOs: 50 + 12 + 15 | SEQ ID NOs: 52 + 5 + 53 + 54 + 55 + 66 + 69 + 58 + 59 |
| SEQ ID NOs: 50 + 13 + 15 | SEQ ID NOs: 52 + 5 + 53 + 54 + 55 + 66 + 69 + 58 + 59 |
| SEQ ID NOs: 50 + 14 + 15 | SEQ ID NOs: 52 + 5 + 53 + 54 + 55 + 66 + 68 + 58 + 59 |
| SEQ ID NOs: 50 + 49 + 15 | SEQ ID NOs: 52 + 5 + 53 + 54 + 55 + 66 + 69 + 58 + 59 |
| SEQ ID NOs: 50 + 8 + 15 | SEQ ID NOs: 52 + 5 + 53 + 54 + 55 + 67 + 57 + 58 + 59 |
| SEQ ID NOs: 50 + 9 + 15 | SEQ ID NOs: 52 + 5 + 53 + 54 + 55 + 67 + 57 + 58 + 59 |
| SEQ ID NOs: 50 + 10 + 15 | SEQ ID NOs: 52 + 5 + 53 + 54 + 55 + 67 + 57 + 58 + 59 |
| SEQ ID NOs: 50 + 11 + 15 | SEQ ID NOs: 52 + 5 + 53 + 54 + 55 + 67 + 57 + 58 + 59 |
| SEQ ID NOs: 50 + 12 + 15 | SEQ ID NOs: 52 + 5 + 53 + 54 + 55 + 67 + 57 + 58 + 59 |
| SEQ ID NOs: 50 + 13 + 15 | SEQ ID NOs: 52 + 5 + 53 + 54 + 55 + 67 + 57 + 58 + 59 |
| SEQ ID NOs: 50 + 14 + 15 | SEQ ID NOs: 52 + 5 + 53 + 54 + 55 + 67 + 57 + 58 + 59 |
| SEQ ID NOs: 50 + 49 + 15 | SEQ ID NOs: 52 + 5 + 53 + 54 + 55 + 67 + 57 + 58 + 59 |
| SEQ ID NOs: 50 + 8 + 15 | SEQ ID NOs: 52 + 5 + 53 + 54 + 55 + 67 + 68 + 58 + 59 |
| SEQ ID NOs: 50 + 9 + 15 | SEQ ID NOs: 52 + 5 + 53 + 54 + 55 + 67 + 68 + 58 + 59 |
| SEQ ID NOs: 50 + 10 + 15 | SEQ ID NOs: 52 + 5 + 53 + 54 + 55 + 67 + 68 + 58 + 59 |
| SEQ ID NOs: 50 + 11 + 15 | SEQ ID NOs: 52 + 5 + 53 + 54 + 55 + 67 + 68 + 58 + 59 |
| SEQ ID NOs: 50 + 12 + 15 | SEQ ID NOs: 52 + 5 + 53 + 54 + 55 + 67 + 68 + 58 + 59 |
| SEQ ID NOs: 50 + 13 + 15 | SEQ ID NOs: 52 + 5 + 53 + 54 + 55 + 67 + 68 + 58 + 59 |
| SEQ ID NOs: 50 + 14 + 15 | SEQ ID NOs: 52 + 5 + 53 + 54 + 55 + 67 + 68 + 58 + 59 |
| SEQ ID NOs: 50 + 49 + 15 | SEQ ID NOs: 52 + 5 + 53 + 54 + 55 + 67 + 68 + 58 + 59 |
| SEQ ID NOs: 50 + 8 + 15 | SEQ ID NOs: 52 + 5 + 53 + 54 + 55 + 67 + 69 + 58 + 59 |
| SEQ ID NOs: 50 + 9 + 15 | SEQ ID NOs: 52 + 5 + 53 + 54 + 55 + 67 + 69 + 58 + 59 |
| SEQ ID NOs: 50 + 10 + 15 | SEQ ID NOs: 52 + 5 + 53 + 54 + 55 + 67 + 69 + 58 + 59 |
| SEQ ID NOs: 50 + 11 + 15 | SEQ ID NOs: 52 + 5 + 53 + 54 + 55 + 67 + 69 + 58 + 59 |
| SEQ ID NOs: 50 + 12 + 15 | SEQ ID NOs: 52 + 5 + 53 + 54 + 55 + 67 + 69 + 58 + 59 |
| SEQ ID NOs: 50 + 13 + 15 | SEQ ID NOs: 52 + 5 + 53 + 54 + 55 + 67 + 69 + 58 + 59 |
| SEQ ID NOs: 50 + 14 + 15 | SEQ ID NOs: 52 + 5 + 53 + 54 + 55 + 67 + 68 + 58 + 59 |
| SEQ ID NOs: 50 + 49 + 15 | SEQ ID NOs: 52 + 5 + 53 + 54 + 55 + 67 + 69 + 58 + 59 |
| SEQ ID NOs: 50 + 8 + 51 | SEQ ID NOs: 16 + 5 + 53 + 54 + 55 + 56 + 57 + 58 + 59 |
| SEQ ID NOs: 50 + 9 + 51 | SEQ ID NOs: 16 + 5 + 53 + 54 + 55 + 56 + 57 + 58 + 59 |
| SEQ ID NOs: 50 + 10 + 51 | SEQ ID NOs: 16 + 5 + 53 + 54 + 55 + 56 + 57 + 58 + 59 |
| SEQ ID NOs: 50 + 11 + 51 | SEQ ID NOs: 16 + 5 + 53 + 54 + 55 + 56 + 57 + 58 + 59 |
| SEQ ID NOs: 50 + 12 + 51 | SEQ ID NOs: 16 + 5 + 53 + 54 + 55 + 56 + 57 + 58 + 59 |
| SEQ ID NOs: 50 + 13 + 51 | SEQ ID NOs: 16 + 5 + 53 + 54 + 55 + 56 + 57 + 58 + 59 |
| SEQ ID NOs: 50 + 14 + 51 | SEQ ID NOs: 16 + 5 + 53 + 54 + 55 + 56 + 57 + 58 + 59 |
| SEQ ID NOs: 50 + 49 + 51 | SEQ ID NOs: 16 + 5 + 53 + 54 + 55 + 56 + 57 + 58 + 59 |
| SEQ ID NOs: 50 + 8 + 51 | SEQ ID NOs: 16 + 5 + 53 + 54 + 55 + 56 + 68 + 58 + 59 |
| SEQ ID NOs: 50 + 9 + 51 | SEQ ID NOs: 16 + 5 + 53 + 54 + 55 + 56 + 68 + 58 + 59 |
| SEQ ID NOs: 50 + 10 + 51 | SEQ ID NOs: 16 + 5 + 53 + 54 + 55 + 56 + 68 + 58 + 59 |
| SEQ ID NOs: 50 + 11 + 51 | SEQ ID NOs: 16 + 5 + 53 + 54 + 55 + 56 + 68 + 58 + 59 |
| SEQ ID NOs: 50 + 12 + 51 | SEQ ID NOs: 16 + 5 + 53 + 54 + 55 + 56 + 68 + 58 + 59 |
| SEQ ID NOs: 50 + 13 + 51 | SEQ ID NOs: 16 + 5 + 53 + 54 + 55 + 56 + 68 + 58 + 59 |
| SEQ ID NOs: 50 + 14 + 51 | SEQ ID NOs: 16 + 5 + 53 + 54 + 55 + 56 + 68 + 58 + 59 |
| SEQ ID NOs: 50 + 49 + 51 | SEQ ID NOs: 16 + 5 + 53 + 54 + 55 + 56 + 68 + 58 + 59 |
| SEQ ID NOs: 50 + 8 + 51 | SEQ ID NOs: 16 + 5 + 53 + 54 + 55 + 56 + 69 + 58 + 59 |
| SEQ ID NOs: 50 + 9 + 51 | SEQ ID NOs: 16 + 5 + 53 + 54 + 55 + 56 + 69 + 58 + 59 |
| SEQ ID NOs: 50 + 10 + 51 | SEQ ID NOs: 16 + 5 + 53 + 54 + 55 + 56 + 69 + 58 + 59 |
| SEQ ID NOs: 50 + 11 + 51 | SEQ ID NOs: 16 + 5 + 53 + 54 + 55 + 56 + 69 + 58 + 59 |
| SEQ ID NOs: 50 + 12 + 51 | SEQ ID NOs: 16 + 5 + 53 + 54 + 55 + 56 + 69 + 58 + 59 |
| SEQ ID NOs: 50 + 13 + 51 | SEQ ID NOs: 16 + 5 + 53 + 54 + 55 + 56 + 69 + 58 + 59 |
| SEQ ID NOs: 50 + 14 + 51 | SEQ ID NOs: 16 + 5 + 53 + 54 + 55 + 56 + 69 + 58 + 59 |
| SEQ ID NOs: 50 + 49 + 51 | SEQ ID NOs: 16 + 5 + 53 + 54 + 55 + 56 + 69 + 58 + 59 |
| SEQ ID NOs: 50 + 8 + 51 | SEQ ID NOs: 16 + 5 + 53 + 54 + 55 + 60 + 57 + 58 + 59 |
| SEQ ID NOs: 50 + 9 + 51 | SEQ ID NOs: 16 + 5 + 53 + 54 + 55 + 60 + 57 + 58 + 59 |
| SEQ ID NOs: 50 + 10 + 51 | SEQ ID NOs: 16 + 5 + 53 + 54 + 55 + 60 + 57 + 58 + 59 |
| SEQ ID NOs: 50 + 11 + 51 | SEQ ID NOs: 16 + 5 + 53 + 54 + 55 + 60 + 57 + 58 + 59 |
| SEQ ID NOs: 50 + 12 + 51 | SEQ ID NOs: 16 + 5 + 53 + 54 + 55 + 60 + 57 + 58 + 59 |
| SEQ ID NOs: 50 + 13 + 51 | SEQ ID NOs: 16 + 5 + 53 + 54 + 55 + 60 + 57 + 58 + 59 |
| SEQ ID NOs: 50 + 14 + 51 | SEQ ID NOs: 16 + 5 + 53 + 54 + 55 + 60 + 57 + 58 + 59 |
| SEQ ID NOs: 50 + 49 + 51 | SEQ ID NOs: 16 + 5 + 53 + 54 + 55 + 60 + 57 + 58 + 59 |
| SEQ ID NOs: 50 + 8 + 51 | SEQ ID NOs: 16 + 5 + 53 + 54 + 55 + 60 + 68 + 58 + 59 |
| SEQ ID NOs: 50 + 9 + 51 | SEQ ID NOs: 16 + 5 + 53 + 54 + 55 + 60 + 68 + 58 + 59 |
| SEQ ID NOs: 50 + 10 + 51 | SEQ ID NOs: 16 + 5 + 53 + 54 + 55 + 60 + 68 + 58 + 59 |
| SEQ ID NOs: 50 + 11 + 51 | SEQ ID NOs: 16 + 5 + 53 + 54 + 55 + 60 + 68 + 58 + 59 |
| SEQ ID NOs: 50 + 12 + 51 | SEQ ID NOs: 16 + 5 + 53 + 54 + 55 + 60 + 68 + 58 + 59 |
| SEQ ID NOs: 50 + 13 + 51 | SEQ ID NOs: 16 + 5 + 53 + 54 + 55 + 60 + 68 + 58 + 59 |
| SEQ ID NOs: 50 + 14 + 51 | SEQ ID NOs: 16 + 5 + 53 + 54 + 55 + 60 + 68 + 58 + 59 |
| SEQ ID NOs: 50 + 49 + 51 | SEQ ID NOs: 16 + 5 + 53 + 54 + 55 + 60 + 68 + 58 + 59 |
| SEQ ID NOs: 50 + 8 + 51 | SEQ ID NOs: 16 + 5 + 53 + 54 + 55 + 60 + 69 + 58 + 59 |
| SEQ ID NOs: 50 + 9 + 51 | SEQ ID NOs: 16 + 5 + 53 + 54 + 55 + 60 + 69 + 58 + 59 |
| SEQ ID NOs: 50 + 10 + 51 | SEQ ID NOs: 16 + 5 + 53 + 54 + 55 + 60 + 69 + 58 + 59 |
| SEQ ID NOs: 50 + 11 + 51 | SEQ ID NOs: 16 + 5 + 53 + 54 + 55 + 60 + 69 + 58 + 59 |
| SEQ ID NOs: 50 + 12 + 51 | SEQ ID NOs: 16 + 5 + 53 + 54 + 55 + 60 + 69 + 58 + 59 |
| SEQ ID NOs: 50 + 13 + 51 | SEQ ID NOs: 16 + 5 + 53 + 54 + 55 + 60 + 69 + 58 + 59 |
| SEQ ID NOs: 50 + 14 + 51 | SEQ ID NOs: 16 + 5 + 53 + 54 + 55 + 60 + 69 + 58 + 59 |
| SEQ ID NOs: 50 + 49 + 51 | SEQ ID NOs: 16 + 5 + 53 + 54 + 55 + 60 + 69 + 58 + 59 |
| SEQ ID NOs: 50 + 8 + 51 | SEQ ID NOs: 16 + 5 + 53 + 54 + 55 + 61 + 57 + 58 + 59 |
| SEQ ID NOs: 50 + 9 + 51 | SEQ ID NOs: 16 + 5 + 53 + 54 + 55 + 61 + 57 + 58 + 59 |
| SEQ ID NOs: 50 + 10 + 51 | SEQ ID NOs: 16 + 5 + 53 + 54 + 55 + 61 + 57 + 58 + 59 |
| SEQ ID NOs: 50 + 11 + 51 | SEQ ID NOs: 16 + 5 + 53 + 54 + 55 + 61 + 57 + 58 + 59 |
| SEQ ID NOs: 50 + 12 + 51 | SEQ ID NOs: 16 + 5 + 53 + 54 + 55 + 61 + 57 + 58 + 59 |
| SEQ ID NOs: 50 + 13 + 51 | SEQ ID NOs: 16 + 5 + 53 + 54 + 55 + 61 + 57 + 58 + 59 |
| SEQ ID NOs: 50 + 14 + 51 | SEQ ID NOs: 16 + 5 + 53 + 54 + 55 + 61 + 57 + 58 + 59 |
| SEQ ID NOs: 50 + 49 + 51 | SEQ ID NOs: 16 + 5 + 53 + 54 + 55 + 61 + 57 + 58 + 59 |
| SEQ ID NOs: 50 + 8 + 51 | SEQ ID NOs: 16 + 5 + 53 + 54 + 55 + 61 + 68 + 58 + 59 |
| SEQ ID NOs: 50 + 9 + 51 | SEQ ID NOs: 16 + 5 + 53 + 54 + 55 + 61 + 68 + 58 + 59 |
| SEQ ID NOs: 50 + 10 + 51 | SEQ ID NOs: 16 + 5 + 53 + 54 + 55 + 61 + 68 + 58 + 59 |
| SEQ ID NOs: 50 + 11 + 51 | SEQ ID NOs: 16 + 5 + 53 + 54 + 55 + 61 + 68 + 58 + 59 |
| SEQ ID NOs: 50 + 12 + 51 | SEQ ID NOs: 16 + 5 + 53 + 54 + 55 + 61 + 68 + 58 + 59 |
| SEQ ID NOs: 50 + 13 + 51 | SEQ ID NOs: 16 + 5 + 53 + 54 + 55 + 61 + 68 + 58 + 59 |
| SEQ ID NOs: 50 + 14 + 51 | SEQ ID NOs: 16 + 5 + 53 + 54 + 55 + 61 + 68 + 58 + 59 |
| SEQ ID NOs: 50 + 49 + 51 | SEQ ID NOs: 16 + 5 + 53 + 54 + 55 + 61 + 68 + 58 + 59 |
| SEQ ID NOs: 50 + 8 + 51 | SEQ ID NOs: 16 + 5 + 53 + 54 + 55 + 61 + 69 + 58 + 59 |
| SEQ ID NOs: 50 + 9 + 51 | SEQ ID NOs: 16 + 5 + 53 + 54 + 55 + 61 + 69 + 58 + 59 |
| SEQ ID NOs: 50 + 10 + 51 | SEQ ID NOs: 16 + 5 + 53 + 54 + 55 + 61 + 69 + 58 + 59 |
| SEQ ID NOs: 50 + 11 + 51 | SEQ ID NOs: 16 + 5 + 53 + 54 + 55 + 61 + 69 + 58 + 59 |
| SEQ ID NOs: 50 + 12 + 51 | SEQ ID NOs: 16 + 5 + 53 + 54 + 55 + 61 + 69 + 58 + 59 |
| SEQ ID NOs: 50 + 13 + 51 | SEQ ID NOs: 16 + 5 + 53 + 54 + 55 + 61 + 69 + 58 + 59 |
| SEQ ID NOs: 50 + 14 + 51 | SEQ ID NOs: 16 + 5 + 53 + 54 + 55 + 61 + 69 + 58 + 59 |
| SEQ ID NOs: 50 + 49 + 51 | SEQ ID NOs: 16 + 5 + 53 + 54 + 55 + 61 + 69 + 58 + 59 |
| SEQ ID NOs: 50 + 8 + 51 | SEQ ID NOs: 16 + 5 + 53 + 54 + 55 + 62 + 57 + 58 + 59 |
| SEQ ID NOs: 50 + 9 + 51 | SEQ ID NOs: 16 + 5 + 53 + 54 + 55 + 62 + 57 + 58 + 59 |
| SEQ ID NOs: 50 + 10 + 51 | SEQ ID NOs: 16 + 5 + 53 + 54 + 55 + 62 + 57 + 58 + 59 |
| SEQ ID NOs: 50 + 11 + 51 | SEQ ID NOs: 16 + 5 + 53 + 54 + 55 + 62 + 57 + 58 + 59 |
| SEQ ID NOs: 50 + 12 + 51 | SEQ ID NOs: 16 + 5 + 53 + 54 + 55 + 62 + 57 + 58 + 59 |
| SEQ ID NOs: 50 + 13 + 51 | SEQ ID NOs: 16 + 5 + 53 + 54 + 55 + 62 + 57 + 58 + 59 |
| SEQ ID NOs: 50 + 14 + 51 | SEQ ID NOs: 16 + 5 + 53 + 54 + 55 + 62 + 57 + 58 + 59 |
| SEQ ID NOs: 50 + 49 + 51 | SEQ ID NOs: 16 + 5 + 53 + 54 + 55 + 62 + 57 + 58 + 59 |
| SEQ ID NOs: 50 + 8 + 51 | SEQ ID NOs: 16 + 5 + 53 + 54 + 55 + 62 + 68 + 58 + 59 |
| SEQ ID NOs: 50 + 9 + 51 | SEQ ID NOs: 16 + 5 + 53 + 54 + 55 + 62 + 68 + 58 + 59 |
| SEQ ID NOs: 50 + 10 + 51 | SEQ ID NOs: 16 + 5 + 53 + 54 + 55 + 62 + 68 + 58 + 59 |
| SEQ ID NOs: 50 + 11 + 51 | SEQ ID NOs: 16 + 5 + 53 + 54 + 55 + 62 + 68 + 58 + 59 |
| SEQ ID NOs: 50 + 12 + 51 | SEQ ID NOs: 16 + 5 + 53 + 54 + 55 + 62 + 68 + 58 + 59 |
| SEQ ID NOs: 50 + 13 + 51 | SEQ ID NOs: 16 + 5 + 53 + 54 + 55 + 62 + 68 + 58 + 59 |
| SEQ ID NOs: 50 + 14 + 51 | SEQ ID NOs: 16 + 5 + 53 + 54 + 55 + 62 + 68 + 58 + 59 |
| SEQ ID NOs: 50 + 49 + 51 | SEQ ID NOs: 16 + 5 + 53 + 54 + 55 + 62 + 68 + 58 + 59 |
| SEQ ID NOs: 50 + 8 + 51 | SEQ ID NOs: 16 + 5 + 53 + 54 + 55 + 62 + 69 + 58 + 59 |
| SEQ ID NOs: 50 + 9 + 51 | SEQ ID NOs: 16 + 5 + 53 + 54 + 55 + 62 + 69 + 58 + 59 |
| SEQ ID NOs: 50 + 10 + 51 | SEQ ID NOs: 16 + 5 + 53 + 54 + 55 + 62 + 69 + 58 + 59 |
| SEQ ID NOs: 50 + 11 + 51 | SEQ ID NOs: 16 + 5 + 53 + 54 + 55 + 62 + 69 + 58 + 59 |
| SEQ ID NOs: 50 + 12 + 51 | SEQ ID NOs: 16 + 5 + 53 + 54 + 55 + 62 + 69 + 58 + 59 |
| SEQ ID NOs: 50 + 13 + 51 | SEQ ID NOs: 16 + 5 + 53 + 54 + 55 + 62 + 69 + 58 + 59 |
| SEQ ID NOs: 50 + 14 + 51 | SEQ ID NOs: 16 + 5 + 53 + 54 + 55 + 62 + 69 + 58 + 59 |
| SEQ ID NOs: 50 + 49 + 51 | SEQ ID NOs: 16 + 5 + 53 + 54 + 55 + 62 + 69 + 58 + 59 |
| SEQ ID NOs: 50 + 8 + 51 | SEQ ID NOs: 16 + 5 + 53 + 54 + 55 + 63 + 57 + 58 + 59 |
| SEQ ID NOs: 50 + 9 + 51 | SEQ ID NOs: 16 + 5 + 53 + 54 + 55 + 63 + 57 + 58 + 59 |
| SEQ ID NOs: 50 + 10 + 51 | SEQ ID NOs: 16 + 5 + 53 + 54 + 55 + 63 + 57 + 58 + 59 |
| SEQ ID NOs: 50 + 11 + 51 | SEQ ID NOs: 16 + 5 + 53 + 54 + 55 + 63 + 57 + 58 + 59 |
| SEQ ID NOs: 50 + 12 + 51 | SEQ ID NOs: 16 + 5 + 53 + 54 + 55 + 63 + 57 + 58 + 59 |
| SEQ ID NOs: 50 + 13 + 51 | SEQ ID NOs: 16 + 5 + 53 + 54 + 55 + 63 + 57 + 58 + 59 |
| SEQ ID NOs: 50 + 14 + 51 | SEQ ID NOs: 16 + 5 + 53 + 54 + 55 + 63 + 57 + 58 + 59 |
| SEQ ID NOs: 50 + 49 + 51 | SEQ ID NOs: 16 + 5 + 53 + 54 + 55 + 63 + 57 + 58 + 59 |
| SEQ ID NOs: 50 + 8 + 51 | SEQ ID NOs: 16 + 5 + 53 + 54 + 55 + 63 + 68 + 58 + 59 |
| SEQ ID NOs: 50 + 9 + 51 | SEQ ID NOs: 16 + 5 + 53 + 54 + 55 + 63 + 68 + 58 + 59 |
| SEQ ID NOs: 50 + 10 + 51 | SEQ ID NOs: 16 + 5 + 53 + 54 + 55 + 63 + 68 + 58 + 59 |
| SEQ ID NOs: 50 + 11 + 51 | SEQ ID NOs: 16 + 5 + 53 + 54 + 55 + 63 + 68 + 58 + 59 |
| SEQ ID NOs: 50 + 12 + 51 | SEQ ID NOs: 16 + 5 + 53 + 54 + 55 + 63 + 68 + 58 + 59 |
| SEQ ID NOs: 50 + 13 + 51 | SEQ ID NOs: 16 + 5 + 53 + 54 + 55 + 63 + 68 + 58 + 59 |
| SEQ ID NOs: 50 + 14 + 51 | SEQ ID NOs: 16 + 5 + 53 + 54 + 55 + 63 + 68 + 58 + 59 |
| SEQ ID NOs: 50 + 49 + 51 | SEQ ID NOs: 16 + 5 + 53 + 54 + 55 + 63 + 68 + 58 + 59 |
| SEQ ID NOs: 50 + 8 + 51 | SEQ ID NOs: 16 + 5 + 53 + 54 + 55 + 63 + 69 + 58 + 59 |
| SEQ ID NOs: 50 + 9 + 51 | SEQ ID NOs: 16 + 5 + 53 + 54 + 55 + 63 + 69 + 58 + 59 |
| SEQ ID NOs: 50 + 10 + 51 | SEQ ID NOs: 16 + 5 + 53 + 54 + 55 + 63 + 69 + 58 + 59 |
| SEQ ID NOs: 50 + 11 + 51 | SEQ ID NOs: 16 + 5 + 53 + 54 + 55 + 63 + 69 + 58 + 59 |
| SEQ ID NOs: 50 + 12 + 51 | SEQ ID NOs: 16 + 5 + 53 + 54 + 55 + 63 + 69 + 58 + 59 |
| SEQ ID NOs: 50 + 13 + 51 | SEQ ID NOs: 16 + 5 + 53 + 54 + 55 + 63 + 69 + 58 + 59 |
| SEQ ID NOs: 50 + 14 + 51 | SEQ ID NOs: 16 + 5 + 53 + 54 + 55 + 63 + 69 + 58 + 59 |
| SEQ ID NOs: 50 + 49 + 51 | SEQ ID NOs: 16 + 5 + 53 + 54 + 55 + 63 + 69 + 58 + 59 |
| SEQ ID NOs: 50 + 8 + 51 | SEQ ID NOs: 16 + 5 + 53 + 54 + 55 + 64 + 57 + 58 + 59 |
| SEQ ID NOs: 50 + 9 + 51 | SEQ ID NOs: 16 + 5 + 53 + 54 + 55 + 64 + 57 + 58 + 59 |
| SEQ ID NOs: 50 + 10 + 51 | SEQ ID NOs: 16 + 5 + 53 + 54 + 55 + 64 + 57 + 58 + 59 |
| SEQ ID NOs: 50 + 11 + 51 | SEQ ID NOs: 16 + 5 + 53 + 54 + 55 + 64 + 57 + 58 + 59 |
| SEQ ID NOs: 50 + 12 + 51 | SEQ ID NOs: 16 + 5 + 53 + 54 + 55 + 64 + 57 + 58 + 59 |
| SEQ ID NOs: 50 + 13 + 51 | SEQ ID NOs: 16 + 5 + 53 + 54 + 55 + 64 + 57 + 58 + 59 |
| SEQ ID NOs: 50 + 14 + 51 | SEQ ID NOs: 16 + 5 + 53 + 54 + 55 + 64 + 57 + 58 + 59 |
| SEQ ID NOs: 50 + 49 + 51 | SEQ ID NOs: 16 + 5 + 53 + 54 + 55 + 64 + 57 + 58 + 59 |
| SEQ ID NOs: 50 + 8 + 51 | SEQ ID NOs: 16 + 5 + 53 + 54 + 55 + 64 + 68 + 58 + 59 |
| SEQ ID NOs: 50 + 9 + 51 | SEQ ID NOs: 16 + 5 + 53 + 54 + 55 + 64 + 68 + 58 + 59 |
| SEQ ID NOs: 50 + 10 + 51 | SEQ ID NOs: 16 + 5 + 53 + 54 + 55 + 64 + 68 + 58 + 59 |
| SEQ ID NOs: 50 + 11 + 51 | SEQ ID NOs: 16 + 5 + 53 + 54 + 55 + 64 + 68 + 58 + 59 |
| SEQ ID NOs: 50 + 12 + 51 | SEQ ID NOs: 16 + 5 + 53 + 54 + 55 + 64 + 68 + 58 + 59 |
| SEQ ID NOs: 50 + 13 + 51 | SEQ ID NOs: 16 + 5 + 53 + 54 + 55 + 64 + 68 + 58 + 59 |
| SEQ ID NOs: 50 + 14 + 51 | SEQ ID NOs: 16 + 5 + 53 + 54 + 55 + 64 + 68 + 58 + 59 |
| SEQ ID NOs: 50 + 49 + 51 | SEQ ID NOs: 16 + 5 + 53 + 54 + 55 + 64 + 68 + 58 + 59 |
| SEQ ID NOs: 50 + 8 + 51 | SEQ ID NOs: 16 + 5 + 53 + 54 + 55 + 64 + 69 + 58 + 59 |
| SEQ ID NOs: 50 + 9 + 51 | SEQ ID NOs: 16 + 5 + 53 + 54 + 55 + 64 + 69 + 58 + 59 |
| SEQ ID NOs: 50 + 10 + 51 | SEQ ID NOs: 16 + 5 + 53 + 54 + 55 + 64 + 69 + 58 + 59 |
| SEQ ID NOs: 50 + 11 + 51 | SEQ ID NOs: 16 + 5 + 53 + 54 + 55 + 64 + 69 + 58 + 59 |
| SEQ ID NOs: 50 + 12 + 51 | SEQ ID NOs: 16 + 5 + 53 + 54 + 55 + 64 + 69 + 58 + 59 |
| SEQ ID NOs: 50 + 13 + 51 | SEQ ID NOs: 16 + 5 + 53 + 54 + 55 + 64 + 69 + 58 + 59 |
| SEQ ID NOs: 50 + 14 + 51 | SEQ ID NOs: 16 + 5 + 53 + 54 + 55 + 64 + 69 + 58 + 59 |
| SEQ ID NOs: 50 + 49 + 51 | SEQ ID NOs: 16 + 5 + 53 + 54 + 55 + 64 + 69 + 58 + 59 |
| SEQ ID NOs: 50 + 8 + 51 | SEQ ID NOs: 16 + 5 + 53 + 54 + 55 + 65 + 57 + 58 + 59 |
| SEQ ID NOs: 50 + 9 + 51 | SEQ ID NOs: 16 + 5 + 53 + 54 + 55 + 65 + 57 + 58 + 59 |
| SEQ ID NOs: 50 + 10 + 51 | SEQ ID NOs: 16 + 5 + 53 + 54 + 55 + 65 + 57 + 58 + 59 |
| SEQ ID NOs: 50 + 11 + 51 | SEQ ID NOs: 16 + 5 + 53 + 54 + 55 + 65 + 57 + 58 + 59 |
| SEQ ID NOs: 50 + 12 + 51 | SEQ ID NOs: 16 + 5 + 53 + 54 + 55 + 65 + 57 + 58 + 59 |
| SEQ ID NOs: 50 + 13 + 51 | SEQ ID NOs: 16 + 5 + 53 + 54 + 55 + 65 + 57 + 58 + 59 |
| SEQ ID NOs: 50 + 14 + 51 | SEQ ID NOs: 16 + 5 + 53 + 54 + 55 + 65 + 57 + 58 + 59 |
| SEQ ID NOs: 50 + 49 + 51 | SEQ ID NOs: 16 + 5 + 53 + 54 + 55 + 65 + 57 + 58 + 59 |
| SEQ ID NOs: 50 + 8 + 51 | SEQ ID NOs: 16 + 5 + 53 + 54 + 55 + 65 + 68 + 58 + 59 |
| SEQ ID NOs: 50 + 9 + 51 | SEQ ID NOs: 16 + 5 + 53 + 54 + 55 + 65 + 68 + 58 + 59 |
| SEQ ID NOs: 50 + 10 + 51 | SEQ ID NOs: 16 + 5 + 53 + 54 + 55 + 65 + 68 + 58 + 59 |
| SEQ ID NOs: 50 + 11 + 51 | SEQ ID NOs: 16 + 5 + 53 + 54 + 55 + 65 + 68 + 58 + 59 |
| SEQ ID NOs: 50 + 12 + 51 | SEQ ID NOs: 16 + 5 + 53 + 54 + 55 + 65 + 68 + 58 + 59 |
| SEQ ID NOs: 50 + 13 + 51 | SEQ ID NOs: 16 + 5 + 53 + 54 + 55 + 65 + 68 + 58 + 59 |
| SEQ ID NOs: 50 + 14 + 51 | SEQ ID NOs: 16 + 5 + 53 + 54 + 55 + 65 + 68 + 58 + 59 |
| SEQ ID NOs: 50 + 49 + 51 | SEQ ID NOs: 16 + 5 + 53 + 54 + 55 + 65 + 68 + 58 + 59 |
| SEQ ID NOs: 50 + 8 + 51 | SEQ ID NOs: 16 + 5 + 53 + 54 + 55 + 65 + 69 + 58 + 59 |
| SEQ ID NOs: 50 + 9 + 51 | SEQ ID NOs: 16 + 5 + 53 + 54 + 55 + 65 + 69 + 58 + 59 |
| SEQ ID NOs: 50 + 10 + 51 | SEQ ID NOs: 16 + 5 + 53 + 54 + 55 + 65 + 69 + 58 + 59 |
| SEQ ID NOs: 50 + 11 + 51 | SEQ ID NOs: 16 + 5 + 53 + 54 + 55 + 65 + 69 + 58 + 59 |
| SEQ ID NOs: 50 + 12 + 51 | SEQ ID NOs: 16 + 5 + 53 + 54 + 55 + 65 + 69 + 58 + 59 |
| SEQ ID NOs: 50 + 13 + 51 | SEQ ID NOs: 16 + 5 + 53 + 54 + 55 + 65 + 69 + 58 + 59 |
| SEQ ID NOs: 50 + 14 + 51 | SEQ ID NOs: 16 + 5 + 53 + 54 + 55 + 65 + 69 + 58 + 59 |
| SEQ ID NOs: 50 + 49 + 51 | SEQ ID NOs: 16 + 5 + 53 + 54 + 55 + 65 + 69 + 58 + 59 |
| SEQ ID NOs: 50 + 8 + 51 | SEQ ID NOs: 16 + 5 + 53 + 54 + 55 + 66 + 57 + 58 + 59 |
| SEQ ID NOs: 50 + 9 + 51 | SEQ ID NOs: 16 + 5 + 53 + 54 + 55 + 66 + 57 + 58 + 59 |
| SEQ ID NOs: 50 + 10 + 51 | SEQ ID NOs: 16 + 5 + 53 + 54 + 55 + 66 + 57 + 58 + 59 |
| SEQ ID NOs: 50 + 11 + 51 | SEQ ID NOs: 16 + 5 + 53 + 54 + 55 + 66 + 57 + 58 + 59 |
| SEQ ID NOs: 50 + 12 + 51 | SEQ ID NOs: 16 + 5 + 53 + 54 + 55 + 66 + 57 + 58 + 59 |
| SEQ ID NOs: 50 + 13 + 51 | SEQ ID NOs: 16 + 5 + 53 + 54 + 55 + 66 + 57 + 58 + 59 |
| SEQ ID NOs: 50 + 14 + 51 | SEQ ID NOs: 16 + 5 + 53 + 54 + 55 + 66 + 57 + 58 + 59 |
| SEQ ID NOs: 50 + 49 + 51 | SEQ ID NOs: 16 + 5 + 53 + 54 + 55 + 66 + 57 + 58 + 59 |
| SEQ ID NOs: 50 + 8 + 51 | SEQ ID NOs: 16 + 5 + 53 + 54 + 55 + 66 + 68 + 58 + 59 |
| SEQ ID NOs: 50 + 9 + 51 | SEQ ID NOs: 16 + 5 + 53 + 54 + 55 + 66 + 68 + 58 + 59 |
| SEQ ID NOs: 50 + 10 + 51 | SEQ ID NOs: 16 + 5 + 53 + 54 + 55 + 66 + 68 + 58 + 59 |
| SEQ ID NOs: 50 + 11 + 51 | SEQ ID NOs: 16 + 5 + 53 + 54 + 55 + 66 + 68 + 58 + 59 |
| SEQ ID NOs: 50 + 12 + 51 | SEQ ID NOs: 16 + 5 + 53 + 54 + 55 + 66 + 68 + 58 + 59 |
| SEQ ID NOs: 50 + 13 + 51 | SEQ ID NOs: 16 + 5 + 53 + 54 + 55 + 66 + 68 + 58 + 59 |
| SEQ ID NOs: 50 + 14 + 51 | SEQ ID NOs: 16 + 5 + 53 + 54 + 55 + 66 + 68 + 58 + 59 |
| SEQ ID NOs: 50 + 49 + 51 | SEQ ID NOs: 16 + 5 + 53 + 54 + 55 + 66 + 68 + 58 + 59 |
| SEQ ID NOs: 50 + 8 + 51 | SEQ ID NOs: 16 + 5 + 53 + 54 + 55 + 66 + 69 + 58 + 59 |
| SEQ ID NOs: 50 + 9 + 51 | SEQ ID NOs: 16 + 5 + 53 + 54 + 55 + 66 + 69 + 58 + 59 |
| SEQ ID NOs: 50 + 10 + 51 | SEQ ID NOs: 16 + 5 + 53 + 54 + 55 + 66 + 69 + 58 + 59 |
| SEQ ID NOs: 50 + 11 + 51 | SEQ ID NOs: 16 + 5 + 53 + 54 + 55 + 66 + 69 + 58 + 59 |
| SEQ ID NOs: 50 + 12 + 51 | SEQ ID NOs: 16 + 5 + 53 + 54 + 55 + 66 + 69 + 58 + 59 |
| SEQ ID NOs: 50 + 13 + 51 | SEQ ID NOs: 16 + 5 + 53 + 54 + 55 + 66 + 69 + 58 + 59 |
| SEQ ID NOs: 50 + 14 + 51 | SEQ ID NOs: 16 + 5 + 53 + 54 + 55 + 66 + 69 + 58 + 59 |
| SEQ ID NOs: 50 + 49 + 51 | SEQ ID NOs: 16 + 5 + 53 + 54 + 55 + 66 + 69 + 58 + 59 |
| SEQ ID NOs: 50 + 8 + 51 | SEQ ID NOs: 16 + 5 + 53 + 54 + 55 + 67 + 57 + 58 + 59 |
| SEQ ID NOs: 50 + 9 + 51 | SEQ ID NOs: 16 + 5 + 53 + 54 + 55 + 67 + 57 + 58 + 59 |
| SEQ ID NOs: 50 + 10 + 51 | SEQ ID NOs: 16 + 5 + 53 + 54 + 55 + 67 + 57 + 58 + 59 |
| SEQ ID NOs: 50 + 11 + 51 | SEQ ID NOs: 16 + 5 + 53 + 54 + 55 + 67 + 57 + 58 + 59 |
| SEQ ID NOs: 50 + 12 + 51 | SEQ ID NOs: 16 + 5 + 53 + 54 + 55 + 67 + 57 + 58 + 59 |
| SEQ ID NOs: 50 + 13 + 51 | SEQ ID NOs: 16 + 5 + 53 + 54 + 55 + 67 + 57 + 58 + 59 |
| SEQ ID NOs: 50 + 14 + 51 | SEQ ID NOs: 16 + 5 + 53 + 54 + 55 + 67 + 57 + 58 + 59 |
| SEQ ID NOs: 50 + 49 + 51 | SEQ ID NOs: 16 + 5 + 53 + 54 + 55 + 67 + 57 + 58 + 59 |
| SEQ ID NOs: 50 + 8 + 51 | SEQ ID NOs: 16 + 5 + 53 + 54 + 55 + 67 + 68 + 58 + 59 |
| SEQ ID NOs: 50 + 9 + 51 | SEQ ID NOs: 16 + 5 + 53 + 54 + 55 + 67 + 68 + 58 + 59 |
| SEQ ID NOs: 50 + 10 + 51 | SEQ ID NOs: 16 + 5 + 53 + 54 + 55 + 67 + 68 + 58 + 59 |
| SEQ ID NOs: 50 + 11 + 51 | SEQ ID NOs: 16 + 5 + 53 + 54 + 55 + 67 + 68 + 58 + 59 |
| SEQ ID NOs: 50 + 12 + 51 | SEQ ID NOs: 16 + 5 + 53 + 54 + 55 + 67 + 68 + 58 + 59 |
| SEQ ID NOs: 50 + 13 + 51 | SEQ ID NOs: 16 + 5 + 53 + 54 + 55 + 67 + 68 + 58 + 59 |
| SEQ ID NOs: 50 + 14 + 51 | SEQ ID NOs: 16 + 5 + 53 + 54 + 55 + 67 + 68 + 58 + 59 |
| SEQ ID NOs: 50 + 49 + 51 | SEQ ID NOs: 16 + 5 + 53 + 54 + 55 + 67 + 68 + 58 + 59 |
| SEQ ID NOs: 50 + 8 + 51 | SEQ ID NOs: 16 + 5 + 53 + 54 + 55 + 67 + 69 + 58 + 59 |
| SEQ ID NOs: 50 + 9 + 51 | SEQ ID NOs: 16 + 5 + 53 + 54 + 55 + 67 + 69 + 58 + 59 |
| SEQ ID NOs: 50 + 10 + 51 | SEQ ID NOs: 16 + 5 + 53 + 54 + 55 + 67 + 69 + 58 + 59 |
| SEQ ID NOs: 50 + 11 + 51 | SEQ ID NOs: 16 + 5 + 53 + 54 + 55 + 67 + 69 + 58 + 59 |
| SEQ ID NOs: 50 + 12 + 51 | SEQ ID NOs: 16 + 5 + 53 + 54 + 55 + 67 + 69 + 58 + 59 |
| SEQ ID NOs: 50 + 13 + 51 | SEQ ID NOs: 16 + 5 + 53 + 54 + 55 + 67 + 69 + 58 + 59 |
| SEQ ID NOs: 50 + 14 + 51 | SEQ ID NOs: 16 + 5 + 53 + 54 + 55 + 67 + 69 + 58 + 59 |
| SEQ ID NOs: 50 + 49 + 51 | SEQ ID NOs: 16 + 5 + 53 + 54 + 55 + 67 + 69 + 58 + 59 |
| SEQ ID NOs: 50 + 8 + 15 | SEQ ID NOs: 16 + 5 + 53 + 54 + 55 + 56 + 57 + 58 + 59 |
| SEQ ID NOs: 50 + 9 + 15 | SEQ ID NOs: 16 + 5 + 53 + 54 + 55 + 56 + 57 + 58 + 59 |
| SEQ ID NOs: 50 + 10 + 15 | SEQ ID NOs: 16 + 5 + 53 + 54 + 55 + 56 + 57 + 58 + 59 |
| SEQ ID NOs: 50 + 11 + 15 | SEQ ID NOs: 16 + 5 + 53 + 54 + 55 + 56 + 57 + 58 + 59 |
| SEQ ID NOs: 50 + 12 + 15 | SEQ ID NOs: 16 + 5 + 53 + 54 + 55 + 56 + 57 + 58 + 59 |
| SEQ ID NOs: 50 + 13 + 15 | SEQ ID NOs: 16 + 5 + 53 + 54 + 55 + 56 + 57 + 58 + 59 |
| SEQ ID NOs: 50 + 14 + 15 | SEQ ID NOs: 16 + 5 + 53 + 54 + 55 + 56 + 57 + 58 + 59 |
| SEQ ID NOs: 50 + 49 + 15 | SEQ ID NOs: 16 + 5 + 53 + 54 + 55 + 56 + 57 + 58 + 59 |
| SEQ ID NOs: 50 + 8 + 15 | SEQ ID NOs: 16 + 5 + 53 + 54 + 55 + 56 + 68 + 58 + 59 |
| SEQ ID NOs: 50 + 9 + 15 | SEQ ID NOs: 16 + 5 + 53 + 54 + 55 + 56 + 68 + 58 + 59 |
| SEQ ID NOs: 50 + 10 + 15 | SEQ ID NOs: 16 + 5 + 53 + 54 + 55 + 56 + 68 + 58 + 59 |
| SEQ ID NOs: 50 + 11 + 15 | SEQ ID NOs: 16 + 5 + 53 + 54 + 55 + 56 + 68 + 58 + 59 |
| SEQ ID NOs: 50 + 12 + 15 | SEQ ID NOs: 16 + 5 + 53 + 54 + 55 + 56 + 68 + 58 + 59 |
| SEQ ID NOs: 50 + 13 + 15 | SEQ ID NOs: 16 + 5 + 53 + 54 + 55 + 56 + 68 + 58 + 59 |
| SEQ ID NOs: 50 + 14 + 15 | SEQ ID NOs: 16 + 5 + 53 + 54 + 55 + 56 + 68 + 58 + 59 |
| SEQ ID NOs: 50 + 49 + 15 | SEQ ID NOs: 16 + 5 + 53 + 54 + 55 + 56 + 68 + 58 + 59 |
| SEQ ID NOs: 50 + 8 + 15 | SEQ ID NOs: 16 + 5 + 53 + 54 + 55 + 56 + 69 + 58 + 59 |
| SEQ ID NOs: 50 + 9 + 15 | SEQ ID NOs: 16 + 5 + 53 + 54 + 55 + 56 + 69 + 58 + 59 |
| SEQ ID NOs: 50 + 10 + 15 | SEQ ID NOs: 16 + 5 + 53 + 54 + 55 + 56 + 69 + 58 + 59 |
| SEQ ID NOs: 50 + 11 + 15 | SEQ ID NOs: 16 + 5 + 53 + 54 + 55 + 56 + 69 + 58 + 59 |
| SEQ ID NOs: 50 + 12 + 15 | SEQ ID NOs: 16 + 5 + 53 + 54 + 55 + 56 + 69 + 58 + 59 |
| SEQ ID NOs: 50 + 13 + 15 | SEQ ID NOs: 16 + 5 + 53 + 54 + 55 + 56 + 69 + 58 + 59 |
| SEQ ID NOs: 50 + 14 + 15 | SEQ ID NOs: 16 + 5 + 53 + 54 + 55 + 56 + 69 + 58 + 59 |
| SEQ ID NOs: 50 + 49 + 15 | SEQ ID NOs: 16 + 5 + 53 + 54 + 55 + 56 + 69 + 58 + 59 |
| SEQ ID NOs: 50 + 8 + 15 | SEQ ID NOs: 16 + 5 + 53 + 54 + 55 + 60 + 57 + 58 + 59 |
| SEQ ID NOs: 50 + 9 + 15 | SEQ ID NOs: 16 + 5 + 53 + 54 + 55 + 60 + 57 + 58 + 59 |
| SEQ ID NOs: 50 + 10 + 15 | SEQ ID NOs: 16 + 5 + 53 + 54 + 55 + 60 + 57 + 58 + 59 |
| SEQ ID NOs: 50 + 11 + 15 | SEQ ID NOs: 16 + 5 + 53 + 54 + 55 + 60 + 57 + 58 + 59 |
| SEQ ID NOs: 50 + 12 + 15 | SEQ ID NOs: 16 + 5 + 53 + 54 + 55 + 60 + 57 + 58 + 59 |
| SEQ ID NOs: 50 + 13 + 15 | SEQ ID NOs: 16 + 5 + 53 + 54 + 55 + 60 + 57 + 58 + 59 |
| SEQ ID NOs: 50 + 14 + 15 | SEQ ID NOs: 16 + 5 + 53 + 54 + 55 + 60 + 57 + 58 + 59 |
| SEQ ID NOs: 50 + 49 + 15 | SEQ ID NOs: 16 + 5 + 53 + 54 + 55 + 60 + 57 + 58 + 59 |
| SEQ ID NOs: 50 + 8 + 15 | SEQ ID NOs: 16 + 5 + 53 + 54 + 55 + 60 + 68 + 58 + 59 |
| SEQ ID NOs: 50 + 9 + 15 | SEQ ID NOs: 16 + 5 + 53 + 54 + 55 + 60 + 68 + 58 + 59 |
| SEQ ID NOs: 50 + 10 + 15 | SEQ ID NOs: 16 + 5 + 53 + 54 + 55 + 60 + 68 + 58 + 59 |
| SEQ ID NOs: 50 + 11 + 15 | SEQ ID NOs: 16 + 5 + 53 + 54 + 55 + 60 + 68 + 58 + 59 |
| SEQ ID NOs: 50 + 12 + 15 | SEQ ID NOs: 16 + 5 + 53 + 54 + 55 + 60 + 68 + 58 + 59 |
| SEQ ID NOs: 50 + 13 + 15 | SEQ ID NOs: 16 + 5 + 53 + 54 + 55 + 60 + 68 + 58 + 59 |
| SEQ ID NOs: 50 + 14 + 15 | SEQ ID NOs: 16 + 5 + 53 + 54 + 55 + 60 + 68 + 58 + 59 |
| SEQ ID NOs: 50 + 49 + 15 | SEQ ID NOs: 16 + 5 + 53 + 54 + 55 + 60 + 68 + 58 + 59 |
| SEQ ID NOs: 50 + 8 + 15 | SEQ ID NOs: 16 + 5 + 53 + 54 + 55 + 60 + 69 + 58 + 59 |
| SEQ ID NOs: 50 + 9 + 15 | SEQ ID NOs: 16 + 5 + 53 + 54 + 55 + 60 + 69 + 58 + 59 |
| SEQ ID NOs: 50 + 10 + 15 | SEQ ID NOs: 16 + 5 + 53 + 54 + 55 + 60 + 69 + 58 + 59 |
| SEQ ID NOs: 50 + 11 + 15 | SEQ ID NOs: 16 + 5 + 53 + 54 + 55 + 60 + 69 + 58 + 59 |
| SEQ ID NOs: 50 + 12 + 15 | SEQ ID NOs: 16 + 5 + 53 + 54 + 55 + 60 + 69 + 58 + 59 |
| SEQ ID NOs: 50 + 13 + 15 | SEQ ID NOs: 16 + 5 + 53 + 54 + 55 + 60 + 69 + 58 + 59 |
| SEQ ID NOs: 50 + 14 + 15 | SEQ ID NOs: 16 + 5 + 53 + 54 + 55 + 60 + 69 + 58 + 59 |
| SEQ ID NOs: 50 + 49 + 15 | SEQ ID NOs: 16 + 5 + 53 + 54 + 55 + 60 + 69 + 58 + 59 |
| SEQ ID NOs: 50 + 8 + 15 | SEQ ID NOs: 16 + 5 + 53 + 54 + 55 + 61 + 57 + 58 + 59 |
| SEQ ID NOs: 50 + 9 + 15 | SEQ ID NOs: 16 + 5 + 53 + 54 + 55 + 61 + 57 + 58 + 59 |
| SEQ ID NOs: 50 + 10 + 15 | SEQ ID NOs: 16 + 5 + 53 + 54 + 55 + 61 + 57 + 58 + 59 |
| SEQ ID NOs: 50 + 11 + 15 | SEQ ID NOs: 16 + 5 + 53 + 54 + 55 + 61 + 57 + 58 + 59 |
| SEQ ID NOs: 50 + 12 + 15 | SEQ ID NOs: 16 + 5 + 53 + 54 + 55 + 61 + 57 + 58 + 59 |
| SEQ ID NOs: 50 + 13 + 15 | SEQ ID NOs: 16 + 5 + 53 + 54 + 55 + 61 + 57 + 58 + 59 |
| SEQ ID NOs: 50 + 14 + 15 | SEQ ID NOs: 16 + 5 + 53 + 54 + 55 + 61 + 57 + 58 + 59 |
| SEQ ID NOs: 50 + 49 + 15 | SEQ ID NOs: 16 + 5 + 53 + 54 + 55 + 61 + 57 + 58 + 59 |
| SEQ ID NOs: 50 + 8 + 15 | SEQ ID NOs: 16 + 5 + 53 + 54 + 55 + 61 + 68 + 58 + 59 |
| SEQ ID NOs: 50 + 9 + 15 | SEQ ID NOs: 16 + 5 + 53 + 54 + 55 + 61 + 68 + 58 + 59 |
| SEQ ID NOs: 50 + 10 + 15 | SEQ ID NOs: 16 + 5 + 53 + 54 + 55 + 61 + 68 + 58 + 59 |
| SEQ ID NOs: 50 + 11 + 15 | SEQ ID NOs: 16 + 5 + 53 + 54 + 55 + 61 + 68 + 58 + 59 |
| SEQ ID NOs: 50 + 12 + 15 | SEQ ID NOs: 16 + 5 + 53 + 54 + 55 + 61 + 68 + 58 + 59 |
| SEQ ID NOs: 50 + 13 + 15 | SEQ ID NOs: 16 + 5 + 53 + 54 + 55 + 61 + 68 + 58 + 59 |
| SEQ ID NOs: 50 + 14 + 15 | SEQ ID NOs: 16 + 5 + 53 + 54 + 55 + 61 + 68 + 58 + 59 |
| SEQ ID NOs: 50 + 49 + 15 | SEQ ID NOs: 16 + 5 + 53 + 54 + 55 + 61 + 68 + 58 + 59 |
| SEQ ID NOs: 50 + 8 + 15 | SEQ ID NOs: 16 + 5 + 53 + 54 + 55 + 61 + 69 + 58 + 59 |
| SEQ ID NOs: 50 + 9 + 15 | SEQ ID NOs: 16 + 5 + 53 + 54 + 55 + 61 + 69 + 58 + 59 |
| SEQ ID NOs: 50 + 10 + 15 | SEQ ID NOs: 16 + 5 + 53 + 54 + 55 + 61 + 69 + 58 + 59 |
| SEQ ID NOs: 50 + 11 + 15 | SEQ ID NOs: 16 + 5 + 53 + 54 + 55 + 61 + 69 + 58 + 59 |
| SEQ ID NOs: 50 + 12 + 15 | SEQ ID NOs: 16 + 5 + 53 + 54 + 55 + 61 + 69 + 58 + 59 |
| SEQ ID NOs: 50 + 13 + 15 | SEQ ID NOs: 16 + 5 + 53 + 54 + 55 + 61 + 69 + 58 + 59 |
| SEQ ID NOs: 50 + 14 + 15 | SEQ ID NOs: 16 + 5 + 53 + 54 + 55 + 61 + 69 + 58 + 59 |
| SEQ ID NOs: 50 + 49 + 15 | SEQ ID NOs: 16 + 5 + 53 + 54 + 55 + 61 + 69 + 58 + 59 |
| SEQ ID NOs: 50 + 8 + 15 | SEQ ID NOs: 16 + 5 + 53 + 54 + 55 + 62 + 57 + 58 + 59 |
| SEQ ID NOs: 50 + 9 + 15 | SEQ ID NOs: 16 + 5 + 53 + 54 + 55 + 62 + 57 + 58 + 59 |
| SEQ ID NOs: 50 + 10 + 15 | SEQ ID NOs: 16 + 5 + 53 + 54 + 55 + 62 + 57 + 58 + 59 |
| SEQ ID NOs: 50 + 11 + 15 | SEQ ID NOs: 16 + 5 + 53 + 54 + 55 + 62 + 57 + 58 + 59 |
| SEQ ID NOs: 50 + 12 + 15 | SEQ ID NOs: 16 + 5 + 53 + 54 + 55 + 62 + 57 + 58 + 59 |
| SEQ ID NOs: 50 + 13 + 15 | SEQ ID NOs: 16 + 5 + 53 + 54 + 55 + 62 + 57 + 58 + 59 |
| SEQ ID NOs: 50 + 14 + 15 | SEQ ID NOs: 16 + 5 + 53 + 54 + 55 + 62 + 57 + 58 + 59 |
| SEQ ID NOs: 50 + 49 + 15 | SEQ ID NOs: 16 + 5 + 53 + 54 + 55 + 62 + 57 + 58 + 59 |
| SEQ ID NOs: 50 + 8 + 15 | SEQ ID NOs: 16 + 5 + 53 + 54 + 55 + 62 + 68 + 58 + 59 |
| SEQ ID NOs: 50 + 9 + 15 | SEQ ID NOs: 16 + 5 + 53 + 54 + 55 + 62 + 68 + 58 + 59 |
| SEQ ID NOs: 50 + 10 + 15 | SEQ ID NOs: 16 + 5 + 53 + 54 + 55 + 62 + 68 + 58 + 59 |
| SEQ ID NOs: 50 + 11 + 15 | SEQ ID NOs: 16 + 5 + 53 + 54 + 55 + 62 + 68 + 58 + 59 |
| SEQ ID NOs: 50 + 12 + 15 | SEQ ID NOs: 16 + 5 + 53 + 54 + 55 + 62 + 68 + 58 + 59 |
| SEQ ID NOs: 50 + 13 + 15 | SEQ ID NOs: 16 + 5 + 53 + 54 + 55 + 62 + 68 + 58 + 59 |
| SEQ ID NOs: 50 + 14 + 15 | SEQ ID NOs: 16 + 5 + 53 + 54 + 55 + 62 + 68 + 58 + 59 |
| SEQ ID NOs: 50 + 49 + 15 | SEQ ID NOs: 16 + 5 + 53 + 54 + 55 + 62 + 68 + 58 + 59 |
| SEQ ID NOs: 50 + 8 + 15 | SEQ ID NOs: 16 + 5 + 53 + 54 + 55 + 62 + 69 + 58 + 59 |
| SEQ ID NOs: 50 + 9 + 15 | SEQ ID NOs: 16 + 5 + 53 + 54 + 55 + 62 + 69 + 58 + 59 |
| SEQ ID NOs: 50 + 10 + 15 | SEQ ID NOs: 16 + 5 + 53 + 54 + 55 + 62 + 69 + 58 + 59 |
| SEQ ID NOs: 50 + 11 + 15 | SEQ ID NOs: 16 + 5 + 53 + 54 + 55 + 62 + 69 + 58 + 59 |
| SEQ ID NOs: 50 + 12 + 15 | SEQ ID NOs: 16 + 5 + 53 + 54 + 55 + 62 + 69 + 58 + 59 |
| SEQ ID NOs: 50 + 13 + 15 | SEQ ID NOs: 16 + 5 + 53 + 54 + 55 + 62 + 69 + 58 + 59 |
| SEQ ID NOs: 50 + 14 + 15 | SEQ ID NOs: 16 + 5 + 53 + 54 + 55 + 62 + 69 + 58 + 59 |
| SEQ ID NOs: 50 + 49 + 15 | SEQ ID NOs: 16 + 5 + 53 + 54 + 55 + 62 + 69 + 58 + 59 |
| SEQ ID NOs: 50 + 8 + 15 | SEQ ID NOs: 16 + 5 + 53 + 54 + 55 + 63 + 57 + 58 + 59 |
| SEQ ID NOs: 50 + 9 + 15 | SEQ ID NOs: 16 + 5 + 53 + 54 + 55 + 63 + 57 + 58 + 59 |
| SEQ ID NOs: 50 + 10 + 15 | SEQ ID NOs: 16 + 5 + 53 + 54 + 55 + 63 + 57 + 58 + 59 |
| SEQ ID NOs: 50 + 11 + 15 | SEQ ID NOs: 16 + 5 + 53 + 54 + 55 + 63 + 57 + 58 + 59 |
| SEQ ID NOs: 50 + 12 + 15 | SEQ ID NOs: 16 + 5 + 53 + 54 + 55 + 63 + 57 + 58 + 59 |
| SEQ ID NOs: 50 + 13 + 15 | SEQ ID NOs: 16 + 5 + 53 + 54 + 55 + 63 + 57 + 58 + 59 |
| SEQ ID NOs: 50 + 14 + 15 | SEQ ID NOs: 16 + 5 + 53 + 54 + 55 + 63 + 57 + 58 + 59 |
| SEQ ID NOs: 50 + 49 + 15 | SEQ ID NOs: 16 + 5 + 53 + 54 + 55 + 63 + 57 + 58 + 59 |
| SEQ ID NOs: 50 + 8 + 15 | SEQ ID NOs: 16 + 5 + 53 + 54 + 55 + 63 + 68 + 58 + 59 |
| SEQ ID NOs: 50 + 9 + 15 | SEQ ID NOs: 16 + 5 + 53 + 54 + 55 + 63 + 68 + 58 + 59 |
| SEQ ID NOs: 50 + 10 + 15 | SEQ ID NOs: 16 + 5 + 53 + 54 + 55 + 63 + 68 + 58 + 59 |
| SEQ ID NOs: 50 + 11 + 15 | SEQ ID NOs: 16 + 5 + 53 + 54 + 55 + 63 + 68 + 58 + 59 |
| SEQ ID NOs: 50 + 12 + 15 | SEQ ID NOs: 16 + 5 + 53 + 54 + 55 + 63 + 68 + 58 + 59 |
| SEQ ID NOs: 50 + 13 + 15 | SEQ ID NOs: 16 + 5 + 53 + 54 + 55 + 63 + 68 + 58 + 59 |
| SEQ ID NOs: 50 + 14 + 15 | SEQ ID NOs: 16 + 5 + 53 + 54 + 55 + 63 + 68 + 58 + 59 |
| SEQ ID NOs: 50 + 49 + 15 | SEQ ID NOs: 16 + 5 + 53 + 54 + 55 + 63 + 68 + 58 + 59 |
| SEQ ID NOs: 50 + 8 + 15 | SEQ ID NOs: 16 + 5 + 53 + 54 + 55 + 63 + 69 + 58 + 59 |
| SEQ ID NOs: 50 + 9 + 15 | SEQ ID NOs: 16 + 5 + 53 + 54 + 55 + 63 + 69 + 58 + 59 |
| SEQ ID NOs: 50 + 10 + 15 | SEQ ID NOs: 16 + 5 + 53 + 54 + 55 + 63 + 69 + 58 + 59 |
| SEQ ID NOs: 50 + 11 + 15 | SEQ ID NOs: 16 + 5 + 53 + 54 + 55 + 63 + 69 + 58 + 59 |
| SEQ ID NOs: 50 + 12 + 15 | SEQ ID NOs: 16 + 5 + 53 + 54 + 55 + 63 + 69 + 58 + 59 |
| SEQ ID NOs: 50 + 13 + 15 | SEQ ID NOs: 16 + 5 + 53 + 54 + 55 + 63 + 69 + 58 + 59 |
| SEQ ID NOs: 50 + 14 + 15 | SEQ ID NOs: 16 + 5 + 53 + 54 + 55 + 63 + 69 + 58 + 59 |
| SEQ ID NOs: 50 + 49 + 15 | SEQ ID NOs: 16 + 5 + 53 + 54 + 55 + 63 + 69 + 58 + 59 |
| SEQ ID NOs: 50 + 8 + 15 | SEQ ID NOs: 16 + 5 + 53 + 54 + 55 + 64 + 57 + 58 + 59 |
| SEQ ID NOs: 50 + 9 + 15 | SEQ ID NOs: 16 + 5 + 53 + 54 + 55 + 64 + 57 + 58 + 59 |
| SEQ ID NOs: 50 + 10 + 15 | SEQ ID NOs: 16 + 5 + 53 + 54 + 55 + 64 + 57 + 58 + 59 |
| SEQ ID NOs: 50 + 11 + 15 | SEQ ID NOs: 16 + 5 + 53 + 54 + 55 + 64 + 57 + 58 + 59 |
| SEQ ID NOs: 50 + 12 + 15 | SEQ ID NOs: 16 + 5 + 53 + 54 + 55 + 64 + 57 + 58 + 59 |
| SEQ ID NOs: 50 + 13 + 15 | SEQ ID NOs: 16 + 5 + 53 + 54 + 55 + 64 + 57 + 58 + 59 |
| SEQ ID NOs: 50 + 14 + 15 | SEQ ID NOs: 16 + 5 + 53 + 54 + 55 + 64 + 57 + 58 + 59 |
| SEQ ID NOs: 50 + 49 + 15 | SEQ ID NOs: 16 + 5 + 53 + 54 + 55 + 64 + 57 + 58 + 59 |
| SEQ ID NOs: 50 + 8 + 15 | SEQ ID NOs: 16 + 5 + 53 + 54 + 55 + 64 + 68 + 58 + 59 |
| SEQ ID NOs: 50 + 9 + 15 | SEQ ID NOs: 16 + 5 + 53 + 54 + 55 + 64 + 68 + 58 + 59 |
| SEQ ID NOs: 50 + 10 + 15 | SEQ ID NOs: 16 + 5 + 53 + 54 + 55 + 64 + 68 + 58 + 59 |
| SEQ ID NOs: 50 + 11 + 15 | SEQ ID NOs: 16 + 5 + 53 + 54 + 55 + 64 + 68 + 58 + 59 |
| SEQ ID NOs: 50 + 12 + 15 | SEQ ID NOs: 16 + 5 + 53 + 54 + 55 + 64 + 68 + 58 + 59 |
| SEQ ID NOs: 50 + 13 + 15 | SEQ ID NOs: 16 + 5 + 53 + 54 + 55 + 64 + 68 + 58 + 59 |
| SEQ ID NOs: 50 + 14 + 15 | SEQ ID NOs: 16 + 5 + 53 + 54 + 55 + 64 + 68 + 58 + 59 |
| SEQ ID NOs: 50 + 49 + 15 | SEQ ID NOs: 16 + 5 + 53 + 54 + 55 + 64 + 68 + 58 + 59 |
| SEQ ID NOs: 50 + 8 + 15 | SEQ ID NOs: 16 + 5 + 53 + 54 + 55 + 64 + 69 + 58 + 59 |
| SEQ ID NOs: 50 + 9 + 15 | SEQ ID NOs: 16 + 5 + 53 + 54 + 55 + 64 + 69 + 58 + 59 |
| SEQ ID NOs: 50 + 10 + 15 | SEQ ID NOs: 16 + 5 + 53 + 54 + 55 + 64 + 69 + 58 + 59 |
| SEQ ID NOs: 50 + 11 + 15 | SEQ ID NOs: 16 + 5 + 53 + 54 + 55 + 64 + 69 + 58 + 59 |
| SEQ ID NOs: 50 + 12 + 15 | SEQ ID NOs: 16 + 5 + 53 + 54 + 55 + 64 + 69 + 58 + 59 |
| SEQ ID NOs: 50 + 13 + 15 | SEQ ID NOs: 16 + 5 + 53 + 54 + 55 + 64 + 69 + 58 + 59 |
| SEQ ID NOs: 50 + 14 + 15 | SEQ ID NOs: 16 + 5 + 53 + 54 + 55 + 64 + 69 + 58 + 59 |
| SEQ ID NOs: 50 + 49 + 15 | SEQ ID NOs: 16 + 5 + 53 + 54 + 55 + 64 + 69 + 58 + 59 |
| SEQ ID NOs: 50 + 8 + 15 | SEQ ID NOs: 16 + 5 + 53 + 54 + 55 + 65 + 57 + 58 + 59 |
| SEQ ID NOs: 50 + 9 + 15 | SEQ ID NOs: 16 + 5 + 53 + 54 + 55 + 65 + 57 + 58 + 59 |
| SEQ ID NOs: 50 + 10 + 15 | SEQ ID NOs: 16 + 5 + 53 + 54 + 55 + 65 + 57 + 58 + 59 |
| SEQ ID NOs: 50 + 11 + 15 | SEQ ID NOs: 16 + 5 + 53 + 54 + 55 + 65 + 57 + 58 + 59 |
| SEQ ID NOs: 50 + 12 + 15 | SEQ ID NOs: 16 + 5 + 53 + 54 + 55 + 65 + 57 + 58 + 59 |
| SEQ ID NOs: 50 + 13 + 15 | SEQ ID NOs: 16 + 5 + 53 + 54 + 55 + 65 + 57 + 58 + 59 |
| SEQ ID NOs: 50 + 14 + 15 | SEQ ID NOs: 16 + 5 + 53 + 54 + 55 + 65 + 57 + 58 + 59 |
| SEQ ID NOs: 50 + 49 + 15 | SEQ ID NOs: 16 + 5 + 53 + 54 + 55 + 65 + 57 + 58 + 59 |
| SEQ ID NOs: 50 + 8 + 15 | SEQ ID NOs: 16 + 5 + 53 + 54 + 55 + 65 + 68 + 58 + 59 |
| SEQ ID NOs: 50 + 9 + 15 | SEQ ID NOs: 16 + 5 + 53 + 54 + 55 + 65 + 68 + 58 + 59 |
| SEQ ID NOs: 50 + 10 + 15 | SEQ ID NOs: 16 + 5 + 53 + 54 + 55 + 65 + 68 + 58 + 59 |
| SEQ ID NOs: 50 + 11 + 15 | SEQ ID NOs: 16 + 5 + 53 + 54 + 55 + 65 + 68 + 58 + 59 |
| SEQ ID NOs: 50 + 12 + 15 | SEQ ID NOs: 16 + 5 + 53 + 54 + 55 + 65 + 68 + 58 + 59 |
| SEQ ID NOs: 50 + 13 + 15 | SEQ ID NOs: 16 + 5 + 53 + 54 + 55 + 65 + 68 + 58 + 59 |
| SEQ ID NOs: 50 + 14 + 15 | SEQ ID NOs: 16 + 5 + 53 + 54 + 55 + 65 + 68 + 58 + 59 |
| SEQ ID NOs: 50 + 49 + 15 | SEQ ID NOs: 16 + 5 + 53 + 54 + 55 + 65 + 68 + 58 + 59 |
| SEQ ID NOs: 50 + 8 + 15 | SEQ ID NOs: 16 + 5 + 53 + 54 + 55 + 65 + 69 + 58 + 59 |
| SEQ ID NOs: 50 + 9 + 15 | SEQ ID NOs: 16 + 5 + 53 + 54 + 55 + 65 + 69 + 58 + 59 |
| SEQ ID NOs: 50 + 10 + 15 | SEQ ID NOs: 16 + 5 + 53 + 54 + 55 + 65 + 69 + 58 + 59 |
| SEQ ID NOs: 50 + 11 + 15 | SEQ ID NOs: 16 + 5 + 53 + 54 + 55 + 65 + 69 + 58 + 59 |
| SEQ ID NOs: 50 + 12 + 15 | SEQ ID NOs: 16 + 5 + 53 + 54 + 55 + 65 + 69 + 58 + 59 |
| SEQ ID NOs: 50 + 13 + 15 | SEQ ID NOs: 16 + 5 + 53 + 54 + 55 + 65 + 69 + 58 + 59 |
| SEQ ID NOs: 50 + 14 + 15 | SEQ ID NOs: 16 + 5 + 53 + 54 + 55 + 65 + 69 + 58 + 59 |
| SEQ ID NOs: 50 + 49 + 15 | SEQ ID NOs: 16 + 5 + 53 + 54 + 55 + 65 + 69 + 58 + 59 |
| SEQ ID NOs: 50 + 8 + 15 | SEQ ID NOs: 16 + 5 + 53 + 54 + 55 + 66 + 57 + 58 + 59 |
| SEQ ID NOs: 50 + 9 + 15 | SEQ ID NOs: 16 + 5 + 53 + 54 + 55 + 66 + 57 + 58 + 59 |
| SEQ ID NOs: 50 + 10 + 15 | SEQ ID NOs: 16 + 5 + 53 + 54 + 55 + 66 + 57 + 58 + 59 |
| SEQ ID NOs: 50 + 11 + 15 | SEQ ID NOs: 16 + 5 + 53 + 54 + 55 + 66 + 57 + 58 + 59 |
| SEQ ID NOs: 50 + 12 + 15 | SEQ ID NOs: 16 + 5 + 53 + 54 + 55 + 66 + 57 + 58 + 59 |
| SEQ ID NOs: 50 + 13 + 15 | SEQ ID NOs: 16 + 5 + 53 + 54 + 55 + 66 + 57 + 58 + 59 |
| SEQ ID NOs: 50 + 14 + 15 | SEQ ID NOs: 16 + 5 + 53 + 54 + 55 + 66 + 57 + 58 + 59 |
| SEQ ID NOs: 50 + 49 + 15 | SEQ ID NOs: 16 + 5 + 53 + 54 + 55 + 66 + 57 + 58 + 59 |
| SEQ ID NOs: 50 + 8 + 15 | SEQ ID NOs: 16 + 5 + 53 + 54 + 55 + 66 + 68 + 58 + 59 |
| SEQ ID NOs: 50 + 9 + 15 | SEQ ID NOs: 16 + 5 + 53 + 54 + 55 + 66 + 68 + 58 + 59 |
| SEQ ID NOs: 50 + 10 + 15 | SEQ ID NOs: 16 + 5 + 53 + 54 + 55 + 66 + 68 + 58 + 59 |
| SEQ ID NOs: 50 + 11 + 15 | SEQ ID NOs: 16 + 5 + 53 + 54 + 55 + 66 + 68 + 58 + 59 |
| SEQ ID NOs: 50 + 12 + 15 | SEQ ID NOs: 16 + 5 + 53 + 54 + 55 + 66 + 68 + 58 + 59 |
| SEQ ID NOs: 50 + 13 + 15 | SEQ ID NOs: 16 + 5 + 53 + 54 + 55 + 66 + 68 + 58 + 59 |
| SEQ ID NOs: 50 + 14 + 15 | SEQ ID NOs: 16 + 5 + 53 + 54 + 55 + 66 + 68 + 58 + 59 |
| SEQ ID NOs: 50 + 49 + 15 | SEQ ID NOs: 16 + 5 + 53 + 54 + 55 + 66 + 68 + 58 + 59 |
| SEQ ID NOs: 50 + 8 + 15 | SEQ ID NOs: 16 + 5 + 53 + 54 + 55 + 67 + 57 + 58 + 59 |
| SEQ ID NOs: 50 + 9 + 15 | SEQ ID NOs: 16 + 5 + 53 + 54 + 55 + 67 + 57 + 58 + 59 |
| SEQ ID NOs: 50 + 10 + 15 | SEQ ID NOs: 16 + 5 + 53 + 54 + 55 + 67 + 57 + 58 + 59 |
| SEQ ID NOs: 50 + 11 + 15 | SEQ ID NOs: 16 + 5 + 53 + 54 + 55 + 67 + 57 + 58 + 59 |
| SEQ ID NOs: 50 + 12 + 15 | SEQ ID NOs: 16 + 5 + 53 + 54 + 55 + 67 + 57 + 58 + 59 |
| SEQ ID NOs: 50 + 13 + 15 | SEQ ID NOs: 16 + 5 + 53 + 54 + 55 + 67 + 57 + 58 + 59 |
| SEQ ID NOs: 50 + 14 + 15 | SEQ ID NOs: 16 + 5 + 53 + 54 + 55 + 67 + 57 + 58 + 59 |
| SEQ ID NOs: 50 + 49 + 15 | SEQ ID NOs: 16 + 5 + 53 + 54 + 55 + 67 + 57 + 58 + 59 |
| SEQ ID NOs: 50 + 8 + 15 | SEQ ID NOs: 16 + 5 + 53 + 54 + 55 + 67 + 68 + 58 + 59 |
| SEQ ID NOs: 50 + 9 + 15 | SEQ ID NOs: 16 + 5 + 53 + 54 + 55 + 67 + 68 + 58 + 59 |
| SEQ ID NOs: 50 + 10 + 15 | SEQ ID NOs: 16 + 5 + 53 + 54 + 55 + 67 + 68 + 58 + 59 |
| SEQ ID NOs: 50 + 11 + 15 | SEQ ID NOs: 16 + 5 + 53 + 54 + 55 + 67 + 68 + 58 + 59 |
| SEQ ID NOs: 50 + 12 + 15 | SEQ ID NOs: 16 + 5 + 53 + 54 + 55 + 67 + 68 + 58 + 59 |
| SEQ ID NOs: 50 + 13 + 15 | SEQ ID NOs: 16 + 5 + 53 + 54 + 55 + 67 + 68 + 58 + 59 |
| SEQ ID NOs: 50 + 14 + 15 | SEQ ID NOs: 16 + 5 + 53 + 54 + 55 + 67 + 68 + 58 + 59 |
| SEQ ID NOs: 50 + 49 + 15 | SEQ ID NOs: 16 + 5 + 53 + 54 + 55 + 67 + 68 + 58 + 59 |
| SEQ ID NOs: 50 + 8 + 15 | SEQ ID NOs: 16 + 5 + 53 + 54 + 55 + 67 + 69 + 58 + 59 |
| SEQ ID NOs: 50 + 9 + 15 | SEQ ID NOs: 16 + 5 + 53 + 54 + 55 + 67 + 69 + 58 + 59 |
| SEQ ID NOs: 50 + 10 + 15 | SEQ ID NOs: 16 + 5 + 53 + 54 + 55 + 67 + 69 + 58 + 59 |
| SEQ ID NOs: 50 + 11 + 15 | SEQ ID NOs: 16 + 5 + 53 + 54 + 55 + 67 + 69 + 58 + 59 |
| SEQ ID NOs: 50 + 12 + 15 | SEQ ID NOs: 16 + 5 + 53 + 54 + 55 + 67 + 69 + 58 + 59 |
| SEQ ID NOs: 50 + 13 + 15 | SEQ ID NOs: 16 + 5 + 53 + 54 + 55 + 67 + 69 + 58 + 59 |
| SEQ ID NOs: 50 + 14 + 15 | SEQ ID NOs: 16 + 5 + 53 + 54 + 55 + 67 + 69 + 58 + 59 |
| SEQ ID NOs: 50 + 49 + 15 | SEQ ID NOs: 16 + 5 + 53 + 54 + 55 + 67 + 69 + 58 + 59 |
| SEQ ID NOs: 50 + 8 + 15 | SEQ ID NOs: 16 + 5 + 53 + 54 + 55 + 60 + 69 + 58 + 59 |
| SEQ ID NOs: 50 + 9 + 15 | SEQ ID NOs: 16 + 5 + 53 + 54 + 55 + 60 + 69 + 58 + 59 |
| SEQ ID NOs: 50 + 10 + 15 | SEQ ID NOs: 16 + 5 + 53 + 54 + 55 + 60 + 69 + 58 + 59 |
| SEQ ID NOs: 50 + 11 + 15 | SEQ ID NOs: 16 + 5 + 53 + 54 + 55 + 60 + 69 + 58 + 59 |
| SEQ ID NOs: 50 + 12 + 15 | SEQ ID NOs: 16 + 5 + 53 + 54 + 55 + 60 + 69 + 58 + 59 |
| SEQ ID NOs: 50 + 13 + 15 | SEQ ID NOs: 16 + 5 + 53 + 54 + 55 + 60 + 69 + 58 + 59 |
| SEQ ID NOs: 50 + 14 + 15 | SEQ ID NOs: 16 + 5 + 53 + 54 + 55 + 60 + 69 + 58 + 59 |
| SEQ ID NOs: 50 + 49 + 15 | SEQ ID NOs: 16 + 5 + 53 + 54 + 55 + 60 + 69 + 58 + 59 |

Preferred bi-specific antibodies or antibody fragments that bind Her2 protein and CD3 protein may be selected from any of the following antibodies or antibody fragments which comprise each specific combination of twelve CDRs H1, H2, H3, L1, L2, L3, L4, L5, L6, L7, L8, and L9 set forth below.

Preferred Anti-Her2/Anti-CD3 Bi-Specific Antibodies

| H1, H2, H3 CDRs | L1, L2, L3, L4, L5, L6, L7, L8, L9 CDRs |
|---|---|
| SEQ ID NOs: 50 + 9 + 51 | SEQ ID NOs: 16 + 5 + 53 + 54 + 55 + 56 + 57 + 58 + 59 |
| SEQ ID NOs: 50 + 12 + 51 | SEQ ID NOs: 16 + 5 + 53 + 54 + 55 + 56 + 57 + 58 + 59 |
| SEQ ID NOs: 50 + 13 + 51 | SEQ ID NOs: 16 + 5 + 53 + 54 + 55 + 56 + 57 + 58 + 59 |
| SEQ ID NOs: 50 + 49 + 51 | SEQ ID NOs: 16 + 5 + 5 + 54 + 55 + 563 + 57 + 58 + 59 |
| SEQ ID NOs: 50 + 49 + 15 | SEQ ID NOs: 16 + 5 + 53 + 54 + 55 + 56 + 57 + 58 + 59 |
| SEQ ID NOs: 50 + 9 + 51 | SEQ ID NOs: 16 + 5 + 53 + 54 + 55 + 56 + 68 + 58 + 59 |
| SEQ ID NOs: 50 + 12 + 51 | SEQ ID NOs: 16 + 5 + 53 + 54 + 55 + 56 + 68 + 58 + 59 |
| SEQ ID NOs: 50 + 13 + 51 | SEQ ID NOs: 16 + 5 + 53 + 54 + 55 + 56 + 68 + 58 + 59 |
| SEQ ID NOs: 50 + 49 + 51 | SEQ ID NOs: 16 + 5 + 53 + 54 + 55 + 56 + 68 + 58 + 59 |
| SEQ ID NOs: 50 + 49 + 15 | SEQ ID NOs: 16 + 5 + 53 + 54 + 55 + 56 + 68 + 58 + 59 |
| SEQ ID NOs: 50 + 9 + 51 | SEQ ID NOs: 16 + 5 + 53 + 54 + 55 + 56 + 69 + 58 + 59 |
| SEQ ID NOs: 50 + 12 + 51 | SEQ ID NOs: 16 + 5 + 53 + 54 + 55 + 56 + 69 + 58 + 59 |
| SEQ ID NOs: 50 + 13 + 51 | SEQ ID NOs: 16 + 5 + 53 + 54 + 55 + 56 + 69 + 58 + 59 |
| SEQ ID NOs: 50 + 49 + 51 | SEQ ID NOs: 16 + 5 + 53 + 54 + 55 + 56 + 69 + 58 + 59 |
| SEQ ID NOs: 50 + 49 + 15 | SEQ ID NOs: 16 + 5 + 53 + 54 + 55 + 56 + 69 + 58 + 59 |
| SEQ ID NOs: 50 + 9 + 51 | SEQ ID NOs: 16 + 5 + 53 + 54 + 55 + 60 + 57 + 58 + 59 |
| SEQ ID NOs: 50 + 12 + 51 | SEQ ID NOs: 16 + 5 + 53 + 54 + 55 + 60 + 57 + 58 + 59 |
| SEQ ID NOs: 50 + 13 + 51 | SEQ ID NOs: 16 + 5 + 53 + 54 + 55 + 60 + 57 + 58 + 59 |
| SEQ ID NOs: 50 + 49 + 51 | SEQ ID NOs: 16 + 5 + 53 + 54 + 55 + 60 + 57 + 58 + 59 |
| SEQ ID NOs: 50 + 49 + 15 | SEQ ID NOs: 16 + 5 + 53 + 54 + 55 + 60 + 57 + 58 + 59 |
| SEQ ID NOs: 50 + 9 + 51 | SEQ ID NOs: 16 + 5 + 53 + 54 + 55 + 60 + 68 + 58 + 59 |
| SEQ ID NOs: 50 + 12 + 51 | SEQ ID NOs: 16 + 5 + 53 + 54 + 55 + 60 + 68 + 58 + 59 |
| SEQ ID NOs: 50 + 13 + 51 | SEQ ID NOs: 16 + 5 + 53 + 54 + 55 + 60 + 68 + 58 + 59 |
| SEQ ID NOs: 50 + 49 + 51 | SEQ ID NOs: 16 + 5 + 53 + 54 + 55 + 60 + 68 + 58 + 59 |
| SEQ ID NOs: 50 + 49 + 15 | SEQ ID NOs: 16 + 5 + 53 + 54 + 55 + 60 + 68 + 58 + 59 |
| SEQ ID NOs: 50 + 9 + 51 | SEQ ID NOs: 16 + 5 + 53 + 54 + 55 + 60 + 69 + 58 + 59 |
| SEQ ID NOs: 50 + 12 + 51 | SEQ ID NOs: 16 + 5 + 53 + 54 + 55 + 60 + 69 + 58 + 59 |
| SEQ ID NOs: 50 + 13 + 51 | SEQ ID NOs: 16 + 5 + 53 + 54 + 55 + 60 + 69 + 58 + 59 |
| SEQ ID NOs: 50 + 49 + 51 | SEQ ID NOs: 16 + 5 + 53 + 54 + 55 + 60 + 69 + 58 + 59 |
| SEQ ID NOs: 50 + 49 + 15 | SEQ ID NOs: 16 + 5 + 53 + 54 + 55 + 60 + 69 + 58 + 59 |
| SEQ ID NOs: 50 + 9 + 51 | SEQ ID NOs: 16 + 5 + 53 + 54 + 55 + 61 + 57 + 58 + 59 |
| SEQ ID NOs: 50 + 12 + 51 | SEQ ID NOs: 16 + 5 + 53 + 54 + 55 + 61 + 57 + 58 + 59 |
| SEQ ID NOs: 50 + 13 + 51 | SEQ ID NOs: 16 + 5 + 53 + 54 + 55 + 61 + 57 + 58 + 59 |
| SEQ ID NOs: 50 + 49 + 51 | SEQ ID NOs: 16 + 5 + 53 + 54 + 55 + 61 + 57 + 58 + 59 |
| SEQ ID NOs: 50 + 49 + 15 | SEQ ID NOs: 16 + 5 + 53 + 54 + 55 + 61 + 57 + 58 + 59 |
| SEQ ID NOs: 50 + 9 + 51 | SEQ ID NOs: 16 + 5 + 53 + 54 + 55 + 61 + 68 + 58 + 59 |
| SEQ ID NOs: 50 + 12 + 51 | SEQ ID NOs: 16 + 5 + 53 + 54 + 55 + 61 + 68 + 58 + 59 |
| SEQ ID NOs: 50 + 13 + 51 | SEQ ID NOs: 16 + 5 + 53 + 54 + 55 + 61 + 68 + 58 + 59 |
| SEQ ID NOs: 50 + 49 + 51 | SEQ ID NOs: 16 + 5 + 53 + 54 + 55 + 61 + 68 + 58 + 59 |
| SEQ ID NOs: 50 + 49 + 15 | SEQ ID NOs: 16 + 5 + 53 + 54 + 55 + 61 + 68 + 58 + 59 |
| SEQ ID NOs: 50 + 9 + 51 | SEQ ID NOs: 16 + 5 + 53 + 54 + 55 + 61 + 69 + 58 + 59 |
| SEQ ID NOs: 50 + 12 + 51 | SEQ ID NOs: 16 + 5 + 53 + 54 + 55 + 61 + 69 + 58 + 59 |
| SEQ ID NOs: 50 + 13 + 51 | SEQ ID NOs: 16 + 5 + 53 + 54 + 55 + 61 + 69 + 58 + 59 |
| SEQ ID NOs: 50 + 49 + 51 | SEQ ID NOs: 16 + 5 + 53 + 54 + 55 + 61 + 69 + 58 + 59 |
| SEQ ID NOs: 50 + 49 + 15 | SEQ ID NOs: 16 + 5 + 53 + 54 + 55 + 61 + 69 + 58 + 59 |
| SEQ ID NOs: 50 + 9 + 51 | SEQ ID NOs: 16 + 5 + 53 + 54 + 55 + 62 + 57 + 58 + 59 |
| SEQ ID NOs: 50 + 12 + 51 | SEQ ID NOs: 16 + 5 + 53 + 54 + 55 + 62 + 57 + 58 + 59 |
| SEQ ID NOs: 50 + 13 + 51 | SEQ ID NOs: 16 + 5 + 53 + 54 + 55 + 62 + 57 + 58 + 59 |
| SEQ ID NOs: 50 + 49 + 51 | SEQ ID NOs: 16 + 5 + 53 + 54 + 55 + 62 + 57 + 58 + 59 |
| SEQ ID NOs: 50 + 49 + 15 | SEQ ID NOs: 16 + 5 + 53 + 54 + 55 + 62 + 57 + 58 + 59 |
| SEQ ID NOs: 50 + 9 + 51 | SEQ ID NOs: 16 + 5 + 53 + 54 + 55 + 62 + 68 + 58 + 59 |
| SEQ ID NOs: 50 + 12 + 51 | SEQ ID NOs: 16 + 5 + 53 + 54 + 55 + 62 + 68 + 58 + 59 |
| SEQ ID NOs: 50 + 13 + 51 | SEQ ID NOs: 16 + 5 + 53 + 54 + 55 + 62 + 68 + 58 + 59 |
| SEQ ID NOs: 50 + 49 + 51 | SEQ ID NOs: 16 + 5 + 53 + 54 + 55 + 62 + 68 + 58 + 59 |
| SEQ ID NOs: 50 + 49 + 15 | SEQ ID NOs: 16 + 5 + 53 + 54 + 55 + 62 + 68 + 58 + 59 |
| SEQ ID NOs: 50 + 9 + 51 | SEQ ID NOs: 16 + 5 + 53 + 54 + 55 + 62 + 69 + 58 + 59 |
| SEQ ID NOs: 50 + 12 + 51 | SEQ ID NOs: 16 + 5 + 53 + 54 + 55 + 62 + 69 + 58 + 59 |
| SEQ ID NOs: 50 + 13 + 51 | SEQ ID NOs: 16 + 5 + 53 + 54 + 55 + 62 + 69 + 58 + 59 |
| SEQ ID NOs: 50 + 49 + 51 | SEQ ID NOs: 16 + 5 + 53 + 54 + 55 + 62 + 69 + 58 + 59 |
| SEQ ID NOs: 50 + 49 + 15 | SEQ ID NOs: 16 + 5 + 53 + 54 + 55 + 62 + 69 + 58 + 59 |
| SEQ ID NOs: 50 + 9 + 51 | SEQ ID NOs: 16 + 5 + 53 + 54 + 55 + 63 + 57 + 58 + 59 |
| SEQ ID NOs: 50 + 12 + 51 | SEQ ID NOs: 16 + 5 + 53 + 54 + 55 + 63 + 57 + 58 + 59 |
| SEQ ID NOs: 50 + 13 + 51 | SEQ ID NOs: 16 + 5 + 53 + 54 + 55 + 63 + 57 + 58 + 59 |
| SEQ ID NOs: 50 + 49 + 51 | SEQ ID NOs: 16 + 5 + 53 + 54 + 55 + 63 + 57 + 58 + 59 |
| SEQ ID NOs: 50 + 49 + 15 | SEQ ID NOs: 16 + 5 + 53 + 54 + 55 + 63 + 57 + 58 + 59 |
| SEQ ID NOs: 50 + 9 + 51 | SEQ ID NOs: 16 + 5 + 53 + 54 + 55 + 63 + 68 + 58 + 59 |
| SEQ ID NOs: 50 + 12 + 51 | SEQ ID NOs: 16 + 5 + 53 + 54 + 55 + 63 + 68 + 58 + 59 |
| SEQ ID NOs: 50 + 13 + 51 | SEQ ID NOs: 16 + 5 + 53 + 54 + 55 + 63 + 68 + 58 + 59 |
| SEQ ID NOs: 50 + 49 + 51 | SEQ ID NOs: 16 + 5 + 53 + 54 + 55 + 63 + 68 + 58 + 59 |
| SEQ ID NOs: 50 + 49 + 15 | SEQ ID NOs: 16 + 5 + 53 + 54 + 55 + 63 + 68 + 58 + 59 |
| SEQ ID NOs: 50 + 9 + 51 | SEQ ID NOs: 16 + 5 + 53 + 54 + 55 + 63 + 69 + 58 + 59 |
| SEQ ID NOs: 50 + 12 + 51 | SEQ ID NOs: 16 + 5 + 53 + 54 + 55 + 63 + 69 + 58 + 59 |
| SEQ ID NOs: 50 + 13 + 51 | SEQ ID NOs: 16 + 5 + 53 + 54 + 55 + 63 + 69 + 58 + 59 |
| SEQ ID NOs: 50 + 49 + 51 | SEQ ID NOs: 16 + 5 + 53 + 54 + 55 + 63 + 69 + 58 + 59 |
| SEQ ID NOs: 50 + 49 + 15 | SEQ ID NOs: 16 + 5 + 53 + 54 + 55 + 63 + 69 + 58 + 59 |
| SEQ ID NOs: 50 + 9 + 51 | SEQ ID NOs: 16 + 5 + 53 + 54 + 55 + 60 + 57 + 58 + 59 |
| SEQ ID NOs: 50 + 12 + 51 | SEQ ID NOs: 16 + 5 + 53 + 54 + 55 + 60 + 57 + 58 + 59 |
| SEQ ID NOs: 50 + 13 + 51 | SEQ ID NOs: 16 + 5 + 53 + 54 + 55 + 60 + 57 + 58 + 59 |
| SEQ ID NOs: 50 + 49 + 51 | SEQ ID NOs: 16 + 5 + 53 + 54 + 55 + 60 + 57 + 58 + 59 |
| SEQ ID NOs: 50 + 49 + 15 | SEQ ID NOs: 16 + 5 + 53 + 54 + 55 + 60 + 57 + 58 + 59 |
| SEQ ID NOs: 50 + 9 + 51 | SEQ ID NOs: 16 + 5 + 53 + 54 + 55 + 64 + 68 + 58 + 59 |
| SEQ ID NOs: 50 + 12 + 51 | SEQ ID NOs: 16 + 5 + 53 + 54 + 55 + 64 + 68 + 58 + 59 |
| SEQ ID NOs: 50 + 13 + 51 | SEQ ID NOs: 16 + 5 + 53 + 54 + 55 + 64 + 68 + 58 + 59 |
| SEQ ID NOs: 50 + 49 + 51 | SEQ ID NOs: 16 + 5 + 53 + 54 + 55 + 64 + 68 + 58 + 59 |
| SEQ ID NOs: 50 + 49 + 15 | SEQ ID NOs: 16 + 5 + 53 + 54 + 55 + 64 + 68 + 58 + 59 |
| SEQ ID NOs: 50 + 9 + 51 | SEQ ID NOs: 16 + 5 + 53 + 54 + 55 + 64 + 69 + 58 + 59 |
| SEQ ID NOs: 50 + 12 + 51 | SEQ ID NOs: 16 + 5 + 53 + 54 + 55 + 64 + 69 + 58 + 59 |
| SEQ ID NOs: 50 + 13 + 51 | SEQ ID NOs: 16 + 5 + 53 + 54 + 55 + 64 + 69 + 58 + 59 |
| SEQ ID NOs: 50 + 49 + 51 | SEQ ID NOs: 16 + 5 + 53 + 54 + 55 + 64 + 69 + 58 + 59 |
| SEQ ID NOs: 50 + 49 + 15 | SEQ ID NOs: 16 + 5 + 53 + 54 + 55 + 64 + 69 + 58 + 59 |
| SEQ ID NOs: 50 + 9 + 51 | SEQ ID NOs: 16 + 5 + 53 + 54 + 55 + 65 + 57 + 58 + 59 |
| SEQ ID NOs: 50 + 12 + 51 | SEQ ID NOs: 16 + 5 + 53 + 54 + 55 + 65 + 57 + 58 + 59 |
| SEQ ID NOs: 50 + 13 + 51 | SEQ ID NOs: 16 + 5 + 53 + 54 + 55 + 65 + 57 + 58 + 59 |
| SEQ ID NOs: 50 + 49 + 51 | SEQ ID NOs: 16 + 5 + 53 + 54 + 55 + 65 + 57 + 58 + 59 |
| SEQ ID NOs: 50 + 49 + 15 | SEQ ID NOs: 16 + 5 + 53 + 54 + 55 + 65 + 57 + 58 + 59 |
| SEQ ID NOs: 50 + 9 + 51 | SEQ ID NOs: 16 + 5 + 53 + 54 + 55 + 65 + 68 + 58 + 59 |
| SEQ ID NOs: 50 + 12 + 51 | SEQ ID NOs: 16 + 5 + 53 + 54 + 55 + 65 + 68 + 58 + 59 |
| SEQ ID NOs: 50 + 13 + 51 | SEQ ID NOs: 16 + 5 + 53 + 54 + 55 + 65 + 68 + 58 + 59 |
| SEQ ID NOs: 50 + 49 + 51 | SEQ ID NOs: 16 + 5 + 53 + 54 + 55 + 65 + 68 + 58 + 59 |
| SEQ ID NOs: 50 + 49 + 15 | SEQ ID NOs: 16 + 5 + 53 + 54 + 55 + 65 + 68 + 58 + 59 |
| SEQ ID NOs: 50 + 9 + 51 | SEQ ID NOs: 16 + 5 + 53 + 54 + 55 + 65 + 69 + 58 + 59 |
| SEQ ID NOs: 50 + 12 + 51 | SEQ ID NOs: 16 + 5 + 53 + 54 + 55 + 65 + 69 + 58 + 59 |
| SEQ ID NOs: 50 + 13 + 51 | SEQ ID NOs: 16 + 5 + 53 + 54 + 55 + 65 + 69 + 58 + 59 |
| SEQ ID NOs: 50 + 49 + 51 | SEQ ID NOs: 16 + 5 + 53 + 54 + 55 + 65 + 69 + 58 + 59 |
| SEQ ID NOs: 50 + 49 + 15 | SEQ ID NOs: 16 + 5 + 53 + 54 + 55 + 65 + 69 + 58 + 59 |
| SEQ ID NOs: 50 + 9 + 51 | SEQ ID NOs: 16 + 5 + 53 + 54 + 55 + 60 + 57 + 58 + 59 |
| SEQ ID NOs: 50 + 12 + 51 | SEQ ID NOs: 16 + 5 + 53 + 54 + 55 + 60 + 57 + 58 + 59 |
| SEQ ID NOs: 50 + 13 + 51 | SEQ ID NOs: 16 + 5 + 53 + 54 + 55 + 60 + 57 + 58 + 59 |
| SEQ ID NOs: 50 + 49 + 51 | SEQ ID NOs: 16 + 5 + 53 + 54 + 55 + 60 + 57 + 58 + 59 |
| SEQ ID NOs: 50 + 49 + 15 | SEQ ID NOs: 16 + 5 + 53 + 54 + 55 + 60 + 57 + 58 + 59 |
| SEQ ID NOs: 50 + 9 + 51 | SEQ ID NOs: 16 + 5 + 53 + 54 + 55 + 66 + 68 + 58 + 59 |
| SEQ ID NOs: 50 + 12 + 51 | SEQ ID NOs: 16 + 5 + 53 + 54 + 55 + 66 + 68 + 58 + 59 |
| SEQ ID NOs: 50 + 13 + 51 | SEQ ID NOs: 16 + 5 + 53 + 54 + 55 + 66 + 68 + 58 + 59 |
| SEQ ID NOs: 50 + 49 + 51 | SEQ ID NOs: 16 + 5 + 53 + 54 + 55 + 66 + 68 + 58 + 59 |
| SEQ ID NOs: 50 + 49 + 15 | SEQ ID NOs: 16 + 5 + 53 + 54 + 55 + 66 + 68 + 58 + 59 |
| SEQ ID NOs: 50 + 9 + 51 | SEQ ID NOs: 16 + 5 + 53 + 54 + 55 + 66 + 69 + 58 + 59 |
| SEQ ID NOs: 50 + 12 + 51 | SEQ ID NOs: 16 + 5 + 53 + 54 + 55 + 66 + 69 + 58 + 59 |
| SEQ ID NOs: 50 + 13 + 51 | SEQ ID NOs: 16 + 5 + 53 + 54 + 55 + 66 + 69 + 58 + 59 |
| SEQ ID NOs: 50 + 49 + 51 | SEQ ID NOs: 16 + 5 + 53 + 54 + 55 + 66 + 69 + 58 + 59 |
| SEQ ID NOs: 50 + 49 + 15 | SEQ ID NOs: 16 + 5 + 53 + 54 + 55 + 66 + 69 + 58 + 59 |
| SEQ ID NOs: 50 + 9 + 51 | SEQ ID NOs: 16 + 5 + 53 + 54 + 55 + 67 + 57 + 58 + 59 |
| SEQ ID NOs: 50 + 12 + 51 | SEQ ID NOs: 16 + 5 + 53 + 54 + 55 + 67 + 57 + 58 + 59 |
| SEQ ID NOs: 50 + 13 + 51 | SEQ ID NOs: 16 + 5 + 53 + 54 + 55 + 67 + 57 + 58 + 59 |
| SEQ ID NOs: 50 + 49 + 51 | SEQ ID NOs: 16 + 5 + 53 + 54 + 55 + 67 + 57 + 58 + 59 |
| SEQ ID NOs: 50 + 49 + 15 | SEQ ID NOs: 16 + 5 + 53 + 54 + 55 + 67 + 57 + 58 + 59 |
| SEQ ID NOs: 50 + 9 + 51 | SEQ ID NOs: 16 + 5 + 53 + 54 + 55 + 67 + 68 + 58 + 59 |
| SEQ ID NOs: 50 + 12 + 51 | SEQ ID NOs: 16 + 5 + 53 + 54 + 55 + 67 + 68 + 58 + 59 |
| SEQ ID NOs: 50 + 13 + 51 | SEQ ID NOs: 16 + 5 + 53 + 54 + 55 + 67 + 68 + 58 + 59 |
| SEQ ID NOs: 50 + 49 + 51 | SEQ ID NOs: 16 + 5 + 53 + 54 + 55 + 67 + 68 + 58 + 59 |
| SEQ ID NOs: 50 + 49 + 15 | SEQ ID NOs: 16 + 5 + 53 + 54 + 55 + 67 + 68 + 58 + 59 |
| SEQ ID NOs: 50 + 9 + 51 | SEQ ID NOs: 16 + 5 + 53 + 54 + 55 + 67 + 69 + 58 + 59 |
| SEQ ID NOs: 50 + 12 + 51 | SEQ ID NOs: 16 + 5 + 53 + 54 + 55 + 67 + 69 + 58 + 59 |
| SEQ ID NOs: 50 + 13 + 51 | SEQ ID NOs: 16 + 5 + 53 + 54 + 55 + 67 + 69 + 58 + 59 |
| SEQ ID NOs: 50 + 49 + 51 | SEQ ID NOs: 16 + 5 + 53 + 54 + 55 + 67 + 69 + 58 + 59 |
| SEQ ID NOs: 50 + 49 + 15 | SEQ ID NOs: 16 + 5 + 53 + 54 + 55 + 67 + 69 + 58 + 59 |

Techniques for making multispecific antibodies include, but are not limited to, recombinant co-expression of two immunoglobulin heavy chain-light chain pairs having different specificities (see Milstein and Cuello, Nature, vol. 305, pp. 537-540, 1983), WO 93/08829, and Traunecker et al., EMBO J. vol. 10, pp. 3655-3659, 1991), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, vol. 229, pp. 81-83, 1985); using leucine zippers to produce bi-specific antibodies (see, e.g., Kostelny et al., J. Immunol., vol. 148, pp. 1547-1553, 1992); using “diabody” technology for making bispecific antibody fragments (see, e.g., Hollinger et al., Proc. Natl. Acad. Sci. USA, vol. 90, pp. 6444-6448, 1993); and using single-chain Fv (scFv) dimers (see, e.g. Gruber et al., J. Immunol., vol. 152, pp. 5368-5374, 1994); and preparing trispecific antibodies as described, e.g., in Tutt et al. J. Immunol., vol. 147, pp. 60-69, 1991.

In one embodiment, the bispecific antibody comprises an antibody or antibody fragment of the present disclosure against HER2 and a second antibody or antibody fragment directed against a tumor-reactive lymphocyte antigen. In another embodiment, the tumor-reactive lymphocyte antigen is CD3.

Engineered antibodies with three or more functional antigen binding sites, including “Octopus antibodies,” are also included herein (see, e.g. US 2006/0025576A1).

The anti-HER2 antibodies or antibody fragments of the invention may be produced using recombinant methods and compositions, which are described in detail in US 2016/0017040.

The physical/chemical properties and/or biological activities of the anti-HER2 antibodies or antibody fragments of the invention may be tested and measured by various assays known in the art. Some of these assays are described in U.S. Pat. No. 8,853,369.

### H. Immunoconjugates

In another aspect, the invention also provides immunoconjugates comprising an anti-HER2 antibody or antibody fragment conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), and radioactive isotopes.

In one embodiment, the immunoconjugate is an antibody-drug conjugate (ADC) in which an antibody or antibody fragment is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res., vol. 53, pp. 3336-3342, 1993; and Lode et al., Cancer Res., vol. 58, pp. 2925-2928, 1998); an anthracycline such as daunomycin or doxorubicin (see Kratz et al., Current Med. Chem., vol. 13, pp. 477-523, 2006; Jeffrey et al., Bioorganic & Med. Chem. Letters, vol. 16, pp. 358-362, 2006; Torgov et al., Bioconj. Chem., vol. 16, pp. 717-721, 2005; Nagy et al., Proc. Natl. Acad. Sci. USA, vol. 97, pp. 829-834, 2000; Dubowchik et al., Bioorg. & Med. Chem. Letters, vol. 12, vol. 1529-1532, 2002; King et al., J. Med. Chem., vol. 45, pp. 4336-4343, 2002; and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody or antibody fragment as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody or antibody fragment as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese and iron.

In some embodiments, the immunoconjugate comprises a radioactive agent, which may be selected from an alpha emitter, a beta emitter and a gamma emitter. Examples of alpha emitters are ²¹¹At, ²¹⁰Bi, ²¹¹Bi, ²²³Ra, ²²⁴Ra, ²²⁵Ac and ²²⁷Th. Examples of beta-emitters are ⁶⁷Cu, ⁹⁰Y, ¹³¹I, ¹⁵³Sm, ¹⁶⁶Ho, and ¹⁸⁶Re. Examples of gamma emitters are ⁶⁰Co, ¹³⁷Ce, ⁵⁵Fe, ⁵⁴Mg, ²⁰³Hg, and ¹³³Ba. In certain embodiments, an immunoconjugate may comprise a highly radioactive atom. Zirconium-89 may be complexed to various metal chelating agents and conjugated to antibodies, e.g., for PET imaging (WO 2011/056983).

The radio- or other labels may be incorporated in the immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the antibody. In some embodiments, yttrium-90 can be attached via a lysine residue of the antibody. In some embodiments, the IODOGEN method (Fraker et al., Biochem. Biophys. Res. Commun., vol. 80, pp. 49-57, 1978) can be used to incorporate iodine-123. “Monoclonal Antibodies in Immunoscintigraphy” (Chatal, CRC Press 1989) describes certain other methods.

Conjugates of an antibody/antibody fragment and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, vol. 238, pp. 1098-, 1987. Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res., vol. 52, pp. 127-131, 1992; U.S. Pat. No. 5,208,020) may be used.

The immunuoconjugates include, but are not limited to, immunoconjugates prepared with cross-linker reagents including, but not limited to, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

An exemplary embodiment of an ADC includes an antibody or antibody fragment (Ab) which targets a tumor cell, a drug moiety (D), and a linker moiety (L) that attaches Ab to D. In some embodiments, the antibody is attached to the linker moiety (L) through one or more amino acid residues, such as lysine and/or cysteine.

An exemplary ADC has Formula I as Ab-(L-D)ₚ, where p is 1 to about 20. In some embodiments, the number of drug moieties that can be conjugated to an antibody is limited by the number of free cysteine residues. In some embodiments, free cysteine residues are introduced into the antibody amino acid sequence by the methods described herein. Exemplary ADC's of Formula I include, but are not limited to, antibodies that have 1, 2, 3, or 4 engineered cysteine amino acids (Lyon et al., Methods in Enzym., vol. 502, pp. 123-138, 2012). In some embodiments, one or more free cysteine residues are already present in an antibody, without the use of engineering, in which case the existing free cysteine residues may be used to conjugate the antibody to a drug. In some embodiments, an antibody is exposed to reducing conditions prior to conjugation of the antibody to generate one or more free cysteine residues.

Linkers are used to conjugate a moiety to the antibody to form an immunoconjugate such as an ADC. Suitable linkers are described in WO 2017/180842. Some drug moieties that may be conjugated to the antibodies are described in WO 2017/180842. Drug moieties also include compounds with nucleolytic activity (e.g., a ribonuclease or a DNA endonuclease).

In certain embodiments, an immunoconjugate may comprise an antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as Serratia protease, thermolysis, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature, vol. 312, pp. 604-608, 1984.

Drug loading in the conjugates is represented by p, the average number of drug moieties per antibody. Drug loading may range from 1 to 20 drug moieties per antibody. The conjugates of the present invention may have a range of drug moieties, from 1 to 20. The average number of drug moieties per antibody use in the preparation of the conjugates from conjugation reactions may be characterized by conventional means such as mass spectroscopy, ELISA assay, and HPLC.

For some antibody-drug conjugates (ADC), the drug loading may be limited by the number of attachment sites on the antibody. For example, where the attachment is a cysteine thiol, as in certain exemplary embodiments above, an antibody may have only one or several cysteine thiol groups, or may have only one or several sufficiently reactive thiol groups through which a linker may be attached. In certain embodiments, higher drug loading, e.g. p>5, may cause aggregation, insolubility, toxicity, or loss of cellular permeability of certain antibody-drug conjugates. In certain embodiments, the average drug loading for an ADC ranges from 1 to about 8; from about 2 to about 6; or from about 3 to about 5. Indeed, it has been shown that for certain ADCs, the optimal ratio of drug moieties per antibody may be less than 8 and may be about 2 to about 5 (U.S. Pat. No. 7,498,298).

In certain embodiments, fewer than the theoretical maximum number of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety. Indeed, most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

### I. Methods and Compositions for Diagnostics and Detection

In certain embodiments, any of the anti-HER2 antibodies or antibody fragments provided herein may be used for detecting the presence of HER2 protein in a biological sample, either quantitatively or qualitatively. In certain embodiments, a biological sample comprises a cell or tissue, such as breast, pancreas, esophagus, lung and/or brain cells or tissue.

A further aspect of the invention relates to an anti-HER2 antibody or antibody fragment of the invention for diagnosing and/or monitoring a cancer or another disease in which HER2 protein expression levels are increased or decreased from a normal physiological level at least one location in the body.

In one embodiment, antibodies or antibody fragments of the invention may be labelled with a detectable molecule or substance, such as a fluorescent molecule, a radioactive molecule or any other label known in the art as above described. For example, an antibody or antibody fragment of the invention may be labelled with a radioactive molecule. For example, suitable radioactive molecules include but are not limited to radioactive atoms used for scintigraphic studies such as ¹²³I, ¹²⁴I, ¹¹¹In, ¹⁸⁶Re, and ¹⁸⁸Re. Antibodies or antibody fragments of the invention may also be labelled with a spin label for nuclear magnetic resonance (NMR) imaging, such as iodine-123, iodine-131, indium-Ill, fluorine-19, carbon-13, nitrogen-15, oxygen-17, gadolinium, manganese or iron. Following administration of the antibody, the distribution of the radiolabeled antibody within the patient is detected. Any suitable known method can be used. Some non-limiting examples include, computed tomography (CT), position emission tomography (PET), magnetic resonance imaging (MRI), fluorescence, chemiluminescence and sonography.

Antibodies or antibody fragments of the invention may be useful for diagnosing and staging of cancer and diseases associated with HER2 protein overexpression. Cancers associated with HER2 protein expression or overexpression may include, but are not necessarily limited to, breast cancer, ovarian cancer, bladder carcinomas, gallbladder cancer, extrahepatic or intrahepatic cholangiocarcinomas, salivary duct carcinomas, gastic cancers including esophageal, esophagogastric junction cancers and gastric adenocarcinomas and gastrointestinal stromal tumors, colon cancer, lung cancers including non-small cell and small cell small-cell lung cancer, pancreatic cancer such as pancreatic adenocarcinams, penile cancer, pituitary cancers, prostate cancers, sarcomas including soft tissue sarcomas, peritoneal sarcomas and retroperitoneal sarcomas, solitary fibrous tumors, thymic cancers, thyroid cancers, cervical cancer, uterine cancer, testicular cancer, endometrial cancer, glioblastomas such as glioblastoma multiforme, gliomas, oligodendrogliomas, head and neck carcinomas, hepatocellular carcinomas, small intestinal malignancies, melanomas, neuroendocrine tumors, or other HER2 protein expressing or overexpressing cancers. HER2 is typically overexpressed in malignancies of epithelial origin and cancers derived from mesenchyme, neuroendocrine tissue, central nervous system, and kidney and thus the antibodies or antibody fragrments of the present invention may be used to treat these types of cancers. Information on various forms of HER2 expression in cancers can be found, for example, in “HER2 expression status in diverse cancers: review of results from 37,992 patients,” Yan, Min et al., Cancer Metastasis Rev., (2015) 34:157-164. Disease associated with HER2 expression or overexpression include Vulvar Paget's disease.

Antibodies or antibody fragments of the invention may be useful for diagnosing diseases other than cancers for which HER2 protein expression is increased or decreased. Typically, such diagnostic methods involve use of a biological sample obtained from the patient. The biological sample encompasses a variety of sample types obtained from a subject that can be used in a diagnostic or monitoring assay. Biological samples include but are not limited to blood and other liquid samples of biological origin, solid tissue samples such as a biopsy specimen or a tissue culture or cells derived therefrom, and the progeny thereof. For example, biological samples include cells obtained from a tissue sample collected from an individual suspected of having a cancer associated with HER2 protein overexpression. Biological samples encompass clinical samples, cells in culture, cell supernatants, cell lysates, serum, plasma, biological fluid, and tissue samples.

In one embodiment, the invention includes a method of diagnosing a cancer associated with HER2 protein expression or overexpression in a subject by detecting HER2 protein on cells from the subject using the antibody of the invention. This method may include steps of:
1) contacting a biological sample of a subject with an antibody or antibody fragment according to the invention under conditions suitable for the antibody or antibody fragment to form complexes with cells in the biological sample that express HER2 protein; and
(b) detecting and/or quantifying said complexes, whereby detection of said complexes is indicative of a cancer associated with HER2 protein overexpression.

To monitor the progress of a cancer, the method according to the invention may be repeated at different times, to determine if antibody binding to the samples increases or decreases, wherefrom it can be determined if the cancer has progressed, regressed or stabilized.

Another embodiment of the invention is a method of diagnosing a disease associated with the expression or overexpression of HER2 protein. Examples of such diseases may include the cancers described above and vulvar Paget's disease.

In one embodiment, an anti-HER2 antibody or antibody fragment for use in a method of diagnosis or detection is provided. In a further aspect, a method of detecting the presence of HER2 protein in a biological sample is provided. In a further aspect, a method of quantifying the amount of HER2 protein in a biological sample is provided. In certain embodiments, the method comprises contacting the biological sample with an anti-HER2 antibody or antibody fragment as described herein under conditions permissive for binding of the anti-HER2 antibody or antibody fragment to HER2 protein and detecting whether a complex is formed between the anti-HER2 antibody or antibody fragment and HER2 protein. Such a method may be carried out in vitro or in vivo. In one embodiment, such methods may be used to select subjects eligible for therapy. In some embodiments, the therapy will include administration of an anti-HER2 antibody or antibody fragment to the subject.

In certain embodiments, labeled anti-HER2 antibodies or antibody fragments are employed. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### J. Pharmaceutical Formulations

The anti-HER2 antibodies or antibody fragments have anti-proliferative activity. Further, these antibodies or antibody fragments, once conjugated to a cytotoxic agent, can further reduce tumor size and may exhibit reduced toxicity. Thus, the anti-HER2 antibodies, fragments or immunoconjugates thereof may be useful for treating proliferative diseases associated with HER2 protein expression. The antibodies, fragments or immunoconjugates may be used alone or in combination with any suitable agent or other conventional treatments.

The anti-HER2 antibody or antibody fragment may be used to treat diseases associated with HER2 protein expression, overexpression or activation. There are no particular limitations on the types of cancer or tissue that can be treated other than the requirement for HER2 protein expression.

Anti-HER2 antibodies or antibody fragments are potential activators of the innate immune response and thus may be used in immunotherapy. The anti-HER2 antibody or antibody fragment of the invention may also be used as adjuvants for immunization such as for vaccines and as anti-infection agents.

In each of the embodiments of the treatment methods described herein, the anti-HER2 antibody, antibody fragment or anti-HER2 antibody or antibody fragment immunoconjugate may be delivered in a manner consistent with conventional methodologies associated with management of the disease or disorder for which treatment is sought. In accordance with the disclosure herein, an effective amount of the antibody, antibody fragment or immunoconjugate is administered to a subject in need of such treatment for a time and under conditions sufficient to prevent or treat the disease or disorder. Thus, an aspect of the invention relates to a method for treating a disease associated with the expression of HER2 protein comprising administering to a subject in need thereof with a therapeutically effective amount of an antibody, antibody fragment or immunoconjugate of the invention.

For administration, the anti-HER2 antibody, antibody fragment or immunoconjugate may be formulated as a pharmaceutical composition. The pharmaceutical composition including anti-HER2 antibody, antibody fragment or immunoconjugate can be formulated according to known methods for preparing pharmaceutical compositions. In such methods, the therapeutic molecule is typically combined with a mixture, solution or composition containing a pharmaceutically acceptable carrier.

A pharmaceutically acceptable carrier is a material that can be tolerated by a recipient patient. Sterile phosphate-buffered saline is one example of a pharmaceutically acceptable carrier. Other suitable pharmaceutically acceptable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc. These considerations can be evaluated by a skilled person to formulate suitable pharmaceutical compositions. The pharmaceutical compositions of the invention can be formulated for topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. Exemplary vehicles may be isotonic, sterile, saline solutions (monosodium or disodium phosphate, sodium, potassium, calcium or magnesium chloride and the like or mixtures of such salts), or dry, especially freeze-dried compositions which upon addition of, for example, sterilized water or physiological saline, permit the constitution of injectable solutions.

In some embodiments, tonicity agents, sometimes known as “stabilizers” are present to adjust or maintain the tonicity of a liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount of from 0.1% to 25% by weight, preferably 1 to 5% of the pharmaceutical composition. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients may include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be employed to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants may be present in a concentration range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride

The doses used for the administration can be adapted as a function of various parameters, such as the mode of administration, the relevant pathology, and/or the desired duration of treatment.

To prepare pharmaceutical compositions, an effective amount of the antibody or antibody fragment may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in a water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

The anti-HER2 antibody or antibody fragment can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with one or more of the other ingredients enumerated above, as may be required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of dimethyl sulfoxide (DMSO) as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. Aqueous solutions are especially suitable for intravenous, intramuscular, subcutaneous and intraperitoneal administration. In this connection, sterile aqueous media which can be employed are known to those of skill in the art. For example, a dose could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage may occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies or antibody fragments into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to degrade in vivo. Biodegradable polyalkyl-cyanoacrylate nanoparticles that meet these requirements are contemplated for use in the present invention.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 A, containing an aqueous solution in the core. The physical characteristics of liposomes depend on pH, ionic strength and the presence of divalent cations

Pharmaceutical formulations containing an anti-HER2 antibody or antibody fragment as described herein may be prepared by mixing such antibody or antibody fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Exemplary pharmaceutically acceptable carriers herein may include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the indication being treated. Preferably, ingredients with complementary activities that do not adversely affect each other may be combined into a single formulation. For example, it may be desirable to provide an EGFR antagonist (such as erlotinib), an anti-angiogenic agent (such as a VEGF antagonist which may be an anti-VEGF antibody) or a chemotherapeutic agent (such as a taxoid or a platinum agent) in addition to the anti-CTLA4 antibody, antibody fragment or immunoconjugate of the present invention. Such active ingredients are suitably present in combination in amounts that are effective for the purpose intended.

In one embodiment, the anti-HER2 antibody, antibody fragment or immunoconjugate of the present invention is combined in a formulation with another antibody or antibody fragment against an antigen selected from CTLA4, PD1, PD-L1, AXL, ROR2, CD3, EpCAM, B7-H3, ROR1, SFRP4 and a WNT protein including WNT1, WNT2, WNT2B, WNT3, WNT4, WNT5A, WNT5B, WNT6, WNT7A, WNT7B, WNT8A, WNT8B, WNT9A, WNT9B, WNT10A, WNT10B, WNT11, WNT16. The combination may be in the form of two separate molecules: the anti-HER2 antibody, antibody fragment or immunoconjugate of the present invention, and the other antibody or antibody fragment. Alternatively, the combination may also be the form of a single molecule with binding affinity to both HER2 protein and the other antigen, thus forming a multispecific (e.g. bispecific) antibody.

Active ingredients may be encapsulated in microcapsules prepared, for example, by coacervation techniques or by interfacial polymerization. For example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions may be employed. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or antibody fragment, which matrices may be in the form of shaped articles, e.g. films, or microcapsules.

The formulations to be used for in vivo administration are generally sterile. Sterility may be readily accomplished, e.g., by filtration through sterile filtration membranes.

### K. Therapeutic Methods and Compositions

Any of the anti-HER2 antibodies or antibody fragments provided herein may be used in therapeutic methods. In one aspect, an anti-HER2 antibody or antibody fragment for use as a medicament is provided. In further aspects, an anti-HER2 antibody or antibody fragment for use in treating cancer is provided. A list of cancers that have been found to express or overexpess HER2 that are suitable targets of therapeutic methods is provided above.

In certain embodiments, an anti-HER2 antibody or antibody fragment for use in a method of treatment is provided. In certain embodiments, the invention provides an anti-HER2 antibody or antibody fragment for use in a method of treating an individual having cancer comprising administering to the individual a therapeutically effective amount of the anti-HER2 antibody or antibody fragment. In one such embodiment, the method further comprises administering to the individual a therapeutically effective amount of at least one additional therapeutic agent, e.g., as described below. In further embodiments, the invention provides an anti-HER2 antibody or antibody fragment for use in inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function and methods of treating these conditions using an anti-HER2 antibody or antibody fragment comprising administering to the individual an effective of the anti-HER2 antibody or antibody fragment to treat the condition. An “individual” according to any of the embodiments of the invention is preferably a human.

In a further aspect, the invention provides for the use of an anti-HER2 antibody or antibody fragment in the manufacture or preparation of a medicament. In one embodiment, the medicament is for treatment of any of the cancers or diseases mentioned above. The medicament is for use in a method of treating cancer comprising administering to an individual having cancer a therapeutically effective amount of the medicament. In one such embodiment, the method further comprises administering to the individual a therapeutically effective amount of at least one additional therapeutic agent, e.g., as described below. In a further embodiment, the medicament is for inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function.

In a further aspect, the invention provides a method for treating a cancer. In one embodiment, the method comprises administering to an individual having such cancer a therapeutically effective amount of an anti-HER2 antibody or antibody fragment. In one such embodiment, the method further comprises administering to the individual a therapeutially effective amount of at least one additional therapeutic agent, as described below. An “individual” according to any of the above embodiments may be a human.

In a further aspect, the invention provides a method for inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function in an individual. In one embodiment, the method comprises administering to the individual a therapeutically effective amount of an anti-HER2 antibody or antibody fragment to inhibit angiogenesis, inhibit cell proliferation, promote immune function, induce inflammatory cytokine section (e.g., from tumor-associated macrophages), inhibit tumor vasculature development (e.g., intratumoral vasculature or tumor-associated vasculature), and/or inhibit tumor stromal function.

In a further aspect, the invention provides pharmaceutical formulations comprising any of the anti-HER2 antibodies or antibody fragments provided herein, e.g., for use in any of the above therapeutic methods and at least one additional therapeutic agent, e.g., as described below.

In each and every treatment described above, the antibodies or antibody fragments of the invention can be used alone, as immunoconjugates or in combination with other agents in a therapy. For instance, an antibody of the invention may be co-administered with at least one additional therapeutic agent. In certain embodiments, an additional therapeutic agent is an anti-angiogenic agent. In certain embodiments, an additional therapeutic agent is a VEGF antagonist (in some embodiments, an anti-VEGF antibody, for example bevacizumab). In certain embodiments, an additional therapeutic agent is an EGFR antagonist (in some embodiment, erlotinib). In certain embodiments, an additional therapeutic agent is a chemotherapeutic agent and/or a cytostatic agent. In certain embodiments, an additional therapeutic agent is a taxoid (e.g., paclitaxel) and/or a platinum agent (e.g., carboplatinum). In certain embodiments the additional therapeutic agent is an agent that enhances the patient's immunity or immune system.

Such combination therapies noted above encompass combined administration (where two or more therapeutic agents are included in the same or separate formulations), and separate administration, in which case, administration of the antibody or antibody fragment can occur prior to, simultaneously, and/or following, administration of the additional therapeutic agent and/or adjuvant. Antibodies or antibody fragments can also be used in combination with radiation therapy.

The anti-HER2 antibodies or antibody fragments may be formulated, dosed, and administered in a manner consistent with good medical practice. Factors for consideration in this context include the disorder being treated, the mammal being treated, the clinical condition of the individual patient, the cause of the disorder, the site of delivery of the agent, the method of administration, the scheduling of administration, and other factors known to medical practitioners. The antibody or antibody fragment need not be but is optionally formulated with one or more agents currently used to prevent or treat the disorder in question. The effective amount of such other agents depends on the amount of antibody or antibody fragment present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antibody fragment (when used alone or in combination with one or more other additional therapeutic agents) will depend on the type of disease to be treated, the type of antibody or antibody fragment, the severity and course of the disease, whether the antibody or antibody fragment is administered for preventive or therapeutic purposes, previous therapy, the patient's clinical history and response to the antibody or antibody fragment, and the discretion of the attending physician. The antibody or antibody fragment is suitably administered to the patient at one time or over a series of treatments. Depending on the type and severity of the disease, about 1 μg of antibody or antibody fragment/kg bodyweight of the patient to 40 mg of antibody or antibody fragment/kg bodyweight of the patient can be an initial candidate dosage for administration to the patient, whether, for example, by one or more separate administrations, or by continuous infusion. One typical daily dosage might range from about 1 μg of antibody or antibody fragment/kg bodyweight of the patient to 100 mg of antibody or antibody fragment/kg bodyweight of the patient or more, depending on the factors mentioned above. For repeated administrations over several days or longer, depending on the condition, the treatment would generally be sustained until a desired suppression of disease symptoms occurs. Such doses may be administered intermittently, e.g. every week or every three weeks (e.g. such that the patient receives from about two to about twenty, or e.g. about six doses of the antibody or antibody fragment). An initial higher dose followed by one or more lower doses may be administered. However, other dosage regimens may be useful. The progress of this therapy can be monitored by conventional techniques and assays.

The dosage of the antibody or antibody fragment will be the same if administered in the form of a bispecific antibody, in combination with another immune checkpoint inhibitor or another antibody or antibody fragment or as an immunoconjugate. Further, a polypeptide having anti-HER2 activity will be administered in the same amounts as the antibody or antibody fragment.

The amount of the antibody or antibody fragment in the single dose of the pharmaceutical formulation will remain the same if administered in the form of a bispecific antibody, in combination with another immune checkpoint inhibitor or as an immunoconjugate, or in combination with another antibody or antibody fragment against another antigen as disclosed herein. Further, a polypeptide having anti-HER2 activity will be included in the single dose of the pharmaceutical formulation in the same amounts as the antibody or antibody fragment.

In one embodiment, the anti-HER2 antibody or antibody fragment may be conjugated to an immune checkpoint inhibitor molecule or may form part of a bispecific antibody with an immune checkpoint inhibitor. The combination can be the anti-HER2 antibody or antibody fragment disclosed in this application and the immune checkpoint inhibitor molecule administered as separate molecules or as a bispecific antibody. Such a bispecific antibody has a binding activity to HER2 protein and a second binding activity to the immune checkpoint.

The immune checkpoint may be selected from CTLA4, LAGS, TIM3, TIGIT, VISTA, BTLA, OX40, CD40, 4-1BB, PD-1, PD-L1, and GITR (Zahavi and Weiner, International Journal of Molecular Sciences, vol. 20, 158, 2019). Additional immune checkppoints include B7-H3, B7-H4, KIR, A2aR, CD27, CD70, DR3, and ICOS (Manni et al, Immune checkpoint blockade and its combination therapy with small-molecule inhibitors for cancer treatment, Bbacan, https://doi.org/10.1016/j.bbcan.2018.12.002, 2018).

The immune checkpoint is preferably CTLA4, PD-1 or PD-L1.

It is understood that any of the above formulations or therapeutic methods may be carried out using an antibody fragment or an immunoconjugate of the invention in place of or in addition to an anti-HER2 antibody.

Enhancing the host's immune function to combat tumors may be used in conjunction with the methods of the present invention. Conventional methods include (i) APC enhancement, such as (a) injection into the tumor of DNA encoding foreign MHC alloantigens, or (b) transfecting biopsied tumor cells with genes that increase the probability of immune antigen recognition (e.g., immune stimulatory cytokines, GM-CSF, co-stimulatory molecules B7.1, B7.2) of the tumor, (iii) adoptive cellular immunotherapy, or treatment with activated tumor-specific T-cells. Adoptive cellular immunotherapy includes isolating tumor-infiltrating host T-lymphocytes, expanding the population in vitro, such as through stimulation by IL-2 or tumor or both. Additionally, isolated T-cells that are dysfunctional may be also be activated by in vitro application of anti-PD-L1 antibodies. T-cells that are so-activated may then be readministered to the host. One or more of these methods may be used in combination with administration of the antibody, antibody fragment or immunoconjugate of the present invention.

Traditional therapies for cancer include the following: (i) radiation therapy (e.g., radiotherapy, X-ray therapy, irradiation) or the use of ionizing radiation to kill cancer cells and shrink tumors. Radiation therapy can be administered either externally via external beam radiotherapy (EBRT) or internally via brachytherapy; (ii) chemotherapy, or the application of cytotoxic drug which generally affect rapidly dividing cells; (iii) targeted therapies, or agents which specifically affect the deregulated proteins of cancer cells (e.g., tyrosine kinase inhibitors imatinib, gefitinib; monoclonal antibodies, photodynamic therapy); (iv) immunotherapy, or enhancement of the host's immune response (e.g., vaccine); (v) hormonal therapy, or blockade of hormone (e.g., when tumor is hormone sensitive), (vi) angiogenesis inhibitor, or blockade of blood vessel formation and growth, and (vii) palliative care, or treatment directed to improving the quality of care to reduce pain, nausea, vomiting, diarrhea and hemorrhage. Pain medication such as morphine and oxycodone, anti-emetics such as ondansetron and aprepitant, can permit more aggressive treatment regimens.

In the treatment of cancer, any of the previously described conventional treatments for the treatment of cancer immunity may be conducted, prior, subsequent or simultaneous with the administration of the anti-HER2 antibodies or antibody fragments. Additionally, the anti-HER2 antibodies or antibody fragments may be administered prior, subsequent or simultaneous with conventional cancer treatments, such as the administration of tumor-binding antibodies (e.g., monoclonal antibodies, toxin-conjugated monoclonal antibodies) and/or the administration of chemotherapeutic agents.

### L. Articles of Manufacture and Kits

In another aspect of the invention, an article of manufacture containing an anti-HER2 antibody or antibody fragment and other materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody or antibody fragment of the invention. The label or package insert indicates that the composition is used for treating the condition of choice. Moreover, the article of manufacture may comprise (a) a first container with a composition contained therein, wherein the composition comprises an antibody or antibody fragment; and (b) a second container with a composition contained therein, wherein the composition comprises a further cytotoxic or otherwise therapeutic agent. The article of manufacture in this embodiment of the invention may further comprise a package insert indicating that the compositions can be used to treat a condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-HER2 antibody or antibody fragment.

Finally, the invention also provides kits comprising at least one antibody or antibody fragment of the invention. Kits containing polypeptide, antibodies or antibody fragments, or antibody drug conjugate of the invention find use in detecting HER2 protein expression (increase or decrease), or in therapeutic or diagnostic assays. Kits of the invention can contain an antibody coupled to a solid support, e.g., a tissue culture plate or beads (e.g., sepharose beads). Kits can be provided which contain antibodies for detection and quantification of HER2 protein in vitro, e.g. in an ELISA or a Western blot. Such antibody useful for detection may be provided with a label such as a fluorescent or radiolabel.

The kits further contain instructions on the use thereof. In some embodiments, the instructions comprise instructions required by the U.S. Food and Drug Administration for in vitro diagnostic kits. In some embodiments, the kits further comprise instructions for diagnosing the presence or absence of cerebrospinal fluid in a sample based on the presence or absence of HER2 protein in said sample. In some embodiments, the kits comprise one or more antibodies or antibody fragments. In other embodiments, the kits further comprise one or more enzymes, enzyme inhibitors or enzyme activators. In still other embodiments, the kits further comprise one or more chromatographic compounds. In yet other embodiments, the kits further comprise one or more compounds used to prepare the sample for spectroscopic assay. In further embodiments, the kits further comprise comparative reference material to interpret the presence or absence of HER2 protein according to intensity, color spectrum, or other physical attributes of an indicator.

The following examples are illustrative, but not limiting, of the anti-HER2 antibodies of the present disclosure. Other suitable modifications and adaptations of the variety of conditions and parameters normally encountered in the field, and which are obvious to those skilled in the art, are within the scope of the disclosure.

### EXAMPLES

### Example 1: Binding Activities of the Humanized Conditionally Active Anti-HER2 Antibodies to Human HER2 Protein

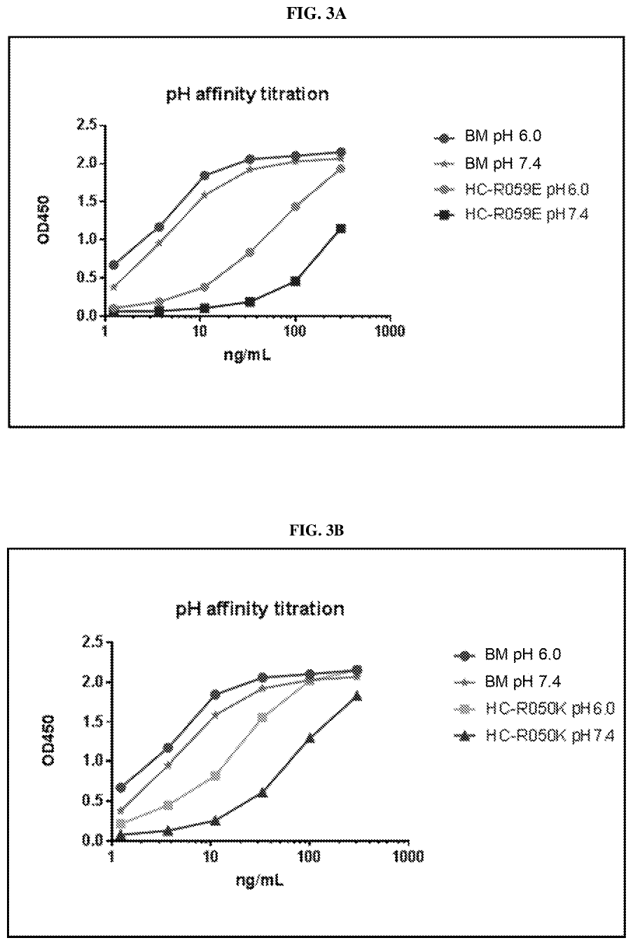

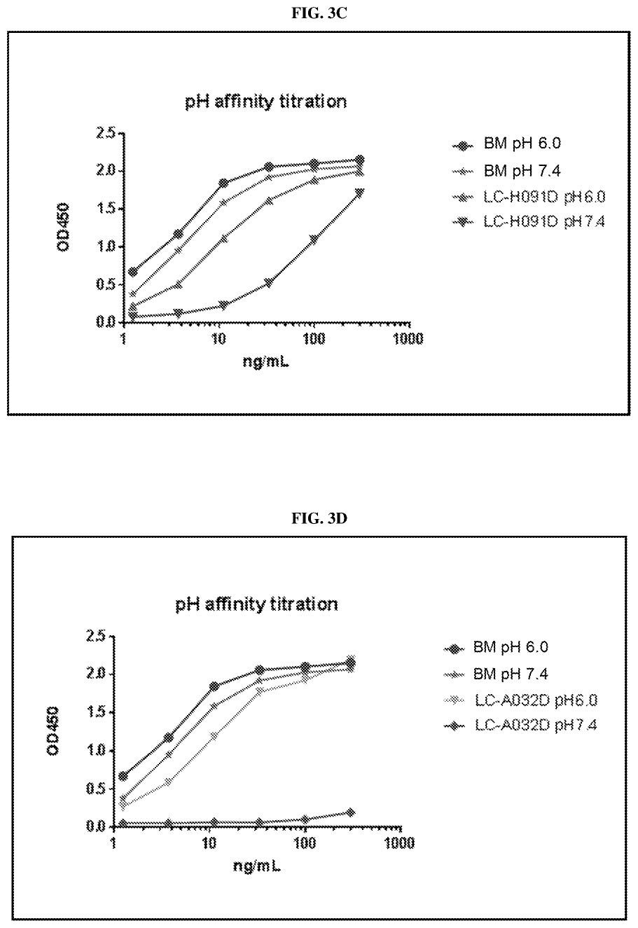

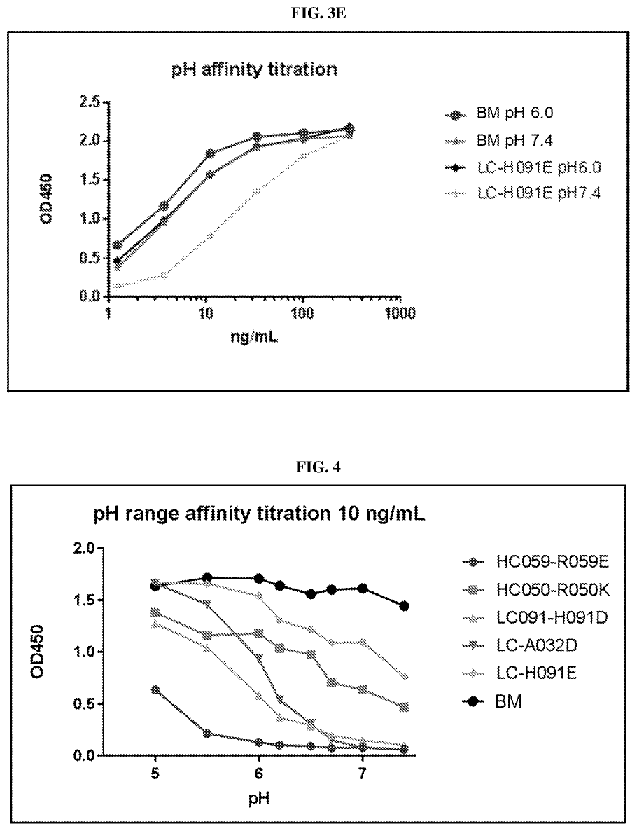

The binding activities of conditionally active anti-HER2 antibodies to human HER2 protein were measured by ELISA, using a Benchmark antibody as a control. The Benchmark antibody is indicated by “BM.” For each of the conditionally active antibodies, one of the heavy chain (HC) and the light chain (LC) is specified in each figure. The unspecified heavy or light chain is the heavy or light chain of the Benchmark antibody. The Y-axis is the optical density (OD) at 450 nm. The X-axis shows the antibody concentration (log ng/mL) with a starting concentration of 300 ng/mL. The results are shown in FIGS. 3A-3E.

The pH affinity ELISA assay was carried out using the following protocol.

### pH Affinity Elisa Assay

1) Coat ELISA plates with 100 μL of 1 μg/mL recombinant human HER2 antigen in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute antibodies in 3-fold dilutions starting at 300 ng/mL in pH 6.0 or pH 7.4 ELISA incubation buffer.
8) Add 100 μL/well of diluted antibodies to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the 3, 3′, 5, 5′ tetramethylbenzidine (TMB) substrate solution into all wells of the plates. Incubate at room temperature for about 2 minutes 15 seconds or 2 minutes.
18) Add 50 μL per well of 1N hydrochloric acid (HCl) into all wells of the plates. Read plates at 450 nm using PerkinElmer, EnSpire 2300 Multilabel Reader.

### Example 2: Binding Activities of the Conditionally Active Anti-HER2 Antibodies

Binding activity of the same HER2 Benchmark antibody and CAB antibodies to human HER2 protein at various pH values were determined by a pH range ELISA assay. The Benchmark antibody is indicated by “BM.” For each of the conditionally active antibodies the heavy chain (HC) and the light chain (LC) are specified in FIG. 4. The unspecified heavy or light chain is the heavy or light chain of the Benchmark antibody. The Y-axis is the optical density (OD) at 450 nm. Antibodies were diluted to 10 ng/mL in various pH ELISA incubation buffers ranging from pH 5.0 to pH 7.4. The X-axis shows the pH of the incubation and wash buffers (pH 5.0, 5.5, 6.0, 6.5, 7.0 and 7.4). The results are shown in FIG. 4.

The pH range ELISA assay was carried out using the following protocol.

### pH Range Elisa Assay

1) Coat ELISA plates with 100 μL of 1 μg/mL recombinant human HER2 antigen in carbonate-bicarbonate coating buffer
2) Cover plates with sealing film and incubate overnight at 4° C.
3) Decant plates and tap out residual liquid on a stack of paper towels
4) Wash wells twice by dispensing 200 μL of various pH incubation buffer to the wells and completely aspirate the contents
5) Add 200 μL of various pH incubation buffers (pH 5.0, 5.5, 6.0, 6.5, 7.0 and 7.4) to the wells. Cover with sealing film and place the plate onto a plate shaker (set to 200 rpm) for 60 minutes at room temperature
6) Decant plates and tap out residual liquid on a stack of paper towels
7) Serially dilute test substances in various pH incubation buffers (pH 5.0, 5.5, 6.0, 6.5, 7.0 and 7.4) to 10 ng/mL
8) Add 100 μL/well of diluted test substances to the plates
9) Cover with sealing film and place the plates onto a plate shaker (set to 200 rpm) for 60 minutes at room temperature.
10) Decant plates and tap out residual liquid on a stack of paper towels.
11) Wash wells three times by dispensing 200 μL of various pH wash buffers (pH 5.0, 5.5, 6.0, 6.5, 7.0 and 7.4) to the wells and completely aspirate the contents
12) Dilute the HRP secondary antibody at 1:2500 in various pH incubation buffers (pH 5.0, 5.5, 6.0, 6.5, 7.0 and 7.4)
13) Add 100 μL horseradish peroxidase (HRP) secondary antibody diluted in various pH incubation buffers (pH 5.0, 5.5, 6.0, 6.5, 7.0 and 7.4) to each well.
14) Cover with sealing film and place the plates onto a plate shaker (set to 200 rpm) for 60 minutes at room temperature.
15) Decant plates and tap out residual liquid on a stack of paper towels.
16) Wash wells three times by dispensing 200 μL of various pH wash buffer (pH 5.0, 5.5, 6.0, 6.5, 7.0 and 7.4) to the wells and completely aspirating the contents
17) Dispense 50 μL per well of the 3, 3′, 5, 5′ tetramethylbenzidine (TMB) substrate solution into all wells of plates. Incubate at room temperature for 3 minutes.
18) Add 50 μL per well of 1N hydrochloric acid (HCl) into all wells of the plates. Read plates at 450 nm using PerkinElmer EnSpire 2300 Multilabel Reader.

### Example 3: Binding Activities of the Conditionally Active Anti-HER2 Antibodies Measured by FACS

Conditionally active anti-HER2 antibodies were analyzed for binding to HER2 protein using the Benchmark antibody BM as a control. The binding activities of these anti-HER2 antibodies to HER2 protein expressing SKBR3 cancer cells were (ATCC, Cat #HTB30) measured by fluorescence activated cell sorting (FACS) at two different pH values of 6.0 and 7.4. Different concentrations of the antibodies of 10 μg/mL, 3.3 μg/mL, 1.1 μg/mL and 0.37 μg/mL were used. Antibodies were first diluted to 10 μg/mL in pH 6.0 or pH 7.4 FACS buffer, then 3-fold serially diluted in pH 6.0 or pH 7.4 FACS buffer. SKBR3 cells (ATCC, Cat #HTB30) were maintained in SKBR3 culture medium (McCoy's+10% FBS). The cells were routinely sub-cultured twice per week. The cells were harvested during exponential growth phase and counted for plating.

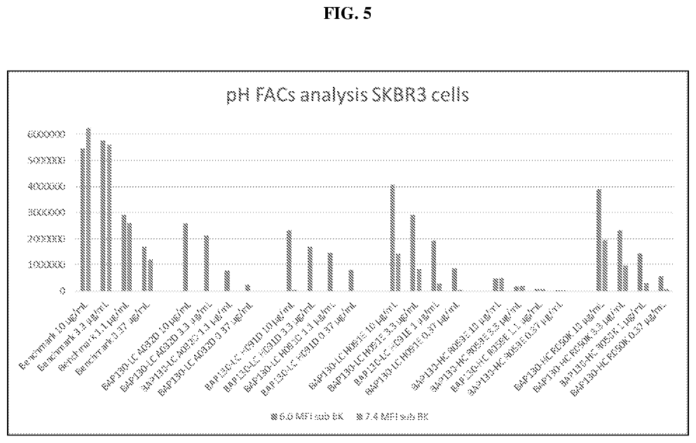

The median fluorescence intensity (MFI) of Alexa Fluor 488 (AF488) in cell singlets was plotted using GraphPad Prism software version 7.03. The conditionally active anti-HER2 antibodies consistently showed a higher binding activity to the HER2 protein expressing SKBR3 cells at pH 6.0 (blue) than at pH 7.4 (orange). See FIG. 5. Y-axis: MFI. X-axis: Test antibodies at different concentrations. MFI sub BK: median fluorescence intensity of test antibodies with the median fluorescence intensity of a secondary antibody only sample subtracted therefrom to obtain the true fluorescence by subtracting the background fluorescence.

The test protocol that was employed is set forth below.

### Cell Staining Using Test Antibodies

1) Seed 3×10⁶ cells to T-75 flasks and culture according to the instructions of vendors.
2) On the day of FACS analysis, remove and discard culture medium.
3) Briefly rinse the cell layer with PBS solution.
4) Add 1.5 mL of Detachin solution to each of the T-75 flasks. Wait until cell layer has dispersed.
5) Add 4.5 mL of culture media for the corresponding cell lines and resuspend cells by gently pipetting.
6) Pool the cells and transfer the cell suspension to a 50-mL conical tube.
7) Count the cells with trypan blue staining before centrifugation at 1500 rpm for 5 min at 4° C.
8) Wash the cells once with phosphate buffered saline (PBS)
9) Resuspend the cells in pH 6.0 or pH 7.4 FACS buffer to 3.5×10⁶ cells/mL.
10) Aliquot 3.5×10⁵ cells in 100 μL pH 6.0 or pH 7.4 FACS buffer in 96-well U-bottom plates.
11) Spin down the cells and discard the buffer.
12) Serially dilute antibodies in 3-fold dilutions starting at 10 μg/mL in pH 6.0 or pH 7.4 FACS buffer.
13) Add 100 μL/well of the diluted antibodies to cells, gently mix well and incubate on ice with shaking (200 rpm) for one hour.
14) Centrifuge the cells at 1500 rpm for 5 min at 4° C. Wash the cells with 150 μL of pH 6.0 or pH 7.4 wash buffer twice.
15) Dilute the goat anti-human IgG AF488 antibody 1:300 in pH 6.0 or pH 7.4 FACS buffers.
16) Add 100 μL of the diluted antibody from step above to the cells and incubate on ice with shaking (200 rpm) for 45 minutes, protected from light.
17) Pellet the cells and wash with 150 μL of pH 6.0 or pH 7.4 wash buffer three times.
18) Fix cells with 4% paraformaldehyde diluted in 1×PBS for 10 min at R.T., then wash cells with 1×PBS.
19) Resuspend the cells in 100 μL of 1×PBS.
20) Analyze the cells by NovoCyte Flow Cytometer using Ex488 nm/Em530 nm. Collect at least 5,000 singlet cells for each data point.

### Example 4: Binding Activities of the Conditionally Active Anti-HER2 Antibodies to Human HER2 Protein

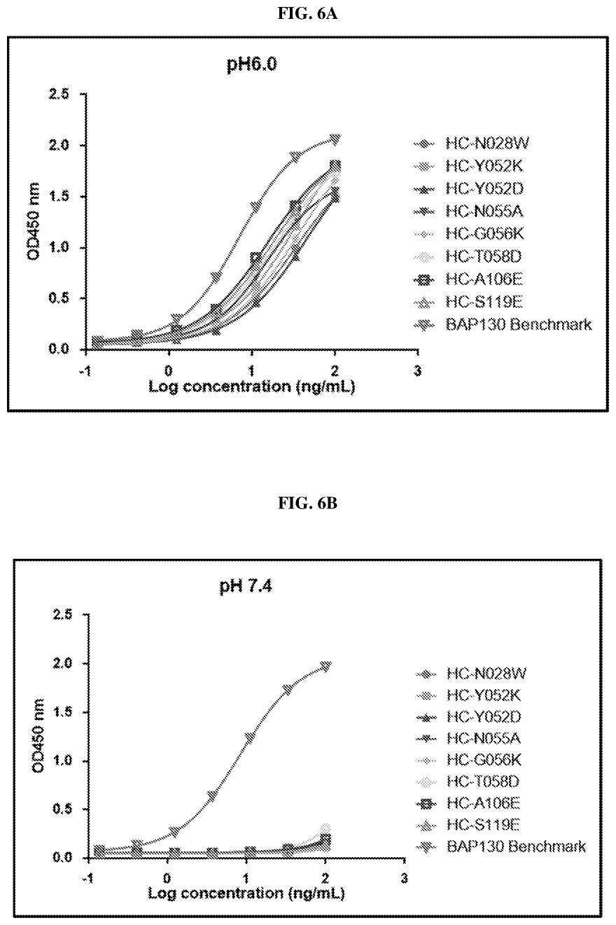

The binding activities of conditionally active anti-HER2 antibodies to human HER2 protein were measured by ELISA, using a Benchmark antibody as a control. The Benchmark antibody is indicated by “BM.” For each of the conditionally active antibodies, the heavy chain (HC) is specified in FIGS. 6A-6B. Each of the tested antibodies had the light chain LC-A032D. The antibodies were first diluted to 100 ng/mL in pH 6.0 or pH 7.4 ELISA incubation buffer. Then 100 ng/mL of antibodies were 3-fold serially diluted in pH 6.0 or pH 7.4 ELISA incubation buffer.

The results are shown in FIGS. 6A-6B. The Y-axis is the optical density (OD) at 450 nm. The X-axis shows the antibody concentration (log ng/mL) with a starting concentration of 100 ng/mL.

### Example 5—Binding Activity of HER2 Antibodies to cynoHER2 Protein at pH 6.0 and pH 7.4 Determined by pH Affinity ELISA Assay

The binding activities of conditionally active anti-HER2 antibodies to cynoHER2 protein were measured by ELISA, using a Benchmark antibody as a control. The Benchmark antibody is indicated by “BM.” For each of the conditionally active antibodies, the heavy chain (HC) is specified in FIGS. 6A-6B. Each of the tested antibodies had the light chain LC-A032D. The antibodies were first diluted to 100 ng/mL in pH 6.0 or pH 7.4 ELISA incubation buffer. Then 100 ng/mL of antibodies were 3-fold serially diluted in pH 6.0 or pH 7.4 ELISA incubation buffer.

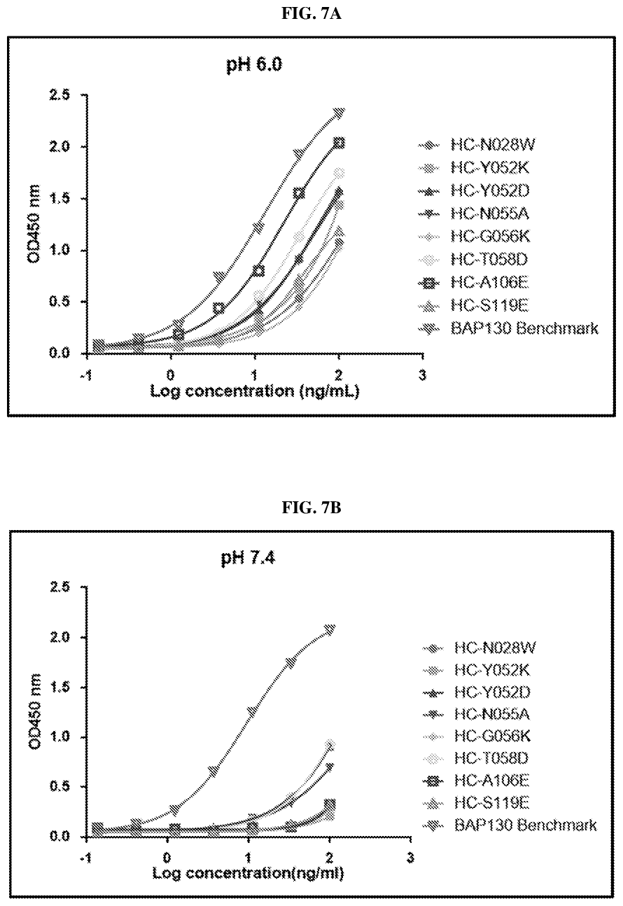

The results are shown in FIGS. 7A-7B. The Y-axis is the optical density (OD) at 450 nm. The X-axis shows the antibody concentration (log ng/mL) with a starting concentration of 100 ng/mL.

The pH affinity ELISA assays of Examples 4-5 were carried out using the following protocol.

### pH Affinity ELISA Assay Used in Examples 4-5

1) Coat ELISA plates with 100 μL of 1 μg/mL recombinant human or cyno HER2 antigen (see figure legends for species information) in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute antibodies in 3-fold dilutions starting at 100 ng/mL in pH 6.0 or pH 7.4 ELISA incubation buffer.
8) Add 100 μL/well of diluted antibodies to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the TMB substrate solution into all wells of the plates. Incubate at room temperature for about 2 minutes 15 seconds or 2 minutes.
18) Add 50 μL per well of 1N HCl into all wells of the plates. Read plates at 450 nm using PerkinElmer, EnSpire 2300 Multilabel Reader.

### Example 6: Binding Activities of the Conditionally Active Anti-HER2 Antibodies to Human HER2 Protein

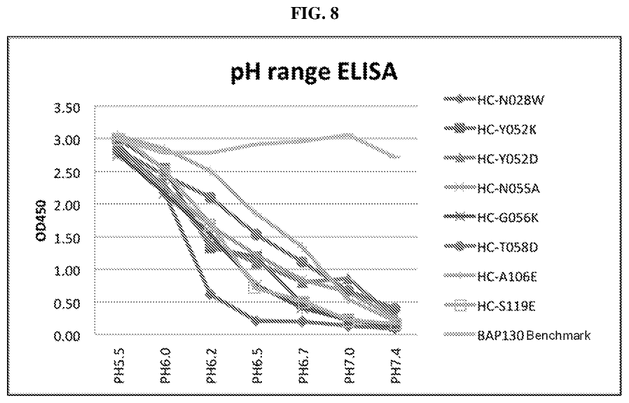

Binding activity of the HER2 Benchmark antibody and CAB antibodies to human HER2 protein at various pH values were determined by a pH range ELISA assay. The Benchmark antibody is indicated by “BM.” For each of the conditionally active antibodies the heavy chain (HC) is specified in FIG. 8. Each of the tested antibodies had the light chain LC-A032D. The Y-axis is the optical density (OD) at 450 nm. Antibodies were diluted to 100 ng/mL in various pH ELISA incubation buffers ranging from pH 5.0 to pH 7.4. The X-axis shows the pH of the incubation and wash buffers (pH 5.0, 5.5, 6.0, 6.5, 7.0 and 7.4).

Average OD values for each pH were plotted against the pH of the buffer using GraphPad Prism 5.03. Curve fitting was done using the 4-parameter model built into the software. Binding activity at pH 6.0 was set to 100%. The results are shown in FIG. 8.

The inflection point of the pH curve (50% binding activity) equals parameter EC50 of the fitting equation. The pH inflection points are shown in Table 2 below.

TABLE 2

| | pH inflection |
|---|---|
| Clone Name | point calculation |
| HC-N028W | 6.053 |
| HC-Y052K | 6.221 |
| HC-Y052O | 6.132 |
| HC-N055A | 6.298 |
| HC-G056K | 6.226 |
| HC-T058D | 5.453 |
| HC-A106E | 6.608 |
| HC-S119E | 6.218 |
| BAP130 Benchmark | n/a |

The pH range ELISA assay was carried out using the following protocol.

### pH Range ELISA Assay

1) Coat ELISA plates with 100 μL of 1 μg/mL recombinant human HER2 antigen in carbonate-bicarbonate coating buffer
2) Cover plates with sealing film and incubate overnight at 4° C.
3) Decant plates and tap out residual liquid on a stack of paper towels
4) Wash wells twice by dispensing 200 μL of various pH incubation buffer to the wells and completely aspirate the contents
5) Add 200 μL of various pH incubation buffers (pH 5.0, 5.5, 6.0, 6.5, 7.0 and 7.4) to the wells. Cover with sealing film and place the plate onto a plate shaker (set to 200 rpm) for 60 minutes at room temperature
6) Decant plates and tap out residual liquid on a stack of paper towels
7) Serially dilute test substances in various pH incubation buffers (pH 5.0, 5.5, 6.0, 6.5, 7.0 and 7.4) to 100 ng/mL
8) Add 100 μL/well of diluted test substances to the plates
9) Cover with sealing film and place the plates onto a plate shaker (set to 200 rpm) for 60 minutes at room temperature.
10) Decant plates and tap out residual liquid on a stack of paper towels.
11) Wash wells three times by dispensing 200 μL of various pH wash buffers (pH 5.0, 5.5, 6.0, 6.5, 7.0 and 7.4) to the wells and completely aspirate the contents
12) Dilute the horseradish peroxidase (HRP) secondary antibody at 1:2500 in various pH incubation buffers (pH 5.0, 5.5, 6.0, 6.5, 7.0 and 7.4)
13) Add 100 μL HRP secondary antibody diluted in various pH incubation buffers (pH 5.0, 5.5, 6.0, 6.5, 7.0 and 7.4) to each well.
14) Cover with sealing film and place the plates onto a plate shaker (set to 200 rpm) for 60 minutes at room temperature.
15) Decant plates and tap out residual liquid on a stack of paper towels.
16) Wash wells three times by dispensing 200 μL of various pH wash buffer (pH 5.0, 5.5, 6.0, 6.5, 7.0 and 7.4) to the wells and completely aspirating the contents
17) Dispense 50 μL per well of the 3, 3′, 5, 5′ tetramethylbenzidine (TMB) substrate solution into all wells of plates. Incubate at room temperature for 3 minutes.
18) Add 50 μL per well of 1N hydrochloric acid (HCl) into all wells of the plates. Read plates at 450 nm using PerkinElmer EnSpire 2300 Multilabel Reader.

### Example 7—In Vivo Efficacy Evaluation of Conditionally Active Antibodies in the Subcutaneous xBT474 CDX Model in BALB/c Nude Mice

In vivo testing of antibodies was carried out in BALB/c nude mice as described below.

TABLE 3
Description of experimental design

| | | | | Dose | | |
|---|---|---|---|---|---|---|
| | | | Dose | Volume^{b} | | |
| Group | N^{a} | Treatment | (mg/kg) | (ml/kg) | Route | Schedule |
| 1 | 8 | Vehicle | — | 10 | IV | Q4D × 4 doses |
| 2 | 8 | LC A032D/HC Benchmark | 3 | 10 | IV | Q4D × 4 doses |
| | | BA-130-00-01 | | | | |
| 3 | 8 | LC A032D/HC Y052K | 3 | 10 | IV | Q4D × 4 doses |
| | | BA-130-03-02 | | | | |
| 4 | 8 | LC A032D/HC G056K | 3 | 10 | IV | Q4D × 4 doses |
| | | BA-130-03-05 | | | | |
| 5 | 8 | LC A032D/HC T058D | 3 | 10 | IV | Q4D × 4 doses |
| | | BA-130-03-06 | | | | |
| 6 | 8 | LC A032D/HC A106E | 3 | 10 | IV | Q4D × 4 doses |
| | | BA-130-03-07 | | | | |
| 7 | 8 | LC A032D/HC S119E | 3 | 10 | IV | Q4D × 4 doses |
| | | BA-130-03-08 | | | | |
| 8 | 8 | B12 Isotype Control | 3 | 10 | IV | Q4D × 4 doses |
| | | Antibody | | | | |

Notes:
^{a}N: number of animals per group.
^{b}Dosing volume was adjusted to 10 μl/g body weight.

### Materials

### Animals

Species: Mus musculus

Strain: BALB/c nude

Age: 6-8 weeks

Sex: Female

Body weight: 18-22 g

Number of animals: 64 mice plus spare

### Experimental Instruments and Reagents

### Instruments

Instrument name: Centrifuge

Supplier: Eppendorf

Equipment type: 5424R

Instrument name: CO₂ incubator

Supplier: Thermo Fisher

Equipment type: Heracell 240i

Instrument name: Balance

Supplier: Changzhou Keyuan electronic instrument co., LTD.

Equipment type: JA20002

Instrument name: Digimatic Caliper

Supplier: MITUTOYO/ABSLUTE

Equipment type: CD-6″ASX

### Reagents

Product identification: Phosphate Buffered Saline (PBS)

Manufacturer: Hyclone

Cat number: SH30256.01

Lot number: AD2158027

Product identification: Hybri-Care

Manufacturer: Gibco

Cat number: ATCC46-X

Lot number: 80719180

Product identification: Penicillin/Streptomycin

Manufacturer: HyClone

Cat number: 15240-062

Lot number: 1989506

Product identification: Trypsin-EDTA

Manufacturer: Gibco

Cat number: 25200-072

Lot number: 2001888

Product identification: Fetal bovine serum

Manufacturer: Hyclone

Cat number: SV30087.03

Lot number: RBC35932

### Experimental Methods and Procedures

### Cell Culture

The xBT474 tumor cells (ATCC® HTB-20TH) were maintained in vitro as a monolayer culture in Hybri-Care medium supplemented with 1.5 g/l sodium bicarbonate, 10% heat inactivated fetal bovine serum, 100 U/ml penicillin and 100 μg/ml streptomycin at 37° C. with 5% CO₂ in air. The tumor cells were routinely subcultured twice weekly by trypsin-EDTA treatment. The cells growing in an exponential growth phase were harvested and counted for tumor inoculation.

### Tumor Inoculation and Animal Grouping

Each mouse was inoculated with 0.36 mg 17-β-estradiol pellet 3 days before subcutaneously cell inoculation on the right flank with xBT474 tumor cells (10×10⁶+Matrigel, 1:1) in 0.2 ml of PBS for tumor development. Treatments were started on day 16 after tumor inoculation when the average tumor size reached approximately 207 mm³. Animals were assigned into groups according to their tumor volume using an Excel-based stratified randomization program. Each group consisted of 8 tumor-bearing mice. The testing articles were administrated according to the experimental design shown in Table 3.

In Vivo Efficacy Evaluation of Conditionally Active Antibodies in the Subcutaneous xBT474 CDX Model in BALB/c Nude Mice
In vivo testing of antibodies was carried out in BALB/c nude mice as described below.

### Table 3.

Tumor size was measured twice weekly in two dimensions using a caliper and was calculated using the formula: Tumor Volume (TV)=0.5 a×b² where a and b are the long and short diameters of the tumor, respectively. The tumor size was then used for calculations of T/C, Tumor growth inhibition (TGI) and Relative Tumor Volume (RTV) values. The T/C value (in percent) is an indication of antitumor effectiveness; T and C are the mean volumes of the treated and control groups, respectively, on a given day. TGI for each treatment group was calculated using the formula: TGI (%)=[1−(Tᵢ−T₀)/(Vᵢ−V₀)]×100; Tᵢ is the average tumor volume of a treatment group on a given day, To is the average tumor volume of the treatment group on day 0, Vᵢ is the average tumor volume of the vehicle control group on the same day as Tᵢ, and V₀ is the average tumor volume of the vehicle group on day 0. Individual RTV was calculated by dividing the tumor volume on a specific day by its volume on day 0. The RTV value of each mouse was calculated individually which was then used for mean RTV calculation for a group.

### Statistical Analysis

The mean tumor volume of each group and SEM at different time points were calculated (Table 4). Statistical analysis of difference in the tumor volume among groups were conducted on the data obtained on Day 23 and Day 27 after the start of treatment.

TABLE 4
Tumor volume

| | Tumor volume (mm³) ^{a} | | | | | | | |
|---|---|---|---|---|---|---|---|---|
| Days | G1^{b} | G2 | G3 | G4 | G5 | G6 | G7 | G8 |
| 0 | 207 ± 12 | 207 ± 12 | 207 ± 12 | 207 ± 12 | 207 ± 13 | 207 ± 13 | 207 ± 13 | 207 ± 12 |
| 2 | 312 ± 21 | 250 ± 19 | 293 ± 24 | 244 ± 11 | 258 ± 19 | 269 ± 26 | 278 ± 20 | 317 ± 18 |
| 6 | 600 ± 61 | 117 ± 5 | 164 ± 14 | 162 ± 24 | 107 ± 10 | 93 ± 9 | 170 ± 31 | 519 ± 37 |
| 9 | 767 ± 73 | 68 ± 9 | 94 ± 11 | 109 ± 15 | 78 ± 4 | 65 ± 6 | 92 ± 9 | 653 ± 52 |
| 13 | 1,046 ± 97 | 27 ± 5 | 56 ± 9 | 58 ± 10 | 24 ± 3 | 25 ± 2 | 31 ± 4 | 806 ± 66 |
| 16 | 1,220 ± 121 | 5 ± 2 | 19 ± 6 | 20 ± 3 | 12 ± 2 | 9 ± 2 | 18 ± 5 | 950 ± 121 |
| 20 | 1,458 ± 136 | 0 ± 0 | 3 ± 2 | 5 ± 2 | 2 ± 2 | 0 ± 0 | 7 ± 4 | 1,104 ± 117 |
| 23 | 1,685 ± 159 | 0 ± 0 | 1 ± 1 | 1 ± 1 | 0 ± 0 | 0 ± 0 | 3 ± 2 | 1,227 ± 128 |
| 27 | — | 0 ± 0 | 0 ± 0 | 0 ± 0 | 0 ± 0 | 0 ± 0 | 1 ± 0 | 1,363 ± 151 |

Notes:
^{a} Mean + SEM G1: Vehicle, G2: LC A032D/HC Benchmark (3 mg/kg), G3: LC A032D/HC Y052K
^{b}(3 mg/kg), G4: LC A032D/HC G056K (3 mg/kg), G5: LC A032D/HC T058D (3 mg/kg), G6: LC A032D/HC A106E (3 mg/kg), G7: LC A032D/HC S119E (3 mg/kg), G8: B12 Isotype Control Antibody (3 mg/kg).

One-way ANOVA was performed to compare the mean tumor volumes and RTVs among groups. A significant F-statistics was obtained and comparisons between groups were carried out with Games-Howell test. All data were analyzed using IBM® SPSS Statistics® software (version 17.0.). p<0.05 was considered to be statistically significant.

### Mortality, Morbidity, and Body Weight Gain or Loss

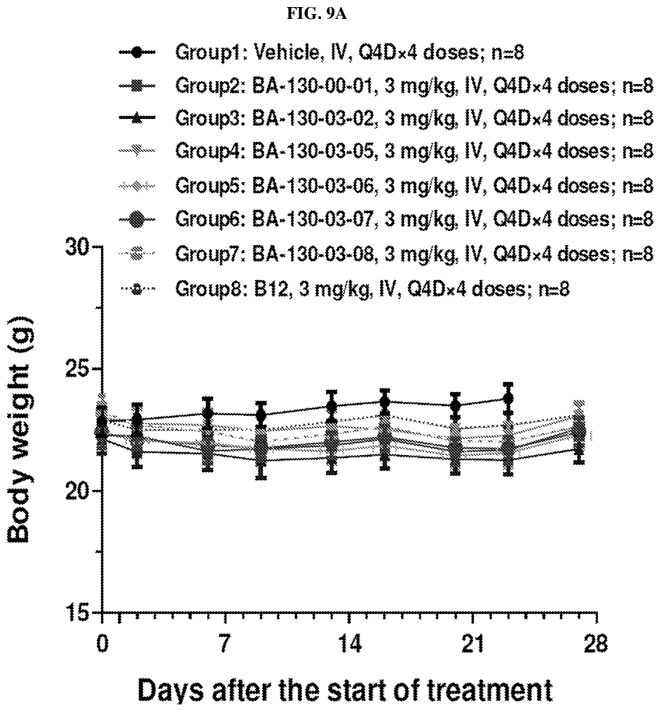

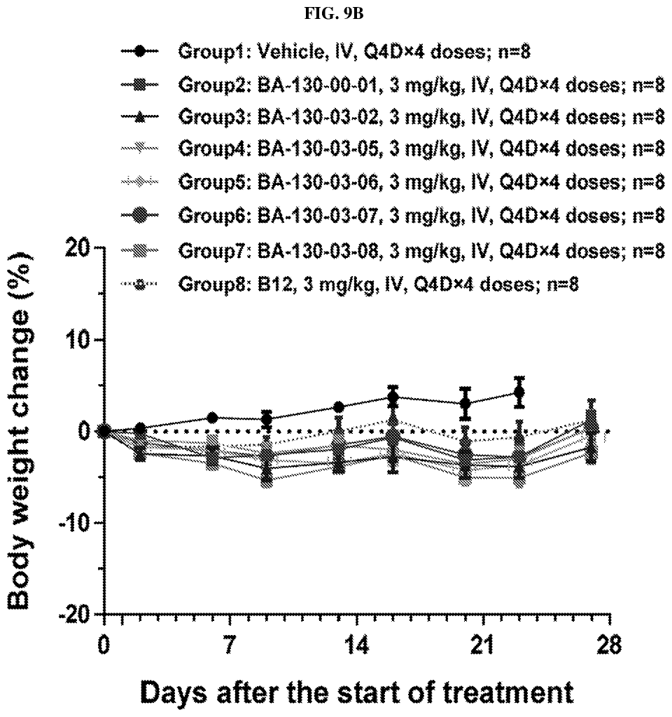

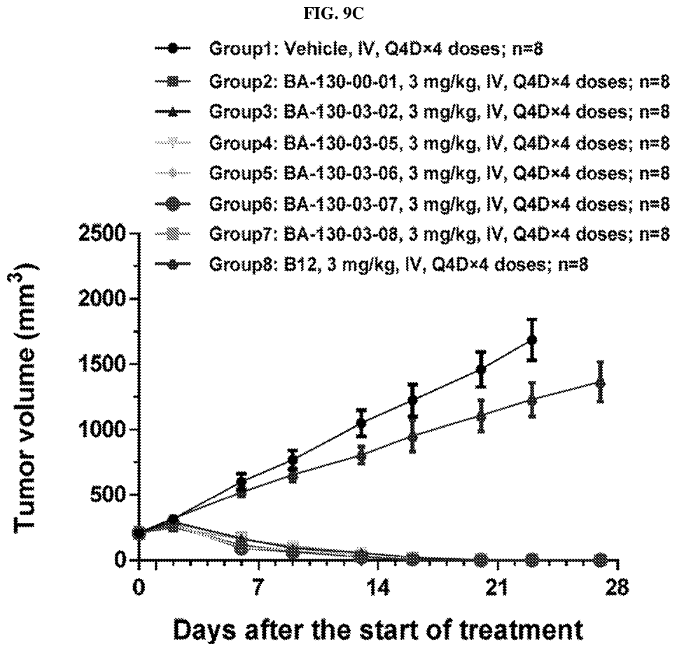

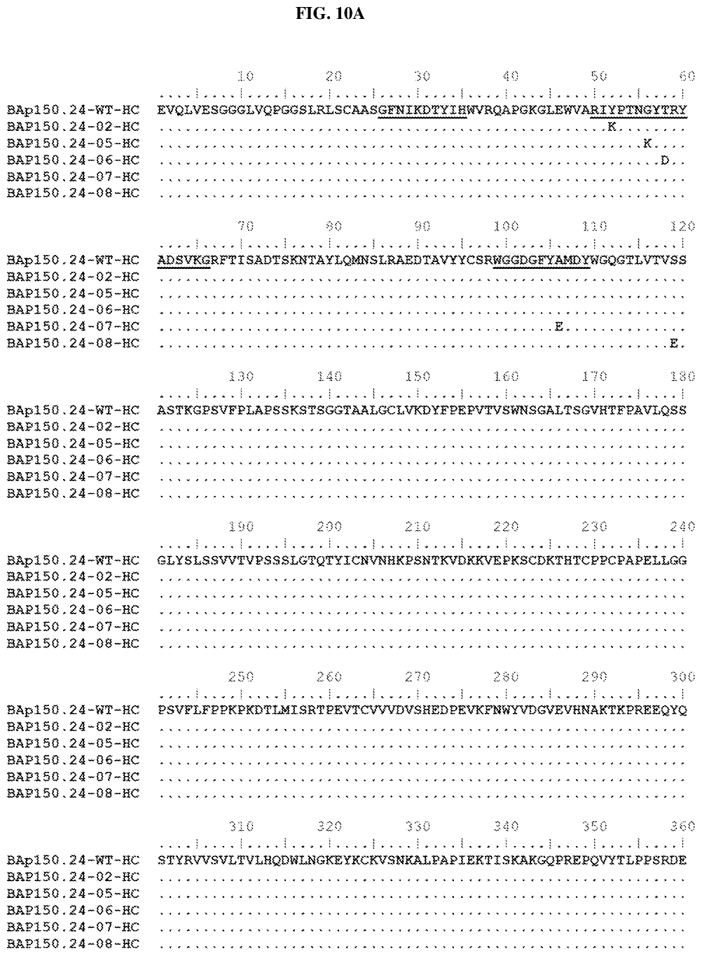

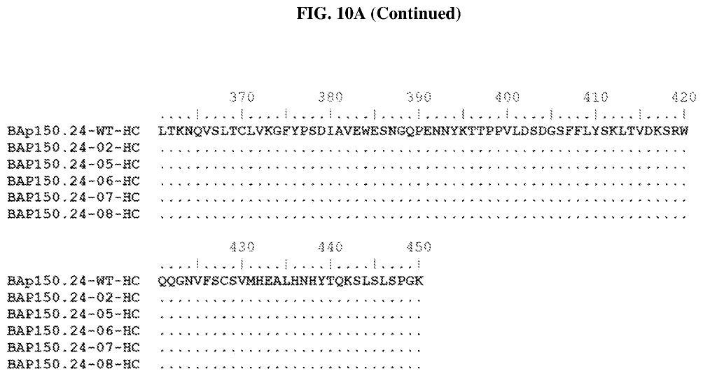

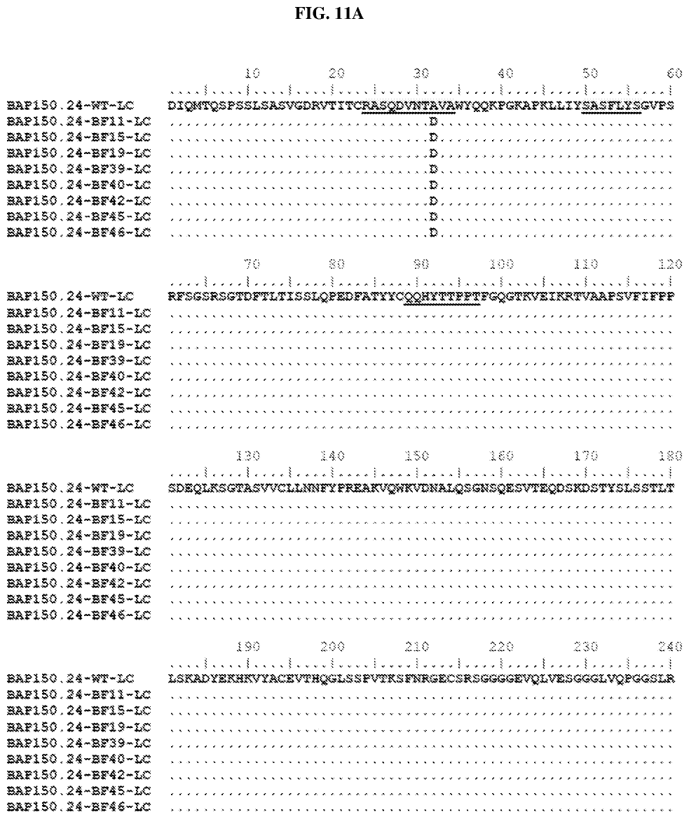

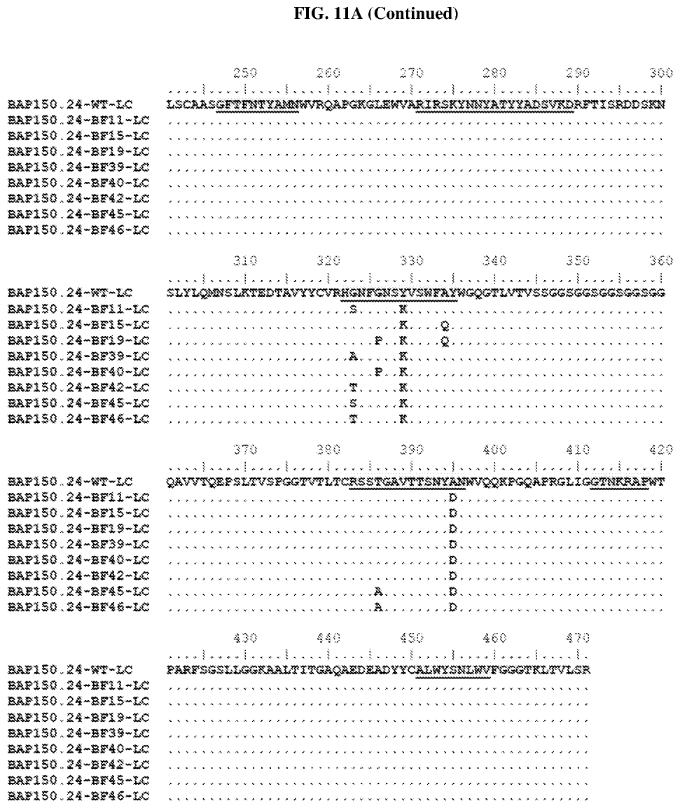

Animal body weight was monitored regularly as an indicator of toxicity. During this study, no group showed significant body weight loss (10% or above) See FIG. 9A. No death or morbidity was observed. Thus, no obvious toxicity was observed in association with the administration of antibodies to tumor-bearing BALB/c nude mice in the current dosing regimen.

The body weights and relative body weight changes of different groups are shown in FIG. 9A and FIG. 9B, respectively. Tumor growth inhibition is shown in Tables 5-6 below. The numbering of the substitutions referenced in Tables 5-6 is based on the BAP-130 benchmark antibody of FIG. 2.

TABLE 5
Tumor growth inhibition calculation (based on Day 23 data)

| | Tumor Size on | T/C ^{b} | TGI | | | |
|---|---|---|---|---|---|---|
| Treatment | day 23 (mm³) ^{a} | (%) | (%) | RTV ^{a} | p ^{c} | p ^{d} |
| Vehicle | 1,685 ± 159 | — | — | 8.08 ± 0.51 | | |
| LC A032D/HC Benchmark | 0 ± 0 | 0 | 114 | 0.00 ± 0.00 | <0.001 | <0.001 |
| 3 mg/kg | | | | | | |
| LC A032D/HC Y052K | 1 ± 1 | 0.03 | 114 | 0.00 ± 0.00 | <0.001 | <0.001 |
| 3 mg/kg | | | | | | |
| LC A032D/HC G056K | 1 ± 1 | 0.03 | 114 | 0.00 ± 0.00 | <0.001 | <0.001 |
| 3 mg/kg | | | | | | |
| LC A032D/HC T058D | 0 ± 0 | 0.12 | 114 | 0.00 ± 0.00 | <0.001 | <0.001 |
| 3 mg/kg | | | | | | |
| LC A032D/HC A106E | 0 ± 0 | 0 | 114 | 0.00 ± 0.00 | <0.001 | <0.001 |
| 3 mg/kg | | | | | | |
| LC A032D/HC S119E | 3 ± 2 | 0.2 | 114 | 0.01 ± 0.01 | <0.001 | <0.001 |
| 3 mg/kg | | | | | | |
| B12 (Isotype Control | 1,227 ± 128 | 73 | 31 | 6.10 ± 0.89 | 0.387 | 0.559 |
| Antibody), 3 mg/kg | | | | | | |

Note:
^{a} Mean + SEM.
^{b} Tumor Growth Inhibition is calculated by dividing the group average tumor volume of the treated group by the group average tumor volume of the vehicle control group (T/C).
^{c} p value calculated based on tumor size of Day 23.
^{d} p value calculated based on RTV of Day 23.

TABLE 6
Tumor growth inhibition calculation (based on Day 27 data)

| | Tumor Size on apep | T/C ^{b} | TGI | | | |
|---|---|---|---|---|---|---|
| Treatment | day 27 (mm³) ^{a} | (%) | (%) | RTV ^{a} | p ^{c} | p ^{d} |
| B12 (Isotype Control | 1363 ± 151 | — | — | 6.79 ± 1.04 | — | — |
| Antibody), 3 mg/kg | | | | | | |
| LC A032D/HC Benchmark | 0 ± 0 | 0 | 118 | 0.00 ± 0.00 | <0.001 | 0.003 |
| 3 mg/kg | | | | | | |
| LC AO32D/HC Y052K | 0 ± 0 | 0 | 118 | 0.00 ± 0.00 | <0.001 | 0.003 |
| 3 mg/kg | | | | | | |
| LC AO32D/HC G056K | 0 ± 0 | 0 | 118 | 0.00 ± 0.00 | <0.001 | 0.003 |
| 3 mg/kg | | | | | | |
| LC AO32D/HC T058D | 0 ± 0 | 0 | 118 | 0.00 ± 0.00 | <0.001 | 0.003 |
| 3 mg/kg | | | | | | |
| LC A032D/HC A106E | 0 ± 0 | 0 | 118 | 0.00 ± 0.00 | <0.001 | 0.003 |
| 3 mg/kg | | | | | | |
| LC AO32D/HCS119E | 1 ± 0 | 0 | 118 | 0.00 ± 0.00 | <0.001 | 0.003 |
| 3 mg/kg | | | | | | |

Note:
^{a} Mean + SEM.
^{b} Tumor Growth Inhibition is calculated by dividing the group average tumor volume of the treated group by the group average tumor volume of the vehicle control group (T/C).
^{c} p value calculated based on tumor size of Day 27.
^{d} p value calculated based on RTV of Day 27.

Tumor growth curves are shown in FIG. 9C.

The mean tumor size of the vehicle treated group reached 1,685 mm³ on Day 23 (RTV=8.08±0.51) after the start of treatment. All tested antibodies (BA-130-00-01, BA-130-03-02, BA-130-03-05, BA-130-03-06, BA-130-03-07, BA-130-03-08) at 3 mg/kg dose level exhibited dramatic anti-tumor activities leading to complete remission in most of treated mice within 16 to 27 days (T/C<1%, TGI>114%, p value<0.001, PG-D23, FIG. 9C). The differences in tumor volume between TA groups and isotype group are also significant (T/C<1%, TGI>118%, p value<0.001, PG-D27).

B12 (isotype control antibody) slightly delayed the tumor growth, but this result was not statistically significant when compared with the vehicle group (T/C=73%, TGI=31%, p value=0.387, PG-D23).

No severe body weight loss or death/morbidity event was observed during the entire study. Thus, no obvious toxicity was observed in association with the administration of the antibodies.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded for each animal in each group.

### Example 8—Multi-Specific Antibodies that Bind CD3 and HER2

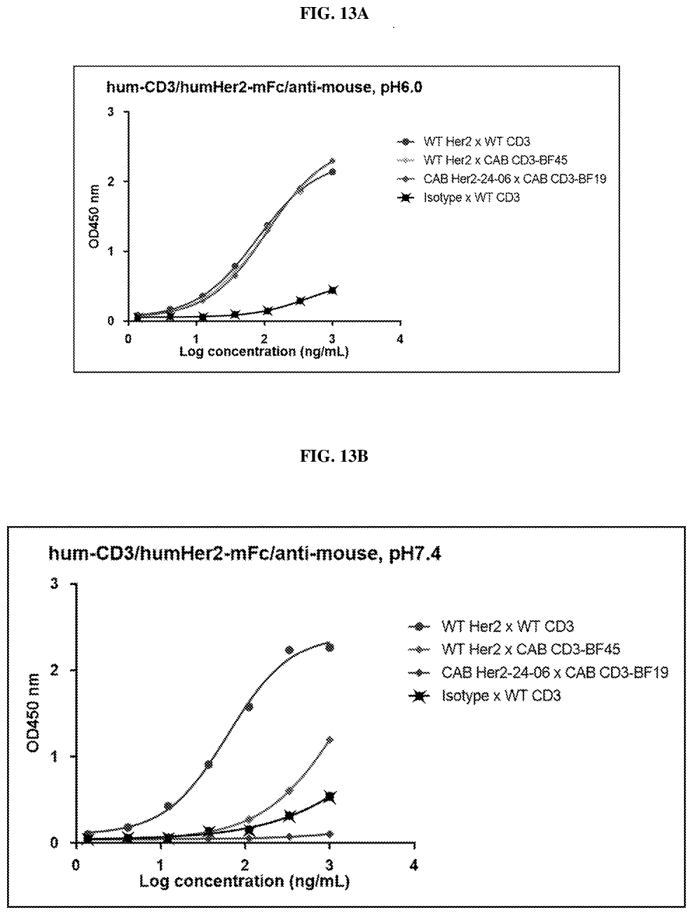

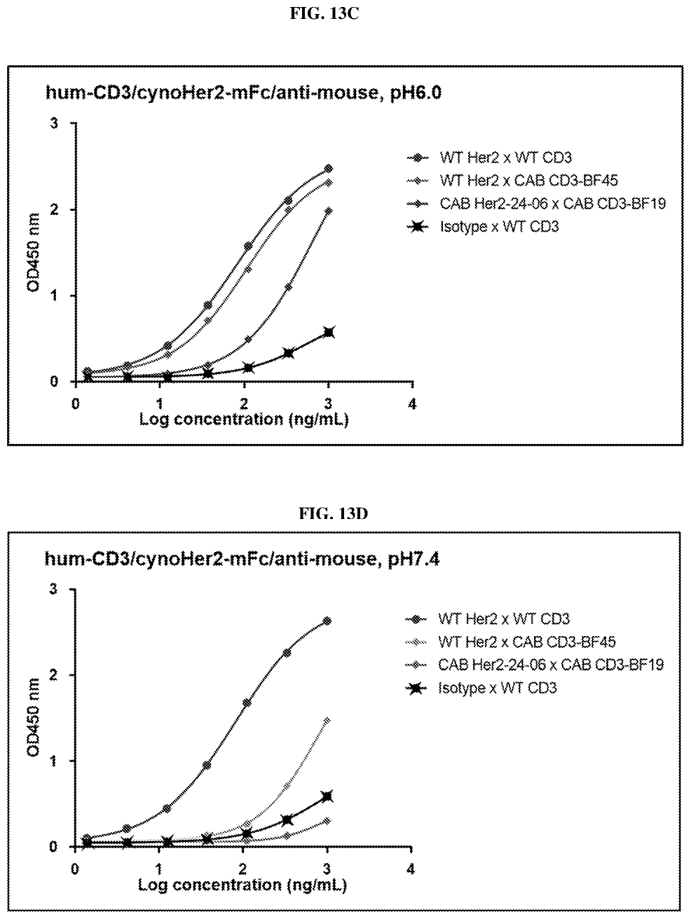

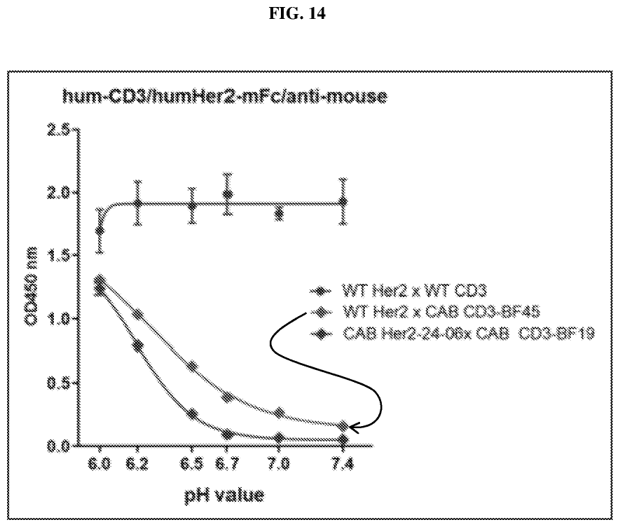

Multi-specific antibodies that bind to CD3 and HER2 were constructed. One multi-specific antibody used a non-conditionally active binding site (scFv antibody) to CD3 (WT-CD3) paired with a non-conditionally active binding site (IgG antibody) to HER2 (WT-HER2) to provide a butterfly configuration WT-HER2×WT-CD3 (FIGS. 12 and 13A-13D). Similarly, a second multi-specific antibody used a non-conditionally active binding site (IgG antibody) to HER2 (WT-HER2) paired with a conditionally active (scFv antibody) to CD3 (CAB CD3) to form a butterfly configuration WT-HER2×CAB-CD3 (FIGS. 12 and 13A-13D). A third multi-specific antibody used a conditionally active binding site (IgG antibody) to HER2 (CAB-HER2) paired with a conditionally active (scFv antibody) to CD3 (CAB-CD3) to form a butterfly configuration CAB-HER2×CAB-CD3 (FIGS. 12 and 13A-13D).

Bispecific antibodies were assayed for their affinity to CD3 and HER2, respectively at pH 6.0 and pH 7.4 using ELISA assay (FIG. 13A-13D). These three multi-specific antibodies were compared to isotype×WT CD3. The ELISA assay of this application used the following protocol:
1. One day before ELISA, a 96 well plate was coated with 100 μl of 0.5 μg/ml recombinant CD3 or HER2 overnight in ELISA coating buffer at 4° C.
2. Dilute samples in ELISA assay buffer.
3. Flicked off buffer from the plate coated with antigen, blot dry on paper towels.
4. Block plate with 200 μl ELISA assay buffer at room temperature for 1 hour.
5. Add 100 μl of diluted samples to each well.
6. Incubate the plate at room temperature for 1 hour.
7. Prepare the secondary antibody in screening buffers according to the layout of the plate.
8. Flicked off buffer from the plate, blot dry on paper towels.
9. Wash the plate for a total of 3 times with ELISA wash buffer.
10. Add 100 μl of 1 ug/ml of human HER2 fused to mouse IgG Fc in ELISA assay buffer to the wells.
11. Incubate the plate at room temperature for 1 hour.
12. Flicked off buffer from the plate, blot dry on paper towels.
13. Wash the plate for a total of 3 times with ELISA wash buffer.
14. Flick off buffers from plate, blot dry on paper towels.
15. Add 100 μl of 1:2500 diluted anti-mouse HRP secondary antibody secondary antibodies to the wells.
16. Incubate the plate at room temperature for 1 hour.
17. Flicked off buffer from the plate, blot dry on paper towels.
18. Wash the plate for a total of 3 times with ELISA wash buffer.
19. Flick off buffers from plate, blot dry on paper towels.
20. Add 50 μl of 3,3′, 5,5′-Tetramethylbenzidine (TMB) substrate according to the plate layout.
21. Stop development with 50 μl 1N HCl.
22. Read at OD450 nm using a plate reader.

### WT/CAB HER2×CAB CD3 Butterfly Bispecific pH Sandwich Elisa Assays

The binding activity of WT HER2×WT CD3, WT HER2×CAB CD3-BF45, and CAB HER2-24-06×CAB CD3-BF19 bispecific antibodies at various pH values, determined by a pH sandwich ELISA assay, are shown in Tables 7 and 8.

TABLE 7
Human CD3 Capture, Human-HER2 mFc Detection

| | EC50 (ng/ml) | | |
|---|---|---|---|
| Clone | pH 6.0 | pH 7.4 | pH 7.4/6.0 |
| WT HER2 × WT CD3 | 77.67 | 62.08 | 0.80 |
| WT HER2 × CAB CD3-BF45 | 109.8 | 1623* | 14.78 |
| CAB HER2-24-06 × CAB CD3-BF19 | 112.4 | 1928* | 17.15 |

*A complete saturation curve was not reached, ED50 value was estimated.

TABLE 8
Human CD3 Capture, cyno-HER2 mFc Detection

| | EC50 (ng/ml) | | |
|---|---|---|---|
| Clone | pH 6.0 | pH 7.4 | pH 7.4/6.0 |
| WT HER2 × WT CD3 | 82.98 | 81.24 | 0.98 |
| WT HER2 × CAB CD3-BF45 | 106.9 | 807* | 7.55 |
| CAB HER2-24-06 × CAB CD3-BF19 | 653.9* | 801.9* | 1.23 |

*A complete saturation curve was not reached, ED50 value was estimated.

### WT/CAB HER2×CAB CD3 Butterfly Bispecific pH Range Elisa Assay

Binding activities of WT HER2×WT CD3, WT HER2×CAB CD3-BF45, and CAB HER2-24-06×CAB CD3-BF-19 bispecific antibodies at various pH values, determined by a pH range ELISA, are shown in Table 9.

TABLE 9
Human CD3 Capture, Human-HER2 mFc/anti-mouse Detection

| Clone | pH Inflection Point |
|---|---|
| WT HER2 × WT CD3 | N/C |
| WT HER2 × CAB CD3-BF45 | 6.287 |
| CAB HER2-24-06 × CAB CD3-BF-19 | 6.193 |

N/C: not calculated

### Example 9—Multi-Specific Antibodies that Bind to CD3 and HER2

In this example, multi-specific antibodies that bind to CD3 and HER2 were constructed, including the heavy and light chains as shown below in Table 10. The multi-specific antibodies were made as described in Example 8 and named as follows:

TABLE 10

| Clone Label | Clone | light chain | heavy chain |
|---|---|---|---|
| CAB HER2-24-02 × CAB CD3-BF11 | BA-150-24-02-BF11 | BAP150.24-BF11-LC | BAP150.24-02-HC |
| | | SEQ ID NO: 41 | SEQ ID NO: 35 |
| CAB HER2-24-02 × CAB CD3-BF15 | BA-150-24-02-BF15 | BAP150.24-BF15-LC | BAP150.24-02-HC |
| | | SEQ ID NO: 42 | SEQ ID NO: 35 |
| CAB HER2-24-02 × CAB CD3-BF19 | BA-150-24-02-BF19 | BAP150.24-BF19-LC | BAP150.24-02-HC |
| | | SEQ ID NO: 43 | SEQ ID NO: 35 |
| CAB HER2-24-02 × CAB CD3-BF39 | BA-150-24-02-BF39 | BAP150.24-BF39-LC | BAP150.24-02-HC |
| | | SEQ ID NO: 44 | SEQ ID NO: 35 |
| CAB HER2-24-02 × CAB CD3-BF40 | BA-150-24-02-BF40 | BAP150.24-BF40-LC | BAP150.24-02-HC |
| | | SEQ ID NO: 45 | SEQ ID NO: 35 |
| CAB HER2-24-02 × CAB CD3-BF42 | BA-150-24-02-BF42 | BAP150.24-BF42-LC | BAP150.24-02-HC |
| | | SEQ ID NO: 46 | SEQ ID NO: 35 |
| CAB HER2-24-02 × CAB CD3-BF45 | BA-150-24-02-BF45 | BAP150.24-BF45-LC | BAP150.24-02-HC |
| | | SEQ ID NO: 47 | SEQ ID NO: 35 |
| CAB HER2-24-02 × CAB CD3-BF46 | BA-150-24-02-BF46 | BAP150.24-BF46-LC | BAP150.24-02-HC |
| | | SEQ ID NO: 48 | SEQ ID NO: 35 |
| CAB HER2-24-05 × CAB CD3-BF11 | BA-150-24-05-BF11 | BAP150.24-BF11-LC | BAP150.24-05-HC |
| | | SEQ ID NO: 41 | SEQ ID NO: 36 |
| CAB HER2-24-05 × CAB CD3-BF15 | BA-150-24-05-BF15 | BAP150.24-BF15-LC | BAP150.24-05-HC |
| | | SEQ ID NO: 42 | SEQ ID NO: 36 |
| CAB HER2-24-05 × CAB CD3-BF19 | BA-150-24-05-BF19 | BAP150.24-BF19-LC | BAP150.24-05-HC |
| | | SEQ ID NO: 43 | SEQ ID NO: 36 |
| CAB HER2-24-05 × CAB CD3-BF40 | BA-150-24-05-BF40 | BAP150.24-BF40-LC | BAP150.24-05-HC |
| | | SEQ ID NO: 45 | SEQ ID NO: 36 |
| CAB HER2-24-05 × CAB CD3-BF42 | BA-150-24-05-BF42 | BAP150.24-BF42-LC | BAP150.24-05-HC |
| | | SEQ ID NO: 46 | SEQ ID NO: 36 |
| CAB HER2-24-05 × CAB CD3-BF45 | BA-150-24-05-BF45 | BAP150.24-BF45-LC | BAP150.24-05-HC |
| | | SEQ ID NO: 47 | SEQ ID NO: 36 |
| CAB HER2-24-05 × CAB CD3-BF46 | BA-150-24-05-BF46 | BAP150.24-BF46-LC | BAP150.24-05-HC |
| | | SEQ ID NO: 48 | SEQ ID NO: 36 |
| CAB HER2-24-06 × CAB CD3-BF11 | BA-150-24-06-BF11 | BAP150.24-BF11-LC | BAP150.24-06-HC |
| | | SEQ ID NO: 41 | SEQ ID NO: 37 |
| CAB HER2-24-06 × CAB CD3-BF15 | BA-150-24-06-BF15 | BAP150.24-BF15-LC | BAP150.24-06-HC |
| | | SEQ ID NO: 42 | SEQ ID NO: 37 |
| CAB HER2-24-06 × CAB CD3-BF19 | BA-150-24-06-BF19 | BAP150.24-BF19-LC | BAP150.24-06-HC |
| | | SEQ ID NO: 43 | SEQ ID NO: 37 |
| CAB HER2-24-06 × CAB CD3-BF39 | BA-150-24-06-BF39 | BAP150.24-BF39-LC | BAP150.24-06-HC |
| | | SEQ ID NO: 44 | SEQ ID NO: 37 |
| CAB HER2-24-06 × CAB CD3-BF40 | BA-150-24-06-BF40 | BAP150.24-BF40-LC | BAP150.24-06-HC |
| | | SEQ ID NO: 45 | SEQ ID NO: 37 |
| CAB HER2-24-06 × CAB CD3-BF42 | BA-150-24-06-BF42 | BAP150.24-BF42-LC | BAP150.24-06-HC |
| | | SEQ ID NO: 46 | SEQ ID NO: 37 |
| CAB HER2-24-06 × CAB CD3-BF45 | BA-150-24-06-BF45 | BAP150.24-BF45-LC | BAP150.24-06-HC |
| | | SEQ ID NO: 47 | SEQ ID NO: 37 |
| CAB HER2-24-06 × CAB CD3-BF46 | BA-150-24-06-BF46 | BAP150.24-BF46-LC | BAP150.24-06-HC |
| | | SEQ ID NO: 48 | SEQ ID NO: 37 |
| CAB HER2-24-07 × CAB CD3-BF11 | BA-150-24-07-BF11 | BAP150.24-BF11-LC | BAP150.24-07-HC |
| | | SEQ ID NO: 41 | SEQ ID NO: 38 |
| CAB HER2-24-07 × CAB CD3-BF15 | BA-150-24-07-BF15 | BAP150.24-BF15-LC | BAP150.24-07-HC |
| | | SEQ ID NO: 42 | SEQ ID NO: 38 |
| CAB HER2-24-07 × CAB CD3-BF19 | BA-150-24-07-BF19 | BAP150.24-BF19-LC | BAP150.24-07-HC |
| | | SEQ ID NO: 43 | SEQ ID NO: 38 |
| CAB HER2-24-07 × CAB CD3-BF39 | BA-150-24-07-BF39 | BAP150.24-BF39-LC | BAP150.24-07-HC |
| | | SEQ ID NO: 44 | SEQ ID NO: 38 |
| CAB HER2-24-07 × CAB CD3-BF40 | BA-150-24-07-BF40 | BAP150.24-BF40-LC | BAP150.24-07-HC |
| | | SEQ ID NO: 45 | SEQ ID NO: 38 |
| CAB HER2-24-07 × CAB CD3-BF42 | BA-150-24-07-BF42 | BAP150.24-BF42-LC | BAP150.24-07-HC |
| | | SEQ ID NO: 46 | SEQ ID NO: 38 |
| CAB HER2-24-07 × CAB CD3-BF45 | BA-150-24-07-BF45 | BAP150.24-BF45-LC | BAP150.24-07-HC |
| | | SEQ ID NO: 47 | SEQ ID NO: 38 |
| CAB HER2-24-07 × CAB CD3-BF46 | BA-150-24-07-BF46 | BAP150.24-BF46-LC | BAP150.24-07-HC |
| | | SEQ ID NO: 48 | SEQ ID NO: 38 |
| CAB HER2-24-08 × CAB CD3-BF11 | BA-150-24-08-BF11 | BAP150.24-BF11-LC | BAP150.24-08-HC |
| | | SEQ ID NO: 41 | SEQ ID NO: 39 |
| CAB HER2-24-08 × CAB CD3-BF15 | BA-150-24-08-BF15 | BAP150.24-BF15-LC | BAP150.24-08-HC |
| | | SEQ ID NO: 42 | SEQ ID NO: 39 |
| CAB HER2-24-08 × CAB CD3-BF19 | BA-150-24-08-BF19 | BAP150.24-BF19-LC | BAP150.24-08-HC |
| | | SEQ ID NO: 43 | SEQ ID NO: 39 |
| CAB HER2-24-08 × CAB CD3-BF39 | BA-150-24-08-BF39 | BAP150.24-BF39-LC | BAP150.24-08-HC |
| | | SEQ ID NO: 44 | SEQ ID NO: 39 |
| CAB HER2-24-08 × CAB CD3-BF42 | BA-150-24-08-BF42 | BAP150.24-BF42-LC | BAP150.24-08-HC |
| | | SEQ ID NO: 46 | SEQ ID NO: 39 |
| CAB HER2-24-08 × CAB CD3-BF45 | BA-150-24-08-BF45 | BAP150.24-BF45-LC | BAP150.24-08-HC |
| | | SEQ ID NO: 47 | SEQ ID NO: 39 |
| CAB HER2-24-08 × CAB CD3-BF46 | BA-150-24-08-BF46 | BAP150.24-BF46-LC | BAP150.24-08-HC |
| | | SEQ ID NO: 48 | SEQ ID NO: 39 |

### Example 10—Surface Plasmon Resonance (SPR) Assays

SPR Analysis. Binding kinetics of anti-CTLA4 antibodies were measured by surface plasmon resonance on a SPR2/4 instrument (Sierra Sensors, Hamburg, Germany) and flat amine sensor chips. The SPR sensor contains four flow cells (FC1-FC4), each of which can be addressed individually or in groups. huHER2-His was immobilized in FC2, cynoHER2-His in FC3, and huCD in FC4. No protein was immobilized in FC1 (control surface). All injections were done at a flow rate of 25 μL/min and 25° C. The sensor surface was activated with 1-ethyl-3-(3-dimethylaminopropyl)-carbodiimide (EDC) and N-hydroxysuccinimide (NHS) (200 mM/50 mM) for 480 seconds. Human HER2-His (0.5 μg/mL in 10 mM NaAc, pH5.5) was injected for 480s and the surface was inactivated by injecting 1M ethanolamine-HCl for 480s. cynoHER2-His and huCD3 were immobilized using the same conditions as described for huHER2-His. The control surface was activated and deactivated using the same conditions, but without injecting protein. PBST buffer (PBS pH7.4 with 0.05% TWEEN20) was used as running buffer for the surface preparation. The running solution was switched to PBST with 30 mM sodium bicarbonate with the pH adjusted as indicated in the figures before the analyte injections. The instrument was equilibrated with the running solution for one hour before the first analyte injection. 100 μL analyte diluted in the corresponding running solution (25 nM, 10 nM, 5 nM, 2.5 nM, 1.25 nM, 0.625 nM, and 0.0 nM) was injected overflow cells 1 to 4. Off-rate was measured for 360s. The chip surface was regenerated after each cycle of interaction analysis by injecting 6 μL of 10 mM glycine (pH 2.0). Flow cell 1 without immobilized protein was used as control surface for reference subtraction. In addition, data with buffer only as analyte (0 nM analyte) was subtracted from each run. Double subtracted data was fitted with the provided analysis software Analyzer R2 (Sierra Sensors) using a 1:1 binding model. A molecular weight of 200 kDa was used to calculate the molar concentrations of the analytes.

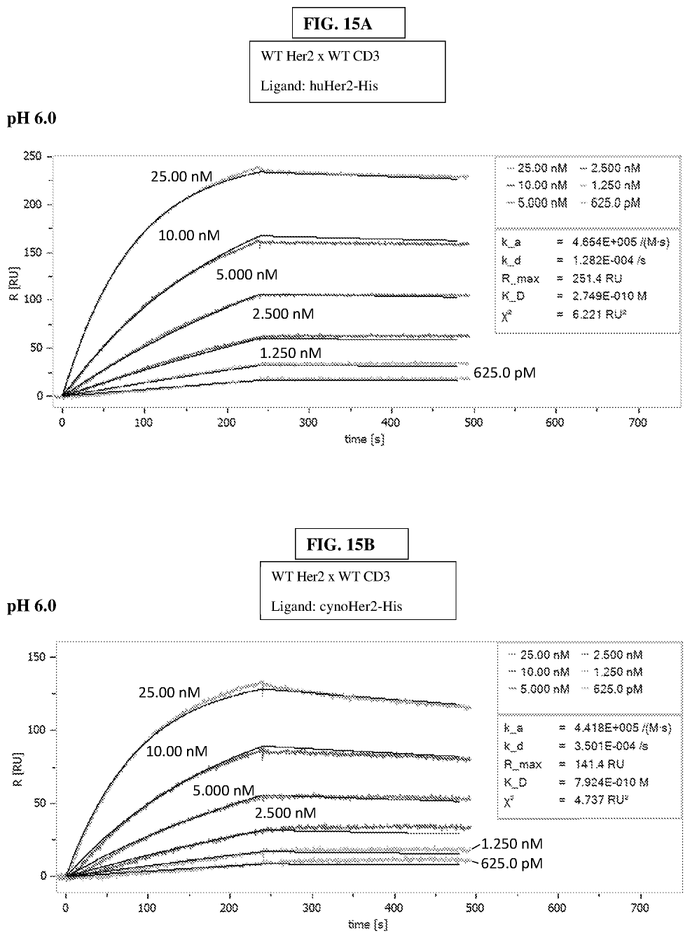

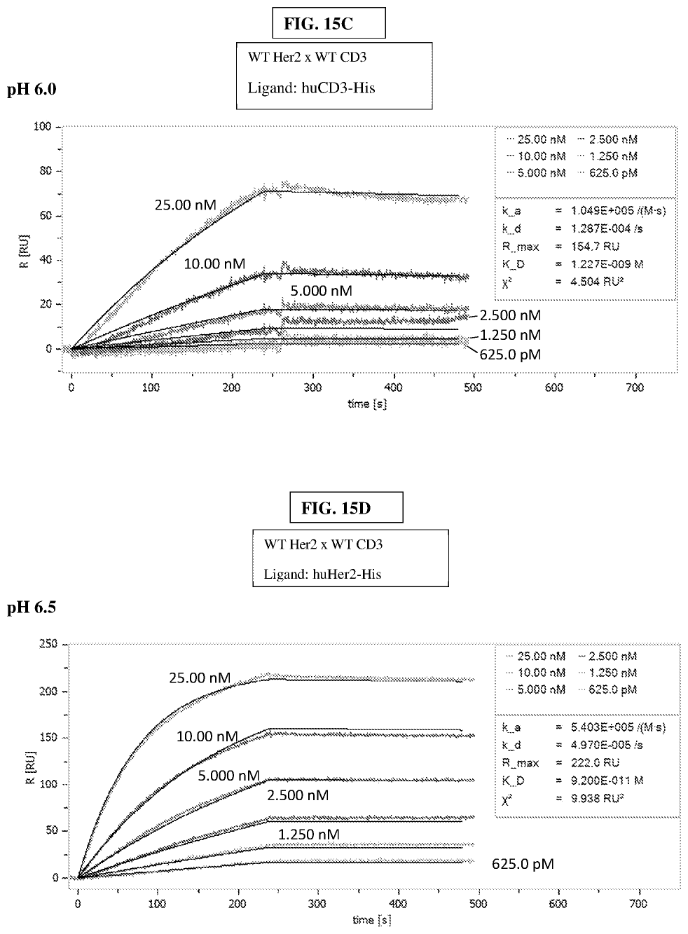

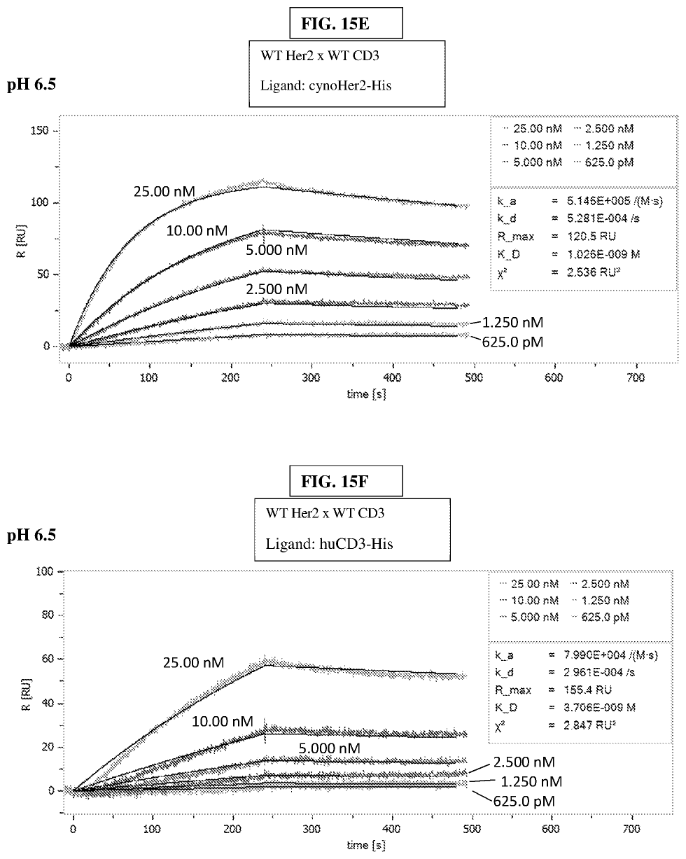

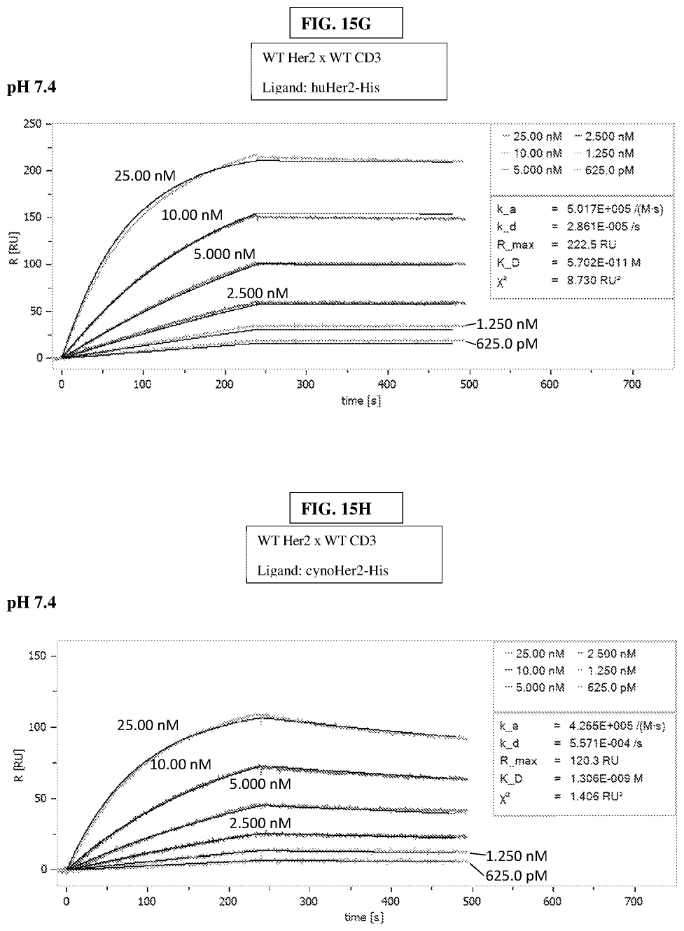

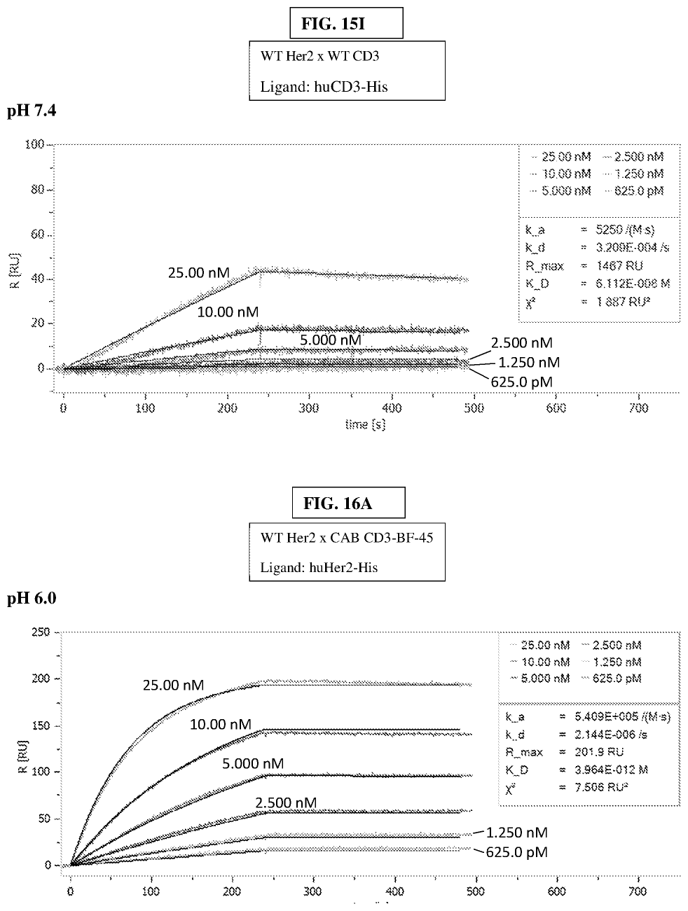

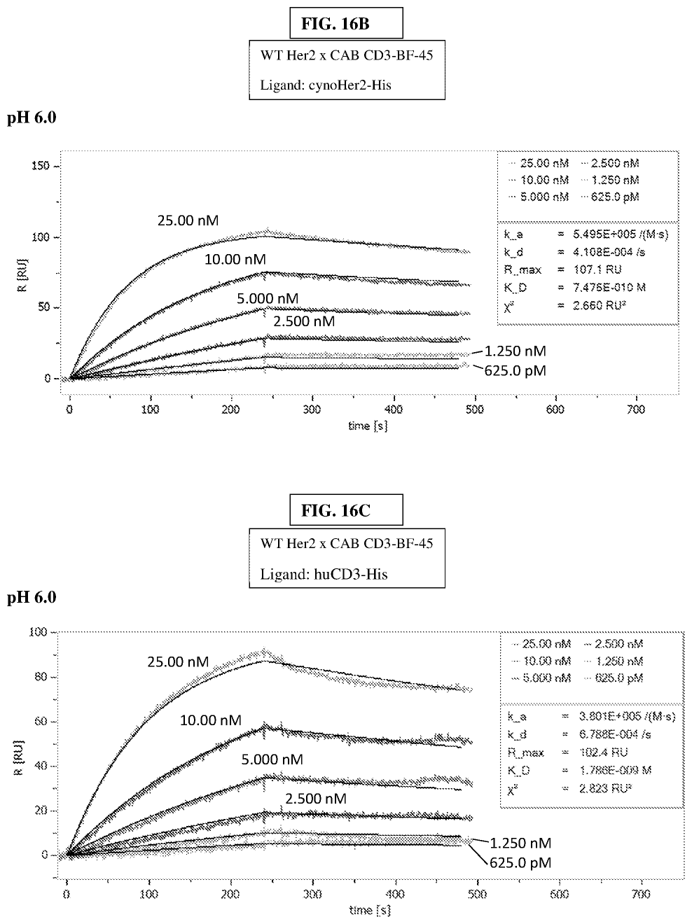

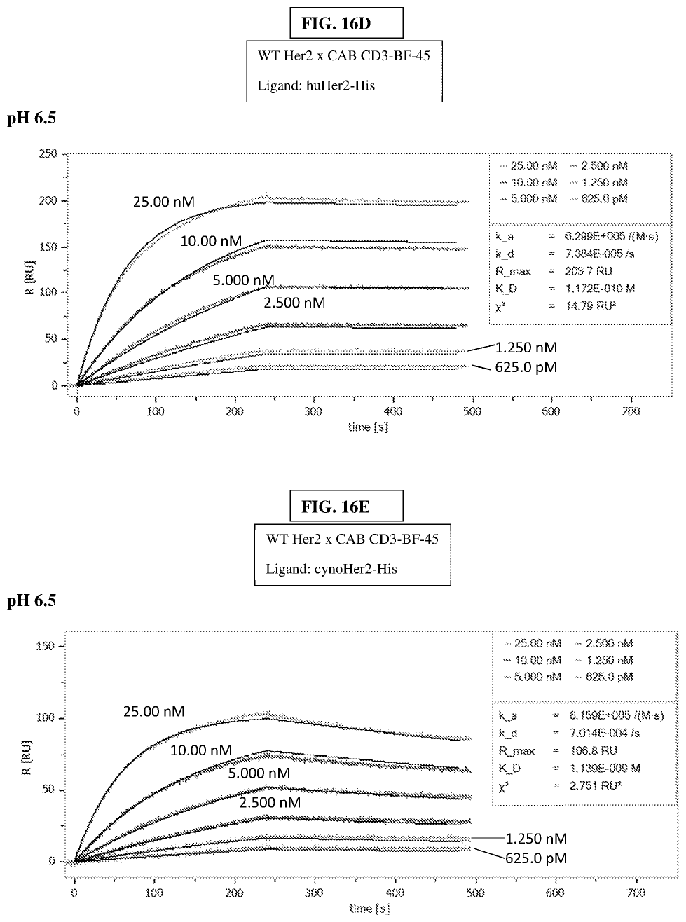

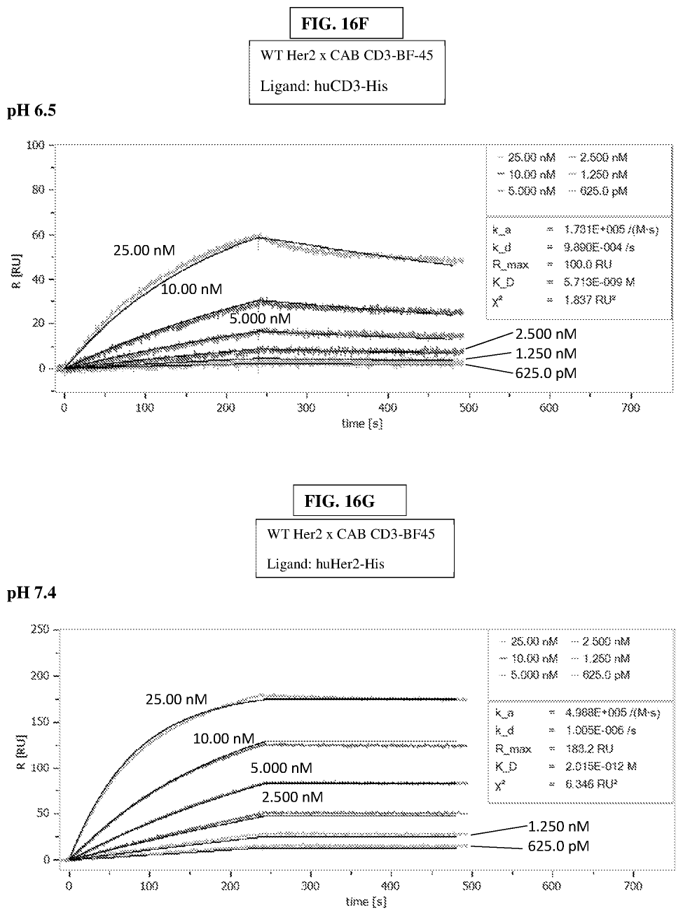

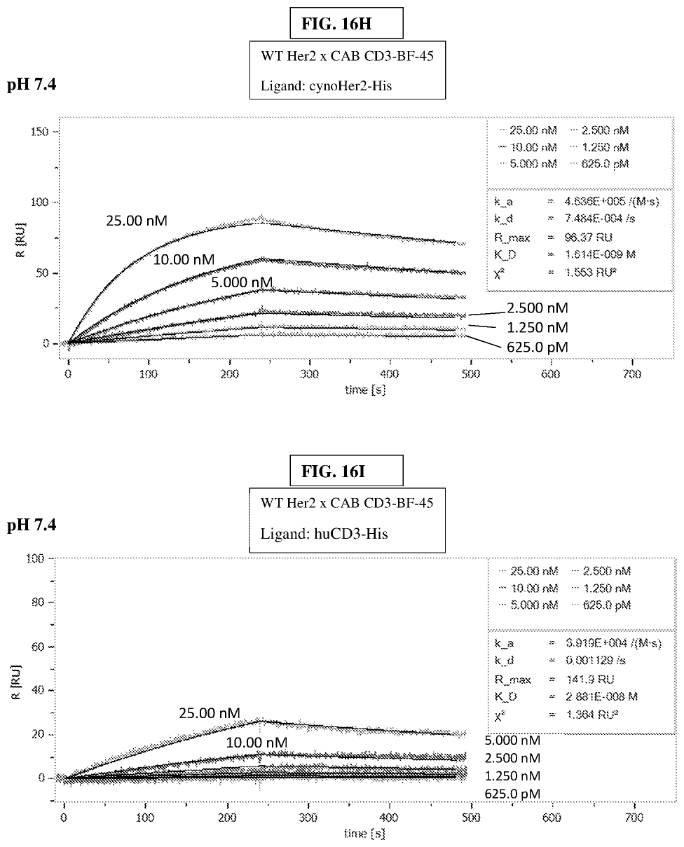

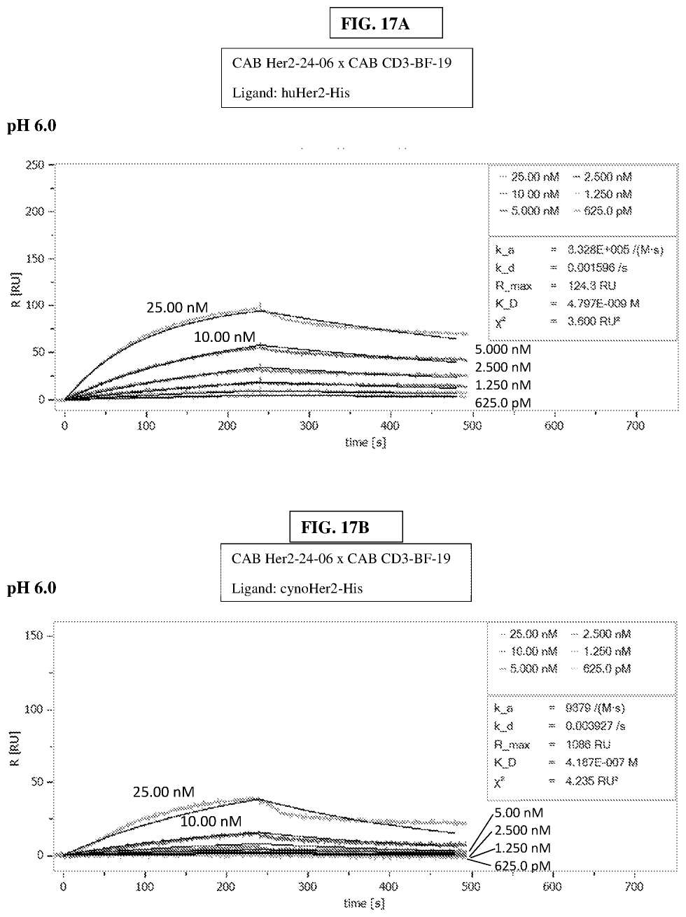

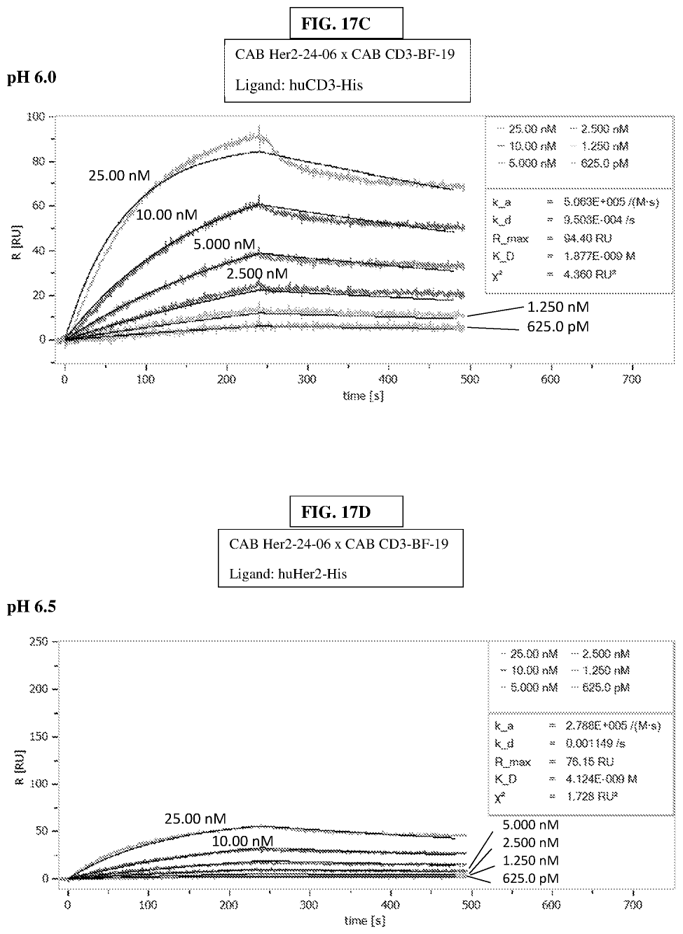

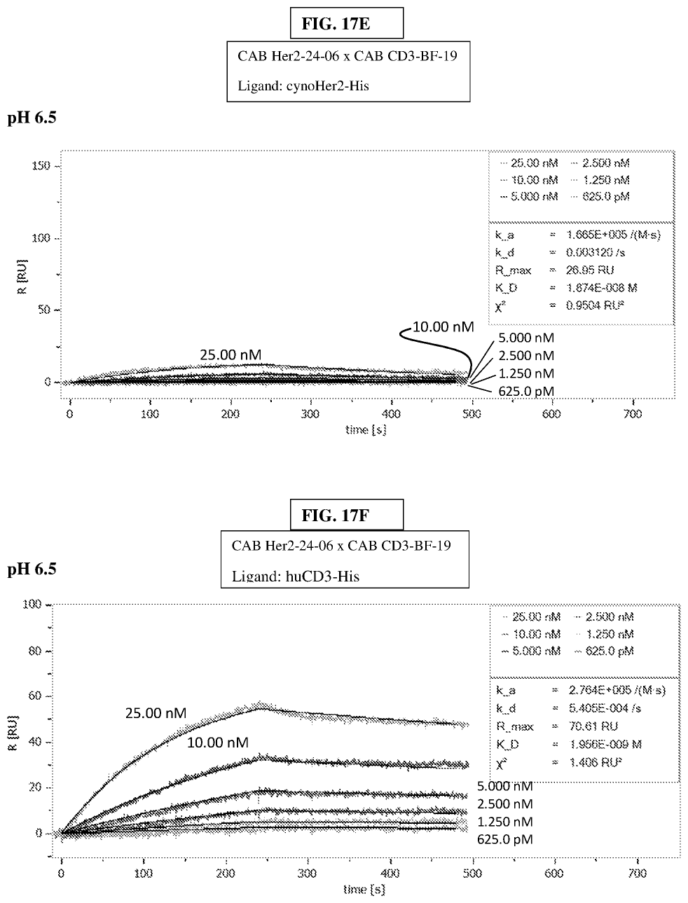

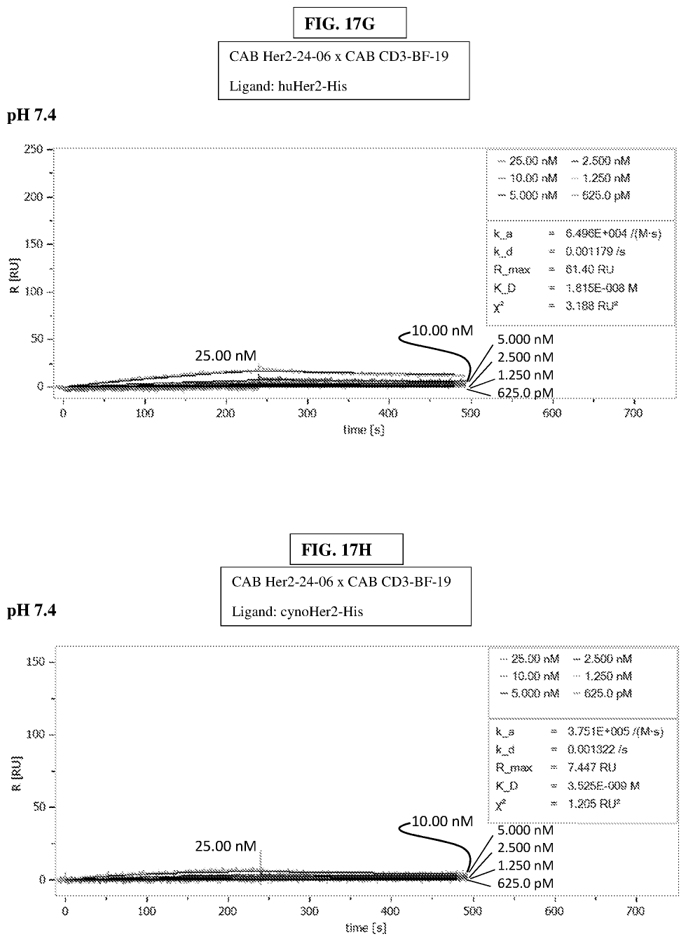

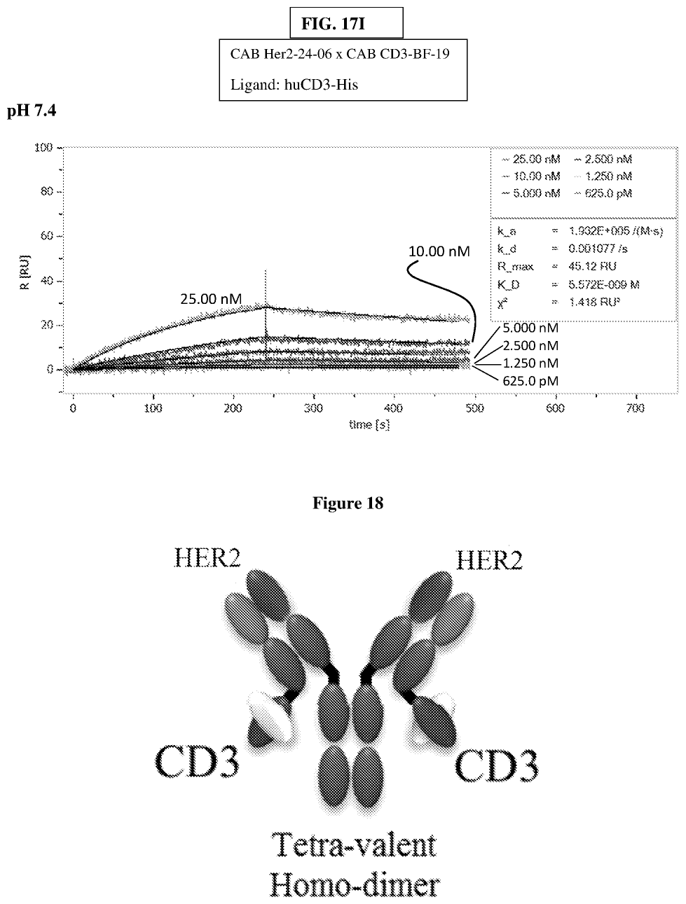

The dissociation constant (K_{d}) was measured using SPR binding analysis for WT HER2×WT CD3, WT HER2×CAB CD3-BF45, and CAB HER2-24-06×CAB CD3-BF19 with ligands huHER2-His, cyno-HER2-His, and huCD3-His at pH 6.0, pH 6.5, and pH 7.4. The results are shown in Table 11 below, and FIGS. 15A-17I.

TABLE 11
WT/CAB Her x CAB CD3 Butterfly Bispecifics SPR Analysis

| | | pH 6.0 | | | pH 6.5 | | | pH 7.4 | | |
|---|---|---|---|---|---|---|---|---|---|---|
| | | Ka [M · s] | Kd[s⁻¹] | KD[M] | Ka [M · s] | Kd[s⁻¹] | KD[M] | Ka [M · s] | Kd[s⁻¹] | KD[M] |
| humHer2 | WT Her2 × WT CD3 | 4.66E+05 | 1.28E−04 | 2.75E−10 | 5.40E+05 | 4.978−05 | 9.20E−11 | 5.02E+05 | 2.86E−05 | 5.70E−11 |
| humHer2 | WT Her2 × CAB CD3-BF45 | 5.41E+05 | 2.14E−06 | 3.56E−12 | 5.30E+05 | 7.388−05 | 1.17E−10 | 4.76E+05 | 1.32E−05 | 2.77E−11 |
| humHer2 | CAB Her2 × CAB CD3-BF19 | 3.33E+05 | 1.60E−03 | 4.80E−09 | 2.79E+05 | 1.158−03 | 4.12E−09 | 6.50E+04 | 1.18E−03 | 1.82E−08 |
| | | pH 6.0 | | | pH 6.5 | | | pH 7.4 | | |
| | | Ka [M · s] | Kd[s⁻¹] | KD[M] | Ka [M · s] | Kd[s⁻¹] | KD[M] | Ka [M · s] | Kd[s⁻¹] | KD[M] |
| cynoHer2 | WT Her × WT CD3 | 4.42E+05 | 3.S0E−04 | 7.92E−10 | 5.35E+05 | 5.28E−04 | 1.03E−09 | 4.27E+05 | 5.57E−04 | 1.31E−09 |
| cynoHer2 | WT Her2 × CAB CD3-BF45 | 5.50E+05 | 4.11E−04 | 7.48E−10 | 6.36E+05 | 7.01E−04 | 1.14E−09 | 4.47E+05 | 8.18E−04 | 1.83E−09 |
| cynoHer2 | CAB Her2 × CAB CD3-BF19 | 9.38E+03 | 3.93E−03 | 4.19E−07 | 1.67E+05 | 3.12E−02 | 1.87E−08 | 3.75E+05 | 1.32E−03 | 3.53E−09 |
| | | pH 6.0 | | | pH 6.5 | | | pH 7.4 | | |
| | | Ka [M · s] | Kd[s⁻¹] | KD[M] | Ka [M · s] | Kd[s⁻¹] | KD[M] | Ka [M · s] | Kd[s⁻¹] | KD[M] |
| humCD3 | WT Her2 × WT CD3 | 1.05E+05 | 1.29E−04 | 1.23E−09 | 7.99E+04 | 2.96E−04 | 3.71E−09 | 5.25E+03 | 3.21E−04 | 6.11E−18 |
| humCD3 | WT Her2 × CAB CD3-BF45 | 3.80E+05 | 6.79E−04 | 1.79E−09 | 1.73E+05 | 9.89E−04 | 5.71E−09 | 6 56E+04 | 1.12E−03 | 1.71E−08 |
| kumCD3 | CAB Her2 × CAS CD3-BF19 | 5.06E+05 | 9.50E−04 | 1.88E−09 | 2.76E+05 | 5.41E−04 | 1.96E−09 | 1.93E+05 | 1.08E−03 | 5.57E−09 |

very low signal

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

All documents mentioned herein are hereby incorporated by reference in their entirety or alternatively to provide the disclosure for which they were specifically relied upon. The applicant(s) do not intend to dedicate any disclosed embodiments to the public, and to the extent any disclosed modifications or alterations may not literally fall within the scope of the claims, they are considered to be part hereof under the doctrine of equivalents.

1. An antibody or antibody fragment that specifically binds to HER2 protein, said antibody or antibody fragment comprising a heavy chain variable region including three complementarity determining regions, said regions having sequences H1, H2, and H3, wherein:
the H1 sequence is
(SEQ ID NO: 1)
GFX₁IKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;
wherein X₁ is N or W, X₂ is R or K, X₃ is Y or K or D, X₄ is N or A, X₅ is G or K, X₆ is T or D, X₇ is R or E and X₈ is A or E; and
a light chain variable region including three complementarity determining regions having sequences L1, L2, and L3, wherein:
the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,
wherein X₉ is A or D and X₁₀ is H or D or E;
provided that when X₁-X₈ are N, R, Y, N, G, T, R and A, respectively, X₉ is not A and X₁₀ is not H.

2. An antibody or antibody fragment that specifically binds to HER2 protein and CD3 protein, said antibody or antibody fragment comprising a heavy chain variable region including three anti-HER2 complementarity determining regions, said regions having sequences H1, H2, and H3, wherein:
the H1 sequence is
(SEQ ID NO: 1)
GFXiIKDTYIH;
the H2 sequence is
(SEQ ID NO: 2)
X₂IX₃PTX₄X₅YX₆X₇YADSVKG;
and
the H3 sequence is
(SEQ ID NO: 3)
WGGDGFYX₈MDY;
wherein X₁ is N or W, X₂ is R or K, X₃ is Y or K or D, X₄ is N or A, X₅ is G or K, X₆ is T or D, X₇ is R or E and X₈ is A or E; and
a light chain variable region including three anti-HER2 complementarity determining regions having sequences L1, L2, and L3 wherein:
the L1 sequence is
(SEQ ID NO: 4)
RASQDVNTX₉VA;
the L2 sequence is
(SEQ ID NO: 5)
SASFLYS;
and
the L3 sequence is
(SEQ ID NO: 6)
QQX₁₀YTTPPT,
wherein X₉ is A or D and X₁₀ is H or D or E; provided that when X₁-X₈ are N, R, Y, N, G, T, R and A, respectively, X₉ is not A and X₁₀ is not H; and
six anti-CD3 complementarity determining regions L4, L5, L6, L7, L8, and L9 wherein:
the L4 sequence is
(SEQ ID NO: 54)
GFTFNTYAMN,
the L5 sequence is
(SEQ ID NO: 55)
RIRSKYNNYATYYADSVKD,
the L6 sequence is
(SEQ ID NO: 70)
HX₁₁NFX₁₂NSKVSWFX₁₃Y,
the L7 sequence is
(SEQ ID NO: 71)
RSSX₁₄GAVTTSNYDN,
the L8 sequence is
(SEQ ID NO: 58)
GTNKRAP,
and
the L9 sequence is
(SEQ ID NO: 59)
ALWYSNLWV,
wherein X₁₁ is G, S, A or T, X₁₂ is G or P, X₁₃ is A or Q, and X₁₄ is T or A.

3. The antibody or antibody fragment of claim 1, wherein the H1 sequence is GFWIKDTYIH (SEQ ID NO: 7) or GFNIKDTYIH (SEQ ID NO: 50).

4. The antibody or antibody fragment of claim 1, wherein the H2 sequence is selected from the group consisting of:
(SEQ ID NO: 8)
KIYPTNGYTRYADSVKG
(SEQ ID NO: 9)
RIKPTNGYTRYADSVKG
(SEQ ID NO: 10)
RIDPTNGYTRYADSVKG
(SEQ ID NO: 11)
RIYPTAGYTRYADSVKG
(SEQ ID NO: 12)
RIYPTNKYTRYADSVKG
(SEQ ID NO: 13)
RIYPTNGYDRYADSVKG
(SEQ ID NO: 14)
RIYPTNGYTEYADSVKG
and
(SEQ ID NO: 49)
RIYPTNGYTRYADSVKG.

5. The antibody or antibody fragment of claim 1, wherein the H3 sequence is WGGDGFYEMDY (SEQ ID NO: 15) or WGGDGFYAMDY (SEQ ID NO: 51).

6. The antibody or antibody fragment of claim 1, wherein the L1 sequence is RASQDVNTDVA (SEQ ID NO: 16) or RASQDVNTAVA (SEQ ID NO: 52).

7. The antibody or antibody fragment of claim 1, wherein the L3 sequence is QQDYTTPPT (SEQ ID NO: 17), QQEYTTPPT (SEQ ID NO: 18) or QQHYTTPPT (SEQ ID NO: 53).

8. The antibody or antibody fragment of claim 2, wherein the L6 sequence is any one of SEQ ID NOs: 56 and 60-67 and the L7 sequence is SEQ ID NO: 57, 68 or 69.

9. An antibody or antibody fragment that specifically binds to HER2 protein, said antibody or antibody fragment comprising a heavy chain variable region and a light chain variable region, wherein the heavy chain variable region is one of:
(SEQ ID NO: 19)
WVRQAPGKGLEWVAKIYPTNGYTRYADSVKGRFTI
SADTSKNTAYLQMNSLRAEDTAVYYCSRWGGDGFY
AMDYWGQGTLVTVSS
(SEQ ID NO: 20)
EVQLVESGGGLVQPGGSLRLSCAASGFNIKDTYIHW
VRQAPGKGLEWVARIYPTNGYTEYADSVKGRFTIS
ADTSKNTAYLQMNSLRAEDTAVYYCSRWGGDGFYA
MDYWGQGTLVTVSS
(SEQ ID NO: 21)
EVQLVESGGGLVQPGGSLRLSCAASGFNIKDTYIH
WVRQAPGKGLEWVARIKPTNGYTRYADSVKGRFTI
SADTSKNTAYLQMNSLRAEDTAVYYCSRWGGDGFY
AMDYWGQGTLVTVSS
(SEQ ID NO: 22)
EVQLVESGGGLVQPGGSLRLSCAASGFNIKDTYIH
WVRQAPGKGLEWVARIDPTNGYTRYADSVKGRFTI
SADTSKNTAYLQMNSLRAEDTAVYYCSRWGGDGFY
AMDYWGQGTLVTVSS
(SEQ ID NO: 23)
EVQLVESGGGLVQPGGSLRLSCAASGFNIKDTYIH
WVRQAPGKGLEWVARIYPTAGYTRYADSVKGRFTI
SADTSKNTAYLQMNSLRAEDTAVYYCSRWGGDGFY
AMDYWGQGTLVTVSS
(SEQ ID NO: 24)
EVQLVESGGGLVQPGGSLRLSCAASGFNIKDTYIH
WVRQAPGKGLEWVARIYPTNKYTRYADSVKGRFTI
SADTSKNTAYLQMNSLRAEDTAVYYCSRWGGDGFY
AMDYWGQGTLVTVSS
(SEQ ID NO: 25)
EVQLVESGGGLVQPGGSLRLSCAASGFNIKDTYIH
WVRQAPGKGLEWVARIYPTNGYDRYADSVKGRFTI
SADTSKNTAYLQMNSLRAEDTAVYYCSRWGGDGFY
AMDYWGQGTLVTVSS
EVQLVESGGGLVQPGGSLRLSCAASGFNIKDTYIH
(SEQ ID NO: 26)
WVRQAPGKGLEWVARIYPTNGYTRYADSVKGRFTI
SADTSKNTAYLQMNSLRAEDTAVYYCSRWGGDGFY
EMDYWGQGTLVTVSS
(SEQ ID NO: 27)
EVQLVESGGGLVQPGGSLRLSCAASGFNIKDTYIH
WVRQAPGKGLEWVARIYPTNGYTRYADSVKGRFTI
SADTSKNTAYLQMNSLRAEDTAVYYCSRWGGDGFY
AMDYWGQGTLVTVES,
or
(SEQ ID NO: 28)
EVQLVESGGGLVQPGGSLRLSCAASGFWIKDTYIH
WVRQAPGKGLEWVARIYPTNGYTRYADSVKGRFTI
SADTSKNTAYLQMNSLRAEDTAVYYCSRWGGDGFY
AMDYWGQGTLVTVSS,
and the light chain variable region is one of:
(SEQ ID NO: 29)
YQQKPGKAPKLLIYSASFLYSGVPSRFSGSRSGTD
FTLTISSLQPEDFATYYCQQHYTTPPTFGQGTKVE
IK
DIQMTQSPSSLSASVGDRVTITCRASQDVNTDVAW
(SEQ ID NO: 30)
YQQKPGKAPKLLIYSASFLYSGVPSRFSGSRSGTD
FTLTISSLQPEDFATYYCQQHYTTPPTFGQGTKVE
IK
DIQMTQSPSSLSASVGDRVTITCRASQDVNTAVAW
(SEQ ID NO: 31)
YQQKPGKAPKLLIYSASFLYSGVPSRFSGSRSGTD
FTLTISSLQPEDFATYYCQQDYTTPPTFGQGTKVE
IK
or
(SEQ ID NO: 32)
DIQMTQSPSSLSASVGDRVTITCRASQDVNTAVAW
YQQKPGKAPKLLIYSASFLYSGVPSRFSGSRSGTD
FTLTISSLQPEDFATYYCQQEYTTPPTFGQGTKVE
IK.

10. An antibody or antibody fragment that specifically binds to HER2 protein, said antibody or antibody fragment comprising a heavy chain variable region and a light chain variable region, wherein the heavy chain variable region is one of:
(SEQ ID NO: 33)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS
(SEQ ID NO: 19)
EVQLVESGGGLVQPGGSLRLSCAASGFNIKDTYIHWVRQAPGKGLEWVAK
IYPTNGYTRYADSVKGRFTISADTSKNTAYLQMNSLRAEDTAVYYCSRWG
GDGFYAMDYWGQGTLVTVSS
(SEQ ID NO: 20)
EVQLVESGGGLVQPGGSLRLSCAASGFNIKDTYIHWVRQAPGKGLEWVAR
IYPTNGYTEYADSVKGRFTISADTSKNTAYLQMNSLRAEDTAVYYCSRWG
GDGFYAMDYWGQGTLVTVSS
(SEQ ID NO: 21)
EVQLVESGGGLVQPGGSLRLSCAASGFNIKDTYIHWVRQAPGKGLEWVAR
IKPTNGYTRYADSVKGRFTISADTSKNTAYLQMNSLRAEDTAVYYCSRWG
GDGFYAMDYWGQGTLVTVSS
(SEQ ID NO: 22)
EVQLVESGGGLVQPGGSLRLSCAASGFNIKDTYIHWVRQAPGKGLEWVAR
IDPTNGYTRYADSVKGRFTISADTSKNTAYLQMNSLRAEDTAVYYCSRWG
GDGFYAMDYWGQGTLVTVSS
(SEQ ID NO: 23)
EVQLVESGGGLVQPGGSLRLSCAASGFNIKDTYIHWVRQAPGKGLEWVAR
IYPTAGYTRYADSVKGRFTISADTSKNTAYLQMNSLRAEDTAVYYCSRWG
GDGFYAMDYWGQGTLVTVSS
(SEQ ID NO: 24)
EVQLVESGGGLVQPGGSLRLSCAASGFNIKDTYIHWVRQAPGKGLEWVAR
IYPTNKYTRYADSVKGRFTISADTSKNTAYLQMNSLRAEDTAVYYCSRWG
GDGFYAMDYWGQGTLVTVSS
(SEQ ID NO: 25)
EVQLVESGGGLVQPGGSLRLSCAASGFNIKDTYIHWVRQAPGKGLEWVAR
IYPTNGYDRYADSVKGRFTISADTSKNTAYLQMNSLRAEDTAVYYCSRWG
GDGFYAMDYWGQGTLVTVSS
(SEQ ID NO: 26)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYEMDYWGQGTLVTVSS
(SEQ ID NO: 27)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVES,
or
(SEQ ID NO: 28)
EVQLVESGGGLVQPGGSLRLSCAASGFWIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS,
and the light chain variable region is one of:
(SEQ ID NO: 30)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIK
(SEQ ID NO: 31)
DIQMTQSPSSLSASVGDRVTITCRASQDVNTAVAWYQQKPGKAPKLLIYS
ASFLYSGVPSRFSGSRSGTDFTLTISSLQPEDFATYYCQQDYTTPPTFGQ
GTKVEIK
or
(SEQ ID NO: 32)
DIQMTQSPSSLSASVGDRVTITCRASQDVNTAVAWYQQKPGKAPKLLIYS
ASFLYSGVPSRFSGSRSGTDFTLTISSLQPEDFATYYCQQEYTTPPTFGQ
GTKVEIK.

11. The antibody or antibody fragment of claim 9, wherein the heavy chain variable region and the light chain variable region are selected from:
(SEQ ID NO: 19)
EVQLVESGGGLVQPGGSLRLSCAASGFNIKDTYIHWVRQAPGKGLEWVAK
IYPTNGYTRYADSVKGRFTISADTSKNTAYLQMNSLRAEDTAVYYCSRWG
GDGFYAMDYWGQGTLVTVSS
and
(SEQ ID NO: 29)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK,
or
(SEQ ID NO: 20)
EVQLVESGGGLVQPGGSLRLSCAASGFNIKDTYIHWVRQAPGKGLEWVAR
IYPTNGYTEYADSVKGRFTISADTSKNTAYLQMNSLRAEDTAVYYCSRWG
GDGFYAMDYWGQGTLVTVSS
and
(SEQ ID NO: 29)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK.

12. The antibody or antibody fragment of claim 10, wherein the heavy chain variable region is:
(SEQ ID NO: 33)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS,
and the light chain variable region is one of:
(SEQ ID NO: 30)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIK,
(SEQ ID NO: 31)
DIQMTQSPSSLSASVGDRVTITCRASQDVNTAVAWYQQKPGKAPKLLIYS
ASFLYSGVPSRFSGSRSGTDFTLTISSLQPEDFATYYCQQDYTTPPTFGQ
GTKVEIK,
or
(SEQ ID NO: 32)
DIQMTQSPSSLSASVGDRVTITCRASQDVNTAVAWYQQKPGKAPKLLIYS
ASFLYSGVPSRFSGSRSGTDFTLTISSLQPEDFATYYCQQEYTTPPTFGQ
GTKVEIK.

13. The antibody or antibody fragment of claim 9, wherein the light chain variable region is:
(SEQ ID NO: 30)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIK;
and the heavy chain variable region is one of:
(SEQ ID NO: 33)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS,
(SEQ ID NO: 19)
EVQLVESGGGLVQPGGSLRLSCAASGFNIKDTYIHWVRQAPGKGLEWVAK
IYPTNGYTRYADSVKGRFTISADTSKNTAYLQMNSLRAEDTAVYYCSRWG
GDGFYAMDYWGQGTLVTVSS
(SEQ ID NO: 20)
EVQLVESGGGLVQPGGSLRLSCAASGFNIKDTYIHWVRQAPGKGLEWVAR
IYPTNGYTEYADSVKGRFTISADTSKNTAYLQMNSLRAEDTAVYYCSRWG
GDGFYAMDYWGQGTLVTVSS
(SEQ ID NO: 21)
EVQLVESGGGLVQPGGSLRLSCAASGFNIKDTYIHWVRQAPGKGLEWVAR
IKPTNGYTRYADSVKGRFTISADTSKNTAYLQMNSLRAEDTAVYYCSRWG
GDGFYAMDYWGQGTLVTVSS
(SEQ ID NO: 22)
EVQLVESGGGLVQPGGSLRLSCAASGFNIKDTYIHWVRQAPGKGLEWVAR
IDPTNGYTRYADSVKGRFTISADTSKNTAYLQMNSLRAEDTAVYYCSRWG
GDGFYAMDYWGQGTLVTVSS
(SEQ ID NO: 23)
EVQLVESGGGLVQPGGSLRLSCAASGFNIKDTYIHWVRQAPGKGLEWVAR
IYPTAGYTRYADSVKGRFTISADTSKNTAYLQMNSLRAEDTAVYYCSRWG
GDGFYAMDYWGQGTLVTVSS
(SEQ ID NO: 24)
EVQLVESGGGLVQPGGSLRLSCAASGFNIKDTYIHWVRQAPGKGLEWVAR
IYPTNKYTRYADSVKGRFTISADTSKNTAYLQMNSLRAEDTAVYYCSRWG
GDGFYAMDYWGQGTLVTVSS
(SEQ ID NO: 25)
EVQLVESGGGLVQPGGSLRLSCAASGFNIKDTYIHWVRQAPGKGLEWVAR
IYPTNGYDRYADSVKGRFTISADTSKNTAYLQMNSLRAEDTAVYYCSRWG
GDGFYAMDYWGQGTLVTVSS
(SEQ ID NO: 26)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYEMDYWGQGTLVTVSS
(SEQ ID NO: 27)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVES,
or
(SEQ ID NO: 28)
EVQLVESGGGLVQPGGSLRLSCAASGFWIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS.

14. An antibody or antibody fragment that specifically binds to HER2 protein, said antibody or antibody fragment comprising a heavy chain variable region and a light chain variable region, wherein the heavy chain variable region is one of:
(SEQ ID NO: 35)
EVQLVESGGGLVQPGGSLRLSCAASGFNIKDTYIHWVRQAPGKGLEWVAR
IKPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYQ
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K;
(SEQ ID NO: 36)
EVQLVESGGGLVQPGGSLRLSCAASGFNIKDTYIHWVRQAPGKGLEWVAR
IYPTNKYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYQ
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K;
(SEQ ID NO: 37)
EVQLVESGGGLVQPGGSLRLSCAASGFNIKDTYIHWVRQAPGKGLEWVAR
IYPTNGYDRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYQ
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K;
(SEQ ID NO: 38)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYEMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYQ
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K;
or
(SEQ ID NO: 39)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVESASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYQ
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K;
and the light chain variable region is one of:
(SEQ ID NO: 41)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHSNFGNSKVSWFQYWGQGTLVTVSSGGSG
GSGGSGGSGGQAVVTQEPSLTVSPGGTVTLTCRSSTGAVTTSNYDNWVQQ
KPGQAPRGLIGGTNKRAPWTPARFSGSLLGGKAALTITGAQAEDEADYYC
ALWYSNLWVFGGGTKLTVLSR;
(SEQ ID NO: 42)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHGNFGNSKVSWFQYWGQGTLVTVSSGGSG
GSGGSGGSGGQAVVTQEPSLTVSPGGTVTLTCRSSTGAVTTSNYDNWVQQ
KPGQAPRGLIGGTNKRAPWTPARFSGSLLGGKAALTITGAQAEDEADYYC
ALWYSNLWVFGGGTKLTVLSR;
(SEQ ID NO: 43)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHGNFPNSKVSWFQYWGQGTLVTVSSGGSG
GSGGSGGSGGQAVVTQEPSLTVSPGGTVTLTCRSSTGAVTTSNYDNWVQQ
KPGQAPRGLIGGTNKRAPWTPARFSGSLLGGKAALTITGAQAEDEADYYC
ALWYSNLWVFGGGTKLTVLSR;
(SEQ ID NO: 44)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHANFGNSKVSWFAYWGQGTLVTVSSGGSG
GSGGSGGSGGQAVVTQEPSLTVSPGGTVTLTCRSSTGAVTTSNYDNWVQQ
KPGQAPRGLIGGTNKRAPWTPARFSGSLLGGKAALTITGAQAEDEADYYC
ALWYSNLWVFGGGTKLTVLSR;
(SEQ ID NO: 45)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHGNFPNSKVSWFAYWGQGTLVTVSSGGSG
GSGGSGGSGGQAVVTQEPSLTVSPGGTVTLTCRSSTGAVTTSNYDNWVQQ
KPGQAPRGLIGGTNKRAPWTPARFSGSLLGGKAALTITGAQAEDEADYYC
ALWYSNLWVFGGGTKLTVLSR;
(SEQ ID NO: 46)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHTNFGNSKVSWFAYWGQGTLVTVSSGGSG
GSGGSGGSGGQAVVTQEPSLTVSPGGTVTLTCRSSTGAVTTSNYDNWVQQ
KPGQAPRGLIGGTNKRAPWTPARFSGSLLGGKAALTITGAQAEDEADYYC
ALWYSNLWVFGGGTKLTVLSR;
(SEQ ID NO: 47)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHSNFGNSKVSWFAYWGQGTLVTVSSGGSG
GSGGSGGSGGQAVVTQEPSLTVSPGGTVTLTCRSSAGAVTTSNYDNWVQQ
KPGQAPRGLIGGTNKRAPWTPARFSGSLLGGKAALTITGAQAEDEADYYC
ALWYSNLWVFGGGTKLTVLSR;
or
(SEQ ID NO: 48)
DIQMTQSPSSLSASVGDRVTITCRASQDVNTDVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECSRSGGGGEVQLVESGGGLVQPGGSLRLSCAASGFTF
NTYAMNWVRQAPGKGLEWVARIRSKYNNYATYYADSVKDRFTISRDDSKN
SLYLQMNSLKTEDTAVYYCVRHTNFGNSKVSWFAYWGQGTLVTVSSGGSG
GSGGSGGSGGQAVVTQEPSLTVSPGGTVTLTCRSSAGAVTTSNYDNWVQQ
KPGQAPRGLIGGTNKRAPWTPARFSGSLLGGKAALTITGAQAEDEADYYC
ALWYSNLWVFGGGTKLTVLSR.

15. The antibody or antibody fragment of claim 14, wherein the heavy chain variable region is SEQ ID NO: 35 and the light chain variable region is any one of SEQ ID NOs: 41-48.

16. The antibody or antibody fragment of claim 14, wherein the heavy chain variable region is SEQ ID NO: 36 and the light chain variable region is any one of SEQ ID NOs: 41-48.

17. The antibody or antibody fragment of claim 14, wherein the heavy chain variable region is SEQ ID NO: 37 and the light chain variable region is any one of SEQ ID NOs: 41-48.

18. The antibody or antibody fragment of claim 14, wherein the heavy chain variable region is SEQ ID NO: 38 and the light chain variable region is any one of SEQ ID NOs: 41-48.

19. The antibody or antibody fragment of claim 14, wherein the heavy chain variable region is SEQ ID NO: 39 and the light chain variable region is any one of SEQ ID Nos: 41-48.

20-22. (canceled)

23. An antibody or antibody fragment comprising a heavy chain variable region including three complementarity determining regions, said regions having sequences H1, H2, and H3, wherein:
(SEQ ID NO: 1)
the H1 sequence is GFX₁IKDTYIH;
(SEQ ID NO: 2)
the H2 sequence is X₂IX₃PTX₄X₅YX₆X₇YADSVKG;
and
(SEQ ID NO: 3)
the H3 sequence is WGGDGFYX₈MDY;
wherein X₁ is N or W, X₂ is R or K, X₃ is Y or K or D, X₄ is N or A, X₅ is G or K, X₆ is T or D, X₇ is R or E and X₈ is A or E; and
a light chain variable region including three complementarity determining regions having sequences L1, L2, and L3, wherein:
(SEQ ID NO: 4)
the L1 sequence is RASQDVNTX₉VA;
(SEQ ID NO: 5)
the L2 sequence is SASFLYS;
and
(SEQ ID NO: 6)
the L3 sequence is QQX₁₀YTTPPT,
wherein X₉ is A or D and X₁₀ is H, D or E;
provided that when X₁-X₈ are N, R, Y, N, G, T, R and A, respectively, X₉ is not A and X₁₀ is not H.

24. A bispecific antibody or antibody fragment that specifically binds to HER2 protein and CD3 protein, said antibody or antibody fragment comprising a heavy chain variable region including three anti-HER2 complementarity determining regions, said regions having sequences H1, H2, and H3, wherein:
(SEQ ID NO: 1)
the H1 sequence is GFX₁IKDTYIH;
(SEQ ID NO: 2)
the H2 sequence is X₂IX₃PTX₄X₅YX₆X₇YADSVKG;
and
(SEQ ID NO: 3)
the H3 sequence is WGGDGFYX₈MDY;
wherein X₁ is N or W, X₂ is R or K, X₃ is Y or K or D, X₄ is N or A, X₅ is G or K, X₆ is T or D, X₇ is R or E and X₈ is A or E; and
a light chain variable region including three anti-HER2 complementarity determining regions having sequences L1, L2, and L3 wherein:
(SEQ ID NO: 4)
the L1 sequence is RASQDVNTX₉VA;
(SEQ ID NO: 5)
the L2 sequence is SASFLYS;
and
(SEQ ID NO: 6)
the L3 sequence is QQX₁₀YTTPPT,
wherein X₉ is A or D and X₁₀ is H or D or E; provided that when X₁-X₈ are N, R, Y, N, G, T, R and A, respectively, X₉ is not A and X₁₀ is not H; and
six anti-CD3 complementarity determining regions L4, L5, L6, L7, L8, and L9 wherein:
(SEQ ID NO: 54)
the L4 sequence is GFTFNTYAMN,
(SEQ ID NO: 55)
the L5 sequence is RIRSKYNNYATYYADSVKD,
(SEQ ID NO: 70)
the L6 sequence is HX₁₁NFX₁₂NSKVSWFX₁₃Y,
(SEQ ID NO: 71)
the L7 sequence is RSSX₁₄GAVTTSNYDN,
(SEQ ID NO: 58)
the L8 sequence is GTNKRAP,
and
(SEQ ID NO: 59)
the L9 sequence is ALWYSNLWV,
wherein X₁₁ is G, S, A or T, X₁₂ is G or P, X₁₃ is A or Q, and X₁₄ is T or A.

25. The antibody or antibody fragment of claim 24, wherein the H1 sequence is GFWIKDTYIH (SEQ ID NO: 7) or GFNIKDTYIH (SEQ ID NO: 50).

26. The antibody or antibody fragment of claim 24, wherein the H2 sequence is selected from the group consisting of:
(SEQ ID NO: 8)
KIYPTNGYTRYADSVKG
(SEQ ID NO: 9)
RIKPTNGYTRYADSVKG
(SEQ ID NO: 10)
RIDPTNGYTRYADSVKG
(SEQ ID NO: 11)
RIYPTAGYTRYADSVKG
(SEQ ID NO: 12)
RIYPTNKYTRYADSVKG
(SEQ ID NO: 13)
RIYPTNGYDRYADSVKG
(SEQ ID NO: 14)
RIYPTNGYTEYADSVKG
and
(SEQ ID NO: 49)
RIYPTNGYTRYADSVKG.

27. The antibody or antibody fragment of claim 24, wherein the H3 sequence is WGGDGFYEMDY (SEQ ID NO: 15) or WGGDGFYAMDY (SEQ ID NO: 51).

28. The antibody or antibody fragment of claim 24, wherein the L1 sequence is RASQDVNTDVA (SEQ ID NO: 16) or RASQDVNTAVA (SEQ ID NO: 52).

29. The antibody or antibody fragment claim 24, wherein the L3 sequence is QQDYTTPPT (SEQ ID NO: 17), QQEYTTPPT (SEQ ID NO: 18), or QQHYTTPPT (SEQ ID NO: 53).

30-42. (canceled)

43. The antibody or antibody fragment of claim 1, wherein the antibody or antibody fragment has a ratio of binding affinity to the HER2 protein at a pH in a tumor microenvironment to a binding affinity to the HER2 protein at a different pH in a non-tumor microenvironment of at least about 1.5:1.

44. An immunoconjugate comprising the antibody or antibody fragment of claim 1.

45. The immunoconjugate of claim 44, wherein the immunoconjugate comprises at least one agent selected from a chemotherapeutic agent, a radioactive atom, a cytostatic agent and a cytotoxic agent.

46. The immunoconjugate of claim 45, comprising at least two said agents.

47. The immunoconjugate of claim 45, wherein the at least one agent is a radioactive agent.

48. The immunoconjugate of claim 47, wherein the radioactive agent is selected from an alpha emitter, a beta emitter and a gamma emitter.

49. The immunoconjugate of claim 45, wherein the antibody or antibody fragment and the at least one agent are covalently bonded to a linker molecule.

50. The immunoconjugate of claim 45, wherein one said at least one agent is selected from maytansinoids, auristatins, dolastatins, calicheamicin, pyrrolobenzodiazepines, and anthracyclines.

51. A pharmaceutical composition comprising
the antibody or antibody fragment of claim 1; and
a pharmaceutically acceptable carrier.

52. The pharmaceutical composition of claim 51, further comprising a tonicity agent.

53. The pharmaceutical composition of claim 51, further comprising an immune checkpoint inhibitor molecule.

54. The pharmaceutical composition of claim 53, wherein the immune checkpoint inhibitor molecule is an antibody or antibody fragment against an immune checkpoint.

55. The pharmaceutical composition of claim 53, wherein the immune checkpoint is selected from CTLA4, LAG3, TIM3, TIGIT, VISTA, BTLA, OX40, CD40, 4-1BB, PD-1, PD-L1, GITR, B7-H3, B7-H4, KIR, A2aR, CD27, CD70, DR3, and ICOS.

56. The pharmaceutical composition of claim 53, wherein the immune checkpoint is CTLA4, PD-1 or PD-L1.

57. The pharmaceutical composition of claim 51, further comprising an antibody or antibody fragment against an antigen selected from CTLA4, PD1, PD-L1, AXL, ROR2, CD3, EpCAM, B7-H3, ROR1, SFRP4 and a WNT protein.

58. A method of treating cancer comprising a step of administering the antibody or antibody fragment of claim 1 to a patient with cancer.

59. A kit for diagnosis or treatment, said kit comprising the antibody or antibody fragment of claim 1, or the immunoconjugate of claim 44 and instructions for using the antibody or antibody fragment for diagnosis or treatment.

60. A pharmaceutical composition comprising
the immunoconjugate of claim 44; and
a pharmaceutically acceptable carrier.

61. A method of treating cancer comprising a step of administering the immunoconjugate of claim 44 to a patient with cancer.

62. A method of treating cancer comprising a step of administering the pharmaceutical composition of claim 51 to a patient with cancer.

63. A method of treating cancer comprising a step of administering the pharmaceutical composition of claim 60 to a patient with cancer.

64. A kit for diagnosis or treatment, said kit comprising the immunoconjugate of claim 44 and instructions for using the immunoconjugate for diagnosis or treatment.

65. A kit for diagnosis or treatment, said kit comprising the pharmaceutical composition of claim 51 and instructions for using the pharmaceutical composition for diagnosis or treatment.

66. An immunoconjugate comprising the antibody or antibody fragment of claim 2.

67. A pharmaceutical composition comprising
the antibody or antibody fragment of claim 2; and
a pharmaceutically acceptable carrier.

68. A pharmaceutical composition comprising
the immunoconjugate of claim 66; and
a pharmaceutically acceptable carrier.
